SEQUENCE LISTING (88 sequences)

SEQ ID NO: 1 - DNA, 52 bp, synthetic construct
  misc_feature 1..52: note = ModA 5' UTR
  source 1..52: mol_type = other DNA
gggcgaactagtattcttctggtccccacagactcagagagaacccgccacc

SEQ ID NO: 2 - RNA, 52 nt, synthetic construct
  misc_feature 1..52: note = ModA 5' UTR
  source 1..52: mol_type = other RNA
gggcgaactagtattcttctggtccccacagactcagagagaacccgccacc

SEQ ID NO: 3 - DNA, 145 bp, synthetic construct
  misc_feature 1..145: note = ModB 5' UTR
  source 1..145: mol_type = other DNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagcc

SEQ ID NO: 4 - RNA, 145 nt, synthetic construct
  misc_feature 1..145: note = ModB 5' UTR
  source 1..145: mol_type = other RNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagcc

SEQ ID NO: 5 - DNA, 52 bp, synthetic construct
  misc_feature 1..52: note = Alternative Mod 5' UTR
  source 1..52: mol_type = other DNA
agacgaactagtattcttctggtccccacagactcagagagaacccgccacc

SEQ ID NO: 6 - RNA, 52 nt, synthetic construct
  misc_feature 1..52: note = Alternative Mod 5' UTR
  source 1..52: mol_type = other RNA
agacgaactagtattcttctggtccccacagactcagagagaacccgccacc

SEQ ID NO: 7 - DNA, 427 bp, synthetic construct
  misc_feature 1..427: note = ModA/B 3' UTR
  source 1..427: mol_type = other DNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 8 - RNA, 427 nt, synthetic construct
  misc_feature 1..427: note = ModA/B 3' UTR
  source 1..427: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 9 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - DNA, 465 bp, Homo sapiens
  source 1..465: mol_type = genomic DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgatga

SEQ ID NO: 11 - DNA, 465 bp, synthetic construct
  misc_feature 1..465: note = Human optimized IL-2
  source 1..465: mol_type = other DNA
atgtacagaatgcagctgctgtcttgcattgctctttctcttgctcttgtgacaaattctgctccaacatcttcttcaacaaagaaaacacagcttcagcttgaacaccttcttcttgatcttcagatgattctgaatggaatcaacaattacaaaaatccaaaactgacaagaatgctgacatttaaattttacatgccaaagaaagcaacagaactgaaacaccttcagtgccttgaagaagaactgaaacctctggaagaagtgctgaatctggctcagagcaaaaattttcacctgagaccaagagatctgatcagcaacatcaatgtgattgtgctggaactgaaaggatctgaaacaacattcatgtgtgaatatgctgatgaaacagcaacaattgtggaatttctgaacagatggatcacattttgccagtcaatcatttcaacactgacatgatga

SEQ ID NO: 12 - RNA, 465 nt, Homo sapiens
  source 1..465: mol_type = genomic RNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgatga

SEQ ID NO: 13 - RNA, 465 nt, synthetic construct
  misc_feature 1..465: note = Human optimized IL-2
  source 1..465: mol_type = other RNA
atgtacagaatgcagctgctgtcttgcattgctctttctcttgctcttgtgacaaattctgctccaacatcttcttcaacaaagaaaacacagcttcagcttgaacaccttcttcttgatcttcagatgattctgaatggaatcaacaattacaaaaatccaaaactgacaagaatgctgacatttaaattttacatgccaaagaaagcaacagaactgaaacaccttcagtgccttgaagaagaactgaaacctctggaagaagtgctgaatctggctcagagcaaaaattttcacctgagaccaagagatctgatcagcaacatcaatgtgattgtgctggaactgaaaggatctgaaacaacattcatgtgtgaatatgctgatgaaacagcaacaattgtggaatttctgaacagatggatcacattttgccagtcaatcatttcaacactgacatgatga

SEQ ID NO: 14 - protein, 539 aa, synthetic construct
  REGION 1..539: note = Human IL-12sc
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSSGGGGSPGGGSSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 15 - DNA, 1623 bp, synthetic construct
  misc_feature 1..1623: note = Human non-optimized IL-12sc
  source 1..1623: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagagggtcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggctctagcggagggggaggctctcctggcgggggatctagcagaaacctccccgtggccactccagacccaggaatgttcccatgccttcaccactcccaaaacctgctgagggccgtcagcaacatgctccagaaggccagacaaactctagaattttacccttgcacttctgaggaaattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctgatga

SEQ ID NO: 16 - DNA, 1623 bp, synthetic construct
  misc_feature 1..1623: note = Human optimized IL-12sc
  source 1..1623: mol_type = other DNA
atgtgtcaccagcagctggtgatctcatggttctccctggtatttctggcatctcctcttgtcgcaatctgggaactgaagaaagacgtgtatgtcgttgagctcgactggtatccggatgcgcctggcgagatggtggtgctgacctgtgacaccccagaggaggatgggatcacttggacccttgatcaatcctccgaagtgctcgggtctggcaagactctgaccatacaagtgaaagagtttggcgatgccgggcagtacacttgccataagggcggagaagttctgtcccactcactgctgctgctgcacaagaaagaggacggaatttggagtaccgatatcctgaaagatcagaaagagcccaagaacaaaaccttcttgcggtgcgaagccaagaactactcagggagatttacttgttggtggctgacgacgatcagcaccgatctgactttctccgtgaaatcaagtaggggatcatctgaccctcaaggagtcacatgtggagcggctactctgagcgctgaacgcgtaagaggggacaataaggagtacgagtatagcgttgagtgccaagaggatagcgcatgccccgccgccgaagaatcattgcccattgaagtgatggtggatgctgtacacaagctgaagtatgagaactacacaagctccttcttcatccgtgacatcatcaaaccagatcctcctaagaacctccagcttaaacctctgaagaactctagacaggtggaagtgtcttgggagtatcccgacacctggtctacaccacattcctacttcagtctcacattctgcgttcaggtacagggcaagtccaaaagggagaagaaggatcgggtctttacagataaaacaagtgccaccgttatatgccggaagaatgcctctatttctgtgcgtgcgcaggacagatactatagcagctcttggagtgaatgggccagtgtcccatgttcagggtcatccggtggtggcggcagccccggaggcggtagctccagaaatctccctgtggctacacctgatccaggcatgtttccctgtttgcaccatagccaaaacctcctgagagcagtcagcaacatgctccagaaagctagacaaacactggaattctacccatgcacctccgaggaaatagatcacgaggatatcactaaggacaaaacaagcactgtcgaagcatgccttcccttggaactgacaaagaacgagagttgccttaattcaagagaaacatctttcattacaaacggtagctgcttggcaagcagaaaaacatcttttatgatggccctttgtctgagcagtatttatgaggatctcaaaatgtaccaggtggagtttaagaccatgaatgccaagctgctgatggacccaaagagacagattttcctcgatcagaatatgctggctgtgattgatgaactgatgcaggccttgaatttcaacagcgaaaccgttccccagaaaagcagtcttgaagaacctgacttttataagaccaagatcaaactgtgtattctcctgcatgcctttagaatcagagcagtcactatagatagagtgatgtcctacctgaatgcttcctgatga

SEQ ID NO: 17 - RNA, 1623 nt, synthetic construct
  misc_feature 1..1623: note = Human non-optimized IL-12sc
  source 1..1623: mol_type = other RNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagagggtcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggctctagcggagggggaggctctcctggcgggggatctagcagaaacctccccgtggccactccagacccaggaatgttcccatgccttcaccactcccaaaacctgctgagggccgtcagcaacatgctccagaaggccagacaaactctagaattttacccttgcacttctgaggaaattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctgatga

SEQ ID NO: 18 - RNA, 1623 nt, synthetic construct
  misc_feature 1..1623: note = Human optimized IL-12sc
  source 1..1623: mol_type = other RNA
atgtgtcaccagcagctggtgatctcatggttctccctggtatttctggcatctcctcttgtcgcaatctgggaactgaagaaagacgtgtatgtcgttgagctcgactggtatccggatgcgcctggcgagatggtggtgctgacctgtgacaccccagaggaggatgggatcacttggacccttgatcaatcctccgaagtgctcgggtctggcaagactctgaccatacaagtgaaagagtttggcgatgccgggcagtacacttgccataagggcggagaagttctgtcccactcactgctgctgctgcacaagaaagaggacggaatttggagtaccgatatcctgaaagatcagaaagagcccaagaacaaaaccttcttgcggtgcgaagccaagaactactcagggagatttacttgttggtggctgacgacgatcagcaccgatctgactttctccgtgaaatcaagtaggggatcatctgaccctcaaggagtcacatgtggagcggctactctgagcgctgaacgcgtaagaggggacaataaggagtacgagtatagcgttgagtgccaagaggatagcgcatgccccgccgccgaagaatcattgcccattgaagtgatggtggatgctgtacacaagctgaagtatgagaactacacaagctccttcttcatccgtgacatcatcaaaccagatcctcctaagaacctccagcttaaacctctgaagaactctagacaggtggaagtgtcttgggagtatcccgacacctggtctacaccacattcctacttcagtctcacattctgcgttcaggtacagggcaagtccaaaagggagaagaaggatcgggtctttacagataaaacaagtgccaccgttatatgccggaagaatgcctctatttctgtgcgtgcgcaggacagatactatagcagctcttggagtgaatgggccagtgtcccatgttcagggtcatccggtggtggcggcagccccggaggcggtagctccagaaatctccctgtggctacacctgatccaggcatgtttccctgtttgcaccatagccaaaacctcctgagagcagtcagcaacatgctccagaaagctagacaaacactggaattctacccatgcacctccgaggaaatagatcacgaggatatcactaaggacaaaacaagcactgtcgaagcatgccttcccttggaactgacaaagaacgagagttgccttaattcaagagaaacatctttcattacaaacggtagctgcttggcaagcagaaaaacatcttttatgatggccctttgtctgagcagtatttatgaggatctcaaaatgtaccaggtggagtttaagaccatgaatgccaagctgctgatggacccaaagagacagattttcctcgatcagaatatgctggctgtgattgatgaactgatgcaggccttgaatttcaacagcgaaaccgttccccagaaaagcagtcttgaagaacctgacttttataagaccaagatcaaactgtgtattctcctgcatgcctttagaatcagagcagtcactatagatagagtgatgtcctacctgaatgcttcctgatga

SEQ ID NO: 19 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 20 - DNA, 570 bp, Homo sapiens
  source 1..570: mol_type = genomic DNA
atggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaaccttttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgatga

SEQ ID NO: 21 - DNA, 570 bp, synthetic construct
  misc_feature 1..570: note = Human optimized IFN-alpha-2b
  source 1..570: mol_type = other DNA
atggccctgacttttgcccttctcgtggctttgttggtgctgagttgcaaatcttcctgtagtgtcggatgtgatctgcctcaaacccacagtctgggatctaggagaacactgatgctgttggcacagatgaggagaattagcctcttttcctgcctgaaggatagacatgacttcggctttccccaagaggagtttggcaatcagttccagaaagcggaaacgattcccgttctgcacgagatgatccagcagatcttcaacctcttttcaaccaaagacagctcagcagcctgggatgagacactgctggacaaattctacacagaactgtatcagcagcttaacgatctggaggcatgcgtgatccaaggggttggtgtgactgaaactccgcttatgaaggaggactccattctggctgtacggaagtacttccagagaataaccctctatctgaaggagaagaagtactcaccatgtgcttgggaagtcgtgagagccgaaatcatgagatccttcagccttagcaccaatctccaggaatctctgagaagcaaagagtgatga

SEQ ID NO: 22 - RNA, 570 nt, Homo sapiens
  source 1..570: mol_type = genomic RNA
atggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaaccttttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgatga

SEQ ID NO: 23 - RNA, 570 nt, synthetic construct
  misc_feature 1..570: note = Human optimized IFN-alpha-2b
  source 1..570: mol_type = other RNA
atggccctgacttttgcccttctcgtggctttgttggtgctgagttgcaaatcttcctgtagtgtcggatgtgatctgcctcaaacccacagtctgggatctaggagaacactgatgctgttggcacagatgaggagaattagcctcttttcctgcctgaaggatagacatgacttcggctttccccaagaggagtttggcaatcagttccagaaagcggaaacgattcccgttctgcacgagatgatccagcagatcttcaacctcttttcaaccaaagacagctcagcagcctgggatgagacactgctggacaaattctacacagaactgtatcagcagcttaacgatctggaggcatgcgtgatccaaggggttggtgtgactgaaactccgcttatgaaggaggactccattctggctgtacggaagtacttccagagaataaccctctatctgaaggagaagaagtactcaccatgtgcttgggaagtcgtgagagccgaaatcatgagatccttcagccttagcaccaatctccaggaatctctgagaagcaaagagtgatga

SEQ ID NO: 24 - protein, 241 aa, synthetic construct
  REGION 1..241: note = Human IL-15 sushi
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPGGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 25 - DNA, 729 bp, synthetic construct
  misc_feature 1..729: note = Human IL-15 sushi
  source 1..729: mol_type = other DNA
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccgggggaggatctggcggcggtgggtctggcgggggatctggcggaggaggaagcttacagaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgatga

SEQ ID NO: 26 - RNA, 729 nt, synthetic construct
  misc_feature 1..729: note = Human IL-15 sushi
  source 1..729: mol_type = other RNA
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccgggggaggatctggcggcggtgggtctggcgggggatctggcggaggaggaagcttacagaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgatga

SEQ ID NO: 27 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 28 - DNA, 438 bp, Homo sapiens
  source 1..438: mol_type = genomic DNA
atgtggctccagagcctgctgctcttgggcactgtggcctgctccatctctgcacccgcccgctcgcccagccccagcacgcagccctgggagcatgtgaatgccatccaggaggcccggcgtctgctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgatga

SEQ ID NO: 29 - RNA, 438 nt, Homo sapiens
  source 1..438: mol_type = genomic RNA
atgtggctccagagcctgctgctcttgggcactgtggcctgctccatctctgcacccgcccgctcgcccagccccagcacgcagccctgggagcatgtgaatgccatccaggaggcccggcgtctgctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgatga

SEQ ID NO: 30 - protein, 161 aa, synthetic construct
  REGION 1..161: note = ModA IL-2 (human IL-2 in combination with a mouse optimized secretion sequence)
MRVTAPRTLILLLSGALALTETWAGSGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 31 - DNA, 959 bp, synthetic construct
  misc_feature 1..959: note = ModA IL-2 (5'UTR-CDS-3'UTR)
  source 1..959: mol_type = other DNA
gggcgaactagtattcttctggtccccacagactcagagagaacccgccaccatgagagtgaccgcccccagaaccctgatcctgctgctgtctggcgccctggccctgacagagacatgggccggaagcggatccgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgactcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagacctggtccagagtcgctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 32 - RNA, 959 nt, synthetic construct
  misc_feature 1..959: note = ModA IL-2
  source 1..959: mol_type = other RNA
gggcgaactagtattcttctggtccccacagactcagagagaacccgccaccatgagagtgaccgcccccagaaccctgatcctgctgctgtctggcgccctggccctgacagagacatgggccggaagcggatccgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgactcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagacctggtccagagtcgctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 33 - protein, 161 aa, synthetic construct
  REGION 1..161: note = ModB IL-2
MGAMAPRTLLLLLAAALAPTQTRAGPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 34 - DNA, 1039 bp, synthetic construct
  misc_feature 1..1039: note = ModB IL-2 (5'UTR-CDS-3'UTR)
  source 1..1039: mol_type = other DNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagccatgggcgccatggcccctagaacattgctcctgctgctggccgctgccctggcccctacacagacaagagctggacctggatccgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgactcgacgtcctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 35 - RNA, 1039 nt, synthetic construct
  misc_feature 1..1039: note = ModB IL-2
  source 1..1039: mol_type = other RNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagccatgggcgccatggcccctagaacattgctcctgctgctggccgctgccctggcccctacacagacaagagctggacctggatccgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgactcgacgtcctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 36 - protein, 544 aa, synthetic construct
  REGION 1..544: note = ModA murine IL-12
MRVTAPRTLILLLSGALALTETWAGSGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSVPGVGVPGVGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 37 - DNA, 2111 bp, synthetic construct
  misc_feature 1..2111: note = ModA murine IL-12 (5'UTR-CDS-3'UTR)
  source 1..2111: mol_type = other DNA
gggcgaactagtattcttctggtccccacagactcagagagaacccgccaccatgagagtgaccgcccccagaaccctgatcctgctgctgtctggcgccctggccctgacagagacatgggccggaagcggatccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagagtccgatcggttcctggagtaggggtacctggagtgggcagggtcataccggtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgtccagcgcctaatagctcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagacctggtccagagtcgctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 38 - RNA, 2111 nt, synthetic construct
  misc_feature 1..2111: note = ModA murine IL-12
  source 1..2111: mol_type = other RNA
gggcgaactagtattcttctggtccccacagactcagagagaacccgccaccatgagagtgaccgcccccagaaccctgatcctgctgctgtctggcgccctggccctgacagagacatgggccggaagcggatccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagagtccgatcggttcctggagtaggggtacctggagtgggcagggtcataccggtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgtccagcgcctaatagctcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagacctggtccagagtcgctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 39 - protein, 544 aa, synthetic construct
  REGION 1..544: note = ModB murine IL-12
MGAMAPRTLLLLLAAALAPTQTRAGPGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSVPGVGVPGVGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 40 - DNA, 2191 bp, synthetic construct
  misc_feature 1..2191: note = ModB murine IL-12 (5'UTR-CDS-3'UTR)
  source 1..2191: mol_type = other DNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagccatgggcgccatggcccctagaacattgctcctgctgctggccgctgccctggcccctacacagacaagagctggacctggatccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagagtccgatcggttcctggagtaggggtacctggagtgggcagggtcataccggtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgtccagcgcctaatagctcgacgtcctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 41 - RNA, 2191 nt, synthetic construct
  misc_feature 1..2191: note = ModB murine IL-12
  source 1..2191: mol_type = other RNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagccatgggcgccatggcccctagaacattgctcctgctgctggccgctgccctggcccctacacagacaagagctggacctggatccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagagtccgatcggttcctggagtaggggtacctggagtgggcagggtcataccggtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgtccagcgcctaatagctcgacgtcctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 42 - protein, 190 aa, synthetic construct
  REGION 1..190: note = ModA murine IFN-alpha-4
MRVTAPRTLILLLSGALALTETWAGSGSCDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKE

SEQ ID NO: 43 - DNA, 1049 bp, synthetic construct
  misc_feature 1..1049: note = ModA murine IFN-alpha-4 (5'UTR-CDS-3'UTR)
  source 1..1049: mol_type = other DNA
gggcgaactagtattcttctggtccccacagactcagagagaacccgccaccatgagagtgaccgcccccagaaccctgatcctgctgctgtctggcgccctggccctgacagagacatgggccggaagcggatcctgtgacctgcctcacacttataacctcgggaacaagagggccttgacagtcctggaagaaatgagaagactcccccctctttcctgcctgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctcctagactcattctgcaatgacctccatcagcagctcaatgatctcaaagcctgtgtgatgcaggaacctcctctgacccaggaagactccctgctggctgtgaggacatacttccacaggatcactgtgtacctgagaaagaagaaacacagcctctgtgcctgggaggtgatcagagcagaagtctggagagccctctcttcctcaaccaacttgctggcaagactgagtgaggagaaggagtgataactcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagacctggtccagagtcgctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 44 - RNA, 1049 nt, synthetic construct
  misc_feature 1..1049: note = ModA murine IFN-alpha-4
  source 1..1049: mol_type = other RNA
gggcgaactagtattcttctggtccccacagactcagagagaacccgccaccatgagagtgaccgcccccagaaccctgatcctgctgctgtctggcgccctggccctgacagagacatgggccggaagcggatcctgtgacctgcctcacacttataacctcgggaacaagagggccttgacagtcctggaagaaatgagaagactcccccctctttcctgcctgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctcctagactcattctgcaatgacctccatcagcagctcaatgatctcaaagcctgtgtgatgcaggaacctcctctgacccaggaagactccctgctggctgtgaggacatacttccacaggatcactgtgtacctgagaaagaagaaacacagcctctgtgcctgggaggtgatcagagcagaagtctggagagccctctcttcctcaaccaacttgctggcaagactgagtgaggagaaggagtgataactcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagacctggtccagagtcgctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 45 - protein, 190 aa, synthetic construct
  REGION 1..190: note = ModB murine IFN-alpha-4
MGAMAPRTLLLLLAAALAPTQTRAGPGSCDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKE

SEQ ID NO: 46 - DNA, 1129 bp, synthetic construct
  misc_feature 1..1129: note = ModB murine IFN-alpha-4 (5'UTR-CDS-3'UTR)
  source 1..1129: mol_type = other DNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagccatgggcgccatggcccctagaacattgctcctgctgctggccgctgccctggcccctacacagacaagagctggacctggatcctgtgacctgcctcacacttataacctcgggaacaagagggccttgacagtcctggaagaaatgagaagactcccccctctttcctgcctgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctcctagactcattctgcaatgacctccatcagcagctcaatgatctcaaagcctgtgtgatgcaggaacctcctctgacccaggaagactccctgctggctgtgaggacatacttccacaggatcactgtgtacctgagaaagaagaaacacagcctctgtgcctgggaggtgatcagagcagaagtctggagagccctctcttcctcaaccaacttgctggcaagactgagtgaggagaaggagtgataactcgacgtcctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 47 - RNA, 1129 nt, synthetic construct
  misc_feature 1..1129: note = ModB murine IFN-alpha-4
  source 1..1129: mol_type = other RNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagccatgggcgccatggcccctagaacattgctcctgctgctggccgctgccctggcccctacacagacaagagctggacctggatcctgtgacctgcctcacacttataacctcgggaacaagagggccttgacagtcctggaagaaatgagaagactcccccctctttcctgcctgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctcctagactcattctgcaatgacctccatcagcagctcaatgatctcaaagcctgtgtgatgcaggaacctcctctgacccaggaagactccctgctggctgtgaggacatacttccacaggatcactgtgtacctgagaaagaagaaacacagcctctgtgcctgggaggtgatcagagcagaagtctggagagccctctcttcctcaaccaacttgctggcaagactgagtgaggagaaggagtgataactcgacgtcctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 48 - protein, 232 aa, synthetic construct
  REGION 1..232: note = ModA murine IL-15 sushi
MGAMAPRTLLLLLAAALAPTQTRAGPGSTTCPPPVSIEHADIRVKNYSVNSRERYVCNSGFKRKAGTSTLIECVINKNTNVAHWTTPSLKCIRDPSLAGGSGGSGGSGGSGGSGGSGGNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 49 - DNA, 1162 bp, synthetic construct
  misc_feature 1..1162: note = ModA murine IL-15 sushi (5'UTR-CDS-3'UTR)
  source 1..1162: mol_type = other DNA
gggcgaactagtattcttctggtccccacagactcagagagaacccgccaccatgggcgccatggcccctagaacattgctcctgctgctggccgctgccctggcccctacacagacaagagctggacctggatccaccacgtgtccacctcccgtatctattgagcatgctgacatccgggtcaagaattacagtgtgaactccagggagaggtatgtctgtaactctggctttaagcggaaagctggaacatccaccctgattgagtgtgtgatcaacaagaacacaaatgttgcccactggacaactcccagcctcaagtgcatcagagacccctccctagctggagggagcggaggctctggcggaagcggcgggtctggaggctccgggggaagcggcggaaattggatcgacgtgcgctacgacctggaaaagatcgagagcctgatccagagcatccacatcgacaccaccctgtacaccgacagcgacttccaccccagctgcaaagtgaccgctatgaactgcttcctgctggaactgcaagtgatcctgcacgagtacagcaacatgaccctgaacgagacagtgcggaacgtgctgtacctggccaacagcaccctgagcagcaacaagaacgtggccgagagcggctgcaaagagtgcgaggaactggaagaaaagaccttcaccgagtttctgcagagcttcatcaggatcgtgcagatgttcatcaacacctcttgatgagtcgacgtcctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 50 - RNA, 1162 nt, synthetic construct
  misc_feature 1..1162: note = ModA murine IL-15 sushi
  source 1..1162: mol_type = other RNA
gggcgaactagtattcttctggtccccacagactcagagagaacccgccaccatgggcgccatggcccctagaacattgctcctgctgctggccgctgccctggcccctacacagacaagagctggacctggatccaccacgtgtccacctcccgtatctattgagcatgctgacatccgggtcaagaattacagtgtgaactccagggagaggtatgtctgtaactctggctttaagcggaaagctggaacatccaccctgattgagtgtgtgatcaacaagaacacaaatgttgcccactggacaactcccagcctcaagtgcatcagagacccctccctagctggagggagcggaggctctggcggaagcggcgggtctggaggctccgggggaagcggcggaaattggatcgacgtgcgctacgacctggaaaagatcgagagcctgatccagagcatccacatcgacaccaccctgtacaccgacagcgacttccaccccagctgcaaagtgaccgctatgaactgcttcctgctggaactgcaagtgatcctgcacgagtacagcaacatgaccctgaacgagacagtgcggaacgtgctgtacctggccaacagcaccctgagcagcaacaagaacgtggccgagagcggctgcaaagagtgcgaggaactggaagaaaagaccttcaccgagtttctgcagagcttcatcaggatcgtgcagatgttcatcaacacctcttgatgagtcgacgtcctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 51 - protein, 232 aa, synthetic construct; identical to SEQ ID NO: 48
  REGION 1..232: note = ModB murine IL-15 sushi

SEQ ID NO: 52 - DNA, 1255 bp, synthetic construct
  misc_feature 1..1255: note = ModB murine IL-15 sushi (5'UTR-CDS-3'UTR)
  source 1..1255: mol_type = other DNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagccatgggcgccatggcccctagaacattgctcctgctgctggccgctgccctggcccctacacagacaagagctggacctggatccaccacgtgtccacctcccgtatctattgagcatgctgacatccgggtcaagaattacagtgtgaactccagggagaggtatgtctgtaactctggctttaagcggaaagctggaacatccaccctgattgagtgtgtgatcaacaagaacacaaatgttgcccactggacaactcccagcctcaagtgcatcagagacccctccctagctggagggagcggaggctctggcggaagcggcgggtctggaggctccgggggaagcggcggaaattggatcgacgtgcgctacgacctggaaaagatcgagagcctgatccagagcatccacatcgacaccaccctgtacaccgacagcgacttccaccccagctgcaaagtgaccgctatgaactgcttcctgctggaactgcaagtgatcctgcacgagtacagcaacatgaccctgaacgagacagtgcggaacgtgctgtacctggccaacagcaccctgagcagcaacaagaacgtggccgagagcggctgcaaagagtgcgaggaactggaagaaaagaccttcaccgagtttctgcagagcttcatcaggatcgtgcagatgttcatcaacacctcttgatgagtcgacgtcctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 53 - RNA, 1255 nt, synthetic construct
  misc_feature 1..1255: note = ModB murine IL-15 sushi
  source 1..1255: mol_type = other RNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagccatgggcgccatggcccctagaacattgctcctgctgctggccgctgccctggcccctacacagacaagagctggacctggatccaccacgtgtccacctcccgtatctattgagcatgctgacatccgggtcaagaattacagtgtgaactccagggagaggtatgtctgtaactctggctttaagcggaaagctggaacatccaccctgattgagtgtgtgatcaacaagaacacaaatgttgcccactggacaactcccagcctcaagtgcatcagagacccctccctagctggagggagcggaggctctggcggaagcggcgggtctggaggctccgggggaagcggcggaaattggatcgacgtgcgctacgacctggaaaagatcgagagcctgatccagagcatccacatcgacaccaccctgtacaccgacagcgacttccaccccagctgcaaagtgaccgctatgaactgcttcctgctggaactgcaagtgatcctgcacgagtacagcaacatgaccctgaacgagacagtgcggaacgtgctgtacctggccaacagcaccctgagcagcaacaagaacgtggccgagagcggctgcaaagagtgcgaggaactggaagaaaagaccttcaccgagtttctgcagagcttcatcaggatcgtgcagatgttcatcaacacctcttgatgagtcgacgtcctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 54 - protein, 141 aa, synthetic construct
  REGION 1..141: note = ModA murine GM-CSF
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 55 - DNA, 908 bp, synthetic construct
  misc_feature 1..908: note = ModA murine GM-CSF (5'UTR-CDS-3'UTR)
  source 1..908: mol_type = other DNA
gggcgaactagtattcttctggtccccacagactcagagagaacccgccaccatgtggctgcagaacctgctgttcctgggcatcgtggtgtacagcctgagcgcccccaccaggagccccatcaccgtgaccaggccctggaagcacgtggaggccatcaaggaggccctgaacctgctggacgacatgcccgtgaccctgaacgaggaggtggaggtggtgagcaacgagttcagcttcaagaagctgacctgcgtgcagaccaggctgaagatcttcgagcagggcctgaggggcaacttcaccaagctgaagggcgccctgaacatgaccgccagctactaccagacctactgcccccccacccccgagaccgactgcgagacccaggtgaccacctacgccgacttcatcgacagcctgaagaccttcctgaccgacatccccttcgagtgcaagaagcccggccagaagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 56 - RNA, 908 nt, synthetic construct
  misc_feature 1..908: note = ModA murine GM-CSF
  source 1..908: mol_type = other RNA
gggcgaactagtattcttctggtccccacagactcagagagaacccgccaccatgtggctgcagaacctgctgttcctgggcatcgtggtgtacagcctgagcgcccccaccaggagccccatcaccgtgaccaggccctggaagcacgtggaggccatcaaggaggccctgaacctgctggacgacatgcccgtgaccctgaacgaggaggtggaggtggtgagcaacgagttcagcttcaagaagctgacctgcgtgcagaccaggctgaagatcttcgagcagggcctgaggggcaacttcaccaagctgaagggcgccctgaacatgaccgccagctactaccagacctactgcccccccacccccgagaccgactgcgagacccaggtgaccacctacgccgacttcatcgacagcctgaagaccttcctgaccgacatccccttcgagtgcaagaagcccggccagaagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 57 - protein, 141 aa, synthetic construct; identical to SEQ ID NO: 54
  REGION 1..141: note = ModB murine GM-CSF

SEQ ID NO: 58 - DNA, 1001 bp, synthetic construct
  misc_feature 1..1001: note = ModB murine GM-CSF (5'UTR-CDS-3'UTR)
  source 1..1001: mol_type = other DNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagccatgtggctgcagaacctgctgttcctgggcatcgtggtgtacagcctgagcgcccccaccaggagccccatcaccgtgaccaggccctggaagcacgtggaggccatcaaggaggccctgaacctgctggacgacatgcccgtgaccctgaacgaggaggtggaggtggtgagcaacgagttcagcttcaagaagctgacctgcgtgcagaccaggctgaagatcttcgagcagggcctgaggggcaacttcaccaagctgaagggcgccctgaacatgaccgccagctactaccagacctactgcccccccacccccgagaccgactgcgagacccaggtgaccacctacgccgacttcatcgacagcctgaagaccttcctgaccgacatccccttcgagtgcaagaagcccggccagaagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 59 - RNA, 1001 nt, synthetic construct
  misc_feature 1..1001: note = ModB murine GM-CSF
  source 1..1001: mol_type = other RNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagccatgtggctgcagaacctgctgttcctgggcatcgtggtgtacagcctgagcgcccccaccaggagccccatcaccgtgaccaggccctggaagcacgtggaggccatcaaggaggccctgaacctgctggacgacatgcccgtgaccctgaacgaggaggtggaggtggtgagcaacgagttcagcttcaagaagctgacctgcgtgcagaccaggctgaagatcttcgagcagggcctgaggggcaacttcaccaagctgaagggcgccctgaacatgaccgccagctactaccagacctactgcccccccacccccgagaccgactgcgagacccaggtgaccacctacgccgacttcatcgacagcctgaagaccttcctgaccgacatccccttcgagtgcaagaagcccggccagaagtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 60 - protein, 187 aa, synthetic construct
  REGION 1..187: note = ModA FLT3L (human FLT3L in combination with a mouse optimized secretion sequence)
MGAMAPRTLLLLLAAALAPTQTRAGPGSTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPP

SEQ ID NO: 61 - DNA, 1027 bp, synthetic construct
  misc_feature 1..1027: note = ModA FLT3L (5'UTR-CDS-3'UTR)
  source 1..1027: mol_type = other DNA
gggcgaactagtattcttctggtccccacagactcagagagaacccgccaccatgggcgccatggcccctagaacattgctcctgctgctggccgctgccctggcccctacacagacaagagctggacctggatccacccaggactgcagcttccagcactcccctatctcctccgacttcgccgtgaagatccgggagctgtccgattacctgctgcaggactaccctgtgaccgtggccagcaacctgcaggacgaagaactgtgtggcggcctgtggcggctggtgctggcccagcggtggatggaacggctgaaaaccgtggccggctccaagatgcagggcctgctcgagcgggtgaacaccgagatccacttcgtgaccaagtgcgccttccagcctcctccttcctgcctgcggttcgtgcagaccaacatctcccggctgctgcaggaaacctccgagcagctggtcgccctgaagccttggatcacccggcagaacttctcccggtgtctggaactccagtgtcagcccgactcctccaccctgcctcctccctggtcccccaggcctctggaagccaccgcccctaccgccccacagcctccttgataggtcgacgtcctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 62 - RNA, 1027 nt, synthetic construct
  misc_feature 1..1027: note = ModA FLT3L
  source 1..1027: mol_type = other RNA
gggcgaactagtattcttctggtccccacagactcagagagaacccgccaccatgggcgccatggcccctagaacattgctcctgctgctggccgctgccctggcccctacacagacaagagctggacctggatccacccaggactgcagcttccagcactcccctatctcctccgacttcgccgtgaagatccgggagctgtccgattacctgctgcaggactaccctgtgaccgtggccagcaacctgcaggacgaagaactgtgtggcggcctgtggcggctggtgctggcccagcggtggatggaacggctgaaaaccgtggccggctccaagatgcagggcctgctcgagcgggtgaacaccgagatccacttcgtgaccaagtgcgccttccagcctcctccttcctgcctgcggttcgtgcagaccaacatctcccggctgctgcaggaaacctccgagcagctggtcgccctgaagccttggatcacccggcagaacttctcccggtgtctggaactccagtgtcagcccgactcctccaccctgcctcctccctggtcccccaggcctctggaagccaccgcccctaccgccccacagcctccttgataggtcgacgtcctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 63 - protein, 187 aa, synthetic construct; identical to SEQ ID NO: 60
  REGION 1..187: note = ModB FLT3L (human FLT3L in combination with a mouse optimized secretion sequence)

SEQ ID NO: 64 - DNA, 1120 bp, synthetic construct
  misc_feature 1..1120: note = ModB FLT3L (5'UTR-CDS-3'UTR)
  source 1..1120: mol_type = other DNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagccatgggcgccatggcccctagaacattgctcctgctgctggccgctgccctggcccctacacagacaagagctggacctggatccacccaggactgcagcttccagcactcccctatctcctccgacttcgccgtgaagatccgggagctgtccgattacctgctgcaggactaccctgtgaccgtggccagcaacctgcaggacgaagaactgtgtggcggcctgtggcggctggtgctggcccagcggtggatggaacggctgaaaaccgtggccggctccaagatgcagggcctgctcgagcgggtgaacaccgagatccacttcgtgaccaagtgcgccttccagcctcctccttcctgcctgcggttcgtgcagaccaacatctcccggctgctgcaggaaacctccgagcagctggtcgccctgaagccttggatcacccggcagaacttctcccggtgtctggaactccagtgtcagcccgactcctccaccctgcctcctccctggtcccccaggcctctggaagccaccgcccctaccgccccacagcctccttgataggtcgacgtcctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 65 - RNA, 1120 nt, synthetic construct
  misc_feature 1..1120: note = ModB murine FLT3L
  source 1..1120: mol_type = other RNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagccatgggcgccatggcccctagaacattgctcctgctgctggccgctgccctggcccctacacagacaagagctggacctggatccacccaggactgcagcttccagcactcccctatctcctccgacttcgccgtgaagatccgggagctgtccgattacctgctgcaggactaccctgtgaccgtggccagcaacctgcaggacgaagaactgtgtggcggcctgtggcggctggtgctggcccagcggtggatggaacggctgaaaaccgtggccggctccaagatgcagggcctgctcgagcgggtgaacaccgagatccacttcgtgaccaagtgcgccttccagcctcctccttcctgcctgcggttcgtgcagaccaacatctcccggctgctgcaggaaacctccgagcagctggtcgccctgaagccttggatcacccggcagaacttctcccggtgtctggaactccagtgtcagcccgactcctccaccctgcctcctccctggtcccccaggcctctggaagccaccgcccctaccgccccacagcctccttgataggtcgacgtcctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 66 - protein, 309 aa, synthetic construct
  REGION 1..309: note = ModA murine 41BBL
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 67 - DNA, 1393 bp, synthetic construct
  misc_feature 1..1393: note = ModA murine 41BBL (5'UTR-CDS-3'UTR)
  source 1..1393: mol_type = other DNA
gggcgaactagtattcttctggtccccacagactcagagagaacccgccaccatggaccagcacacacttgatgtggaggataccgcggatgccagacatccagcaggtacttcgtgcccctcggatgcggcgctcctcagagataccgggctcctcgcggacgctgcgctcctctcagatactgtgcgccccacaaatgccgcgctccccacggatgctgcctaccctgcggttaatgttcgggatcgcgaggccgcgtggccgcctgcactgaacttctgttcccgccacccaaagctctatggcctagtcgctttggttttgctgcttctgatcgccgcctgtgttcctatcttcacccgcaccgagcctcggccagcgctcacaatcaccacctcgcccaacctgggtacccgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggagctcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatgatagggatccgatctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 68 - RNA, 1393 nt, synthetic construct
  misc_feature 1..1393: note = ModA murine 41BBL
  source 1..1393: mol_type = other RNA
gggcgaactagtattcttctggtccccacagactcagagagaacccgccaccatggaccagcacacacttgatgtggaggataccgcggatgccagacatccagcaggtacttcgtgcccctcggatgcggcgctcctcagagataccgggctcctcgcggacgctgcgctcctctcagatactgtgcgccccacaaatgccgcgctccccacggatgctgcctaccctgcggttaatgttcgggatcgcgaggccgcgtggccgcctgcactgaacttctgttcccgccacccaaagctctatggcctagtcgctttggttttgctgcttctgatcgccgcctgtgttcctatcttcacccgcaccgagcctcggccagcgctcacaatcaccacctcgcccaacctgggtacccgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggagctcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatgatagggatccgatctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 69 - protein, 309 aa, synthetic construct; identical to SEQ ID NO: 66
  REGION 1..309: note = ModB murine 41BBL

SEQ ID NO: 70 - DNA, 1486 bp, synthetic construct
  misc_feature 1..1486: note = ModB murine 41BBL (5'UTR-CDS-3'UTR)
  source 1..1486: mol_type = other DNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagccatggaccagcacacacttgatgtggaggataccgcggatgccagacatccagcaggtacttcgtgcccctcggatgcggcgctcctcagagataccgggctcctcgcggacgctgcgctcctctcagatactgtgcgccccacaaatgccgcgctccccacggatgctgcctaccctgcggttaatgttcgggatcgcgaggccgcgtggccgcctgcactgaacttctgttcccgccacccaaagctctatggcctagtcgctttggttttgctgcttctgatcgccgcctgtgttcctatcttcacccgcaccgagcctcggccagcgctcacaatcaccacctcgcccaacctgggtacccgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggagctcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatgatagggatccgatctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 71 - RNA, 1486 nt, synthetic construct
  misc_feature 1..1486: note = ModB murine 41BBL
  source 1..1486: mol_type = other RNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagccatggaccagcacacacttgatgtggaggataccgcggatgccagacatccagcaggtacttcgtgcccctcggatgcggcgctcctcagagataccgggctcctcgcggacgctgcgctcctctcagatactgtgcgccccacaaatgccgcgctccccacggatgctgcctaccctgcggttaatgttcgggatcgcgaggccgcgtggccgcctgcactgaacttctgttcccgccacccaaagctctatggcctagtcgctttggttttgctgcttctgatcgccgcctgtgttcctatcttcacccgcaccgagcctcggccagcgctcacaatcaccacctcgcccaacctgggtacccgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggagctcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatgatagggatccgatctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 72 - protein, 935 aa, synthetic construct
  REGION 1..935: note = ModA murine CD27L-CD40L
MRVTAPRTLILLLSGALALTETWAGSGSHPEPHTAELQLNLTVPRKDPTLRWGAGPALGRSFTHGPELEEGHLRIHQDGLYRLHIQVTLANCSSPGSTLQHRATLAVGICSPAAHGISLLRGRFGQDCTVALQRLTYLVHGDVLCTNLTLPLLPSRNADETFFGVQWICPGGGSGGGHPEPHTAELQLNLTVPRKDPTLRWGAGPALGRSFTHGPELEEGHLRIHQDGLYRLHIQVTLANCSSPGSTLQHRATLAVGICSPAAHGISLLRGRFGQDCTVALQRLTYLVHGDVLCTNLTLPLLPSRNADETFFGVQWICPGGGSGGGHPEPHTAELQLNLTVPRKDPTLRWGAGPALGRSFTHGPELEEGHLRIHQDGLYRLHIQVTLANCSSPGSTLQHRATLAVGICSPAAHGISLLRGRFGQDCTVALQRLTYLVHGDVLCTNLTLPLLPSRNADETFFGVQWICPGGGGSGGGGSGGGGSGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGSGGGGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGSGGGGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 73 - DNA, 3281 bp, synthetic construct
  misc_feature 1..3281: note = ModA murine CD27L-CD40L (5'UTR-CDS-3'UTR)
  source 1..3281: mol_type = other DNA
gggcgaactagtattcttctggtccccacagactcagagagaacccgccaccatgagagtgaccgcccccagaaccctgatcctgctgctgtctggcgccctggccctgacagagacatgggccggaagcggatcccaccccgagccccacaccgccgaactgcagctgaacctgaccgtgcccagaaaggaccccaccctgagatggggagctggccctgctctgggcagatcctttacacacggccccgagctggaagaaggccacctgagaatccaccaggacggcctgtacagactgcacatccaagtgaccctggccaactgcagcagccctggctctaccctgcagcacagagccacactggccgtgggcatctgtagccctgctgctcacggaatcagcctgctgagaggcagattcggccaggactgtaccgtggccctgcagaggctgacctatctggtgcatggcgacgtgctgtgcaccaacctgacactgcctctgctgcccagcagaaacgccgacgaaacattctttggagtgcagtggatttgtcctggcggagggtccgggggaggacacccagaacctcatacagctgaactgcagctgaacctgaccgtgcccagaaaggaccccaccctgagatggggagctggccctgctctgggcagatcctttacacacggccccgagctggaagaaggccacctgagaatccaccaggacggcctgtacagactgcacatccaagtgaccctggccaactgcagcagccctggctctaccctgcagcacagagccacactggccgtgggcatctgtagccctgctgctcacggaatcagcctgctgagaggcagattcggccaggactgtaccgtggccctgcagaggctgacctatctggtgcatggcgacgtgctgtgcaccaacctgacactgcctctgctgcccagcagaaacgccgacgaaacattctttggagtgcagtggatttgtcctgggggaggctccggaggcggacaccctgaacctcatacagctgaactgcagctgaacctgaccgtgcccagaaaggaccccaccctgagatggggagctggccctgctctgggcagatcctttacacacggccccgagctggaagaaggccacctgagaatccaccaggacggcctgtacagactgcacatccaagtgaccctggccaactgcagcagccctggctctaccctgcagcacagagccacactggccgtgggcatctgtagccctgctgctcacggaatcagcctgctgagaggcagattcggccaggactgtaccgtggccctgcagaggctgacctatctggtgcatggcgacgtgctgtgcaccaacctgacactgcctctgctgcccagcagaaacgccgacgagaccttcttcggcgtccagtggatctgccccggaggcggtggtagtggaggtggcgggtccggtggaggtggaagcggcgacgaggacccccagatcgccgcccacgtggtgtctgaggccaacagcaacgccgcctctgtgctgcagtgggccaagaaaggctactacaccatgaagtccaacctcgtgatgctggaaaacggcaagcagctgaccgtgaagcgcgagggcctgtactatgtgtacacccaagtgacattctgcagcaaccgcgagcccagcagccagaggccttttatcgtgggcctgtggctgaagcctagcagcggcagcgagagaatcctgctgaaggccgccaacacccacagcagctctcagctgtgcgagcagcagtctgtgcacctgggaggcgtgttcgagctgcaagctggcgcttccgtgttcgtgaacgtgaccgaggccagccaagtgatccacagagtgggcttcagcagctttggactgctcaaactgggcggagggtccggcggaggcggagatgaagatcctcagattgctgcccacgtggtgtctgaggccaacagcaacgccgcctctgtgctgcagtgggccaagaaaggctactacaccatgaagtccaacctcgtgatgctggaaaacggcaagcagctgaccgtgaagcgcgagggcctgtactatgtgtacacccaagtgacattctgcagcaaccgcgagcccagcagccagaggccttttatcgtgggcctgtggctgaagcctagcagcggcagcgagagaatcctgctgaaggccgccaacacccacagcagctctcagctgtgcgagcagcagtctgtgcacctgggaggcgtgttcgagctgcaagctggcgcttccgtgttcgtgaacgtgaccgaggccagccaagtgatccacagagtgggcttcagcagctttggactgctcaaactgggaggcggctccggaggcggaggagatgaagatcctcagattgctgcccacgtggtgtctgaggccaacagcaacgccgcctctgtgctgcagtgggccaagaaaggctactacaccatgaagtccaacctcgtgatgctggaaaacggcaagcagctgaccgtgaagcgcgagggcctgtactatgtgtacacccaagtgacattctgcagcaaccgcgagcccagcagccagaggccttttatcgtgggcctgtggctgaagcctagcagcggcagcgagagaatcctgctgaaggccgccaacacccacagcagctctcagctgtgcgagcagcagtctgtgcacctgggaggcgtgttcgagctgcaagctggcgcttccgtgttcgtgaacgtgaccgaggccagccaagtgatccacagagtgggcttctcctccttcggcctcctgaagctgtgactcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagacctggtccagagtcgctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 74 - RNA, 3281 nt, synthetic construct
  misc_feature 1..3281: note = ModA murine CD27L-CD40L
  source 1..3281: mol_type = other RNA
gggcgaactagtattcttctggtccccacagactcagagagaacccgccaccatgagagtgaccgcccccagaaccctgatcctgctgctgtctggcgccctggccctgacagagacatgggccggaagcggatcccaccccgagccccacaccgccgaactgcagctgaacctgaccgtgcccagaaaggaccccaccctgagatggggagctggccctgctctgggcagatcctttacacacggccccgagctggaagaaggccacctgagaatccaccaggacggcctgtacagactgcacatccaagtgaccctggccaactgcagcagccctggctctaccctgcagcacagagccacactggccgtgggcatctgtagccctgctgctcacggaatcagcctgctgagaggcagattcggccaggactgtaccgtggccctgcagaggctgacctatctggtgcatggcgacgtgctgtgcaccaacctgacactgcctctgctgcccagcagaaacgccgacgaaacattctttggagtgcagtggatttgtcctggcggagggtccgggggaggacacccagaacctcatacagctgaactgcagctgaacctgaccgtgcccagaaaggaccccaccctgagatggggagctggccctgctctgggcagatcctttacacacggccccgagctggaagaaggccacctgagaatccaccaggacggcctgtacagactgcacatccaagtgaccctggccaactgcagcagccctggctctaccctgcagcacagagccacactggccgtgggcatctgtagccctgctgctcacggaatcagcctgctgagaggcagattcggccaggactgtaccgtggccctgcagaggctgacctatctggtgcatggcgacgtgctgtgcaccaacctgacactgcctctgctgcccagcagaaacgccgacgaaacattctttggagtgcagtggatttgtcctgggggaggctccggaggcggacaccctgaacctcatacagctgaactgcagctgaacctgaccgtgcccagaaaggaccccaccctgagatggggagctggccctgctctgggcagatcctttacacacggccccgagctggaagaaggccacctgagaatccaccaggacggcctgtacagactgcacatccaagtgaccctggccaactgcagcagccctggctctaccctgcagcacagagccacactggccgtgggcatctgtagccctgctgctcacggaatcagcctgctgagaggcagattcggccaggactgtaccgtggccctgcagaggctgacctatctggtgcatggcgacgtgctgtgcaccaacctgacactgcctctgctgcccagcagaaacgccgacgagaccttcttcggcgtccagtggatctgccccggaggcggtggtagtggaggtggcgggtccggtggaggtggaagcggcgacgaggacccccagatcgccgcccacgtggtgtctgaggccaacagcaacgccgcctctgtgctgcagtgggccaagaaaggctactacaccatgaagtccaacctcgtgatgctggaaaacggcaagcagctgaccgtgaagcgcgagggcctgtactatgtgtacacccaagtgacattctgcagcaaccgcgagcccagcagccagaggccttttatcgtgggcctgtggctgaagcctagcagcggcagcgagagaatcctgctgaaggccgccaacacccacagcagctctcagctgtgcgagcagcagtctgtgcacctgggaggcgtgttcgagctgcaagctggcgcttccgtgttcgtgaacgtgaccgaggccagccaagtgatccacagagtgggcttcagcagctttggactgctcaaactgggcggagggtccggcggaggcggagatgaagatcctcagattgctgcccacgtggtgtctgaggccaacagcaacgccgcctctgtgctgcagtgggccaagaaaggctactacaccatgaagtccaacctcgtgatgctggaaaacggcaagcagctgaccgtgaagcgcgagggcctgtactatgtgtacacccaagtgacattctgcagcaaccgcgagcccagcagccagaggccttttatcgtgggcctgtggctgaagcctagcagcggcagcgagagaatcctgctgaaggccgccaacacccacagcagctctcagctgtgcgagcagcagtctgtgcacctgggaggcgtgttcgagctgcaagctggcgcttccgtgttcgtgaacgtgaccgaggccagccaagtgatccacagagtgggcttcagcagctttggactgctcaaactgggaggcggctccggaggcggaggagatgaagatcctcagattgctgcccacgtggtgtctgaggccaacagcaacgccgcctctgtgctgcagtgggccaagaaaggctactacaccatgaagtccaacctcgtgatgctggaaaacggcaagcagctgaccgtgaagcgcgagggcctgtactatgtgtacacccaagtgacattctgcagcaaccgcgagcccagcagccagaggccttttatcgtgggcctgtggctgaagcctagcagcggcagcgagagaatcctgctgaaggccgccaacacccacagcagctctcagctgtgcgagcagcagtctgtgcacctgggaggcgtgttcgagctgcaagctggcgcttccgtgttcgtgaacgtgaccgaggccagccaagtgatccacagagtgggcttctcctccttcggcctcctgaagctgtgactcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagacctggtccagagtcgctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 75 - protein, 935 aa, synthetic construct
  REGION 1..935: note = ModB murine CD27L-CD40L
MGAMAPRTLLLLLAAALAPTQTRAGPGSHPEPHTAELQLNLTVPRKDPTLRWGAGPALGRSFTHGPELEEGHLRIHQDGLYRLHIQVTLANCSSPGSTLQHRATLAVGICSPAAHGISLLRGRFGQDCTVALQRLTYLVHGDVLCTNLTLPLLPSRNADETFFGVQWICPGGGSGGGHPEPHTAELQLNLTVPRKDPTLRWGAGPALGRSFTHGPELEEGHLRIHQDGLYRLHIQVTLANCSSPGSTLQHRATLAVGICSPAAHGISLLRGRFGQDCTVALQRLTYLVHGDVLCTNLTLPLLPSRNADETFFGVQWICPGGGSGGGHPEPHTAELQLNLTVPRKDPTLRWGAGPALGRSFTHGPELEEGHLRIHQDGLYRLHIQVTLANCSSPGSTLQHRATLAVGICSPAAHGISLLRGRFGQDCTVALQRLTYLVHGDVLCTNLTLPLLPSRNADETFFGVQWICPGGGGSGGGGSGGGGSGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGSGGGGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGSGGGGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 76 - DNA, 3361 bp, synthetic construct
  misc_feature 1..3361: note = ModB murine CD27L-CD40L (5'UTR-CDS-3'UTR)
  source 1..3361: mol_type = other DNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagccatgggcgccatggcccctagaacattgctcctgctgctggccgctgccctggcccctacacagacaagagctggacctggatcccaccccgagccccacaccgccgaactgcagctgaacctgaccgtgcccagaaaggaccccaccctgagatggggagctggccctgctctgggcagatcctttacacacggccccgagctggaagaaggccacctgagaatccaccaggacggcctgtacagactgcacatccaagtgaccctggccaactgcagcagccctggctctaccctgcagcacagagccacactggccgtgggcatctgtagccctgctgctcacggaatcagcctgctgagaggcagattcggccaggactgtaccgtggccctgcagaggctgacctatctggtgcatggcgacgtgctgtgcaccaacctgacactgcctctgctgcccagcagaaacgccgacgaaacattctttggagtgcagtggatttgtcctggcggagggtccgggggaggacacccagaacctcatacagctgaactgcagctgaacctgaccgtgcccagaaaggaccccaccctgagatggggagctggccctgctctgggcagatcctttacacacggccccgagctggaagaaggccacctgagaatccaccaggacggcctgtacagactgcacatccaagtgaccctggccaactgcagcagccctggctctaccctgcagcacagagccacactggccgtgggcatctgtagccctgctgctcacggaatcagcctgctgagaggcagattcggccaggactgtaccgtggccctgcagaggctgacctatctggtgcatggcgacgtgctgtgcaccaacctgacactgcctctgctgcccagcagaaacgccgacgaaacattctttggagtgcagtggatttgtcctgggggaggctccggaggcggacaccctgaacctcatacagctgaactgcagctgaacctgaccgtgcccagaaaggaccccaccctgagatggggagctggccctgctctgggcagatcctttacacacggccccgagctggaagaaggccacctgagaatccaccaggacggcctgtacagactgcacatccaagtgaccctggccaactgcagcagccctggctctaccctgcagcacagagccacactggccgtgggcatctgtagccctgctgctcacggaatcagcctgctgagaggcagattcggccaggactgtaccgtggccctgcagaggctgacctatctggtgcatggcgacgtgctgtgcaccaacctgacactgcctctgctgcccagcagaaacgccgacgagaccttcttcggcgtccagtggatctgccccggaggcggtggtagtggaggtggcgggtccggtggaggtggaagcggcgacgaggacccccagatcgccgcccacgtggtgtctgaggccaacagcaacgccgcctctgtgctgcagtgggccaagaaaggctactacaccatgaagtccaacctcgtgatgctggaaaacggcaagcagctgaccgtgaagcgcgagggcctgtactatgtgtacacccaagtgacattctgcagcaaccgcgagcccagcagccagaggccttttatcgtgggcctgtggctgaagcctagcagcggcagcgagagaatcctgctgaaggccgccaacacccacagcagctctcagctgtgcgagcagcagtctgtgcacctgggaggcgtgttcgagctgcaagctggcgcttccgtgttcgtgaacgtgaccgaggccagccaagtgatccacagagtgggcttcagcagctttggactgctcaaactgggcggagggtccggcggaggcggagatgaagatcctcagattgctgcccacgtggtgtctgaggccaacagcaacgccgcctctgtgctgcagtgggccaagaaaggctactacaccatgaagtccaacctcgtgatgctggaaaacggcaagcagctgaccgtgaagcgcgagggcctgtactatgtgtacacccaagtgacattctgcagcaaccgcgagcccagcagccagaggccttttatcgtgggcctgtggctgaagcctagcagcggcagcgagagaatcctgctgaaggccgccaacacccacagcagctctcagctgtgcgagcagcagtctgtgcacctgggaggcgtgttcgagctgcaagctggcgcttccgtgttcgtgaacgtgaccgaggccagccaagtgatccacagagtgggcttcagcagctttggactgctcaaactgggaggcggctccggaggcggaggagatgaagatcctcagattgctgcccacgtggtgtctgaggccaacagcaacgccgcctctgtgctgcagtgggccaagaaaggctactacaccatgaagtccaacctcgtgatgctggaaaacggcaagcagctgaccgtgaagcgcgagggcctgtactatgtgtacacccaagtgacattctgcagcaaccgcgagcccagcagccagaggccttttatcgtgggcctgtggctgaagcctagcagcggcagcgagagaatcctgctgaaggccgccaacacccacagcagctctcagctgtgcgagcagcagtctgtgcacctgggaggcgtgttcgagctgcaagctggcgcttccgtgttcgtgaacgtgaccgaggccagccaagtgatccacagagtgggcttctcctccttcggcctcctgaagctgtgactcgacgtcctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 77 - RNA, 3361 nt, synthetic construct
  misc_feature 1..3361: note = ModB murine CD27L-CD40L
  source 1..3361: mol_type = other RNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagccatgggcgccatggcccctagaacattgctcctgctgctggccgctgccctggcccctacacagacaagagctggacctggatcccaccccgagccccacaccgccgaactgcagctgaacctgaccgtgcccagaaaggaccccaccctgagatggggagctggccctgctctgggcagatcctttacacacggccccgagctggaagaaggccacctgagaatccaccaggacggcctgtacagactgcacatccaagtgaccctggccaactgcagcagccctggctctaccctgcagcacagagccacactggccgtgggcatctgtagccctgctgctcacggaatcagcctgctgagaggcagattcggccaggactgtaccgtggccctgcagaggctgacctatctggtgcatggcgacgtgctgtgcaccaacctgacactgcctctgctgcccagcagaaacgccgacgaaacattctttggagtgcagtggatttgtcctggcggagggtccgggggaggacacccagaacctcatacagctgaactgcagctgaacctgaccgtgcccagaaaggaccccaccctgagatggggagctggccctgctctgggcagatcctttacacacggccccgagctggaagaaggccacctgagaatccaccaggacggcctgtacagactgcacatccaagtgaccctggccaactgcagcagccctggctctaccctgcagcacagagccacactggccgtgggcatctgtagccctgctgctcacggaatcagcctgctgagaggcagattcggccaggactgtaccgtggccctgcagaggctgacctatctggtgcatggcgacgtgctgtgcaccaacctgacactgcctctgctgcccagcagaaacgccgacgaaacattctttggagtgcagtggatttgtcctgggggaggctccggaggcggacaccctgaacctcatacagctgaactgcagctgaacctgaccgtgcccagaaaggaccccaccctgagatggggagctggccctgctctgggcagatcctttacacacggccccgagctggaagaaggccacctgagaatccaccaggacggcctgtacagactgcacatccaagtgaccctggccaactgcagcagccctggctctaccctgcagcacagagccacactggccgtgggcatctgtagccctgctgctcacggaatcagcctgctgagaggcagattcggccaggactgtaccgtggccctgcagaggctgacctatctggtgcatggcgacgtgctgtgcaccaacctgacactgcctctgctgcccagcagaaacgccgacgagaccttcttcggcgtccagtggatctgccccggaggcggtggtagtggaggtggcgggtccggtggaggtggaagcggcgacgaggacccccagatcgccgcccacgtggtgtctgaggccaacagcaacgccgcctctgtgctgcagtgggccaagaaaggctactacaccatgaagtccaacctcgtgatgctggaaaacggcaagcagctgaccgtgaagcgcgagggcctgtactatgtgtacacccaagtgacattctgcagcaaccgcgagcccagcagccagaggccttttatcgtgggcctgtggctgaagcctagcagcggcagcgagagaatcctgctgaaggccgccaacacccacagcagctctcagctgtgcgagcagcagtctgtgcacctgggaggcgtgttcgagctgcaagctggcgcttccgtgttcgtgaacgtgaccgaggccagccaagtgatccacagagtgggcttcagcagctttggactgctcaaactgggcggagggtccggcggaggcggagatgaagatcctcagattgctgcccacgtggtgtctgaggccaacagcaacgccgcctctgtgctgcagtgggccaagaaaggctactacaccatgaagtccaacctcgtgatgctggaaaacggcaagcagctgaccgtgaagcgcgagggcctgtactatgtgtacacccaagtgacattctgcagcaaccgcgagcccagcagccagaggccttttatcgtgggcctgtggctgaagcctagcagcggcagcgagagaatcctgctgaaggccgccaacacccacagcagctctcagctgtgcgagcagcagtctgtgcacctgggaggcgtgttcgagctgcaagctggcgcttccgtgttcgtgaacgtgaccgaggccagccaagtgatccacagagtgggcttcagcagctttggactgctcaaactgggaggcggctccggaggcggaggagatgaagatcctcagattgctgcccacgtggtgtctgaggccaacagcaacgccgcctctgtgctgcagtgggccaagaaaggctactacaccatgaagtccaacctcgtgatgctggaaaacggcaagcagctgaccgtgaagcgcgagggcctgtactatgtgtacacccaagtgacattctgcagcaaccgcgagcccagcagccagaggccttttatcgtgggcctgtggctgaagcctagcagcggcagcgagagaatcctgctgaaggccgccaacacccacagcagctctcagctgtgcgagcagcagtctgtgcacctgggaggcgtgttcgagctgcaagctggcgcttccgtgttcgtgaacgtgaccgaggccagccaagtgatccacagagtgggcttctcctccttcggcctcctgaagctgtgactcgacgtcctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctcgagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 78 - RNA, 110 nt, synthetic construct
  misc_feature 1..110: note = Poly-A
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 79 - protein, 444 aa, synthetic construct
  REGION 1..444: note = Anti-PD1 Mab heavy chain
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - protein, 214 aa, synthetic construct
  REGION 1..214: note = Anti-PD1 Mab light chain
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 8 aa, synthetic construct
  REGION 1..8: note = HCDR1
GFTFSNFG

SEQ ID NO: 82 - protein, 8 aa, synthetic construct
  REGION 1..8: note = HCDR2
ISGGGRDT

SEQ ID NO: 83 - protein, 10 aa, synthetic construct
  REGION 1..10: note = HCDR3
VKWGNIYFDY

SEQ ID NO: 84 - protein, 6 aa, synthetic construct
  REGION 1..6: note = LCDR1
LSINTF

SEQ ID NO: 85 - skipped

SEQ ID NO: 86 - protein, 9 aa, synthetic construct
  REGION 1..9: note = LCDR3
QQSSNTPFT

SEQ ID NO: 87 - protein, 117 aa, synthetic construct [sequence omitted]

SEQ ID NO: 88 - protein, 107 aa, synthetic construct [sequence omitted]

(19) United States
(12) Patent Application Publication
(10) Pub. No.: US 2024/0173382 A1
(43) Pub. Date: May 30, 2024

(54) Therapeutic RNA

(71) Applicant: Sanofi, Paris (FR)

(72) Inventors: Friederike Gieseke, Mainz (DE); Ugur Sahin, Mainz (DE); Timothy R. Wagenaar, Sudbury, MA (US); Dmitri G. Wiederschain, Bridgewater, NJ (US); Christian Hotz, Mainz (DE)

(73) Assignee: Sanofi, Paris (FR)

(21) Appl. No.: 18/516,006

(22) Filed: Nov. 21, 2023

Related U.S. Application Data
(62) Division of application No. 17/245,605, filed on Apr. 30, 2021, now Pat. No. 11,865,159.
Application No. 17/245,605 is a division of application No. 16/552,248, filed on Aug. 27, 2019, now abandoned.
Application No. 16/552,248 is a continuation of application No. PCT/US2018/019878, filed on Feb. 27, 2018.
(60) Provisional application No. 62/597,527, filed on Dec. 12, 2017.
(60) Provisional application No. 62/464,981, filed on Feb. 28, 2017.

(30) Foreign Application Priority Data
Aug. 23, 2017 (EP) EP17306089.8

(51) Int. Cl. A61K 38/20; A61K 9/00; A61K 38/19; A61K 38/21; A61K 39/395; A61K 48/00; A61P 35/00

(52) U.S. Cl. CPC A61K 38/208; A61K 9/0019; A61K 38/193; A61K 38/2086; A61K 38/212; A61K 39/3955; A61K 48/00; A61P 35/00

(57) ABSTRACT

This disclosure relates to the field of therapeutic RNAs for treatment of solid tumor cancers.

This application is a Divisional of U.S. application Ser. No. 17/245,605, filed on Apr. 30, 2021, which is a Divisional of U.S. application Ser. No. 16/552,248, filed on Aug. 27, 2019, which is a Continuation of International Application No. PCT/US2018/019878, filed on Feb. 27, 2018, which claims the benefit of priority to U.S. Provisional Application No. 62/464,981, filed Feb. 28, 2017; U.S. Provisional Application No. 62/597,527, filed Dec. 12, 2017; and European Patent Application No. 17306089.8, filed Aug. 23, 2017; all of the contents of which are incorporated by reference in their entirety.

The instant application contains a Sequence Listing which has been submitted electronically in XML format and is hereby incorporated by reference in its entirety. The XML copy, created on Nov. 21, 2023, is named 01183-0001-02US-ST26.xml and is 177,463 bytes in size.

This disclosure relates to the field of therapeutic RNA to treat solid tumors. The National Cancer Institute defines solid tumors as abnormal masses of tissue that do not normally contain cysts or liquid areas. Solid tumors include benign and malignant (cancerous) sarcomas, carcinomas, and lymphomas, and can be physically located in any tissue or organ including the brain, ovary, breast, colon, and other tissues. Cancer is often divided into two main types: solid tumor cancer and hematological (blood) cancers. It is estimated that more than 1.5 million cases of cancer are diagnosed in the United States each year, and more than 500,000 people in the United States will die each year from cancer.

Solid tumor cancers are particularly difficult to treat. Current treatments include surgery, radiotherapy, immunotherapy and chemotherapy. Surgery alone may be an appropriate treatment for small localized tumors, but large invasive tumors and most metastatic malignancies are usually unresectable by surgery. Other common treatments such as radiotherapy and chemotherapy are associated with undesirable side effects and damage to healthy cells.

While surgery and current therapies sometimes are able to kill the bulk of the solid tumor, additional cells (including potentially cancer stem cells) may survive therapy. These cells, over time, can form a new tumor leading to cancer recurrence. In spite of multimodal conventional therapies, disease-free survival is less than 25% for many types of solid tumors. Solid tumors that are resistant to multi-modal therapy or that have recurred following therapy are even more difficult to treat, and long-term survival is less than 10%.

Disclosed herein are compositions, uses, and methods that can overcome present shortcomings in treatment of solid tumors. Administration of therapeutic RNAs disclosed herein can reduce tumor size, extend survival time, and/or protect against metastasis and/or recurrence of the tumor.

### SUMMARY

Embodiment 1. A composition comprising RNA encoding an IL-12sc protein that is at least 99%, 98%, 97%, 96%, 95%, 90%, 85%, or 80% identical to the amino acids of SEQ ID NO: 14 and RNA encoding a GM-CSF protein that is at least 99%, 98%, 97%, 96%, 95%, 90%, 85%, or 80% identical to the amino acids of SEQ ID NO: 27.
Embodiment 2. The composition of embodiment 1, further comprising RNA encoding an IL-15 sushi protein that is at least 99%, 98%, 97%, 96%, 95%, 90%, 85%, or 80% identical to the amino acids of SEQ ID NO: 24.
Embodiment 3. The composition of embodiment 1, further comprising RNA encoding an IL-2 protein that is at least 99%, 98%, 97%, 96%, 95%, 90%, 85%, or 80% identical to the amino acids of SEQ ID NO: 9.
Embodiment 4. The composition of embodiment 1, further comprising RNA encoding an IFNα2b protein that is at least 99%, 98%, 97%, 96%, 95%, 90%, 85%, or 80% identical to the amino acids of SEQ ID NO: 19.
Embodiment 5. A composition comprising:
   a. RNA encoding an IL-12sc protein that is at least 99%, 98%, 97%, 96%, 95%, 90%, 85%, or 80% identical to the amino acids of SEQ ID NO: 14;
   b. RNA encoding a GM-CSF protein that is at least 99%, 98%, 97%, 96%, 95%, 90%, 85%, or 80% identical to the amino acids of SEQ ID NO: 27; and
   c. RNA encoding an IFNα2b protein that is at least 99%, 98%, 97%, 96%, 95%, 90%, 85%, or 80% identical to the amino acids of SEQ ID NO: 19.
Embodiment 6. The composition of embodiment 5, further comprising RNA encoding an IL-15sushi protein that is at least 99%, 98%, 97%, 96%, 95%, 90%, 85%, or 80% identical to the amino acids of SEQ ID NO: 24.
Embodiment 7. The composition of embodiment 5, further comprising RNA encoding an IL-2 protein that is at least 99%, 98%, 97%, 96%, 95%, 90%, 85%, or 80% identical to the amino acids of SEQ ID NO: 9.
Embodiment 8. The composition of any one of embodiments 1-7, wherein at least one RNA comprises a modified nucleobase in place of at least one uridine.
Embodiment 9. The composition of any one of embodiments 1-7, wherein each RNA comprises a modified nucleobase in place of each uridine.
Embodiment 10. The composition of any one of embodiments 8-9, wherein the modified nucleobase is pseudouridine (ψ), N1-methyl-pseudouridine (m¹ψ) or 5-methyl-uridine (m⁵U).
Embodiment 11. The composition of embodiment 10, wherein the modified nucleobase is N1-methyl-pseudouridine (m¹ψ).
Embodiment 12. The composition of any one of embodiments 1-11, wherein at least one RNA further comprises a 5′ cap.
Embodiment 13. The composition of any one of embodiments 1-11, wherein each RNA further comprises a 5′ cap.
Embodiment 14. The composition of any one of embodiments 12-13, wherein the 5′ cap is m₂^{7,3′-O}Gppp(m₁^{2′-O})ApG or 3′-O-Me-m⁷G(5′)ppp(5′)G.
Embodiment 15. The composition of any one of embodiments 1-14, wherein at least one RNA further comprises a 5′ UTR.
Embodiment 16. The composition of any one of embodiments 1-14, wherein each RNA further comprises a 5′ UTR.
Embodiment 17. The composition of any one of embodiments 15-16, wherein the 5′ UTR comprises or consists of the nucleotides of SEQ ID NOs: 2, 4, or 6, or nucleotides having at least 99%, 98%, 97%, 96%, 95%, 90%, or 85% identity to SEQ ID NOs: 2, 4, or 6.
Embodiment 18. The composition of any one of embodiments 1-17, wherein at least one RNA further comprises a 3′ UTR.
Embodiment 19. The composition of any one of embodiments 1-17, wherein each RNA further comprises a 3′ UTR.
Embodiment 20. The composition of any one of embodiments 18-19, wherein the 3′ UTR comprises or consists of the nucleotides of SEQ ID NO: 8, or nucleotides having at least 99%, 98%, 97%, 96%, 95%, 90%, or 85% identity to SEQ ID NO: 8.
Embodiment 21. The composition of any one of embodiments 1-20, wherein at least one RNA further comprises a poly-A tail.
Embodiment 22. The composition of any one of embodiments 1-20, wherein each RNA further comprises a poly-A tail.
Embodiment 23. The composition of any one of embodiments 21-22, wherein the poly-A tail comprises at least 100 nucleotides.
Embodiment 24. The composition of any one of embodiments 1-23, wherein at least one RNA comprises a 5′ cap, 5′ UTR, 3′ UTR, and poly-A tail.
Embodiment 25. The composition of any one of embodiments 1-23, wherein each RNA comprises a 5′ cap, 5′ UTR, 3′ UTR, and poly-A tail.
Embodiment 26. The composition of any one of embodiments 24-25, wherein
   a. the 5′ cap is m₂^{7,3′-O}Gppp(m₁^{2′-O})ApG or 3′-O-Me-m⁷G(5′)ppp(5′)G;
   b. the 5′ UTR comprises or consists of the nucleotides of SEQ ID NOs: 2, 4, or 6, or nucleotides having at least 99%, 98%, 97%, 96%, 95%, 90%, or 85% identity to SEQ ID NOs: 2, 4, or 6;
   c. the 3′ UTR comprises or consists of the nucleotides of SEQ ID NO: 8, or nucleotides having at least 99%, 98%, 97%, 96%, 95%, 90%, or 85% identity to SEQ ID NO: 8; and
   d. the poly-A tail comprises at least 100 nucleotides.
Embodiment 27. A method for treating or preventing cancer, reducing the size of a tumor, preventing the reoccurrence of cancer in remission, or preventing cancer metastasis in a subject comprising administering the composition of any one of embodiments 1-26 to the subject.
Embodiment 28. A method for treating or preventing cancer, reducing the size of a tumor, preventing the reoccurrence of cancer in remission, or preventing cancer metastasis in a subject comprising administering to the subject:
   a. an RNA encoding an IL-12sc protein that is at least 99%, 98%, 97%, 96%, 95%, 90%, 85%, or 80% identical to the amino acids of SEQ ID NO: 14, and/or comprising nucleotides having at least 99%, 98%, 97%, 96%, 95%, 90%, 85%, or 80% identity to the nucleotides of SEQ ID NOs: 17 or 18; and
   b. an RNA encoding a GM-CSF protein that is at least 99%, 98%, 97%, 96%, 95%, 90%, 85%, or 80% identical to the amino acids of SEQ ID NO: 27, and/or comprising nucleotides having at least 99%, 98%, 97%, 96%, 95%, 90%, 85%, or 80% identity to the nucleotides of SEQ ID NO: 29,
      thereby treating or preventing cancer, reducing the size of a tumor, preventing the reoccurrence of cancer in remission, or preventing cancer metastasis in the subject.
Embodiment 29. The method of embodiment 28, further comprising administering RNA encoding an IFNα2b protein that is at least 99%, 98%, 97%, 96%, 95%, 90%, 85%, or 80% identical to the amino acids of SEQ ID NO: 19, and/or comprising nucleotides having at least 99%, 98%, 97%, 96%, 95%, 90%, 85%, or 80% identity to the nucleotides of SEQ ID NOs: 22 or 23.
Embodiment 30. The method of embodiment 28, further comprising administering RNA encoding an IL-15 sushi protein that is at least 99%, 98%, 97%, 96%, 95%, 90%, 85%, or 80% identical to the amino acids of SEQ ID NO: 24, and/or comprising nucleotides having at least 99%, 98%, 97%, 96%, 95%, 90%, 85%, or 80% identity to the nucleotides of SEQ ID NO: 26.
Embodiment 31. The method of embodiment 28, further comprising administering RNA encoding an IL-2 protein that is at least 99%, 98%, 97%, 96%, 95%, 90%, 85%, or 80% identical to the amino acids of SEQ ID NO: 9, and/or comprising nucleotides having at least 99%, 98%, 97%, 96%, 95%, 90%, 85%, or 80% identity to the nucleotides of SEQ ID NOs: 12 or 13.
Embodiment 32. The method of embodiment 28, further comprising administering RNA encoding
   a. an IL-15 sushi protein that is at least 99%, 98%, 97%, 96%, 95%, 90%, 85%, or 80% identical to the amino acids of SEQ ID NO: 24, and/or comprising nucleotides having at least 99%, 98%, 97%, 96%, 95%, 90%, 85%, or 80% identity to the nucleotides of SEQ ID NO: 26; and
   b. an IFNα2b protein that is at least 99%, 98%, 97%, 96%, 95%, 90%, 85%, or 80% identical to the amino acids of SEQ ID NO: 19, and/or comprising nucleotides having at least 99%, 98%, 97%, 96%, 95%, 90%, 85%, or 80% identity to the nucleotides of SEQ ID NOs: 22 or 23.
Embodiment 33. The method of embodiment 28, further comprising administering RNA encoding
   a. an IL-2 protein that is at least 99%, 98%, 97%, 96%, 95%, 90%, 85%, or 80% identical to the amino acids of SEQ ID NO: 9, and/or comprising nucleotides having at least 99%, 98%, 97%, 96%, 95%, 90%, 85%, or 80% identity to the nucleotides of SEQ ID NOs: 12 or 13; and
   b. an IFNα2b protein that is at least 99%, 98%, 97%, 96%, 95%, 90%, 85%, or 80% identical to the amino acids of SEQ ID NO: 19, and/or comprising nucleotides having at least 99%, 98%, 97%, 96%, 95%, 90%, 85%, or 80% identity to the nucleotides of SEQ ID NOs: 22 or 23.
Embodiment 34. The method of any one of embodiments 27-33, wherein the cancer is a sarcoma, carcinoma, or lymphoma.
Embodiment 35. The method of any one of embodiments 27-33, wherein the cancer is a solid tumor.
Embodiment 36. The method of embodiment 35, wherein the solid tumor is in the lung, colon, ovary, cervix, uterus, peritoneum, testicles, penis, tongue, lymph node, pancreas bone, breast, prostate, soft tissue, connective tissue, kidney, liver, brain, thyroid, or skin.
Embodiment 37. The method of embodiment 35, wherein the solid tumor is an epithelial tumor, Hodgkin lymphoma (HL), non-Hodgkin lymphoma, prostate tumor, ovarian tumor, renal cell tumor, gastrointestinal tract tumor, hepatic tumor, colorectal tumor, tumor with vasculature, mesothelioma tumor, pancreatic tumor, breast tumor, sarcoma tumor, lung tumor, colon tumor, brain tumor, melanoma tumor, small cell lung tumor, neuroblastoma, testicular tumor, carcinoma, adenocarcinoma, glioma tumor, seminoma tumor, retinoblastoma, or osteosarcoma tumor.
Embodiment 38. The method of any one of embodiments 27-37, wherein the composition is administered intra-tumorally or peri-tumorally.
Embodiment 39. The method of embodiment 38, wherein the injected tumor and a non-injected tumor are both reduced in size after intra- or peri-tumoral injection into or near the first tumor.
Embodiment 40. The method of any one of embodiments 27-39, wherein the subject is human.
Embodiment 41. The method of any one of embodiments 27-40, wherein another therapy is also administered.
Embodiment 42. The method of embodiment 41, wherein the other therapy is surgery to excise, resect, or debulk the tumor.
Embodiment 43. The method of embodiment 41, wherein the other therapy is immunotherapy, radiotherapy or chemotherapy.
Embodiment 44. The method of any one of embodiments 27-43, wherein at least one RNA comprises a modified nucleobase in place of at least one uridine.
Embodiment 45. The method of any one of embodiments 27-43, wherein each RNA comprises a modified nucleobase in place of each uridine.
Embodiment 46. The method of any one of embodiments 44-45, wherein the modified nucleobase is pseudouridine (ψ), N1-methyl-pseudouridine (m¹ψ), or 5-methyl-uridine (m⁵U).
Embodiment 47. The method of embodiment 46, wherein the modified nucleobase is N1-methyl-pseudouridine (m¹ψ).
Embodiment 48. The method of any one of embodiments 27-47, wherein at least one RNA further comprises a 5′ cap.
Embodiment 49. The method of any one of embodiments 27-47, wherein each RNA further comprises a 5′ cap.
Embodiment 50. The method of any one of embodiments 48-49, wherein the 5′ cap is m₂^{7,3′-O}Gppp(m₁^{2′-O})ApG or 3′-O-Me-m⁷G(5′)ppp(5′)G.
Embodiment 51. The method of any one of embodiments 27-50, wherein at least one RNA further comprises a 5′ UTR.
Embodiment 52. The method of any one of embodiments 27-50, wherein each RNA further comprises a 5′ UTR.
Embodiment 53. The method of any one of embodiments 51-52, wherein the 5′ UTR comprises or consists of the nucleotides of SEQ ID NOs: 2, 4, or 6, or nucleotides having at least 99%, 98%, 97%, 96%, 95%, 90%, or 85% identity to SEQ ID NOs: 2, 4, or 6.
Embodiment 54. The method of any one of embodiments 27-54, wherein at least one RNA further comprises a 3′ UTR.
Embodiment 55. The method of any one of embodiments 27-54, wherein each RNA further comprises a 3′ UTR.
Embodiment 56. The method of any one of embodiments 54-55, wherein the 3′ UTR comprises or consists of the nucleotides of SEQ ID NO: 8, or nucleotides having at least 99%, 98%, 97%, 96%, 95%, 90%, or 85% identity to SEQ ID NO: 8.
Embodiment 57. The method of any one of embodiments 27-56, wherein at least one RNA further comprises a poly-A tail.
Embodiment 58. The method of any one of embodiments 27-56, wherein each RNA further comprises a poly-A tail.
Embodiment 59. The method of any one of embodiments 57-58, wherein the poly-A tail comprises at least 100 nucleotides.
Embodiment 60. The method of any one of embodiments 27-59, wherein at least one RNA comprises a 5′ cap, 5′ UTR, 3′ UTR, and poly-A tail.
Embodiment 61. The method of any one of embodiments 27-59, wherein each RNA comprises a 5′ cap, 5′ UTR, 3′ UTR, and poly-A tail.
Embodiment 62. The method of any one of embodiments 60-61, wherein
   a. the 5′ cap is m₂^{7,3′-O}Gppp(m₁^{2′-O})ApG or 3′-O-Me-m⁷G(5′)ppp(5′)G;
   b. the 5′ UTR comprises or consists of the nucleotides of SEQ ID NOs: 2, 4, or 6, or nucleotides having at least 99%, 98%, 97%, 96%, 95%, 90%, or 85% identity to SEQ ID NOs: 2, 4, or 6;
   c. the 3′ UTR comprises or consists of the nucleotides of SEQ ID NO: 8, or nucleotides having at least 99%, 98%, 97%, 96%, 95%, 90%, or 85% identity to SEQ ID NO: 8; and
   d. the poly-A tail comprises at least 100 nucleotides.
Embodiment 63. A codon-optimized DNA comprising or consisting of contiguous nucleotides having at least 83% identity to SEQ ID NO: 11.
Embodiment 64. The DNA of embodiment 63, comprising or consisting of contiguous nucleotides having at least 99%, 98%, 97%, 96%, 95%, 90%, or 85% identity to SEQ ID NO: 11.
Embodiment 65. A codon-optimized RNA comprising or consisting of contiguous nucleotides having at least 83% identity to SEQ ID NO: 13.
Embodiment 66. The RNA of embodiment 65, comprising or consisting of contiguous nucleotides having at least 99%, 98%, 97%, 96%, 95%, 90%, or 85% identity to SEQ ID NO: 13.
Embodiment 67. An RNA produced from the DNA of any one of embodiments 63 or 64.
Embodiment 68. A codon-optimized DNA comprising or consisting of:
   a. contiguous nucleotides having at least 78% identity to nucleotides 1-984 of SEQ ID NO: 16;
   b. contiguous nucleotides having at least 81% identity to nucleotides 1027-1623 of SEQ ID NO: 16; and
   c. nucleotides encoding a linker between the nucleotides of a) and b).
Embodiment 69. The DNA of embodiment 68, wherein the linker comprises nucleotides 985-1026 of SEQ ID NO: 16.
Embodiment 70. The DNA of any one of embodiments 68 and 69, wherein part a) comprises contiguous nucleotides having at least 99%, 98%, 97%, 96%, 95%, 90%, 85%, 80%, or 75% identity to nucleotides 1-984 of SEQ ID NO: 16; and part b) comprises contiguous nucleotides having at least 99%, 98%, 97%, 96%, 95%, 90%, or 85% identity to nucleotides 1027-1623 of SEQ ID NO: 16.
Embodiment 71. A codon-optimized RNA comprising or consisting of:
   a. contiguous nucleotides having at least 78% identity to nucleotides 1-984 of SEQ ID NO: 18;
   b. contiguous nucleotides having at least 81% identity to nucleotides 1027-1623 of SEQ ID NO: 18; and
   c. nucleotides encoding a linker between the nucleotides of a) and b).
Embodiment 72. The RNA of embodiment 71, wherein the linker comprises nucleotides 985-1026 of SEQ ID NO: 18.
Embodiment 73. The RNA of any one of embodiments 71 and 72, wherein part a) comprises contiguous nucleotides having at least 99%, 98%, 97%, 96%, 95%, 90%, 85%, 80%, or 75% identity to nucleotides 1-984 of SEQ ID NO: 18; and part b) comprises contiguous nucleotides having at least 99%, 98%, 97%, 96%, 95%, 90%, or 85% identity to nucleotides 1027-1623 of SEQ ID NO: 18.
Embodiment 74. An RNA produced from the DNA of any one of embodiments 68-70.
Embodiment 75. A codon-optimized DNA comprising or consisting of contiguous nucleotides having at least 80% identity to SEQ ID NO: 21.
Embodiment 76. The DNA of embodiment 75, comprising or consisting of contiguous nucleotides having at least 99%, 98%, 97%, 96%, 95%, 90%, or 85% identity to SEQ ID NO: 21.
Embodiment 77. A codon-optimized RNA comprising or consisting of contiguous nucleotides having at least 80% identity to SEQ ID NO: 23.
Embodiment 78. The RNA of embodiment 77, comprising or consisting of contiguous nucleotides having at least 99%, 98%, 97%, 96%, 95%, 90%, or 85% identity to SEQ ID NO: 23.
Embodiment 79. An RNA produced from the DNA of any one of embodiments 75-76.
Embodiment 80. A DNA comprising or consisting of:
   a. contiguous nucleotides comprising or consisting of contiguous nucleotides having at least 99%, 98%, 97%, 96%, 95%, 90%, or 85% identity to nucleotides 1-321 of SEQ ID NO: 25;
   b. contiguous nucleotides comprising or consisting of contiguous nucleotides having at least 99%, 98%, 97%, 96%, 95%, 90%, or 85% identity to nucleotides 382-729 of SEQ ID NO: 25; and
   c. nucleotides encoding a linker between the nucleotides of a) and b).
Embodiment 81. An RNA produced from the DNA of embodiment 80.
Embodiment 82. A RNA comprising or consisting of:
   a. contiguous nucleotides comprising or consisting of contiguous nucleotides having at least 99%, 98%, 97%, 96%, 95%, 90%, or 85% identity to nucleotides 1-321 of SEQ ID NO: 26;
   b. contiguous nucleotides comprising or consisting of contiguous nucleotides having at least 99%, 98%, 97%, 96%, 95%, 90%, or 85% identity to nucleotides 382-729 of SEQ ID NO: 26; and
   c. nucleotides encoding a linker between the nucleotides of a) and b).
Embodiment 83. A DNA comprising or consisting of contiguous nucleotides having at least 99%, 98%, 97%, 96%, 95%, 90%, or 85% identity to SEQ ID NO: 28.
Embodiment 84. An RNA produced from the DNA of embodiment 83.
Embodiment 85. An RNA comprising or consisting of contiguous nucleotides having at least 99%, 98%, 97%, 96%, 95%, 90%, or 85% identity to SEQ ID NO: 29.
Embodiment 86. The RNA of any one of embodiments 67, 74, 79, 81, and 84 wherein the RNA is transcribed from the DNA in vitro.
Embodiment 87. The RNA of any one of embodiments 65-67, 71-74, 78-79, 81-82, and 84-85, wherein at least one uridine is replaced with a modified nucleobase.
Embodiment 88. The RNA of any one of embodiments 65-67, 71-74, 78-79, 81-82, and 84-85, wherein each uridine is replaced with a modified nucleobase.
Embodiment 89. The RNA of embodiment 87 or 88, wherein the modified nucleobase is pseudouridine (ψ), N1-methyl-pseudouridine (m¹ψ), or 5-methyl-uridine (m⁵U).
Embodiment 90. The RNA of embodiment 89, wherein the modified nucleobase is N1-methyl-pseudouridine (m¹ψ).
Embodiment 91. The RNA of any one of embodiments 65-67, 71-74, 78-79, 81-82, and 84-90, further comprising a 5′ cap.
Embodiment 92. The RNA of embodiment 91, wherein the 5′ cap is m₂^{7,3′-O}Gppp(m₁^{2′-O})ApG or 3′-O-Me-m⁷G(5′)ppp(5′)G.
Embodiment 93. The RNA of any one of embodiments 65-67, 71-74, 78-79, 81-82, and 84-92, further comprising a 5′ UTR.
Embodiment 94. The RNA of embodiment 93, wherein the 5′ UTR comprises or consists of the nucleotides of SEQ ID NOs: 2, 4, or 6, or nucleotides having at least 99%, 98%, 97%, 96%, 95%, 90%, or 85% identity to SEQ ID NOs: 2, 4, or 6.
Embodiment 95. The RNA of any one of embodiments 65-67, 71-74, 78-79, 81-82, and 84-94, further comprising a 3′ UTR.
Embodiment 96. The RNA of embodiment 95, wherein the 3′ UTR comprises or consists of the nucleotides of SEQ ID NO: 8, or nucleotides having at least 99%, 98%, 97%, 96%, 95%, 90%, or 85% identity to SEQ ID NO: 8.
Embodiment 97. The RNA of any one of embodiments 65-67, 71-74, 78-79, 81-82, and 84-96, further comprising a poly-A tail.
Embodiment 98. The RNA of embodiment 97, wherein the poly-A tail comprises at least 100 nucleotides.
Embodiment 99. The RNA of any one of embodiments 65-67, 71-74, 78-79, 81-82, and 84-97, further comprising a 5′ cap, 5′ UTR, 3′ UTR, and poly-A tail.
Embodiment 100. The RNA of embodiment 99, wherein
   a. the 5′ cap is m₂^{7,3′-O}Gppp(m₁^{2′-O})ApG or 3′-O-Me-m⁷G(5′)ppp(5′)G;
   b. the 5′ UTR comprises or consists of the nucleotides of SEQ ID NOs: 2, 4, or 6, or nucleotides having at least 99%, 98%, 97%, 96%, 95%, 90%, or 85% identity to SEQ ID NOs: 2, 4, or 6;
   c. the 3′ UTR comprises or consists of the nucleotides of SEQ ID NO: 8, or nucleotides having at least 99%, 98%, 97%, 96%, 95%, 90%, or 85% identity to SEQ ID NO: 8 and;
   d. the poly-A tail comprises at least 100 nucleotides.
Embodiment 101. A DNA comprising or consisting of:
   a. contiguous nucleotides comprising or consisting of nucleotides having at least 99%, 98%, 97%, 96%, 95%, 90%, 85% or 83% identity to SEQ ID NO: 11; and
   b. contiguous nucleotides comprising or consisting of nucleotides having at least 99%, 98%, 97%, 96%, 95%, 90%, 85% or 80% identity to SEQ ID NOs: 1, 3, or 5, wherein when transcribed, the nucleotides of parts a) and b) form a single transcript.
Embodiment 102. A DNA comprising or consisting of:
   a. contiguous nucleotides comprising or consisting of nucleotides having at least 99%, 98%, 97%, 96%, 95%, 90%, 85%, 80% or 78% identity to nucleotides 1-984 of SEQ ID NO: 16;
   b. contiguous nucleotides comprising or consisting of nucleotides having at least 99%, 98%, 97%, 96%, 95%, 90%, 85%, 80% or 81% identity to nucleotides 1027-1623 of SEQ ID NO: 16; and
   c. contiguous nucleotides comprising or consisting of nucleotides having at least 99%, 98%, 97%, 96%, 95%, 90%, 85% or 80% identity to SEQ ID NOs: 1, 3, or 5, wherein when transcribed, the nucleotides of part a), b) and c) form a single transcript.
Embodiment 103. A DNA comprising or consisting of:
   a. contiguous nucleotides comprising or consisting of nucleotides having at least 99%, 98%, 97%, 96%, 95%, 90%, 85% or 80% identity to SEQ ID NO: 21; and
   b. contiguous nucleotides comprising or consisting of nucleotides having at least 99%, 98%, 97%, 96%, 95%, 90%, 85% or 80% identity to SEQ ID NOs: 1, 3, or 5, wherein when transcribed, the nucleotides of part a) and b) form a single transcript.
Embodiment 104. A DNA comprising or consisting of:
   a. contiguous nucleotides comprising or consisting of nucleotides having at least 99%, 98%, 97%, 96%, 95%, 90%, 85% or 80% identity to SEQ ID NO: 28; and
   b. contiguous nucleotides comprising or consisting of nucleotides having at least 99%, 98%, 97%, 96%, 95%, 90%, 85% or 80% identity to SEQ ID NOs: 1, 3, or 5, wherein when transcribed, the nucleotides of part a) and b) form a single transcript.
Embodiment 105. A DNA comprising or consisting of:
   a. contiguous nucleotides comprising or consisting of nucleotides having at least 99%, 98%, 97%, 96%, 95%, 90%, 85% or 80% identity to nucleotides 1-321 of SEQ ID NO: 25;
   b. contiguous nucleotides comprising or consisting of nucleotides having at least 99%, 98%, 97%, 96%, 95%, 90%, 85% or 80% identity to nucleotides 382-729 of SEQ ID NO: 25; and
   c. contiguous nucleotides having at least 80% identity to SEQ ID NOs: 1, 3, or 5, wherein when transcribed, the nucleotides of part a), b), and c) form a single transcript.
Embodiment 106. A DNA comprising or consisting of:
   a. contiguous nucleotides comprising or consisting of nucleotides having at least 99%, 98%, 97%, 96%, 95%, 90%, 85% or 83% identity to SEQ ID NO: 11; and
   b. contiguous nucleotides comprising or consisting of nucleotides having at least 99%, 98%, 97%, 96%, 95%, 90%, 85% or 80% identity to SEQ ID NO: 7, wherein when transcribed, the nucleotides of part a) and b) form a single transcript.
Embodiment 107. A DNA comprising or consisting of:
   a. contiguous nucleotides comprising or consisting of nucleotides having at least 99%, 98%, 97%, 96%, 95%, 90%, 85%, 80% or 78% identity to nucleotides 1-984 of SEQ ID NO: 16;
   b. contiguous nucleotides comprising or consisting of nucleotides having at least 99%, 98%, 97%, 96%, 95%, 90%, 85%, 80% or 81% identity to nucleotides 1027-1623 of SEQ ID NO: 16; and
   c. contiguous nucleotides comprising or consisting of nucleotides having at least 99%, 98%, 97%, 96%, 95%, 90%, 85% or 80% identity to SEQ ID NO: 7, wherein when transcribed, the nucleotides of part a), b) and c) form a single transcript.
Embodiment 108. A DNA comprising or consisting of:
   a. contiguous nucleotides comprising or consisting of nucleotides having at least 99%, 98%, 97%, 96%, 95%, 90%, 85% or 80% identity to SEQ ID NO: 21; and
   b. contiguous nucleotides comprising or consisting of nucleotides having at least 99%, 98%, 97%, 96%, 95%, 90%, 85% or 80% identity to SEQ ID NO: 7, wherein when transcribed, the nucleotides of part a) and b) form a single transcript.
Embodiment 109. A DNA comprising or consisting of:
   a. contiguous nucleotides comprising or consisting of nucleotides having at least 99%, 98%, 97%, 96%, 95%, 90%, 85% or 80% identity to SEQ ID NO: 28; and
   b. contiguous nucleotides comprising or consisting of nucleotides having at least 99%, 98%, 97%, 96%, 95%, 90%, 85% or 80% identity to SEQ ID NO: 7, wherein when transcribed, the nucleotides of part a) and b) form a single transcript.
Embodiment 110. A DNA comprising or consisting of:
   a. contiguous nucleotides comprising or consisting of nucleotides having at least 99%, 98%, 97%, 96%, 95%, 90%, 85% or 80% identity to nucleotides 1-321 of SEQ ID NO: 25;
   b. contiguous nucleotides comprising or consisting of nucleotides having at least 99%, 98%, 97%, 96%, 95%, 90%, 85% or 80% identity to nucleotides 382-729 of SEQ ID NO: 25; and
   c. contiguous nucleotides comprising or consisting of nucleotides having at least 99%, 98%, 97%, 96%, 95%, 90%, 85% or 80% identity to SEQ ID NO: 7, wherein when transcribed, the nucleotides of part a), b), and c) form a single transcript.
Embodiment 111. A DNA comprising or consisting of:
   a. contiguous nucleotides comprising or consisting of nucleotides having at least 99%, 98%, 97%, 96%, 95%, 90%, 85% or 83% identity to SEQ ID NO: 11;
   b. contiguous nucleotides comprising or consisting of nucleotides having at least 99%, 98%, 97%, 96%, 95%, 90%, 85% or 80% identity to SEQ ID NOs: 1, 3, or 5; and
   c. contiguous nucleotides comprising or consisting of nucleotides having at least 99%, 98%, 97%, 96%, 95%, 90%, 85% or 80% identity to SEQ ID NO: 7, wherein when transcribed, the nucleotides of part a), b), and c) form a single transcript.
Embodiment 112. A DNA comprising or consisting of:
   a. contiguous nucleotides comprising or consisting of nucleotides having at least 99%, 98%, 97%, 96%, 95%, 90%, 85%, 80% or 78% identity to nucleotides 1-984 of SEQ ID NO: 16;
   b. contiguous nucleotides comprising or consisting of nucleotides having at least 99%, 98%, 97%, 96%, 95%, 90%, 85%, 80% or 81% identity to nucleotides 1027-1623 of SEQ ID NO: 16;
   c. contiguous nucleotides comprising or consisting of nucleotides having at least 99%, 98%, 97%, 96%, 95%, 90%, 85% or 80% identity to SEQ ID NOs: 1, 3, or 5; and
   d. contiguous nucleotides comprising or consisting of nucleotides having at least 99%, 98%, 97%, 96%, 95%, 90%, 85% or 80% identity to SEQ ID NO: 7, wherein when transcribed, the nucleotides of part a), b), c), and d) form a single transcript.
Embodiment 113. A DNA comprising or consisting of:
   a. contiguous nucleotides comprising or consisting of nucleotides having at least 99%, 98%, 97%, 96%, 95%, 90%, 85% or 80% identity to SEQ ID NO: 21;
   b. contiguous nucleotides comprising or consisting of nucleotides having at least 99%, 98%, 97%, 96%, 95%, 90%, 85% or 80% identity to SEQ ID NOs: 1, 3, or 5, wherein the nucleotides of part b) regulate the expression of the nucleotides of part a); and
   c. contiguous nucleotides comprising or consisting of nucleotides having at least 99%, 98%, 97%, 96%, 95%, 90%, 85% or 80% identity to SEQ ID NO: 7, wherein when transcribed, the nucleotides of part a), b) and c) form a single transcript.
Embodiment 114. A DNA comprising or consisting of:
   a. contiguous nucleotides comprising or consisting of nucleotides having at least 99%, 98%, 97%, 96%, 95%, 90%, 85% or 80% identity to SEQ ID NO: 28;
   b. contiguous nucleotides comprising or consisting of nucleotides having at least 99%, 98%, 97%, 96%, 95%, 90%, 85% or 80% identity to SEQ ID NOs: 1, 3, or 5; and
   c. contiguous nucleotides comprising or consisting of nucleotides having at least 99%, 98%, 97%, 96%, 95%, 90%, 85% or 80% identity to SEQ ID NO: 7, wherein when transcribed, the nucleotides of part a), b), and c) form a single transcript.
Embodiment 115. A DNA comprising or consisting of:
   a. contiguous nucleotides comprising or consisting of nucleotides having at least 99%, 98%, 97%, 96%, 95%, 90%, 85% or 80% identity to nucleotides 1-321 of SEQ ID NO: 25;
   b. contiguous nucleotides comprising or consisting of nucleotides having at least 99%, 98%, 97%, 96%, 95%, 90%, 85% or 80% identity to nucleotides 382-729 of SEQ ID NO: 25;
   c. contiguous nucleotides comprising or consisting of nucleotides having at least 99%, 98%, 97%, 96%, 95%, 90%, 85% or 80% identity to SEQ ID NOs: 1, 3, or 5; and
   d. contiguous nucleotides comprising or consisting of nucleotides having at least 99%, 98%, 97%, 96%, 95%, 90%, 85% or 80% identity to SEQ ID NO: 7, wherein when transcribed, the nucleotides of part a), b), c), and d) form a single transcript.
Embodiment 116. An RNA produced from any one of the DNAs of embodiments 101-115.
Embodiment 117. The RNA of embodiment 116, wherein at least one uridine is replaced with a modified nucleobase.
Embodiment 118. The RNA of embodiment 116, wherein each uridine is replaced with a modified nucleobase.
Embodiment 119. The RNA of any one of embodiments 117-118, wherein the modified nucleobase is pseudouridine (ψ), N1-methyl-pseudouridine (m¹ψ), or 5-methyl-uridine (m⁵U).
Embodiment 120. The RNA of embodiment 119, wherein the modified nucleobase is N1-methyl-pseudouridine (m¹ψ).
Embodiment 121. The RNA of any one of embodiments 116-120, wherein the RNA further comprises a 5′ cap.
Embodiment 122. The RNA of embodiment 121, wherein the 5′ cap is m₂^{7,3′-O}Gppp(m₁^{2′-O})ApG or 3′-O-Me-m⁷G(5′)ppp(5′)G.
Embodiment 123. The RNA of any of embodiments 116-122, wherein the RNA is substantially free of double-stranded RNA.
Embodiment 124. The RNA of any of embodiments 116-122, wherein double stranded RNA has been removed from the RNA.
Embodiment 125. The RNA of any one of embodiments 123-124, wherein the RNA has been purified via HPLC or cellulose-based chromatography.
Embodiment 126. A pharmaceutical formulation comprising any one of the DNA or RNAs of embodiments 63-125 and a pharmaceutically acceptable excipient.
Embodiment 127. A method for treating or preventing cancer, reducing the size of a tumor, preventing the reoccurrence of cancer in remission, or preventing cancer metastasis in a subject comprising administering any one or more of the RNAs or DNAs of embodiments 63-125, or the pharmaceutical formulation of embodiment 126.
Embodiment 128. A method of producing a polypeptide encoding IL-2, IL-12sc, IL-15 sushi, GM-CSF and IFNα2b in vivo comprising administering to a subject one or more of the DNAs or RNAs of any one of embodiment 63-125, the composition of any one of embodiments 1-26, or the pharmaceutical formulation of embodiment 126.
Embodiment 129. A composition comprising at least two RNAs, wherein the RNAs encode different proteins, and wherein the RNAs are selected from the RNAs of any one of embodiments 65-67, 71-74, 78-79, 81-82, 84-100, and 116-125.
Embodiment 130. The composition of embodiment 129, wherein the composition comprises two RNAs encoding GM-CSF and IL-12sc.
Embodiment 131. The composition of embodiment 129, wherein the composition comprises three RNAs encoding GM-CSF, IL-12sc, and IFNα2b.
Embodiment 132. The composition of embodiment 129, wherein the composition comprises three RNAs encoding GM-CSF, IL-2, and IFNα2b.
Embodiment 133. The composition of embodiment 129, wherein the composition comprises four RNAs encoding GM-CSF, IL-12sc, IL-2 and IFNα2b.
Embodiment 134. The composition of embodiment 129, wherein the composition comprises four RNAs encoding GM-CSF, IL-12sc, IL-15 sushi and IFNα2b.
Embodiment 135. The composition of any one of embodiments 130-134, wherein the RNA encoding GM-CSF is selected from the RNA of any one of embodiments 84-85.
Embodiment 136. The composition of any one of embodiments 130-134, wherein the RNA encoding IL-12sc is selected from the RNA of any one of embodiments 71-74.
Embodiment 137. The composition of any one of embodiments 131-134, wherein the RNA encoding IFNα2b is selected from the RNA of any one of embodiments 77-79.
Embodiment 138. The composition of embodiment 134, wherein the RNA encoding IL-15 sushi is selected from the RNA of any one of embodiments 81-82.
Embodiment 139. The composition of any one of embodiments 132-133, wherein the RNA encoding IL-2 is selected from the RNA of any one of embodiments 65-67.
Embodiment 140. A method of treating or preventing solid tumor cancer comprising administering a therapeutically effective amount of each of the following RNAs directly into a tumor:
   a. an RNA comprising an RNA encoding IL-12sc (SEQ ID NOs: 17 or 18);
   b. an RNA comprising an RNA encoding GM-CSF (SEQ ID NO: 29); and
   c. an RNA comprising an RNA encoding IFNα2b (SEQ ID NOs: 22 or 23),
      wherein each RNA comprises a modified nucleobase in place of each uridine, and wherein each RNA comprises a 5′ UTR (SEQ ID NOs: 2, 4, or 6), a 3′ UTR (SEQ ID NO: 8), a 5′ cap, and a poly-A tail.
Embodiment 141. A method of treating or preventing solid tumor cancer comprising administering a therapeutically effective amount of each of the following RNAs directly into a tumor:
   a. an RNA comprising an RNA encoding IL-12sc (SEQ ID NOs: 17 or 18);
   b. an RNA comprising an RNA encoding GM-CSF (SEQ ID NO: 29);
   c. an RNA comprising an RNA encoding IFNα2b (SEQ ID NOs: 22 or 23); and
   d. an RNA comprising an RNA encoding IL-2 (SEQ ID NOs: 12 or 13),
      wherein each RNA comprises a modified nucleobase in place of each uridine, and wherein each RNA comprises a 5′ UTR (SEQ ID NOs: 2, 4, or 6), a 3′ UTR (SEQ ID NO: 8), a 5′ cap, and a poly-A tail.
Embodiment 142. A method of treating or preventing solid tumor cancer comprising administering a therapeutically effective amount of each of the following RNAs directly into a tumor:
   a. an RNA comprising an RNA encoding IL-12sc (SEQ ID NOs: 17 or 18);
   b. an RNA comprising an RNA encoding GM-CSF (SEQ ID NO: 29);
   c. an RNA comprising an RNA encoding IFNα2b (SEQ ID NOs: 22 or 23); and
   d. an RNA comprising an RNA encoding IL-15 sushi (SEQ ID NO: 26),
      wherein each RNA comprises a modified nucleobase in place of each uridine, and wherein each RNA comprises a 5′ UTR (SEQ ID NOs: 2, 4, or 6), a 3′ UTR (SEQ ID NO: 8), a 5′ cap, and a poly-A tail.
Embodiment 143. A composition comprising an RNA encoding an IL-2 protein having at least 95% identity to the amino acids of SEQ ID NO: 9, wherein each uridine is replaced with a modified nucleobase, and further comprising a 5′ UTR (SEQ ID NOs: 2, 4, or 6), a 3′ UTR (SEQ ID NO: 8), a 5′ cap, and a poly-A tail.
Embodiment 144. A composition comprising an RNA encoding an IL-12sc protein having at least 95% identity to the amino acids of SEQ ID NO: 14, wherein each uridine is replaced with a modified nucleobase, and further comprising a 5′ UTR (SEQ ID NOs: 2, 4, or 6), a 3′ UTR (SEQ ID NO: 8), a 5′ cap, and a poly-A tail.
Embodiment 145. A composition comprising an RNA encoding a GM-CSF protein having at least 95% identity to the amino acids of SEQ ID NO: 27, wherein each uridine is replaced with a modified nucleobase, and further comprising a 5′ UTR (SEQ ID NOs: 2, 4, or 6), a 3′ UTR (SEQ ID NO: 8), a 5′ cap, and a poly-A tail.
Embodiment 146. A composition comprising an RNA encoding an IFNα2b protein having at least 95% identity to the amino acids of SEQ ID NO: 19, wherein each uridine is replaced with a modified nucleobase, and further comprising a 5′ UTR (SEQ ID NOs: 2, 4, or 6), a 3′ UTR (SEQ ID NO: 8), a 5′ cap, and a poly-A tail.
Embodiment 147. A composition comprising an RNA encoding an IL-15 sushi protein having at least 95% identity to the amino acids of SEQ ID NO: 24, wherein each uridine is replaced with a modified nucleobase, and further comprising a 5′ UTR (SEQ ID NOs: 2, 4, or 6), a 3′ UTR (SEQ ID NO: 8), a 5′ cap, and a poly-A tail.
Embodiment 148. An IL-12sc RNA composition comprising or consisting of nucleotides having at least 95% identity to SEQ ID NOs: 17 or 18, wherein each uridine is replaced with a modified nucleobase and further comprising a 5′ UTR (SEQ ID NOs: 2, 4, or 6), a 3′ UTR (SEQ ID NO: 8), a 5′ cap, and a poly-A tail.
Embodiment 149. A GM-CSF RNA composition comprising or consisting of nucleotides having at least 95% identity to SEQ ID NO: 29, wherein each uridine is replaced with a modified nucleobase, and further comprising a 5′ UTR (SEQ ID NOs: 2, 4, or 6), a 3′ UTR (SEQ ID NO: 8), a 5′ cap, and a poly-A tail.
Embodiment 150. An IFNα2b RNA composition comprising or consisting of nucleotides having at least 95% identity to SEQ ID NOs: 22 or 23, wherein each uridine is replaced with a uridine analog, and further comprising a 5′ UTR (SEQ ID NOs: 2, 4, or 6), a 3′ UTR (SEQ ID NO: 8), a 5′ cap, and a poly-A tail.
Embodiment 151. An IL-15 sushi RNA composition comprising or consisting of nucleotides having at least 95% identity to SEQ ID NO: 26, wherein each uridine is replaced with a modified nucleobase, and further comprising a 5′ UTR (SEQ ID NOs: 2, 4, or 6), a 3′ UTR (SEQ ID NO: 8), a 5′ cap, and a poly-A tail.
Embodiment 152. An IL-2 RNA composition comprising or consisting of nucleotides having at least 95% identity to SEQ ID NOs: 12 or 13, wherein each uridine is replaced with a modified nucleobase, and further comprising a 5′ UTR (SEQ ID NOs: 2, 4, or 6), a 3′ UTR (SEQ ID NO: 8), a 5′ cap, and a poly-A tail.
Embodiment 153. An IL-12sc RNA composition comprising or consisting of the nucleotides of SEQ ID NOs: 17 or 18, wherein each uridine is replaced with a modified nucleobase, and further comprising a 5′ UTR (SEQ ID NOs: 2, 4, or 6), a 3′ UTR (SEQ ID NO: 8), a 5′ cap, and a poly-A tail.
Embodiment 154. A GM-CSF RNA composition comprising or consisting of the nucleotides of SEQ ID NO: 29, wherein each uridine is replaced with a modified nucleobase, and further comprising a 5′ UTR (SEQ ID NOs: 2, 4, or 6), a 3′ UTR (SEQ ID NO: 8), a 5′ cap, and a poly-A tail.
Embodiment 155. An IFNα2b RNA composition comprising or consisting of the nucleotides of SEQ ID NOs: 22 or 23, wherein each uridine is replaced with a modified nucleobase, and further comprising a 5′ UTR (SEQ ID NOs: 2, 4, or 6), a 3′ UTR (SEQ ID NO: 8), a 5′ cap, and a poly-A tail.
Embodiment 156. An IL-15 sushi RNA composition comprising or consisting of the nucleotides of SEQ ID NO: 26, wherein each uridine is replaced with a modified nucleobase, and further comprising a 5′ UTR (SEQ ID NOs: 2, 4, or 6), a 3′ UTR (SEQ ID NO: 8), a 5′ cap, and a poly-A tail.
Embodiment 157. An IL-2 RNA composition comprising or consisting of the nucleotides of SEQ ID NOs: 12 or 13, wherein each uridine is replaced with a modified nucleobase, and further comprising a 5′ UTR (SEQ ID NOs: 2, 4, or 6), a 3′ UTR (SEQ ID NO: 8), a 5′ cap, and a poly-A tail.
Embodiment 158. The composition of any of embodiments 143-157, wherein the modified nucleobase is pseudouridine (ψ), N1-methyl-pseudouridine (m¹ψ), or 5-methyl-uridine (m⁵U).
Embodiment 159. A composition comprising an RNA encoding IFNα2b, wherein the RNA is altered to have reduced immunogenicity as compared to un-altered RNA.
Embodiment 160. The composition of embodiment 159, wherein the alteration comprises substitution of at least one uridine with a modified nucleobase.
Embodiment 161. The composition of embodiment 159, wherein the modified nucleobase is pseudouridine (ψ), N1-methyl-pseudouridine (m¹ψ), or 5-methyl-uridine (m⁵U).
Embodiment 162. The composition of embodiment 161, wherein the modified nucleobase is N1-methyl-pseudouridine (m¹ψ).
Embodiment 163. The composition of any one of embodiments 159-162, wherein the alteration comprises a reduction in the amount of double-stranded RNA.
Embodiment 164. The composition of embodiment 163, wherein the reduction in double-stranded RNA is the result of purification via HPLC or cellulose-based chromatography.
Embodiment 165. The composition of any one of embodiments 159-164, wherein the alteration reduces RNA recognition by an innate immune system as compared to un-altered RNA.
Embodiment 166. The composition of any one of embodiments 159-165, wherein the alteration comprises addition of a 5′ cap to the RNA.
Embodiment 167. The composition of embodiment 166, wherein the 5′ cap is m₂^{7,3′-O}Gppp(m₁^{2′-O})ApG or 3′-O-Me-m⁷G(5′)ppp(5′)G.
Embodiment 168. The composition of any one of embodiments 159-167, further comprising a second RNA encoding a peptide or protein of interest.
Embodiment 169. The composition of embodiment 168, wherein the peptide or protein of interest is a peptide or protein selected or derived from cytokines, chemokines, suicide gene products, immunogenic proteins or peptides, apoptosis inducers, angiogenesis inhibitors, heat shock proteins, tumor antigens, β-catenin inhibitors, activators of the STING pathway, activators of the retinoic inducible gene (RIG)-I pathway, agonists of toll-like receptor (TLR) pathways, checkpoint modulators, innate immune activators, antibodies, dominant negative receptors and decoy receptors, inhibitors of myeloid derived suppressor cells (MDSCs), IDO pathway inhibitors, and proteins or peptides that bind inhibitors of apoptosis.
Embodiment 170. The composition of embodiment 168 or 169, wherein the second RNA is altered to have reduced immunogenicity as compared to un-altered RNA.
Embodiment 171. The composition of embodiment 170, wherein the alteration comprises substitution of at least one uridine with a modified nucleobase.
Embodiment 172. The composition of embodiment 171, wherein the modified nucleobase is pseudouridine (ψ), N1-methyl-pseudouridine (m¹ψ) or 5-methyl-uridine (m⁵U).
Embodiment 173. The composition of embodiment 172, wherein the modified nucleobase is N1-methyl-pseudouridine (m¹ψ).
Embodiment 174. The composition of any one of embodiments 168-173, wherein the alteration comprises a reduction in the amount of double-stranded RNA.
Embodiment 175. The composition of embodiment 174, wherein the reduction in double-stranded RNA is the result of purification via HPLC or cellulose-based chromatography.
Embodiment 176. The composition of any one of embodiments 168-173, wherein the alteration comprises addition of a 5′ cap to the RNA.
Embodiment 177. The composition of embodiment 176, wherein the 5′ cap is m₂^{7,3′-O}Gppp(m₁^{2′-O})ApG or 3′-O-Me-m⁷G(5′)ppp(5′)G.
Embodiment 178. The composition of any one of embodiments 168-177, wherein the second RNA comprises:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18;
   b. an RNA encoding a GM-CSF protein having at least 95% identity to the amino acid sequence of SEQ ID NO: 27, and/or comprising nucleotides having at least 95% identity to the nucleotides of SEQ ID NO: 29;
   c. an RNA encoding an IL-15 sushi protein having at least 95% identity to the amino acid sequence of SEQ ID NO: 24, and/or comprising nucleotides having at least 95% identity to the nucleotides of SEQ ID NO: 26; or
   d. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 179. A method for treating or preventing cancer, reducing the size of a tumor, preventing the reoccurrence of cancer in remission, or preventing cancer metastasis in a subject comprising administering any one of the compositions of embodiments 159-178.
Embodiment 180. The composition of any one of embodiments 159-178 for use in a method of treating or preventing cancer, reducing the size of a tumor, preventing the reoccurrence of cancer in remission, or preventing cancer metastasis.
Embodiment 181. A composition comprising an RNA encoding an IL-12sc protein having at least 95% identity to the amino acid sequence of SEQ ID NO: 14.
Embodiment 182. A composition comprising an RNA encoding a GM-CSF protein having at least 95% identity to the amino acid sequence of SEQ ID NO: 27.
Embodiment 183. A composition comprising an RNA encoding an IFNα2b protein having at least 95% identity to the amino acid sequence of SEQ ID NO: 19.
Embodiment 184. A composition comprising an RNA encoding an IL-15 sushi protein having at least 95% identity to the amino acid sequence of SEQ ID NO: 24.
Embodiment 185. A composition comprising an RNA encoding an IL-2 protein having at least 95% identity to the amino acid sequence of SEQ ID NO: 9.
Embodiment 186. A composition comprising an RNA encoding an IL-12sc protein, wherein the RNA comprises nucleotides having at least 95% identity to the nucleotides of SEQ ID NOs: 17 or 18.
Embodiment 187. A composition comprising an RNA encoding a GM-CSF protein, wherein the RNA comprises nucleotides having at least 95% identity to the nucleotides of SEQ ID NO: 29.
Embodiment 188. A composition comprising an RNA encoding an IFNα2b protein, wherein the RNA comprises nucleotides having at least 95% identity to the nucleotides of SEQ ID NOs: 22 or 23.
Embodiment 189. A composition comprising an RNA encoding an IL-15 sushi protein, wherein the RNA comprises nucleotides having at least 95% identity to the nucleotides of SEQ ID NO: 26.
Embodiment 190. A composition comprising an RNA encoding an IL-2 protein, wherein the RNA comprises nucleotides having at least 95% identity to the nucleotides of SEQ ID NOs: 12 or 13.
Embodiment 191. A composition comprising any two of the following RNAs:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18;
   b. an RNA encoding a GM-CSF protein having at least 95% identity to the amino acid sequence of SEQ ID NO: 27, and/or comprising nucleotides having at least 95% identity to the nucleotides of SEQ ID NO: 29;
   c. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23;
   d. an RNA encoding an IL-15 sushi protein having at least 95% identity to the amino acid sequence of SEQ ID NO: 24, and/or comprising nucleotides having at least 95% identity to the nucleotides of SEQ ID NO: 26; and
   e. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 192. A composition comprising:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18; and
   b. an RNA encoding a GM-CSF protein having at least 95% identity to the amino acid sequence of SEQ ID NO: 27, and/or comprising nucleotides having at least 95% identity to the nucleotides of SEQ ID NO: 29.
Embodiment 193. A composition comprising:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18; and
   b. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23.
Embodiment 194. A composition comprising:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18; and
   b. an RNA encoding an IL-15 sushi protein having at least 95% identity to the amino acid sequence of SEQ ID NO: 24, and/or comprising nucleotides having at least 95% identity to the nucleotides of SEQ ID NO: 26.
Embodiment 195. A composition comprising:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18; and
   b. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 196. A composition comprising:
   a. an RNA encoding a GM-CSF protein having at least 95% identity to the amino acid sequence of SEQ ID NO: 27, and/or comprising nucleotides having at least 95% identity to the nucleotides of SEQ ID NO: 29; and
   b. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23.
Embodiment 197. A composition comprising:
   a. an RNA encoding a GM-CSF protein having at least 95% identity to the amino acid sequence of SEQ ID NO: 27, and/or comprising nucleotides having at least 95% identity to the nucleotides of SEQ ID NO: 29; and
   b. an RNA encoding an IL-15 sushi protein having at least 95% identity to the amino acid sequence of SEQ ID NO: 24, and/or comprising nucleotides having at least 95% identity to the nucleotides of SEQ ID NO: 26.
Embodiment 198. A composition comprising:
   a. an RNA encoding a GM-CSF protein having at least 95% identity to the amino acid sequence of SEQ ID NO: 27, and/or comprising nucleotides having at least 95% identity to the nucleotides of SEQ ID NO: 29; and
   b. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 199. A composition comprising:
   a. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23; and
   b. an RNA encoding an IL-15 sushi protein having at least 95% identity to the amino acid sequence of SEQ ID NO: 24, and/or nucleotides having at least 95% identity to the nucleotides of SEQ ID NO: 26.
Embodiment 200. A composition comprising:
   a. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23; and
   b. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 201. A composition comprising:
   a. an RNA encoding an IL-15 sushi protein having at least 95% identity to the amino acid sequence of SEQ ID NO: 24, and/or comprising nucleotides having at least 95% identity to the nucleotides of SEQ ID NO: 26; and
   b. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 202. A composition comprising any three of the following:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18;
   b. an RNA encoding a GM-CSF protein having at least 95% identity to the amino acid sequence of SEQ ID NO: 27, and/or comprising nucleotides having at least 95% identity to the nucleotides of SEQ ID NO: 29;
   c. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23;
   d. an RNA encoding an IL-15 sushi protein having at least 95% identity to the amino acid sequence of SEQ ID NO: 24, and/or comprising nucleotides having at least 95% identity to the nucleotides of SEQ ID NO: 26; and
   a. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 203. A composition comprising:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18;
   b. an RNA encoding a GM-CSF protein having at least 95% identity to the amino acid sequence of SEQ ID NO: 27, and/or comprising nucleotides having at least 95% identity to the nucleotides of SEQ ID NO: 29; and
   c. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23.
Embodiment 204. A composition comprising:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18;
   b. an RNA encoding a GM-CSF protein having at least 95% identity to the amino acid sequence of SEQ ID NO: 27, and/or comprising nucleotides having at least 95% identity to the nucleotides of SEQ ID NO: 29; and
   c. an RNA encoding an IL-15 sushi protein having at least 95% identity to the amino acid sequence of SEQ ID NO: 24, and/or comprising nucleotides having at least 95% identity to the nucleotides of SEQ ID NO: 26.
Embodiment 205. A composition comprising:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18;
   b. an RNA encoding a GM-CSF protein having at least 95% identity to the amino acid sequence of SEQ ID NO: 27, and/or comprising nucleotides having at least 95% identity to the nucleotides of SEQ ID NO: 29; and
   c. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 206. A composition comprising:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18;
   b. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23; and
   c. an RNA encoding an IL-15 sushi protein having at least 95% identity to the amino acid sequence of SEQ ID NO: 24, and/or comprising nucleotides having at least 95% identity to the nucleotides of SEQ ID NO: 26.
Embodiment 207. A composition comprising:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18;
   b. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23; and
   c. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 208. A composition comprising:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18;
   b. an RNA encoding an IL-15 sushi protein having at least 95% identity to the amino acid sequence of SEQ ID NO: 24, and/or comprising nucleotides having at least 95% identity to the nucleotides of SEQ ID NO: 26; and
   c. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 209. A composition comprising:
   a. an RNA encoding a GM-CSF protein having at least 95% identity to the amino acid sequence of SEQ ID NO: 27, and/or comprising nucleotides having at least 95% identity to the nucleotides of SEQ ID NO: 29;
   b. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23; and
   c. an RNA encoding an IL-15 sushi protein having at least 95% identity to the amino acid sequence of SEQ ID NO: 24, and/or comprising nucleotides having at least 95% identity to the nucleotides of SEQ ID NO: 26.
Embodiment 210. A composition comprising:
   a. an RNA encoding a GM-CSF protein having at least 95% identity to the amino acid sequence of SEQ ID NO: 27, and/or comprising nucleotides having at least 95% identity to the nucleotides of SEQ ID NO: 29;
   b. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23; and
   c. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 211. A composition comprising:
   a. an RNA encoding a GM-CSF protein having at least 95% identity to the amino acid sequence of SEQ ID NO: 27, and/or comprising nucleotides having at least 95% identity to the nucleotides of SEQ ID NO: 29;
   b. an RNA encoding an IL-15 sushi protein having at least 95% identity to the amino acid sequence of SEQ ID NO: 24, and/or comprising nucleotides having at least 95% identity to the nucleotides of SEQ ID NO: 26; and
   c. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 212. A composition comprising:
   a. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23;
   b. an RNA encoding an IL-15 sushi protein having at least 95% identity to the amino acid sequence of SEQ ID NO: 24, and/or comprising nucleotides having at least 95% identity to the nucleotides of SEQ ID NO: 26; and
   c. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 213. A composition comprising any four of the following:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18;
   b. an RNA encoding a GM-CSF protein having at least 95% identity to the amino acid sequence of SEQ ID NO: 27, and/or comprising nucleotides having at least 95% identity to the nucleotides of SEQ ID NO: 29;
   c. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23;
   d. an RNA encoding an IL-15 sushi protein having at least 95% identity to the amino acid sequence of SEQ ID NO: 24, and/or comprising nucleotides having at least 95% identity to the nucleotides of SEQ ID NO: 26; and
   e. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 214. A composition comprising:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18;
   b. an RNA encoding a GM-CSF protein having at least 95% identity to the amino acid sequence of SEQ ID NO: 27, and/or comprising nucleotides having at least 95% identity to the nucleotides of SEQ ID NO: 29;
   c. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23; and
   d. an RNA encoding an IL-15 sushi protein having at least 95% identity to the amino acid sequence of SEQ ID NO: 24, and/or comprising nucleotides having at least 95% identity to the nucleotides of SEQ ID NO: 26.
Embodiment 215. A composition comprising:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18;
   b. an RNA encoding a GM-CSF protein having at least 95% identity to the amino acid sequence of SEQ ID NO: 27, and/or comprising nucleotides having at least 95% identity to the nucleotides of SEQ ID NO: 29;
   c. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23; and
   d. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 216. A composition comprising:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18;
   b. an RNA encoding a GM-CSF protein having at least 95% identity to the amino acid sequence of SEQ ID NO: 27, and/or comprising nucleotides having at least 95% identity to the nucleotides of SEQ ID NO: 29;
   c. an RNA encoding an IL-15 sushi protein having at least 95% identity to the amino acid sequence of SEQ ID NO: 24, and/or comprising nucleotides having at least 95% identity to the nucleotides of SEQ ID NO: 26; and
   d. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 217. A composition comprising:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18;
   b. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23;
   c. an RNA encoding an IL-15 sushi protein having at least 95% identity to the amino acid sequence of SEQ ID NO: 24, and/or comprising nucleotides having at least 95% identity to the nucleotides of SEQ ID NO: 26; and
   d. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 218. A composition comprising:
   a. an RNA encoding a GM-CSF protein having at least 95% identity to the amino acid sequence of SEQ ID NO: 27, and/or comprising nucleotides having at least 95% identity to the nucleotides of SEQ ID NO: 29;
   b. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23;
   c. an RNA encoding an IL-15 sushi protein having at least 95% identity to the amino acid sequence of SEQ ID NO: 24, and/or comprising nucleotides having at least 95% identity to the nucleotides of SEQ ID NO: 26; and
   d. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 219. A composition comprising:
   a. an RNA encoding an IL-12sc protein having at least 95% identity to the amino acid sequence of SEQ ID NO: 14, and/or comprising nucleotides having at least 95% identity to the nucleotides of SEQ ID NOs: 17 or 18;
   b. an RNA encoding a GM-CSF protein having at least 95% identity to the amino acid sequence of SEQ ID NO: 27, and/or comprising nucleotides having at least 95% identity to the nucleotides of SEQ ID NO: 29;
   c. an RNA encoding an IFNα2b protein having at least 95% identity to the amino acid sequence of SEQ ID NO: 19, and/or comprising nucleotides having at least 95% identity to the nucleotides of SEQ ID NOs: 22 or 23;
   d. an RNA encoding an IL-15 sushi protein having at least 95% identity to the amino acid sequence of SEQ ID NO: 24, and/or comprising nucleotides having at least 95% identity to the nucleotides of SEQ ID NO: 26; and
   e. an RNA encoding an IL-2 protein having at least 95% identity to the amino acid sequence of SEQ ID NO: 9, and/or comprising nucleotides having at least 95% identity to the nucleotides of SEQ ID NOs: 12 or 13.
Embodiment 220. A pharmaceutical formulation comprising any one of the compositions of embodiments 181-219.
Embodiment 221. A pharmaceutical formulation comprising any one of the compositions of embodiments 181-219 and a pharmaceutically acceptable excipient.
Embodiment 222. The composition of any one of embodiments 181-219, or the pharmaceutical formulation of embodiment 220 or 221, for use in a method of treating or preventing cancer.
Embodiment 223. The composition of any one of embodiments 181-219, or the pharmaceutical formulation of embodiment 220 or 221 for use in a method of reducing the size of a tumor.
Embodiment 224. The composition of any one of embodiments 181-219, or the pharmaceutical formulation of embodiment 220 or 221 for use in a method of preventing the reoccurrence of cancer in remission.
Embodiment 225. The composition of any one of embodiments 181-219, or the pharmaceutical formulation of embodiment 220 or 221 for use in a method of preventing cancer metastasis.
Embodiment 226. A method for treating or preventing cancer comprising administering the composition of any one of embodiments 181-219, or the pharmaceutical formulation of embodiment 220 or 221.
Embodiment 227. A method for reducing the size of a tumor comprising administering the composition of any one of embodiments 181-219, or the pharmaceutical formulation of embodiment 220 or 221.
Embodiment 228. A method for preventing the reoccurrence of cancer in remission comprising administering the composition of any one of embodiments 181-219, or the pharmaceutical formulation of embodiment 220 or 221.
Embodiment 229. A method for preventing cancer metastasis comprising administering the composition of any one of embodiments 181-219, or the pharmaceutical formulation of embodiment 220 or 221.

Further embodiments of the present invention are as follows:
Embodiment A 1. A medical preparation comprising RNA encoding an IL-12sc protein and RNA encoding a GM-CSF protein.
Embodiment A 2. The medical preparation of embodiment A 1, further comprising RNA encoding an IL-15 sushi protein.
Embodiment A 3. The medical preparation of embodiment A 1 or 2, further comprising RNA encoding an IL-2 protein.
Embodiment A 4. The medical preparation of any one of embodiments A 1 to 3, further comprising RNA encoding an IFNα protein.
Embodiment A 5. The medical preparation of embodiment A 4, wherein the IFNα protein is an IFNα2b protein.
Embodiment A 6. The medical preparation of embodiment A 2, comprising RNA encoding an IL-12sc protein, RNA encoding a GM-CSF protein, and RNA encoding an IL-15 sushi protein.
Embodiment A 7. The medical preparation of embodiment A 3, comprising RNA encoding an IL-12sc protein, RNA encoding a GM-CSF protein, and RNA encoding an IL-2 protein.
Embodiment A 8. The medical preparation of embodiment A 4 or 5, comprising RNA encoding an IL-12sc protein, RNA encoding a GM-CSF protein, and RNA encoding an IFNα protein.
Embodiment A 9. The medical preparation of embodiment A 4 or 5, comprising RNA encoding an IL-12sc protein, RNA encoding a GM-CSF protein, RNA encoding an IL-15 sushi protein, and RNA encoding an IFNα protein.
Embodiment A 10. The medical preparation of embodiment A 4 or 5, comprising RNA encoding an IL-12sc protein, RNA encoding a GM-CSF protein, RNA encoding an IL-2 protein, and RNA encoding an IFNα protein.
Embodiment A 11. The medical preparation of any one of embodiments A 1-10, wherein (i) the RNA encoding an IL-12sc protein comprises the nucleotide sequence of SEQ ID NO: 17 or 18, or a nucleotide sequence having at least 99%, 98%, 97%, 96%, 95%, 90%, 85%, or 80% identity to the nucleotide sequence of SEQ ID NO: 17 or 18 and/or (ii) the IL-12sc protein comprises the amino acid sequence of SEQ ID NO: 14, or an amino acid sequence having at least 99%, 98%, 97%, 96%, 95%, 90%, 85%, or 80% identity to the amino acid sequence of SEQ ID NO: 14.
Embodiment A 12. The medical preparation of any one of embodiments A 1-11, wherein (i) the RNA encoding a GM-CSF protein comprises the nucleotide sequence of SEQ ID NO: 29, or a nucleotide sequence having at least 99%, 98%, 97%, 96%, 95%, 90%, 85%, or 80% identity to the nucleotide sequence of SEQ ID NO: 29 and/or (ii) the GM-CSF protein comprises the amino acid sequence of SEQ ID NO: 27, or an amino acid sequence having at least 99%, 98%, 97%, 96%, 95%, 90%, 85%, or 80% identity to the amino acid sequence of SEQ ID NO: 27.
Embodiment A 13. The medical preparation of any one of embodiments A 2-12, wherein (i) the RNA encoding an IL-15 sushi protein comprises the nucleotide sequence of SEQ ID NO: 26, or a nucleotide sequence having at least 99%, 98%, 97%, 96%, 95%, 90%, 85%, or 80% identity to the nucleotide sequence of SEQ ID NO: 26 and/or (ii) the IL-15 sushi protein comprises the amino acid sequence of SEQ ID NO: 24, or an amino acid sequence having at least 99%, 98%, 97%, 96%, 95%, 90%, 85%, or 80% identity to the amino acid sequence of SEQ ID NO: 24.
Embodiment A 14. The medical preparation of any one of embodiments A 3-13, wherein (i) the RNA encoding an IL-2 protein comprises the nucleotide sequence of SEQ ID NO: 12 or 13, or a nucleotide sequence having at least 99%, 98%, 97%, 96%, 95%, 90%, 85%, or 80% identity to the nucleotide sequence of SEQ ID NO: 12 or 13 and/or (ii) the IL-2 protein comprises the amino acid sequence of SEQ ID NO: 9, or an amino acid sequence having at least 99%, 98%, 97%, 96%, 95%, 90%, 85%, or 80% identity to the amino acid sequence of SEQ ID NO: 9.
Embodiment A 15. The medical preparation of any one of embodiments 4-14, wherein (i) the RNA encoding an IFNα protein comprises the nucleotide sequence of SEQ ID NO: 22 or 23, or a nucleotide sequence having at least 99%, 98%, 97%, 96%, 95%, 90%, 85%, or 80% identity to the nucleotide sequence of SEQ ID NO: 22 or 23 and/or (ii) the IFNα protein comprises the amino acid sequence of SEQ ID NO: 19, or an amino acid sequence having at least 99%, 98%, 97%, 96%, 95%, 90%, 85%, or 80% identity to the amino acid sequence of SEQ ID NO: 19.
Embodiment A 16. The medical preparation of any one of embodiments A 1-15, wherein at least one RNA comprises a modified nucleobase in place of at least one uridine.
Embodiment A 17. The medical preparation of any one of embodiments A 1-16, wherein each RNA comprises a modified nucleobase in place of at least one uridine.
Embodiment A 18. The medical preparation of any one of embodiments A 1-17, wherein each RNA comprises a modified nucleobase in place of each uridine.
Embodiment A 19. The medical preparation of any one of embodiments A 16-18, wherein the modified nucleobase is pseudouridine (ψ), N1-methyl-pseudouridine (m1ψ) or 5-methyl-uridine (m5U).
Embodiment A 20. The medical preparation of embodiment A 19, wherein the modified nucleobase is N1-methyl-pseudouridine (m¹ψ).
Embodiment A 21. The medical preparation of any one of embodiments A 1-20, wherein at least one RNA comprises a 5′ cap.
Embodiment A 22. The medical preparation of any one of embodiments A 1-21, wherein each RNA comprises a 5′ cap.
Embodiment A 23. The medical preparation of embodiment A 21 or 22, wherein the 5′ cap is m₂^{7,3′-O}Gppp(m₁^{2′-O})ApG or 3′-O-Me-m⁷G(5′)ppp(5′)G.
Embodiment A 24. The medical preparation of any one of embodiments A 1-23, wherein at least one RNA comprises a 5′ UTR.
Embodiment A 25. The medical preparation of any one of embodiments A 1-24, wherein each RNA comprises a 5′ UTR.
Embodiment A 26. The medical preparation of embodiment A 24 or 25, wherein the 5′ UTR comprises a nucleotide sequence selected from the group consisting of SEQ ID NOs: 2, 4, and 6, or a nucleotide sequence having at least 99%, 98%, 97%, 96%, 95%, 90%, 85%, or 80% identity to a nucleotide sequence selected from the group consisting of SEQ ID NOs: 2, 4, and 6.
Embodiment A 27. The medical preparation of any one of embodiments A 1-26, wherein at least one RNA comprises a 3′ UTR.
Embodiment A 28. The medical preparation of any one of embodiments A 1-27, wherein each RNA comprises a 3′ UTR.
Embodiment A 29. The medical preparation of embodiment A 27 or 28, wherein the 3′ UTR comprises the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8.
Embodiment A 30. The medical preparation of any one of embodiments A 1-29, wherein at least one RNA comprises a poly-A tail.
Embodiment A 31. The medical preparation of any one of embodiments A 1-30, wherein each RNA comprises a poly-A tail.
Embodiment A 32. The medical preparation of embodiment A 30 or 31, wherein the poly-A tail comprises at least 100 nucleotides.
Embodiment A 33. The medical preparation of any one of embodiments A 1-32, wherein at least one RNA comprises a 5′ cap, a 5′ UTR, a 3′ UTR, and a poly-A tail.
Embodiment A 34. The medical preparation of any one of embodiments A 1-33, wherein each RNA comprises a 5′ cap, a 5′ UTR, a 3′ UTR, and a poly-A tail.
Embodiment A 35. The medical preparation of embodiment A 33 or 34, wherein
   a. the 5′ cap is m₂^{7,3′-O}Gppp(m₁^{2′-O})ApG or 3′-O-Me-m⁷G(5′)ppp(5′)G;
   b. the 5′ UTR comprises a nucleotide sequence selected from the group consisting of SEQ ID NOs: 2, 4, and 6, or a nucleotide sequence having at least 99%, 98%, 97%, 96%, 95%, 90%, 85%, or 80% identity to a nucleotide sequence selected from the group consisting of SEQ ID NOs: 2, 4, and 6;
   c. the 3′ UTR comprises the nucleotide sequence of SEQ ID NO: 8, or a nucleotide sequence having at least 99%, 98%, 97%, 96%, 95%, 90%, 85%, or 80% identity to the nucleotide sequence of SEQ ID NO:8; and
   d. the poly-A tail comprises at least 100 nucleotides.
Embodiment A 36. The medical preparation of any one of embodiments A 1 to 35, wherein the RNA is mRNA.
Embodiment A 37. The medical preparation of any one of embodiments A 1 to 36, which comprises a further therapeutic agent.
Embodiment A 38. The medical preparation of embodiment A 37, wherein the further therapeutic agent is an anti-cancer therapeutic agent.
Embodiment A 39. The medical preparation of embodiment A 37 or 38, wherein the further therapeutic agent is a checkpoint modulator.
Embodiment A 40. The medical preparation of embodiment A 39, wherein the checkpoint modulator is an anti-PD1 antibody, an anti-CTLA-4 antibody, or a combination of an anti-PD1 antibody and an anti-CTLA-4 antibody.
Embodiment A 41. The medical preparation of any one of embodiments A 1 to 40, which is a kit comprising at least two containers, each container comprising at least one of said RNAs.
Embodiment A 42. The medical preparation of embodiment A 41, which comprises each RNA in a separate container.
Embodiment A 43. The medical preparation of embodiment A 41 or 42, wherein the further therapeutic agent is in a container not comprising the RNA.
Embodiment A 44. The medical preparation of any one of embodiments A 41-43, further comprising instructions for use of the medical preparation for treating or preventing cancer.
Embodiment A 45. The medical preparation of any one of embodiments A 1 to 40, which is a pharmaceutical composition comprising the RNAs.
Embodiment A 46. The medical preparation of embodiment A 45, wherein the pharmaceutical composition further comprises one or more pharmaceutically acceptable carriers, diluents and/or excipients.
Embodiment A 47. The medical preparation of any one of embodiments A 1 to 46, wherein the RNA is present in a form selected from a liquid form, a solid form, or a combination thereof.
Embodiment A 48. The medical preparation of embodiment A 47, wherein the solid form is a frozen form or a dehydrated form.
Embodiment A 49. The medical preparation of embodiment A 48, wherein the dehydrated form is a freeze-dried or spray-dried form.
Embodiment A 50. The medical preparation of any one of embodiments A 1 to 49 for pharmaceutical use.
Embodiment A 51. The medical preparation of embodiment A 50, wherein the pharmaceutical use comprises a therapeutic or prophylactic treatment of a disease or disorder.
Embodiment A 52. The medical preparation of any one of embodiments A 1 to 51 for use in a method for treating or preventing cancer.
Embodiment A 53. The medical preparation of any one of embodiments A 38-52, wherein the cancer is a sarcoma, carcinoma, or lymphoma.
Embodiment A 54. The medical preparation of any one of embodiments A 38-53, wherein the cancer is a solid tumor.
Embodiment A 55. The medical preparation of embodiment A 54, wherein the solid tumor is in the lung, colon, ovary, cervix, uterus, peritoneum, testicles, penis, tongue, lymph node, pancreas, bone, breast, prostate, soft tissue, connective tissue, kidney, liver, brain, thyroid, or skin.
Embodiment A 56. The medical preparation of embodiment A 54 or 55, wherein the solid tumor is an epithelial tumor, Hodgkin lymphoma (HL), non-Hodgkin lymphoma, prostate tumor, ovarian tumor, renal cell tumor, gastrointestinal tract tumor, hepatic tumor, colorectal tumor, tumor with vasculature, mesothelioma tumor, pancreatic tumor, breast tumor, sarcoma tumor, lung tumor, colon tumor, brain tumor, melanoma tumor, small cell lung tumor, neuroblastoma tumor, testicular tumor, carcinoma tumor, adenocarcinoma tumor, glioma tumor, seminoma tumor, retinoblastoma, or osteosarcoma tumor.
Embodiment A 57. The medical preparation of any one of embodiments A 1-56, wherein the RNA is for intra-tumoral or peri-tumoral administration.
Embodiment A 58. The medical preparation of any one of embodiments A 37-57, wherein the further therapeutic agent is for systemic administration.
Embodiment A 59. The medical preparation of any one of embodiments A 1-58, which is for administration to a human.
Embodiment A 60. The medical preparation of any one of embodiments A 44 and 47-59, wherein treating or preventing cancer comprises reducing the size of a tumor, preventing the reoccurrence of cancer in remission, or preventing cancer metastasis in a subject.

Further embodiments of the present invention are as follows:
Embodiment B 1. RNA for use in a method for treating or preventing cancer in a subject, wherein the method comprises administering RNA encoding an IL-12sc protein and RNA encoding a GM-CSF protein.
Embodiment B 2. The RNA of Embodiment B 1, wherein the method further comprises administering RNA encoding an IL-15 sushi protein.
Embodiment B 3. The RNA of Embodiment B 1 or 2, wherein the method further comprises administering RNA encoding an IL-2 protein.
Embodiment B 4. The RNA of any one of embodiments B 1 to 3, wherein the method further comprises administering RNA encoding an IFNα protein.
Embodiment B 5. The RNA of Embodiment B 4, wherein the IFNα protein is an IFNα2b protein.
Embodiment B 6. The RNA of Embodiment B 2, wherein the method comprises administering RNA encoding an IL-12sc protein, RNA encoding a GM-CSF protein, and RNA encoding an IL-15 sushi protein.
Embodiment B 7. The RNA of Embodiment B 3, wherein the method comprises administering RNA encoding an IL-12sc protein, RNA encoding a GM-CSF protein, and RNA encoding an IL-2 protein.
Embodiment B 8. The RNA of Embodiment B 4 or 5, wherein the method comprises administering RNA encoding an IL-12sc protein, RNA encoding a GM-CSF protein, and RNA encoding an IFNα protein.
Embodiment B 9. The RNA of Embodiment B 4 or 5, wherein the method comprises administering RNA encoding an IL-12sc protein, RNA encoding a GM-CSF protein, RNA encoding an IL-15 sushi protein, and RNA encoding an IFNα protein.
Embodiment B 10. The RNA of Embodiment B 4 or 5, wherein the method comprises administering RNA encoding an IL-12sc protein, RNA encoding a GM-CSF protein, RNA encoding an IL-2 protein, and RNA encoding an IFNα protein.
Embodiment B 11. The RNA of any one of embodiments B 1-10, wherein (i) the RNA encoding an IL-12sc protein comprises the nucleotide sequence of SEQ ID NO: 17 or 18, or a nucleotide sequence having at least 99%, 98%, 97%, 96%, 95%, 90%, 85%, or 80% identity to the nucleotide sequence of SEQ ID NO: 17 or 18 and/or (ii) the IL-12sc protein comprises the amino acid sequence of SEQ ID NO: 14, or an amino acid sequence having at least 99%, 98%, 97%, 96%, 95%, 90%, 85%, or 80% identity to the amino acid sequence of SEQ ID NO: 14.
Embodiment B 12. The RNA of any one of embodiments B 1-11, wherein (i) the RNA encoding a GM-CSF protein comprises the nucleotide sequence of SEQ ID NO: 29, or a nucleotide sequence having at least 99%, 98%, 97%, 96%, 95%, 90%, 85%, or 80% identity to the nucleotide sequence of SEQ ID NO: 29 and/or (ii) the GM-CSF protein comprises the amino acid sequence of SEQ ID NO: 27, or an amino acid sequence having at least 99%, 98%, 97%, 96%, 95%, 90%, 85%, or 80% identity to the amino acid sequence of SEQ ID NO: 27.
Embodiment B 13. The RNA of any one of embodiments B 2-12, wherein (i) the RNA encoding an IL-15 sushi protein comprises the nucleotide sequence of SEQ ID NO: 26, or a nucleotide sequence having at least 99%, 98%, 97%, 96%, 95%, 90%, 85%, or 80% identity to the nucleotide sequence of SEQ ID NO: 26 and/or (ii) the IL-15 sushi protein comprises the amino acid sequence of SEQ ID NO: 24, or an amino acid sequence having at least 99%, 98%, 97%, 96%, 95%, 90%, 85%, or 80% identity to the amino acid sequence of SEQ ID NO: 24.
Embodiment B 14. The RNA of any one of embodiments B 3-13, wherein (i) the RNA encoding an IL-2 protein comprises the nucleotide sequence of SEQ ID NO: 12 or 13, or a nucleotide sequence having at least 99%, 98%, 97%, 96%, 95%, 90%, 85%, or 80% identity to the nucleotide sequence of SEQ ID NO: 12 or 13 and/or (ii) the IL-2 protein comprises the amino acid sequence of SEQ ID NO: 9, or an amino acid sequence having at least 99%, 98%, 97%, 96%, 95%, 90%, 85%, or 80% identity to the amino acid sequence of SEQ ID NO: 9.
Embodiment B 15. The RNA of any one of embodiments B 4-14, wherein (i) the RNA encoding an IFNα protein comprises the nucleotide sequence of SEQ ID NO: 22 or 23, or a nucleotide sequence having at least 99%, 98%, 97%, 96%, 95%, 90%, 85%, or 80% identity to the nucleotide sequence of SEQ ID NO: 22 or 23 and/or (ii) the IFNα protein comprises the amino acid sequence of SEQ ID NO: 19, or an amino acid sequence having at least 99%, 98%, 97%, 96%, 95%, 90%, 85%, or 80% identity to the amino acid sequence of SEQ ID NO: 19.
Embodiment B 16. The RNA of any one of embodiments B 1-15, wherein at least one RNA comprises a modified nucleobase in place of at least one uridine.
Embodiment B 17. The RNA of any one of embodiments B 1-16, wherein each RNA comprises a modified nucleobase in place of at least one uridine.
Embodiment B 18. The RNA of any one of embodiments B 1-17, wherein each RNA comprises a modified nucleobase in place of each uridine.
Embodiment B 19. The RNA of any one of embodiments B 16-18, wherein the modified nucleobase is pseudouridine (ψ), N1-methyl-pseudouridine (m1ψ) or 5-methyl-uridine (m5U).
Embodiment B 20. The RNA of Embodiment B 19, wherein the modified nucleobase is N1-methyl-pseudouridine (m¹ψ).
Embodiment B 21. The RNA of any one of embodiments B 1-20, wherein at least one RNA comprises a 5′ cap.
Embodiment B 22. The RNA of any one of embodiments B 1-21, wherein each RNA comprises a 5′ cap.
Embodiment B 23. The RNA of Embodiment B 21 or 22, wherein the 5′ cap is m₂^{7,3′-O}Gppp(m₁^{2′-O})ApG or 3′-O-Me-m⁷G(5′)ppp(5′)G.
Embodiment B 24. The RNA of any one of embodiments B 1-23, wherein at least one RNA comprises a 5′ UTR.
Embodiment B 25. The RNA of any one of embodiments B 1-24, wherein each RNA comprises a 5′ UTR.
Embodiment B 26. The RNA of Embodiment B 24 or 25, wherein the 5′ UTR comprises a nucleotide sequence selected from the group consisting of SEQ ID NOs: 2, 4, and 6, or a nucleotide sequence having at least 99%, 98%, 97%, 96%, 95%, 90%, 85%, or 80% identity to a nucleotide sequence selected from the group consisting of SEQ ID NOs: 2, 4, and 6.
Embodiment B 27. The RNA of any one of embodiments B 1-26, wherein at least one RNA comprises a 3′ UTR.
Embodiment B 28. The RNA of any one of embodiments B 1-27, wherein each RNA comprises a 3′ UTR.
Embodiment B 29. The RNA of embodiment B 27 or 28, wherein the 3′ UTR comprises the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8.
Embodiment B 30. The RNA of any one of embodiments B 1-29, wherein at least one RNA comprises a poly-A tail.
Embodiment B 31. The RNA of any one of embodiments B 1-30, wherein each RNA comprises a poly-A tail.
Embodiment B 32. The RNA of embodiment B 30 or 31, wherein the poly-A tail comprises at least 100 nucleotides.
Embodiment B 33. The RNA of any one of embodiments B 1-32, wherein at least one RNA comprises a 5′ cap, a 5′ UTR, a 3′ UTR, and a poly-A tail.
Embodiment B 34. The RNA of any one of embodiments B 1-33, wherein each RNA comprises a 5′ cap, a 5′ UTR, a 3′ UTR, and a poly-A tail.
Embodiment B 35. The RNA of embodiment B 33 or 34, wherein
   a. the 5′ cap is m₂^{7,3′-O}Gppp(m₁^{2′-O})ApG or 3′-O-Me-m⁷G(5′)ppp(5′)G;
   b. the 5′ UTR comprises a nucleotide sequence selected from the group consisting of SEQ ID NOs: 2, 4, and 6, or a nucleotide sequence having at least 99%, 98%, 97%, 96%, 95%, 90%, 85%, or 80% identity to a nucleotide sequence selected from the group consisting of SEQ ID NOs: 2, 4, and 6;
   c. the 3′ UTR comprises the nucleotide sequence of SEQ ID NO: 8, or a nucleotide sequence having at least 99%, 98%, 97%, 96%, 95%, 90%, 85%, or 80% identity to the nucleotide sequence of SEQ ID NO:8; and
   d. the poly-A tail comprises at least 100 nucleotides.
Embodiment B 36. The RNA of any one of embodiments B 1 to 35, wherein the RNA is mRNA.
Embodiment B 37. The RNA of any one of embodiments B 1 to 36, wherein the method further comprises administering a further therapy.
Embodiment B 38. The RNA of embodiment B 37, wherein the further therapy comprises one or more selected from the group consisting of: (i) surgery to excise, resect, or debulk a tumor, (ii) immunotherapy, (iii) radiotherapy, and (iv) chemotherapy.
Embodiment B 39. The RNA of embodiment B 37 or 38, wherein the further therapy comprises administering a further therapeutic agent.
Embodiment B 40. The RNA of embodiment B 39, wherein the further therapeutic agent is an anti-cancer therapeutic agent.
Embodiment B 41. The RNA of embodiment B 39 or 40, wherein the further therapeutic agent is a checkpoint modulator.
Embodiment B 42. The RNA of embodiment B 41, wherein the checkpoint modulator is an anti-PD1 antibody, an anti-CTLA-4 antibody, or a combination of an anti-PD1 antibody and an anti-CTLA-4 antibody.
Embodiment B 43. The RNA of any one of embodiments B 1-42, wherein the cancer is a sarcoma, carcinoma, or lymphoma.
Embodiment B 44. The RNA of any one of embodiments B 1-43, wherein the cancer is a solid tumor.
Embodiment B 45. The RNA of embodiment B 44, wherein the solid tumor is in the lung, colon, ovary, cervix, uterus, peritoneum, testicles, penis, tongue, lymph node, pancreas, bone, breast, prostate, soft tissue, connective tissue, kidney, liver, brain, thyroid, or skin.
Embodiment B 46. The RNA of embodiment B 44 or 45, wherein the solid tumor is an epithelial tumor, Hodgkin lymphoma (HL), non-Hodgkin lymphoma, prostate tumor, ovarian tumor, renal cell tumor, gastrointestinal tract tumor, hepatic tumor, colorectal tumor, tumor with vasculature, mesothelioma tumor, pancreatic tumor, breast tumor, sarcoma tumor, lung tumor, colon tumor, brain tumor, melanoma tumor, small cell lung tumor, neuroblastoma tumor, testicular tumor, carcinoma tumor, adenocarcinoma tumor, glioma tumor, seminoma tumor, retinoblastoma, or osteosarcoma tumor.
Embodiment B 47. The RNA of any one of embodiments B 1-46, wherein the RNA is administered intra-tumorally or peri-tumorally.
Embodiment B 48. The RNA of any one of embodiments B 39 to 47, wherein the further therapeutic agent is administered systemically.
Embodiment B 49. The RNA of any one of embodiments B 1-48, wherein the subject is a human.
Embodiment B 50. The RNA of any one of embodiments B 1-49, wherein the RNAs are administered at the same time.
Embodiment B 51. The RNA of any one of embodiments B 1-50, wherein the RNAs are administered by administering a composition comprising a combination of the RNAs.
Embodiment B 52. The RNA of any one of embodiments B 1-49, wherein at least two of the RNAs are administered at different times.
Embodiment B 53. The RNA of any one of embodiments B 1-49 and 52, wherein the RNAs are administered by administering at least two compositions, each composition comprising at least one of said RNAs.
Embodiment B 54. The RNA of any one of embodiments B 1 to 53, wherein treating or preventing cancer comprises reducing the size of a tumor, preventing the reoccurrence of cancer in remission, or preventing cancer metastasis in a subject.
Embodiment B 55. The RNA of any one of embodiments B 1 to 54, which is or comprises one or more of the RNAs administered in said method.
Embodiment B 56. The RNA of embodiment B 55, which is or comprises one or more selected from the group consisting of the RNA encoding an IL-12sc protein, the RNA encoding a GM-CSF protein, the RNA encoding an IL-15 sushi protein, the RNA encoding an IL-2 protein, and the RNA encoding an IFNα protein.
Embodiment B 57. The RNA of embodiment B 55 or 56, which is or comprises the RNA encoding an IL-12sc protein.
Embodiment B 58. The RNA of embodiment B 55 or 56, which is or comprises the RNA encoding a GM-CSF protein.
Embodiment B 59. The RNA of embodiment B 55 or 56, which is or comprises the RNA encoding an IL-15 sushi protein.
Embodiment B 60. The RNA of embodiment B 55 or 56, which is or comprises the RNA encoding an IL-2 protein.
Embodiment B 61. The RNA of embodiment B 55 or 56, which is or comprises the RNA encoding an IFNα protein.
Embodiment B 62. The RNA of embodiment B 55 or 56, which is or comprises the RNA encoding an IL-12sc protein and the RNA encoding a GM-CSF protein.
Embodiment B 63. The RNA of embodiment B 55 or 56, which is or comprises the RNA encoding an IL-12sc protein, the RNA encoding a GM-CSF protein, and the RNA encoding an IL-15 sushi protein.
Embodiment B 64. The RNA of embodiment B 55 or 56, which is or comprises the RNA encoding an IL-12sc protein, the RNA encoding a GM-CSF protein, and the RNA encoding an IL-2 protein.
Embodiment B 65. The RNA of embodiment B 55 or 56, which is or comprises the RNA encoding an IL-12sc protein, the RNA encoding a GM-CSF protein, and the RNA encoding an IFNα protein.
Embodiment B 66. The RNA of embodiment B 55 or 56, which is or comprises the RNA encoding an IL-12sc protein, the RNA encoding a GM-CSF protein, the RNA encoding an IL-15 sushi protein, and the RNA encoding an IFNα protein.
Embodiment B 67. The RNA of embodiment B 55 or 56, which is or comprises the RNA encoding an IL-12sc protein, the RNA encoding a GM-CSF protein, the RNA encoding an IL-2 protein, and the RNA encoding an IFNα protein.

### FIGURE LEGENDS

FIGS. 1A-1G shows results of experiments where B16F10 tumor bearing mice were injected intratumorally with mRNA on days 8, 10, 12, 14 and individual tumor growth was monitored to day 41. FIG. 1A and FIG. 1D show results when using IL-2, IL-12sc, and GM-CSF (ModA) mRNA. FIG. 1B and FIG. 1E show IL-2, IL-12sc, and GM-CSF (ModB) mRNA. FIG. 1C and FIG. 1F show results when using luciferase mRNA (ModA). FIG. 1G shows results when using luciferase mRNA (ModB). (N=10 mice/group for A-C and N=9 mice/group for D-G).

FIGS. 2A-2D show results of experiments where CT26 tumor bearing mice were injected intratumorally with mRNA on day 19, 21, 24, 26, 28 and 31 and individual tumor growth was monitored to day 48. FIG. 2A shows GM-CSF, IL-2, IL-12sc (ModA). FIG. 2B shows GM-CSF, IL-2, IL-12sc (ModB). FIG. 2C shows luciferase mRNA (ModA) as a control. FIG. 2D shows Ringer's solution as a control.

FIGS. 3A-3C show results of experiments where CT26 tumor bearing mice were injected intratumorally with mRNA on day 13, 15, 18, 20 and 22 and tumor growth was monitored to day 42. FIG. 3A shows IL-2, GM-CSF, IL-12sc (ModB). FIG. 3B shows IL-15 sushi, GM-CSF, IL-12sc (ModB). FIG. 3C shows luciferase mRNA (ModB) as a control.

FIGS. 4A-4F show results of experiments where B16F10 tumor bearing mice were injected intratumorally with cytokine mRNA mixtures on days 11, 13, 15, 17 and individual tumor growth was monitored to day 45. FIG. 4A shows IL-2, IL-12sc, and GM-CSF (ModA). FIG. 4B is a duplicate in the same experiment as described in FIG. 5A, showing IL-2, IL-12sc, and GM-CSF (ModB). FIG. 4C shows IL-15 sushi, IL-12sc, and GM-CSF (ModA). FIG. 4D is a duplicate in the same experiment as described in FIG. 5B, showing IL-15 sushi, IL-12sc, and GM-CSF (ModB). FIG. 4E shows control luciferase mRNA (ModA). FIG. 4F is a duplicate in the same experiment as described in FIGS. 5D and 6D showing control luciferase mRNA (ModB). (N=8 mice/group).

FIGS. 5A-5D show results of experiments where B16F10 tumor bearing mice were injected intratumorally with cytokine mRNA mixtures on days 11, 13, 15, 17 and individual tumor growth was monitored to day 45. FIG. 5A is a duplicate in the same experiment as described in FIG. 4B, showing IL-2, IL-12sc, and GM-CSF (ModB). FIG. 5B is a duplicate in the same experiment as described in FIG. 4D, showing IL-15 sushi, IL-12sc, and GM-CSF (ModB). FIG. 5C is a duplicate in the same experiment as described in FIG. 6C, showing IL-2, IL-12sc, GM-CSF, and IFNα (ModB). FIG. 5D is a duplicate in the same experiment as described in FIGS. 4F and 6D, showing luciferase mRNA (ModB) as control. (N=8 mice/group).

FIGS. 6A and 6B show results of experiments where CT26 tumor bearing mice were injected intratumorally with cytokine mRNA mixtures on days 13, 15, 17, 19, 21, 23 and individual tumor growth was plotted. FIG. 6A is a duplicate in the same experiment as described in FIG. 7A, showing GM-CSF, IL-2, IL-12sc, IFNα (ModB). FIG. 6B is a duplicate in the same experiment as described in FIG. 7C, showing luciferase mRNA (ModB). N=8 mice/group.

FIGS. 6C and 6D show results of experiments where B16F10 tumor bearing mice were injected intratumorally with mRNA on days 11, 13, 15, 17 and individual tumor growth was plotted. FIG. 6C is a duplicate in the same experiment as described in FIG. 5C, showing GM-CSF, IL-2, IL-12sc, IFNα (ModB). FIG. 6D is a duplicate in the same experiment as described in FIGS. 4F and 5D, showing luciferase mRNA (ModB). N=8 mice/group.

FIGS. 6E and 6F show results of experiments where MC38 tumor bearing mice were injected intratumorally with cytokine mRNA mixtures on days 11, 15, 19, 23 and individual tumor growth was plotted. FIG. 6E shows GM-CSF, IL-2, IL-12sc, IFNα (ModB). FIG. 6F shows luciferase mRNA (ModB). N=5 mice/group.

FIGS. 7A-7F show results of experiments where CT26 tumor bearing mice were injected intratumorally with cytokine mRNA mixtures on days 13, 15, 17, 19, 21, 23 and individual tumor growth was plotted. FIG. 7A is a duplicate in the same experiment as described in FIG. 6A, showing IL-2, IL-12sc, GM-CSF, IFNα (ModB). FIG. 7B shows IL-15 sushi, IL-12sc, GM-CSF, IFNα (ModB). FIG. 7C is a duplicate in the same experiment as described in FIG. 6B, showing a luciferase mRNA (ModB) control. In a repeat study of similar design, CT26 tumor bearing mice were injected intratumorally with cytokine mRNA mixtures on days 19, 21, 23, 26, 28 and 30 and individual tumor growth was plotted. FIG. 7D is a duplicate of the same experiment as described in FIG. 9A, showing IL-2, IL-12sc, GM-CSF, IFNα (ModB). FIG. 7E shows IL-15 sushi, IL-12sc, GM-CSF, IFNα (ModB). FIG. 7F is a duplicate of the same experiment as described in FIG. 9F, showing a luciferase mRNA (ModB) control. N=8 mice/group for FIGS. A-C and N=10-11 mice/group for FIGS. D-F.

FIGS. 8A-8H show results of experiments where CT26 tumor bearing mice were injected intratumorally with mRNA on days 12, 15, 19 and 22 and individual tumor growth was monitored and plotted to day 35. FIG. 8A shows IL-15 sushi, IL-12sc, GM-CSF, IFNα (ModB). FIG. 8B shows IL-15 sushi, IL-12sc, IFNα (ModB). FIG. 8C shows IL-15 sushi, GM-CSF, IFNα (ModB). FIG. 8D shows GM-CSF, IL-12sc, IFNα (ModB). FIG. 8E shows IL-15 sushi, GM-CSF, IL-12sc (ModB). FIG. 8F shows a luciferase mRNA (ModB) control. (N=10/group). FIGS. 8G and 8H show tumor growth kinetics of the study shown in FIGS. 8A-8F. FIG. 8G shows mean tumor volumes up to day 33 for all treatment groups. FIG. 8H shows tumor growth repression. T/C (Tumor/Control based on mean tumor volume) was calculated up to day 19.

FIGS. 9A-9F show experiments where CT26 tumor bearing mice were injected intratumorally with mRNA on days 19, 21, 23, 26, 28 and 30 and tumor growth was monitored to day 50. FIG. 9A is a duplicate in the same experiment as described in FIG. 7D, showing GM-CSF, IL-2, IL-12sc, IFNα (ModB). FIG. 9B shows IL-2, IL-12sc, IFNα (ModB). FIG. 9C shows GM-CSF, IL-2, IFNα (ModB). FIG. 9D shows GM-CSF, IL-12sc, IFNα (ModB). FIG. 9E shows GM-CSF, IL-2, IL-12sc (ModB). FIG. 9F is a duplicate in the same experiment as described in FIG. 7F, showing shows luciferase mRNA (ModA) as control. (N=11/group for FIG. 9A-E; luciferase mRNA group N=10 for FIG. 9F).

FIGS. 10A-10B shows tumor growth kinetics of the study shown in FIG. 9. FIG. 10A shows mean tumor volumes up to day 36 for all treatment groups. FIG. 10B shows tumor growth repression. T/C (Tumor/Control based on mean tumor volume) was calculated up to day 30.

FIG. 11 shows a bar graph of data from the experiments shown in FIG. 9 showing mRNA mixtures with significant reduction in tumor volume, where the number of mice in each of the treatment groups with significant tumor reduction was compared to the luciferase control group based on Z score of tumor volume and the ratio between tumor volume change and the mean of the control group.

FIGS. 12A-12D show the results of experiments where mice that were 1) tumor naïve, or 2) had been previously injected subcutaneously with 5×10⁵ B16F10 cells and rejected the original tumor following intratumoral cytokine mRNA treatment. Both groups were re-challenged with B16F10 tumors. FIG. 12A shows tumor naïve host mice. FIG. 12B shows mice that had previously rejected B16F10 tumors following intratumoral cytokine mRNA treatment with GM-CSF, IL-15sushi, IL-12sc, IFNα (ModB). Mice were monitored for 55 days following B16F10 injection and tumor growth for each mouse was plotted. All nine naïve mice engrafted with B16F10 cells developed tumors (FIG. 12A), whereas all eight tumor-free mice rejected the B16F10 cells and did not exhibit growth of B16F10 tumors (FIG. 12B). The graph in FIG. 12B has no visible data trace because all observations were zero, i.e., overlapping the horizontal axis. FIG. 12C shows an example of localized vitiligo at the tumor site. FIG. 12D shows the results of experiments where mice that were tumor naïve (triangle symbol), or had been previously injected subcutaneously with CT26 tumor cells and rejected the original tumor following intratumoral cytokine mRNA treatment (circle symbol). Both groups were re-challenged with either CT26 tumor cells (CT26-WT) or with CT26-Agp70 tumor cells, in which the gp70-epitope had been knocked out. Mice were monitored for 21 days following tumor cell injection. All nine but one naïve mice engrafted with CT26-WT cells and all naïve mice engrafted with CT26-Agp70 cells developed tumors, whereas all three tumor-free mice rejected the CT26 tumor cells and did not exhibit growth of CT26 and CT26-Agp70 tumors, respectively.

FIGS. 13A-13D show the results of experiments where mice were implanted with B16F10 tumor cells on day 0 on the right (injected) and left flanks (uninjected) (FIG. 13A). Mice received a series of 4 intratumoral injections with ModB cytokine mRNA (IL-15 sushi, IL-12sc, GM-CSF and IFNα) or ModB control mRNA (luciferase) in the right tumor on days 11, 15, 19, and 23. Mean tumor volumes+/−SEM (n=12) are shown for the injected (FIG. 13B) and the contralateral uninjected tumors (FIG. 13C). Median survival is shown in FIG. 13D.

FIGS. 14A-14F show results of experiments where human HEK293 (FIG. 14B) and melanoma cell lines (A101D (FIG. 14C), A2058 (FIG. 14D), A375 (FIG. 14E), and Hs294T (FIG. 14F)) were transfected with human cytokine mRNA mixture (IL-12sc, GM-CSF, IL-15 sushi and IFNα2b) in a range of mRNA doses. Supernatants were collected 24 hrs after transfection and protein concentrations were determined with cytokine specific ELISAs. FIG. 14A shows a schematic of the experiment.

FIGS. 15A-15B show a schematic (FIG. 15A) and results (FIG. 15B) from a study where a human cytokine mRNA mixture encoding IL-15 sushi, IL-12sc, GM-CSF and IFNα2b, or individual cytokine mRNAs, were transfected in HEK293 cells and the conditioned media was collected at 24 hrs, diluted and added to human PBMCs. IFNγ was measured in the PBMC culture supernatant at 24 hrs. (N=6 donors, Mean).

FIGS. 16A-16E show the results of experiments where immune compromised mice bearing human A375 tumor xenografts received a single injection with the ModB mRNA mixture encoding the human cytokines (IL-15 sushi, IL-12sc, GM-CSF and IFNα2b; “the IL15 sushi mixture”) or (IL-2, IL-12sc, GM-CSF and IFNα2b; “the IL2 mixture”). Tumor cell lysates were prepared at 2 hrs, 4 hrs, 8 hrs, 24 hrs, 48 hrs, and 72 hrs after injection and the concentration of each cytokine was measured with respect to the total protein in the tumor lysates (n=3 mice/time point, +/−SEM). FIG. 16A shows IFNα2b, FIG. 16B shows IL-2, FIG. 16C shows IL-12sc, FIG. 16D shows IL-15 sushi, and FIG. 16E shows GM-CSF.

FIGS. 17A-17C show the results of experiments where mRNA was isolated from A375 tumors at 2 hrs, 4 hrs, 8 hrs, 24 hrs, 48 hrs, and 72 hrs after injection of ModB cytokine mRNA mixture (IL-15 sushi, IL-12sc, GM-CSF, IFNα2b) or (IL-2, IL-12sc, GM-CSF, IFNα2b). Expression of interferon alpha response genes were monitored by qPCR. FIG. 17A shows human ISG15, FIG. 17B shows human ISG54, and FIG. 17C shows human MX1.

FIGS. 18A-18E show the results of experiments where mice were implanted with B16F10 tumor cells and treated with mRNA mixtures (FLT3L, IL-2, 41BBL, and CD27L-CD40L) with or without IFNα. mRNA mixtures without IFNα in standard (ModA, FIG. 18B) and modified forms (ModB, FIG. 18C) were compared to those including IFNα in standard (ModA, FIG. 18D) and modified forms (ModB, FIG. 18E). FIG. 18A is a negative control where Ringer's media without mRNA was provided.

FIGS. 19A-19E show the results of experiments where mice were implanted with tumors on one flank and received an IV injection of luciferase-expressing tumor cells that homed to the lung (FIG. 19A). Mice in the treatment group received intratumoral injections of mRNA mixtures IL-15 sushi, IL-12sc, GM-CSF and IFNα into the flank tumor only while tumors in the lung were untreated. FIG. 19B shows exemplarily bioluminescence measurements in lungs and pictures of the according lungs taken out on the same day (day 20); tumor nodes are visual as black marks; FIG. 19C shows mean tumor volume of flank tumors as determined by caliper measurements; FIG. 19D shows total flux analysis of bioluminescence measurements on day 20; FIG. 19E shows lung weights.

FIGS. 20A-20G show the results of experiments designed to assess the effect of intratumoral injection of mRNA mixtures in combination with systemic administration of antibodies in dual flank tumor models. Mice implanted with either the B16F10 tumor on the left and right flank or MC38 tumors on the left and right flank received intratumoral injections with an mRNA mixture of IL-15 sushi, IL-12sc, GM-CSF and IFNα (Mod B) into only one flank tumor, while the other flank tumor was untreated. Mice also received intraperitoneal (systemic) injection of an anti-PD1 antibody. FIG. 20 shows overall survival in the B16F10 (FIG. 20A) and MC38 (FIG. 20B) tumor models. FIGS. 20C-G show the results of an experiment evaluating the anti-PD-1 antibody where mice were implanted with B16F10 tumors on one flank and received an IV injection of luciferase-expressing B16F10 tumor cells that homed to the lung. Mice received three intratumoral injections with an mRNA mixture of IL-15 sushi, IL-12sc, GM-CSF and IFNα and also received three intraperitoneal (systemic) injection of an anti-PD-1 antibody. Tumor growth of the SC tumors is depicted in FIG. 20C-F. FIG. 20C shows control mRNA and control antibody; FIG. 20D shows control mRNA plus anti-PD1 antibody; FIG. 20E shows cytokine mRNA mixture plus isotype control antibody. FIG. 20F shows cytokine mRNA plus anti-PD-1 antibody. FIG. 20G shows percent survival of all four treatment groups until day 70 after IV tumor inoculation; the treatment group that received mRNA plus anti-PD-1 antibody showed strongest anti-tumoral activity with 6 out of 15 mice being tumor-free on day 40 after tumor inoculation.

FIGS. 21A-21I show the results of additional experiments designed to assess the effect of intratumoral injection of mRNA mixtures in combination with systemic administration of antibodies. Mice bearing CT26 tumors received intratumoral injections with an mRNA mixture of IL-15 sushi, IL-12sc, GM-CSF and IFNα. Mice also received intraperitoneal (systemic) injection of an anti-CTLA-4 antibody. FIG. 21A shows that the combination therapy of intratumoral cytokine mRNA and IP-injected anti-CTLA-4 resulted in strongest anti-tumoral activity with 12 out of 16 mice being tumor-free on day 55 after tumor inoculation. FIG. 21B shows cytokine mRNA mixture plus isotype control antibody; FIG. 21C shows control mRNA plus anti-CTLA-4 antibody; FIG. 21D shows control mRNA and control antibody. FIGS. 21E-21I show the results of additional experiments designed to assess the effect of intratumoral injection of mRNA mixtures in combination with anti-CTLA-4 antibody in B16F10 tumor model. Mice bearing B16F10 tumors received intratumoral injections with an mRNA mixture of IL-15 sushi, IL-12sc, GM-CSF and IFNα. Mice also received intraperitoneal (systemic) injection of an anti-CTLA-4 antibody. FIG. 21E shows that the combination therapy of intratumoral cytokine mRNA and IP-injected anti-CTLA-4 resulted in strongest anti-tumoral activity with 6 out of 9 mice being tumor-free on day 70 after tumor inoculation. FIG. 21F shows cytokine mRNA mixture plus isotype control antibody; FIG. 21G shows control mRNA plus anti-CTLA-4 antibody; FIG. 21H shows control mRNA and control antibody. FIG. 21I shows percent survival of all four treatment groups until day 70 after tumor inoculation.

FIGS. 22A-22D shows the results of experiments designed to evaluate the effect of intratumoral injection of cytokine mRNA (Mod B) in human tumor xenografts of different human cancers. Intratumoral expression of each of the 4 mRNA encoded cytokines is shown: IL-12sc (FIG. 22A), IFNα2b (FIG. 22B), GM-CSF (FIG. 22C), and IL-15 sushi (FIG. 22D).

FIGS. 23A-23D show the results of experiments designed to evaluate the effect of different intratumoral mRNA doses on the expression of the encoded cytokines: IL-15 sushi (FIG. 23A), IL-12sc (FIG. 23B), GM-CSF (FIG. 23C) and IFNα2b (FIG. 23D).

FIGS. 24A-24G show the results of experiments where mice were implanted with B16F10 tumor, and treated with four intratumoral injections of cytokine mRNA mixture of IL-15 sushi, IL-12sc, GM-CSF, IFNα (ModB) or mRNA encoding a single cytokine. Tumor volume out to approximately day 70 was measured. FIG. 24A shows luciferase control; FIG. 24B shows the four-cytokine mixture; FIG. 24C shows IL-12sc mRNA only; FIG. 24D shows GM-CSF mRNA only; FIG. 24E shows IFNα mRNA only; and FIG. 24F shows IL-15 sushi only. FIG. 24G shows overall survival of B16F10 tumors treated with cytokine mRNA mixture or individual mRNA encoded cytokines. Survival data is from experiment presented in FIG. 24A-F.

FIG. 25 shows CD8+ immune cell infiltrate in subcutaneous tumors after control mRNA (“placebo”) and cytokine mRNA treatment.

FIGS. 26A-26C show results of measurements of CD8+ T cells specific for the gp70 tumor antigen of gp70 in blood of CT26 tumor bearing mice that had received intratumoral administration of cytokine mRNA treatment and control mRNA, respectively. FIG. 26C shows exemplarily a FACS histogram of CD8+ T cells stained with anti-mouse CD8 antibody and with the gp70-specific tetramer derived from an animal that had received control mRNA and FIG. 26B shows the example from one animal treated with cytokine mRNA. FIG. 26C shows the analysis of percentage of gp70-specific CD8+ T-cells in blood 13 days after treatment start from 9 mice that had received four injections of control mRNA and 10 mice that had received 4 injections of cytokine mRNA.

FIGS. 27A-27C show experiments where mice bearing B16F10 tumors on the left and right flanks received a single intratumoral mRNA injection with cytokine mRNA or control mRNA in only one tumor. On day 7 following the mRNA injection the left and right tumors were collected and subjected to RNA sequencing. FIG. 27B shows the results of ingenuity pathway analysis comparing the gene expression changes between the cytokine mRNA treatment vs control mRNA treated tumors. Causal network analysis for treated tumor side (Column 1) and untreated tumor side (Column 2) was performed and Activation Z score (Top half) and Inhibition Z score (Lower half) was analyzed to define pathways up and down regulated, respectively. FIG. 27 shows cluster analysis of injected and non-injected tumors was performed based on 327 interferon gamma regulated genes. Both the injected and non-injected tumors of mice treated with cytokine mRNA showed upregulation of multiple IFN gamma genes in comparison to mice treated with control mRNA.

FIGS. 28A-28D show fluorescence micrographs of cells from a B16F10 dualtumor model. Panel A shows the injected tumor treated with cytokine mRNA and panel B shows the corresponding uninjected tumor. Panel C shows the injected tumor treated with control mRNA and panel D shows the corresponding uninjected tumor. The slides were stained for CD4+, CD8+, and FoxP3+ cells.

FIGS. 28E-G show frequency of CD4+, CD8+ and FOXP3+ cells quantified in the immunofluorescent images. The frequency of CD4+ and CD8+ cells/mm² is presented in FIGS. 28E and 28F. The ratio of the CD8+ frequency divided by FOXP3+ frequency is presented in FIG. 28G.

FIGS. 29A-29G show mice with a single B16F10 tumor received a single injection with either mRNA encoding the Thy1.1 cell surface protein or vehicle alone (Ringer's solution). At approximately 16-18 hours following intratumoral injection the tumor was excised, digested, stained with a panel of antibodies and analyzed by flow cytometry. The cell type and frequency of cells expressing Thy1.1 were characterized.

FIGS. 30A-30F show expression of the indicated proteins following various doses of cytokine mRNA or luciferase control mRNA detected in tumor lysates as described in Example 15. “IFNy” in FIG. 30E indicates IFNγ.

FIGS. 31A-31B show flow cytometry results for CD8+ and FOXP3+ (Treg) cells following control or cytokine mRNA treatments as described in Example 15. The observed ratio of CD8+ to Treg cells is shown in each panel.

FIGS. 31C-31D show flow cytometry results for polyfunctional CD8+ T cells following control or cytokine mRNA treatments as described in Example 15. The proportion of polyfunctional CD8+ T cells is shown in each panel.

FIG. 31E shows the level of PD-L1 on infiltrating myeloid cells following control or cytokine mRNA treatments as described in Example 15.

FIG. 31F shows the level of PD-1 on infiltrating CD8+ cells following control or cytokine mRNA treatments as described in Example 15.

FIGS. 31G-H show the frequency of intratumoral Granzyme B CD8+ T cells following control or cytokine mRNA treatments as described in Example 15.

FIGS. 32A-32B show luciferase expression in various tissues following intratumoral injection of 50 μg mRNA encoding firefly luciferase as described in Example 16.

FIG. 33 shows data relating to an experiment essentially as shown in FIG. 12.

FIG. 34 shows the effect of depleting CD8+ T cells, CD4+ T cells or NK cells before treatment with cytokine mRNAs on survival in mice bearing B16F10 tumors as described in Example 17.

FIG. 35 shows survival of WT and IFNγ KO mice implanted with B16F10 tumor cells as described in Example 1 and treated with control or cytokine mRNAs as described in Example 18.

FIG. 36 shows a “peri-tumorally,” or “peri-tumoral,” area that is about 2-mm wide and is adjacent to the invasive front of the tumor periphery. The peri-tumoral area comprises host tissue.

### DESCRIPTION OF THE SEQUENCES

Tables 1 and 2 provide a listing of certain sequences referenced herein.

TABLE 1
DESCRIPTION OF THE SEQUENCES (human sequences)

| | | |
|---|---|---|
| SEQ | | |
| ID | | |
| NO: | Description | SEQUENCE |

5′ UTR

| | | |
|---|---|---|
| 1 | ModA 5′ UTR | GGGCGAACTAGTATTCTTCTGGTCCCCACAGACTCAGAGAGAACCCGCCACC |
| | (DNA) | |
| 2 | ModA 5′ UTR | GGGCGAACUAGUAUUCUUCUGGUCCCCACAGACUCAGAGAGAACCCGCCACC |
| | (RNA) | |
| 3 | ModB 5′ UTR | GGAATAAACTAGTCTCAACACAACATATACAAAACAAACGAATCTCAAGCAATCAAGCATTCTACTTCTATTGCAGCAATTTAAATCA |
| | (DNA) | TTTCTTTTAAAGCAAAAGCAATTTTCTGAAAATTTTCACCATTTACGAACGATAGCC |
| 4 | ModB 5′ UTR | GGAAUAAACUAGUCUCAACACAACAUAUACAAAACAAACGAAUCUCAAGCAAUCAAGCAUUCUACUUCUAUUGCAGCAAUUUAAAUCA |
| | (RNA) | UUUCUUUUAAAGCAAAAGCAAUUUUCUGAAAAUUUUCACCAUUUACGAACGAUAGCC |
| 5 | Alternative | AGACGAACTAGTATTCTTCTGGTCCCCACAGACTCAGAGAGAACCCGCCACC |
| | Mod 5′ UTR | |
| | (DNA) | |
| 6 | Alternative | AGACGAACUAGUAUUCUUCUGGUCCCCACAGACUCAGAGAGAACCCGCCACC |
| | Mod 5′ UTR | |
| | (RNA) | |

3′ UTR

| | | |
|---|---|---|
| 7 | ModA/B 3′ | CTCGAGCTGGTACTGCATGCACGCAATGCTAGCTGCCCCTTTCCCGTCCTGGGTACCCCGAGTCTCCCCCGACCTCGGGTCCCAGGTA |
| | UTR (DNA) | TGCTCCCACCTCCACCTGCCCCACTCACCACCTCTGCTAGTTCCAGACACCTCCCAAGCACGCAGCAATGCAGCTCAAAACGCTTAGC |
| | | CTAGCCACACCCCCACGGGAAACAGCAGTGATTAACCTTTAGCAATAAACGAAAGTTTAACTAAGCTATACTAACCCCAGGGTTGGTC |
| | | AATTTCGTGCCAGCCACACCGAGACCTGGTCCAGAGTCGCTAGCCGCGTCGCTAAAAAAAAAAAAAAAAAAAAAAAAAAAAAAGCATA |
| | | TGACTAAAAAAAAAAAAAAAAAAAAAAAAAAAAAAAAAAAAAAAAAAAAAAAAAAAAAAAAAAAAAAAAAAAAAA |
| 8 | ModA/B 3′ | CUCGAGCUGGUACUGCAUGCACGCAAUGCUAGCUGCCCCUUUCCCGUCCUGGGUACCCCGAGUCUCCCCCGACCUCGGGUCCCAGGUA |
| | UTR (RNA) | UGCUCCCACCUCCACCUGCCCCACUCACCACCUCUGCUAGUUCCAGACACCUCCCAAGCACGCAGCAAUGCAGCUCAAAACGCUUAGC |
| | | CUAGCCACACCCCCACGGGAAACAGCAGUGAUUAACCUUUAGCAAUAAACGAAAGUUUAACUAAGCUAUACUAACCCCAGGGUUGGUC |
| | | AAUUUCGUGCCAGCCACACCGAGACCUGGUCCAGAGUCGCUAGCCGCGUCGCUAAAAAAAAAAAAAAAAAAAAAAAAAAAAAAGCAUA |
| | | UGACUAAAAAAAAAAAAAAAAAAAAAAAAAAAAAAAAAAAAAAAAAAAAAAAAAAAAAAAAAAAAAAAAAAAAAA |

IL-2

| | | |
|---|---|---|
| 9 | Human IL-2 | MYRMQLLSCIALSLALVTNSAPTSSSTKKTQLQLEHLLLDLQMILNGINNYKNPKLTRMLTFKFYMPKKATELKHLQCLEEELKPLEE |
| | (amino | VLNLAQSKNFHLRPRDLISNINVIVLELKGSETTFMCEYADETATIVEFLNRWITFCQSIISTLT |
| | acid) | |
| 10 | Human non- | ATGTACAGGATGCAACTCCTGTCTTGCATTGCACTAAGTCTTGCACTTGTCACAAACAGTGCACCTACTTCAAGTTCTACAAAGAAAA |
| | optimized | CACAGCTACAACTGGAGCATTTACTGCTGGATTTACAGATGATTTTGAATGGAATTAATAATTACAAGAATCCCAAACTCACCAGGAT |
| | IL-2 (CDS | GCTCACATTTAAGTTTTACATGCCCAAGAAGGCCACAGAACTGAAACATCTTCAGTGTCTAGAAGAAGAACTCAAACCTCTGGAGGAA |
| | DNA) | GTGCTAAATTTAGCTCAAAGCAAAAACTTTCACTTAAGACCCAGGGACTTAATCAGCAATATCAACGTAATAGTTCTGGAACTAAAGG |
| | | GATCTGAAACAACATTCATGTGTGAATATGCTGATGAGACAGCAACCATTGTAGAATTTCTGAACAGATGGATTACCTTTTGTCAAAG |
| | | CATCATCTCAACACTGACTTGATGA |
| 11 | Human | ATGTACAGAATGCAGCTGCTGTCTTGCATTGCTCTTTCTCTTGCTCTTGTGACAAATTCTGCTCCAACATCTTCTTCAACAAAGAAAA |
| | optimized | CACAGCTTCAGCTTGAACACCTTCTTCTTGATCTTCAGATGATTCTGAATGGAATCAACAATTACAAAAATCCAAAACTGACAAGAAT |
| | IL-2 (CDS | GCTGACATTTAAATTTTACATGCCAAAGAAAGCAACAGAACTGAAACACCTTCAGTGCCTTGAAGAAGAACTGAAACCTCTGGAAGAA |
| | DNA) | GTGCTGAATCTGGCTCAGAGCAAAAATTTTCACCTGAGACCAAGAGATCTGATCAGCAACATCAATGTGATTGTGCTGGAACTGAAAG |
| | | GATCTGAAACAACATTCATGTGTGAATATGCTGATGAAACAGCAACAATTGTGGAATTTCTGAACAGATGGATCACATTTTGCCAGTC |
| | | AATCATTTCAACACTGACATGATGA |
| 12 | Human non- | AUGUACAGGAUGCAACUCCUGUCUUGCAUUGCACUAAGUCUUGCACUUGUCACAAACAGUGCACCUACUUCAAGUUCUACAAAGAAAA |
| | optimized | CACAGCUACAACUGGAGCAUUUACUGCUGGAUUUACAGAUGAUUUUGAAUGGAAUUAAUAAUUACAAGAAUCCCAAACUCACCAGGAU |
| | IL-2 (RNA | GCUCACAUUUAAGUUUUACAUGCCCAAGAAGGCCACAGAACUGAAACAUCUUCAGUGUCUAGAAGAAGAACUCAAACCUCUGGAGGAA |
| | encoding | GUGCUAAAUUUAGCUCAAAGCAAAAACUUUCACUUAAGACCCAGGGACUUAAUCAGCAAUAUCAACGUAAUAGUUCUGGAACUAAAGG |
| | CDS) | GAUCUGAAACAACAUUCAUGUGUGAAUAUGCUGAUGAGACAGCAACCAUUGUAGAAUUUCUGAACAGAUGGAUUACCUUUUGUCAAAG |
| | | CAUCAUCUCAACACUGACUUGAUGA |
| 13 | Human | AUGUACAGAAUGCAGCUGCUGUCUUGCAUUGCUCUUUCUCUUGCUCUUGUGACAAAUUCUGCUCCAACAUCUUCUUCAACAAAGAAAA |
| | optimized | CACAGCUUCAGCUUGAACACCUUCUUCUUGAUCUUCAGAUGAUUCUGAAUGGAAUCAACAAUUACAAAAAUCCAAAACUGACAAGAAU |
| | IL-2 (RNA | GCUGACAUUUAAAUUUUACAUGCCAAAGAAAGCAACAGAACUGAAACACCUUCAGUGCCUUGAAGAAGAACUGAAACCUCUGGAAGAA |
| | encoding | GUGCUGAAUCUGGCUCAGAGCAAAAAUUUUCACCUGAGACCAAGAGAUCUGAUCAGCAACAUCAAUGUGAUUGUGCUGGAACUGAAAG |
| | CDS) | GAUCUGAAACAACAUUCAUGUGUGAAUAUGCUGAUGAAACAGCAACAAUUGUGGAAUUUCUGAACAGAUGGAUCACAUUUUGCCAGUC |
| | | AAUCAUUUCAACACUGACAUGAUGA |

IL-12sc

| | | |
|---|---|---|
| 14 | Human IL- | MCHQQLVISWFSLVFLASPLVAIWELKKDVYVVELDWYPDAPGEMVVLTCDTPEEDGITWTLDQSSEVLGSGKTLTIQVKEFGDAGQY |
| | 12sc (amino | TCHKGGEVLSHSLLLLHKKEDGIWSTDILKDQKEPKNKTFLRCEAKNYSGRFTCWWLTTISTDLTFSVKSSRGSSDPQGVTCGAATLS |
| | acid) | AERVRGDNKEYEYSVECQEDSACPAAEESLPIEVMVDAVHKLKYENYTSSFFIRDIIKPDPPKNLQLKPLKNSRQVEVSWEYPDTWST |
| | | PHSYFSLTFCVQVQGKSKREKKDRVFTDKTSATVICRKNASISVRAQDRYYSSSWSEWASVPCSGSSGGGGSPGGGSSRNLPVATPDP |
| | | GMFPCLHHSQNLLRAVSNMLQKARQTLEFYPCTSEEIDHEDITKDKTSTVEACLPLELTKNESCLNSRETSFITNGSCLASRKTSFMM |
| | | ALCLSSIYEDLKMYQVEFKTMNAKLLMDPKRQIFLDQNMLAVIDELMQALNFNSETVPQKSSLEEPDFYKTKIKLCILLHAFRIRAVT |
| | | IDRVMSYLNAS |
| 15 | Human non- | ATGTGTCACCAGCAGTTGGTCATCTCTTGGTTTTCCCTGGTTTTTCTGGCATCTCCCCTCGTGGCCATATGGGAACTGAAGAAAGATG |
| | optimized | TTTATGTCGTAGAATTGGATTGGTATCCGGATGCCCCTGGAGAAATGGTGGTCCTCACCTGTGACACCCCTGAAGAAGATGGTATCAC |
| | IL-12sc | CTGGACCTTGGACCAGAGCAGTGAGGTCTTAGGCTCTGGCAAAACCCTGACCATCCAAGTCAAAGAGTTTGGAGATGCTGGCCAGTAC |
| | (CDS DNA) | ACCTGTCACAAAGGAGGCGAGGTTCTAAGCCATTCGCTCCTGCTGCTTCACAAAAAGGAAGATGGAATTTGGTCCACTGATATTTTAA |
| | Sequence | AGGACCAGAAAGAACCCAAAAATAAGACCTTTCTAAGATGCGAGGCCAAGAATTATTCTGGACGTTTCACCTGCTGGTGGCTGACGAC |
| | annotation | AATCAGTACTGATTTGACATTCAGTGTCAAAAGCAGCAGAGGGTCTTCTGACCCCCAAGGGGTGACGTGCGGAGCTGCTACACTCTCT |
| | CAPS: p40 | GCAGAGAGAGTCAGAGGGGACAACAAGGAGTATGAGTACTCAGTGGAGTGCCAGGAGGACAGTGCCTGCCCAGCTGCTGAGGAGAGTC |
| | domain; | TGCCCATTGAGGTCATGGTGGATGCCGTTCACAAGCTCAAGTATGAAAACTACACCAGCAGCTTCTTCATCAGGGACATCATCAAACC |
| | CAPS: | TGACCCACCCAAGAACTTGCAGCTGAAGCCATTAAAGAATTCTCGGCAGGTGGAGGTCAGCTGGGAGTACCCTGACACCTGGAGTACT |
| | linker; | CCACATTCCTACTTCTCCCTGACATTCTGCGTTCAGGTCCAGGGCAAGAGCAAGAGAGAAAAGAAAGATAGAGTCTTCACGGACAAGA |
| | CAPS: p35. | CCTCAGCCACGGTCATCTGCCGCAAAAATGCCAGCATTAGCGTGCGGGCCCAGGACCGCTACTATAGCTCATCTTGGAGCGAATGGGC |
| | | ATCTGTGCCCTGCAGTGGCTCTAGCGGAGGGGGAGGCTCTCCTGGCGGGGGATCTAGCAGAAACCTCCCCGTGGCCACTCCAGACCCA |
| | | GGAATGTTCCCATGCCTTCACCACTCCCAAAACCTGCTGAGGGCCGTCAGCAACATGCTCCAGAAGGCCAGACAAACTCTAGAATTTT |
| | | ACCCTTGCACTTCTGAGGAAATTGATCATGAAGATATCACAAAAGATAAAACCAGCACAGTGGAGGCCTGTTTACCATTGGAATTAAC |
| | | CAAGAATGAGAGTTGCCTAAATTCCAGAGAGACCTCTTTCATAACTAATGGGAGTTGCCTGGCCTCCAGAAAGACCTCTTTTATGATG |
| | | GCCCTGTGCCTTAGTAGTATTTATGAAGACTTGAAGATGTACCAGGTGGAGTTCAAGACCATGAATGCAAAGCTTCTGATGGATCCTA |
| | | AGAGGCAGATCTTTCTAGATCAAAACATGCTGGCAGTTATTGATGAGCTGATGCAGGCCCTGAATTTCAACAGTGAGACTGTGCCACA |
| | | AAAATCCTCCCTTGAAGAACCGGATTTTTATAAAACTAAAATCAAGCTCTGCATACTTCTTCATGCTTTCAGAATTCGGGCAGTGACT |
| | | ATTGATAGAGTGATGAGCTATCTGAATGCTTCCTGATGA |
| 16 | Human | ATGTGTCACCAGCAGCTGGTGATCTCATGGTTCTCCCTGGTATTTCTGGCATCTCCTCTTGTCGCAATCTGGGAACTGAAGAAAGACG |
| | optimized | TGTATGTCGTTGAGCTCGACTGGTATCCGGATGCGCCTGGCGAGATGGTGGTGCTGACCTGTGACACCCCAGAGGAGGATGGGATCAC |
| | IL-12sc | TTGGACCCTTGATCAATCCTCCGAAGTGCTCGGGTCTGGCAAGACTCTGACCATACAAGTGAAAGAGTTTGGCGATGCCGGGCAGTAC |
| | (CDS DNA) | ACTTGCCATAAGGGCGGAGAAGTTCTGTCCCACTCACTGCTGCTGCTGCACAAGAAAGAGGACGGAATTTGGAGTACCGATATCCTGA |
| | Sequence | AAGATCAGAAAGAGCCCAAGAACAAAACCTTCTTGCGGTGCGAAGCCAAGAACTACTCAGGGAGATTTACTTGTTGGTGGCTGACGAC |
| | annotation | GATCAGCACCGATCTGACTTTCTCCGTGAAATCAAGTAGGGGATCATCTGACCCTCAAGGAGTCACATGTGGAGCGGCTACTCTGAGC |
| | CAPS: p40 | GCTGAACGCGTAAGAGGGGACAATAAGGAGTACGAGTATAGCGTTGAGTGCCAAGAGGATAGCGCATGCCCCGCCGCCGAAGAATCAT |
| | domain; | TGCCCATTGAAGTGATGGTGGATGCTGTACACAAGCTGAAGTATGAGAACTACACAAGCTCCTTCTTCATCCGTGACATCATCAAACC |
| | CAPS: | AGATCCTCCTAAGAACCTCCAGCTTAAACCTCTGAAGAACTCTAGACAGGTGGAAGTGTCTTGGGAGTATCCCGACACCTGGTCTACA |
| | linker; | CCACATTCCTACTTCAGTCTCACATTCTGCGTTCAGGTACAGGGCAAGTCCAAAAGGGAGAAGAAGGATCGGGTCTTTACAGATAAAA |
| | CAPS: p35. | CAAGTGCCACCGTTATATGCCGGAAGAATGCCTCTATTTCTGTGCGTGCGCAGGACAGATACTATAGCAGCTCTTGGAGTGAATGGGC |
| | | CAGTGTCCCATGTTCAGGGTCATCCGGTGGTGGCGGCAGCCCCGGAGGCGGTAGCTCCAGAAATCTCCCTGTGGCTACACCTGATCCA |
| | | GGCATGTTTCCCTGTTTGCACCATAGCCAAAACCTCCTGAGAGCAGTCAGCAACATGCTCCAGAAAGCTAGACAAACACTGGAATTCT |
| | | ACCCATGCACCTCCGAGGAAATAGATCACGAGGATATCACTAAGGACAAAACAAGCACTGTCGAAGCATGCCTTCCCTTGGAACTGAC |
| | | AAAGAACGAGAGTTGCCTTAATTCAAGAGAAACATCTTTCATTACAAACGGTAGCTGCTTGGCAAGCAGAAAAACATCTTTTATGATG |
| | | GCCCTTTGTCTGAGCAGTATTTATGAGGATCTCAAAATGTACCAGGTGGAGTTTAAGACCATGAATGCCAAGCTGCTGATGGACCCAA |
| | | AGAGACAGATTTTCCTCGATCAGAATATGCTGGCTGTGATTGATGAACTGATGCAGGCCTTGAATTTCAACAGCGAAACCGTTCCCCA |
| | | GAAAAGCAGTCTTGAAGAACCTGACTTTTATAAGACCAAGATCAAACTGTGTATTCTCCTGCATGCCTTTAGAATCAGAGCAGTCACT |
| | | ATAGATAGAGTGATGTCCTACCTGAATGCTTCCTGATGA |
| 17 | Human non- | AUGUGUCACCAGCAGUUGGUCAUCUCUUGGUUUUCCCUGGUUUUUCUGGCAUCUCCCCUCGUGGCCAUAUGGGAACUGAAGAAAGAUG |
| | optimized | UUUAUGUCGUAGAAUUGGAUUGGUAUCCGGAUGCCCCUGGAGAAAUGGUGGUCCUCACCUGUGACACCCCUGAAGAAGAUGGUAUCAC |
| | IL-12sc | CUGGACCUUGGACCAGAGCAGUGAGGUCUUAGGCUCUGGCAAAACCCUGACCAUCCAAGUCAAAGAGUUUGGAGAUGCUGGCCAGUAC |
| | (RNA | ACCUGUCACAAAGGAGGCGAGGUUCUAAGCCAUUCGCUCCUGCUGCUUCACAAAAAGGAAGAUGGAAUUUGGUCCACUGAUAUUUUAA |
| | encoding | AGGACCAGAAAGAACCCAAAAAUAAGACCUUUCUAAGAUGCGAGGCCAAGAAUUAUUCUGGACGUUUCACCUGCUGGUGGCUGACGAC |
| | CDS) | AAUCAGUACUGAUUUGACAUUCAGUGUCAAAAGCAGCAGAGGGUCUUCUGACCCCCAAGGGGUGACGUGCGGAGCUGCUACACUCUCU |
| | | GCAGAGAGAGUCAGAGGGGACAACAAGGAGUAUGAGUACUCAGUGGAGUGCCAGGAGGACAGUGCCUGCCCAGCUGCUGAGGAGAGUC |
| | | UGCCCAUUGAGGUCAUGGUGGAUGCCGUUCACAAGCUCAAGUAUGAAAACUACACCAGCAGCUUCUUCAUCAGGGACAUCAUCAAACC |
| | | UGACCCACCCAAGAACUUGCAGCUGAAGCCAUUAAAGAAUUCUCGGCAGGUGGAGGUCAGCUGGGAGUACCCUGACACCUGGAGUACU |
| | | CCACAUUCCUACUUCUCCCUGACAUUCUGCGUUCAGGUCCAGGGCAAGAGCAAGAGAGAAAAGAAAGAUAGAGUCUUCACGGACAAGA |
| | | CCUCAGCCACGGUCAUCUGCCGCAAAAAUGCCAGCAUUAGCGUGCGGGCCCAGGACCGCUACUAUAGCUCAUCUUGGAGCGAAUGGGC |
| | | AUCUGUGCCCUGCAGUGGCUCUAGCGGAGGGGGAGGCUCUCCUGGCGGGGGAUCUAGCAGAAACCUCCCCGUGGCCACUCCAGACCCA |
| | | GGAAUGUUCCCAUGCCUUCACCACUCCCAAAACCUGCUGAGGGCCGUCAGCAACAUGCUCCAGAAGGCCAGACAAACUCUAGAAUUUU |
| | | ACCCUUGCACUUCUGAGGAAAUUGAUCAUGAAGAUAUCACAAAAGAUAAAACCAGCACAGUGGAGGCCUGUUUACCAUUGGAAUUAAC |
| | | CAAGAAUGAGAGUUGCCUAAAUUCCAGAGAGACCUCUUUCAUAACUAAUGGGAGUUGCCUGGCCUCCAGAAAGACCUCUUUUAUGAUG |
| | | GCCCUGUGCCUUAGUAGUAUUUAUGAAGACUUGAAGAUGUACCAGGUGGAGUUCAAGACCAUGAAUGCAAAGCUUCUGAUGGAUCCUA |
| | | AGAGGCAGAUCUUUCUAGAUCAAAACAUGCUGGCAGUUAUUGAUGAGCUGAUGCAGGCCCUGAAUUUCAACAGUGAGACUGUGCCACA |
| | | AAAAUCCUCCCUUGAAGAACCGGAUUUUUAUAAAACUAAAAUCAAGCUCUGCAUACUUCUUCAUGCUUUCAGAAUUCGGGCAGUGACU |
| | | AUUGAUAGAGUGAUGAGCUAUCUGAAUGCUUCCUGAUGA |
| 18 | Human | AUGUGUCACCAGCAGCUGGUGAUCUCAUGGUUCUCCCUGGUAUUUCUGGCAUCUCCUCUUGUCGCAAUCUGGGAACUGAAGAAAGACG |
| | optimized | UGUAUGUCGUUGAGCUCGACUGGUAUCCGGAUGCGCCUGGCGAGAUGGUGGUGCUGACCUGUGACACCCCAGAGGAGGAUGGGAUCAC |
| | IL-12sc | UUGGACCCUUGAUCAAUCCUCCGAAGUGCUCGGGUCUGGCAAGACUCUGACCAUACAAGUGAAAGAGUUUGGCGAUGCCGGGCAGUAC |
| | (RNA | ACUUGCCAUAAGGGCGGAGAAGUUCUGUCCCACUCACUGCUGCUGCUGCACAAGAAAGAGGACGGAAUUUGGAGUACCGAUAUCCUGA |
| | encoding | AAGAUCAGAAAGAGCCCAAGAACAAAACCUUCUUGCGGUGCGAAGCCAAGAACUACUCAGGGAGAUUUACUUGUUGGUGGCUGACGAC |
| | CDS) | GAUCAGCACCGAUCUGACUUUCUCCGUGAAAUCAAGUAGGGGAUCAUCUGACCCUCAAGGAGUCACAUGUGGAGCGGCUACUCUGAGC |
| | | GCUGAACGCGUAAGAGGGGACAAUAAGGAGUACGAGUAUAGCGUUGAGUGCCAAGAGGAUAGCGCAUGCCCCGCCGCCGAAGAAUCAU |
| | | UGCCCAUUGAAGUGAUGGUGGAUGCUGUACACAAGCUGAAGUAUGAGAACUACACAAGCUCCUUCUUCAUCCGUGACAUCAUCAAACC |
| | | AGAUCCUCCUAAGAACCUCCAGCUUAAACCUCUGAAGAACUCUAGACAGGUGGAAGUGUCUUGGGAGUAUCCCGACACCUGGUCUACA |
| | | CCACAUUCCUACUUCAGUCUCACAUUCUGCGUUCAGGUACAGGGCAAGUCCAAAAGGGAGAAGAAGGAUCGGGUCUUUACAGAUAAAA |
| | | CAAGUGCCACCGUUAUAUGCCGGAAGAAUGCCUCUAUUUCUGUGCGUGCGCAGGACAGAUACUAUAGCAGCUCUUGGAGUGAAUGGGC |
| | | CAGUGUCCCAUGUUCAGGGUCAUCCGGUGGUGGCGGCAGCCCCGGAGGCGGUAGCUCCAGAAAUCUCCCUGUGGCUACACCUGAUCCA |
| | | GGCAUGUUUCCCUGUUUGCACCAUAGCCAAAACCUCCUGAGAGCAGUCAGCAACAUGCUCCAGAAAGCUAGACAAACACUGGAAUUCU |
| | | ACCCAUGCACCUCCGAGGAAAUAGAUCACGAGGAUAUCACUAAGGACAAAACAAGCACUGUCGAAGCAUGCCUUCCCUUGGAACUGAC |
| | | AAAGAACGAGAGUUGCCUUAAUUCAAGAGAAACAUCUUUCAUUACAAACGGUAGCUGCUUGGCAAGCAGAAAAACAUCUUUUAUGAUG |
| | | GCCCUUUGUCUGAGCAGUAUUUAUGAGGAUCUCAAAAUGUACCAGGUGGAGUUUAAGACCAUGAAUGCCAAGCUGCUGAUGGACCCAA |
| | | AGAGACAGAUUUUCCUCGAUCAGAAUAUGCUGGCUGUGAUUGAUGAACUGAUGCAGGCCUUGAAUUUCAACAGCGAAACCGUUCCCCA |
| | | GAAAAGCAGUCUUGAAGAACCUGACUUUUAUAAGACCAAGAUCAAACUGUGUAUUCUCCUGCAUGCCUUUAGAAUCAGAGCAGUCACU |
| | | AUAGAUAGAGUGAUGUCCUACCUGAAUGCUUCCUGAUGA |

IFNalpha2b (IFNα2b)

| | | |
|---|---|---|
| 19 | Human | MALTFALLVALLVLSCKSSCSVGCDLPQTHSLGSRRTLMLLAQMRRISLFSCLKDRHDFGFPQEEFGNQFQKAETIPVLHEMIQQIFN |
| | IFNα2b | LFSTKDSSAAWDETLLDKFYTELYQQLNDLEACVIQGVGVTETPLMKEDSILAVRKYFQRITLYLKEKKYSPCAWEVVRAEIMRSFSL |
| | (amino | STNLQESLRSKE |
| | acid) | |
| 20 | Human non- | ATGGCCTTGACCTTTGCTTTACTGGTGGCCCTCCTGGTGCTCAGCTGCAAGTCAAGCTGCTCTGTGGGCTGTGATCTGCCTCAAACCC |
| | optimized | ACAGCCTGGGTAGCAGGAGGACCTTGATGCTCCTGGCACAGATGAGGAGAATCTCTCTTTTCTCCTGCTTGAAGGACAGACATGACTT |
| | IFNα2b (CDS | TGGATTTCCCCAGGAGGAGTTTGGCAACCAGTTCCAAAAGGCTGAAACCATCCCTGTCCTCCATGAGATGATCCAGCAGATCTTCAAC |
| | DNA) | CTTTTCAGCACAAAGGACTCATCTGCTGCTTGGGATGAGACCCTCCTAGACAAATTCTACACTGAACTCTACCAGCAGCTGAATGACC |
| | | TGGAAGCCTGTGTGATACAGGGGGTGGGGGTGACAGAGACTCCCCTGATGAAGGAGGACTCCATTCTGGCTGTGAGGAAATACTTCCA |
| | | AAGAATCACTCTCTATCTGAAAGAGAAGAAATACAGCCCTTGTGCCTGGGAGGTTGTCAGAGCAGAAATCATGAGATCTTTTTCTTTG |
| | | TCAACAAACTTGCAAGAAAGTTTAAGAAGTAAGGAATGATGA |
| 21 | Human | ATGGCCCTGACTTTTGCCCTTCTCGTGGCTTTGTTGGTGCTGAGTTGCAAATCTTCCTGTAGTGTCGGATGTGATCTGCCTCAAACCC |
| | optimized | ACAGTCTGGGATCTAGGAGAACACTGATGCTGTTGGCACAGATGAGGAGAATTAGCCTCTTTTCCTGCCTGAAGGATAGACATGACTT |
| | IFNα2b (CDS | CGGCTTTCCCCAAGAGGAGTTTGGCAATCAGTTCCAGAAAGCGGAAACGATTCCCGTTCTGCACGAGATGATCCAGCAGATCTTCAAC |
| | DNA) | CTCTTTTCAACCAAAGACAGCTCAGCAGCCTGGGATGAGACACTGCTGGACAAATTCTACACAGAACTGTATCAGCAGCTTAACGATC |
| | | TGGAGGCATGCGTGATCCAAGGGGTTGGTGTGACTGAAACTCCGCTTATGAAGGAGGACTCCATTCTGGCTGTACGGAAGTACTTCCA |
| | | GAGAATAACCCTCTATCTGAAGGAGAAGAAGTACTCACCATGTGCTTGGGAAGTCGTGAGAGCCGAAATCATGAGATCCTTCAGCCTT |
| | | AGCACCAATCTCCAGGAATCTCTGAGAAGCAAAGAGTGATGA |
| 22 | Human non- | AUGGCCUUGACCUUUGCUUUACUGGUGGCCCUCCUGGUGCUCAGCUGCAAGUCAAGCUGCUCUGUGGGCUGUGAUCUGCCUCAAACCC |
| | optimized | ACAGCCUGGGUAGCAGGAGGACCUUGAUGCUCCUGGCACAGAUGAGGAGAAUCUCUCUUUUCUCCUGCUUGAAGGACAGACAUGACUU |
| | IFNα2b (RNA | UGGAUUUCCCCAGGAGGAGUUUGGCAACCAGUUCCAAAAGGCUGAAACCAUCCCUGUCCUCCAUGAGAUGAUCCAGCAGAUCUUCAAC |
| | encoding | CUUUUCAGCACAAAGGACUCAUCUGCUGCUUGGGAUGAGACCCUCCUAGACAAAUUCUACACUGAACUCUACCAGCAGCUGAAUGACC |
| | CDS) | UGGAAGCCUGUGUGAUACAGGGGGUGGGGGUGACAGAGACUCCCCUGAUGAAGGAGGACUCCAUUCUGGCUGUGAGGAAAUACUUCCA |
| | | AAGAAUCACUCUCUAUCUGAAAGAGAAGAAAUACAGCCCUUGUGCCUGGGAGGUUGUCAGAGCAGAAAUCAUGAGAUCUUUUUCUUUG |
| | | UCAACAAACUUGCAAGAAAGUUUAAGAAGUAAGGAAUGAUGA |
| 23 | Human | AUGGCCCUGACUUUUGCCCUUCUCGUGGCUUUGUUGGUGCUGAGUUGCAAAUCUUCCUGUAGUGUCGGAUGUGAUCUGCCUCAAACCC |
| | optimized | ACAGUCUGGGAUCUAGGAGAACACUGAUGCUGUUGGCACAGAUGAGGAGAAUUAGCCUCUUUUCCUGCCUGAAGGAUAGACAUGACUU |
| | IFNα2b (RNA | CGGCUUUCCCCAAGAGGAGUUUGGCAAUCAGUUCCAGAAAGCGGAAACGAUUCCCGUUCUGCACGAGAUGAUCCAGCAGAUCUUCAAC |
| | encoding | CUCUUUUCAACCAAAGACAGCUCAGCAGCCUGGGAUGAGACACUGCUGGACAAAUUCUACACAGAACUGUAUCAGCAGCUUAACGAUC |
| | CDS) | UGGAGGCAUGCGUGAUCCAAGGGGUUGGUGUGACUGAAACUCCGCUUAUGAAGGAGGACUCCAUUCUGGCUGUACGGAAGUACUUCCA |
| | | GAGAAUAACCCUCUAUCUGAAGGAGAAGAAGUACUCACCAUGUGCUUGGGAAGUCGUGAGAGCCGAAAUCAUGAGAUCCUUCAGCCUU |
| | | AGCACCAAUCUCCAGGAAUCUCUGAGAAGCAAAGAGUGAUGA |

IL-15 sushi

| | | |
|---|---|---|
| 24 | Human IL-15 | MAPRRARGCRTLGLPALLLLLLLRPPATRGITCPPPMSVEHADIWVKSYSLYSRERYICNSGFKRKAGTSSLTECVLNKATNVAHWTT |
| | sushi | PSLKCIRDPALVHQRPAPPGGGSGGGGSGGGSGGGGSLQNWVNVISDLKKIEDLIQSMHIDATLYTESDVHPSCKVTAMKCFLLELQV |
| | (amino | ISLESGDASIHDTVENLIILANNSLSSNGNVTESGCKECEELEEKNIKEFLQSFVHIVQMFINTS |
| | acid) | |
| 25 | Human IL-15 | ATGGCCCCGCGGCGGGCGCGCGGCTGCCGGACCCTCGGTCTCCCGGCGCTGCTACTGCTGCTGCTGCTCCGGCCGCCGGCGACGCGGG |
| | sushi (CDS | GCATCACGTGCCCTCCCCCCATGTCCGTGGAACACGCAGACATCTGGGTCAAGAGCTACAGCTTGTACTCCAGGGAGCGGTACATTTG |
| | DNA) | TAACTCTGGTTTCAAGCGTAAAGCCGGCACGTCCAGCCTGACGGAGTGCGTGTTGAACAAGGCCACGAATGTCGCCCACTGGACAACC |
| | Sequence | CCCAGTCTCAAATGCATTAGAGACCCTGCCCTGGTTCACCAAAGGCCAGCGCCACCCGGGGGAGGATCTGGCGGCGGTGGGTCTGGCG |
| | annotations | GGGGATCTGGCGGAGGAGGAAGCTTACAGAACTGGGTGAATGTAATAAGTGATTTGAAAAAAATTGAAGATCTTATTCAATCTATGCA |
| | CAPS: IL-15 | TATTGATGCTACTTTATATACGGAAAGTGATGTTCACCCCAGTTGCAAAGTAACAGCAATGAAGTGCTTTCTCTTGGAGTTACAAGTT |
| | sushi; | ATTTCACTTGAGTCCGGAGATGCAAGTATTCATGATACAGTAGAAAATCTGATCATCCTAGCAAACAACAGTTTGTCTTCTAATGGGA |
| | CAPS: | ATGTAACAGAATCTGGATGCAAAGAATGTGAGGAACTGGAGGAAAAAAATATTAAAGAATTTTTGCAGAGTTTTGTACATATTGTCCA |
| | linker; | AATGTTCATCAACACTTCTTGATGA |
| | CAPS: | |
| | mature IL- | |
| | 15 | |
| 26 | Human IL-15 | AUGGCCCCGCGGCGGGCGCGCGGCUGCCGGACCCUCGGUCUCCCGGCGCUGCUACUGCUGCUGCUGCUCCGGCCGCCGGCGACGCGGG |
| | sushi (RNA | GCAUCACGUGCCCUCCCCCCAUGUCCGUGGAACACGCAGACAUCUGGGUCAAGAGCUACAGCUUGUACUCCAGGGAGCGGUACAUUUG |
| | encoding | UAACUCUGGUUUCAAGCGUAAAGCCGGCACGUCCAGCCUGACGGAGUGCGUGUUGAACAAGGCCACGAAUGUCGCCCACUGGACAACC |
| | CDS) | CCCAGUCUCAAAUGCAUUAGAGACCCUGCCCUGGUUCACCAAAGGCCAGCGCCACCCGGGGGAGGAUCUGGCGGCGGUGGGUCUGGCG |
| | | GGGGAUCUGGCGGAGGAGGAAGCUUACAGAACUGGGUGAAUGUAAUAAGUGAUUUGAAAAAAAUUGAAGAUCUUAUUCAAUCUAUGCA |
| | | UAUUGAUGCUACUUUAUAUACGGAAAGUGAUGUUCACCCCAGUUGCAAAGUAACAGCAAUGAAGUGCUUUCUCUUGGAGUUACAAGUU |
| | | AUUUCACUUGAGUCCGGAGAUGCAAGUAUUCAUGAUACAGUAGAAAAUCUGAUCAUCCUAGCAAACAACAGUUUGUCUUCUAAUGGGA |
| | | AUGUAACAGAAUCUGGAUGCAAAGAAUGUGAGGAACUGGAGGAAAAAAAUAUUAAAGAAUUUUUGCAGAGUUUUGUACAUAUUGUCCA |
| | | AAUGUUCAUCAACACUUCUUGAUGA |

GM-CSF

| | | |
|---|---|---|
| 27 | Human GM- | MWLQSLLLLGTVACSISAPARSPSPSTQPWEHVNAIQEARRLLNLSRDTAAEMNETVEVISEMFDLQEPTCLQTRLELYKQGLRGSLT |
| | CSF (amino | KLKGPLTMMASHYKQHCPPTPETSCATQIITFESFKENLKDFLLVIPFDCWEPVQE |
| | acid) | |
| 28 | Human GM- | ATGTGGCTCCAGAGCCTGCTGCTCTTGGGCACTGTGGCCTGCTCCATCTCTGCACCCGCCCGCTCGCCCAGCCCCAGCACGCAGCCCT |
| | CSF (CDS | GGGAGCATGTGAATGCCATCCAGGAGGCCCGGCGTCTGCTGAACCTGAGTAGAGACACTGCTGCTGAGATGAATGAAACAGTAGAAGT |
| | DNA) | CATCTCAGAAATGTTTGACCTCCAGGAGCCGACCTGCCTACAGACCCGCCTGGAGCTGTACAAGCAGGGCCTGCGGGGCAGCCTCACC |
| | | AAGCTCAAGGGCCCCTTGACCATGATGGCCAGCCACTACAAGCAGCACTGCCCTCCAACCCCGGAAACTTCCTGTGCAACCCAGATTA |
| | | TCACCTTTGAAAGTTTCAAAGAGAACCTGAAGGACTTTCTGCTTGTCATCCCCTTTGACTGCTGGGAGCCAGTCCAGGAGTGATGA |
| 29 | Human GM- | AUGUGGCUCCAGAGCCUGCUGCUCUUGGGCACUGUGGCCUGCUCCAUCUCUGCACCCGCCCGCUCGCCCAGCCCCAGCACGCAGCCCU |
| | CSF (RNA | GGGAGCAUGUGAAUGCCAUCCAGGAGGCCCGGCGUCUGCUGAACCUGAGUAGAGACACUGCUGCUGAGAUGAAUGAAACAGUAGAAGU |
| | encoding | CAUCUCAGAAAUGUUUGACCUCCAGGAGCCGACCUGCCUACAGACCCGCCUGGAGCUGUACAAGCAGGGCCUGCGGGGCAGCCUCACC |
| | CDS) | AAGCUCAAGGGCCCCUUGACCAUGAUGGCCAGCCACUACAAGCAGCACUGCCCUCCAACCCCGGAAACUUCCUGUGCAACCCAGAUUA |
| | | UCACCUUUGAAAGUUUCAAAGAGAACCUGAAGGACUUUCUGCUUGUCAUCCCCUUUGACUGCUGGGAGCCAGUCCAGGAGUGAUGA |

TABLE 2
DESCRIPTION OF THE SEQUENCES (mouse sequences and other sequences of the invention)

| | | |
|---|---|---|
| SEQ | | |
| ID | Des- | |
| NO: | cription | SEQUENCE |

IL-2 mouse

| | | |
|---|---|---|
| 30 | ModA | MRVTAPRTLILLLSGALALTETWAGSGSAPTSSSTKKTQLQLEHLLLDLQMILNGINNYKNPKLTRMLTFKFYMPKKATELKHLQCLE |
| | IL-2 | EELKPLEEVLNLAQSKNFHLRPRDLISNINVIVLELKGSETTFMCEYADETATIVEFLNRWITFCQSIISTLT |
| | (amino | |
| | acid, | |
| | human | |
| | IL-2 in | |
| | combi- | |
| | nation | |
| | with a | |
| | mouse | |
| | opti- | |
| | mized | |
| | secre- | |
| | tion | |
| | se- | |
| | quence) | |
| 31 | ModA | GGGCGAACTAGTATTCTTCTGGTCCCCACAGACTCAGAGAGAACCCGCCACCATGAGAGTGACCGCCCCCAGAACCCTGATCCTGCTG |
| | IL-2 | CTGTCTGGCGCCCTGGCCCTGACAGAGACATGGGCCGGAAGCGGATCCGCACCTACTTCAAGTTCTACAAAGAAAACACAGCTACAAC |
| | (DNA: 5′ | TGGAGCATTTACTTCTGGATTTACAGATGATTTTGAATGGAATTAATAATTACAAGAATCCCAAACTCACCAGGATGCTCACATTTAA |
| | UTR- | GTTTTACATGCCCAAGAAGGCCACAGAACTGAAACATCTTCAGTGTCTAGAAGAAGAACTCAAACCTCTGGAGGAAGTGCTAAATTTA |
| | CDS-3′ | GCTCAAAGCAAAAACTTTCACTTAAGACCCAGGGACTTAATCAGCAATATCAACGTAATAGTTCTGGAACTAAAGGGATCTGAAACAA |
| | UTR) | CATTCATGTGTGAATATGCTGATGAGACAGCAACCATTGTAGAATTTCTGAACAGATGGATTACCTTTTGTCAAAGCATCATCTCAAC |
| | | ACTGACTTGACTCGAGAGCTCGCTTTCTTGCTGTCCAATTTCTATTAAAGGTTCCTTTGTTCCCTAAGTCCAACTACTAAACTGGGGG |
| | | ATATTATGAAGGGCCTTGAGCATCTGGATTCTGCCTAATAAAAAACATTTATTTTCATTGCTGCGTCGAGAGCTCGCTTTCTTGCTGT |
| | | CCAATTTCTATTAAAGGTTCCTTTGTTCCCTAAGTCCAACTACTAAACTGGGGGATATTATGAAGGGCCTTGAGCATCTGGATTCTGC |
| | | CTAATAAAAAACATTTATTTTCATTGCTGCGTCGAGACCTGGTCCAGAGTCGCTAGCAAAAAAAAAAAAAAAAAAAAAAAAAAAAAAG |
| | | CATATGACTAAAAAAAAAAAAAAAAAAAAAAAAAAAAAAAAAAAAAAAAAAAAAAAAAAAAAAAAAAAAAAAAAAAAAA |
| 32 | ModA | GGGCGAACUAGUAUUCUUCUGGUCCCCACAGACUCAGAGAGAACCCGCCACCAUGAGAGUGACCGCCCCCAGAACCCUGAUCCUGCUG |
| | IL-2 | CUGUCUGGCGCCCUGGCCCUGACAGAGACAUGGGCCGGAAGCGGAUCCGCACCUACUUCAAGUUCUACAAAGAAAACACAGCUACAAC |
| | (RNA) | UGGAGCAUUUACUUCUGGAUUUACAGAUGAUUUUGAAUGGAAUUAAUAAUUACAAGAAUCCCAAACUCACCAGGAUGCUCACAUUUAA |
| | | GUUUUACAUGCCCAAGAAGGCCACAGAACUGAAACAUCUUCAGUGUCUAGAAGAAGAACUCAAACCUCUGGAGGAAGUGCUAAAUUUA |
| | | GCUCAAAGCAAAAACUUUCACUUAAGACCCAGGGACUUAAUCAGCAAUAUCAACGUAAUAGUUCUGGAACUAAAGGGAUCUGAAACAA |
| | | CAUUCAUGUGUGAAUAUGCUGAUGAGACAGCAACCAUUGUAGAAUUUCUGAACAGAUGGAUUACCUUUUGUCAAAGCAUCAUCUCAAC |
| | | ACUGACUUGACUCGAGAGCUCGCUUUCUUGCUGUCCAAUUUCUAUUAAAGGUUCCUUUGUUCCCUAAGUCCAACUACUAAACUGGGGG |
| | | AUAUUAUGAAGGGCCUUGAGCAUCUGGAUUCUGCCUAAUAAAAAACAUUUAUUUUCAUUGCUGCGUCGAGAGCUCGCUUUCUUGCUGU |
| | | CCAAUUUCUAUUAAAGGUUCCUUUGUUCCCUAAGUCCAACUACUAAACUGGGGGAUAUUAUGAAGGGCCUUGAGCAUCUGGAUUCUGC |
| | | CUAAUAAAAAACAUUUAUUUUCAUUGCUGCGUCGAGACCUGGUCCAGAGUCGCUAGCAAAAAAAAAAAAAAAAAAAAAAAAAAAAAAG |
| | | CAUAUGACUAAAAAAAAAAAAAAAAAAAAAAAAAAAAAAAAAAAAAAAAAAAAAAAAAAAAAAAAAAAAAAAAAAAAAA |
| 33 | ModB | MGAMAPRTLLLLLAAALAPTQTRAGPGSAPTSSSTKKTQLQLEHLLLDLQMILNGINNYKNPKLTRMLTFKFYMPKKATELKHLQCLE |
| | IL-2 | EELKPLEEVLNLAQSKNFHLRPRDLISNINVIVLELKGSETTFMCEYADETATIVEFLNRWITFCQSIISTLT |
| | (amino | |
| | acid) | |
| 34 | ModB | GGAATAAACTAGTCTCAACACAACATATACAAAACAAACGAATCTCAAGCAATCAAGCATTCTACTTCTATTGCAGCAATTTAAATCA |
| | IL-2 | TTTCTTTTAAAGCAAAAGCAATTTTCTGAAAATTTTCACCATTTACGAACGATAGCCATGGGCGCCATGGCCCCTAGAACATTGCTCC |
| | (DNA: | TGCTGCTGGCCGCTGCCCTGGCCCCTACACAGACAAGAGCTGGACCTGGATCCGCACCTACTTCAAGTTCTACAAAGAAAACACAGCT |
| | 5′ UTR- | ACAACTGGAGCATTTACTTCTGGATTTACAGATGATTTTGAATGGAATTAATAATTACAAGAATCCCAAACTCACCAGGATGCTCACA |
| | CDS-3′ | TTTAAGTTTTACATGCCCAAGAAGGCCACAGAACTGAAACATCTTCAGTGTCTAGAAGAAGAACTCAAACCTCTGGAGGAAGTGCTAA |
| | UTR) | ATTTAGCTCAAAGCAAAAACTTTCACTTAAGACCCAGGGACTTAATCAGCAATATCAACGTAATAGTTCTGGAACTAAAGGGATCTGA |
| | | AACAACATTCATGTGTGAATATGCTGATGAGACAGCAACCATTGTAGAATTTCTGAACAGATGGATTACCTTTTGTCAAAGCATCATC |
| | | TCAACACTGACTTGACTCGACGTCCTGGTACTGCATGCACGCAATGCTAGCTGCCCCTTTCCCGTCCTGGGTACCCCGAGTCTCCCCC |
| | | GACCTCGGGTCCCAGGTATGCTCCCACCTCCACCTGCCCCACTCACCACCTCTGCTAGTTCCAGACACCTCCCAAGCACGCAGCAATG |
| | | CAGCTCAAAACGCTTAGCCTAGCCACACCCCCACGGGAAACAGCAGTGATTAACCTTTAGCAATAAACGAAAGTTTAACTAAGCTATA |
| | | CTAACCCCAGGGTTGGTCAATTTCGTGCCAGCCACACCCTCGAGCTAGCAAAAAAAAAAAAAAAAAAAAAAAAAAAAAAGCATATGAC |
| | | TAAAAAAAAAAAAAAAAAAAAAAAAAAAAAAAAAAAAAAAAAAAAAAAAAAAAAAAAAAAAAAAAAAAAAA |
| 35 | ModB | GGAAUAAACUAGUCUCAACACAACAUAUACAAAACAAACGAAUCUCAAGCAAUCAAGCAUUCUACUUCUAUUGCAGCAAUUUAAAUCA |
| | IL-2 | UUUCUUUUAAAGCAAAAGCAAUUUUCUGAAAAUUUUCACCAUUUACGAACGAUAGCCAUGGGCGCCAUGGCCCCUAGAACAUUGCUCC |
| | (RNA) | UGCUGCUGGCCGCUGCCCUGGCCCCUACACAGACAAGAGCUGGACCUGGAUCCGCACCUACUUCAAGUUCUACAAAGAAAACACAGCU |
| | | ACAACUGGAGCAUUUACUUCUGGAUUUACAGAUGAUUUUGAAUGGAAUUAAUAAUUACAAGAAUCCCAAACUCACCAGGAUGCUCACA |
| | | UUUAAGUUUUACAUGCCCAAGAAGGCCACAGAACUGAAACAUCUUCAGUGUCUAGAAGAAGAACUCAAACCUCUGGAGGAAGUGCUAA |
| | | AUUUAGCUCAAAGCAAAAACUUUCACUUAAGACCCAGGGACUUAAUCAGCAAUAUCAACGUAAUAGUUCUGGAACUAAAGGGAUCUGA |
| | | AACAACAUUCAUGUGUGAAUAUGCUGAUGAGACAGCAACCAUUGUAGAAUUUCUGAACAGAUGGAUUACCUUUUGUCAAAGCAUCAUC |
| | | UCAACACUGACUUGACUCGACGUCCUGGUACUGCAUGCACGCAAUGCUAGCUGCCCCUUUCCCGUCCUGGGUACCCCGAGUCUCCCCC |
| | | GACCUCGGGUCCCAGGUAUGCUCCCACCUCCACCUGCCCCACUCACCACCUCUGCUAGUUCCAGACACCUCCCAAGCACGCAGCAAUG |
| | | CAGCUCAAAACGCUUAGCCUAGCCACACCCCCACGGGAAACAGCAGUGAUUAACCUUUAGCAAUAAACGAAAGUUUAACUAAGCUAUA |
| | | CUAACCCCAGGGUUGGUCAAUUUCGUGCCAGCCACACCCUCGAGCUAGCAAAAAAAAAAAAAAAAAAAAAAAAAAAAAAGCAUAUGAC |
| | | UAAAAAAAAAAAAAAAAAAAAAAAAAAAAAAAAAAAAAAAAAAAAAAAAAAAAAAAAAAAAAAAAAAAAAA |

IL-12 mouse

| | | |
|---|---|---|
| 36 | ModA | MRVTAPRTLILLLSGALALTETWAGSGSMWELEKDVYVVEVDWTPDAPGETVNLTCDTPEEDDITWTSDQRHGVIGSGKTLTITVKEF |
| | murine | LDAGQYTCHKGGETLSHSHLLLHKKENGIWSTEILKNFKNKTFLKCEAPNYSGRFTCSWLVQRNMDLKFNIKSSSSSPDSRAVTCGMA |
| | IL-12 | SLSAEKVTLDQRDYEKYSVSCQEDVTCPTAEETLPIELALEARQQNKYENYSTSFFIRDIIKPDPPKNLQMKPLKNSQVEVSWEYPDS |
| | (amino | WSTPHSYFSLKFFVRIQRKKEKMKETEEGCNQKGAFLVEKTSTEVQCKGGNVCVQAQDRYYNSSCSKWACVPCRVRSVPGVGVPGVGR |
| | acid) | VIPVSGPARCLSQSRNLLKTTDDMVKTAREKLKHYSCTAEDIDHEDITRDQTSTLKTCLPLELHKNESCLATRETSSTTRGSCLPPQK |
| | | TSLMMTLCLGSIYEDLKMYQTEFQAINAALQNHNHQQIILDKGMLVAIDELMQSLNHNGETLRQKPPVGEADPYRVKMKLCILLHAFS |
| | | TRVVTINRVMGYLSSA |
| 37 | ModA | GGGCGAACTAGTATTCTTCTGGTCCCCACAGACTCAGAGAGAACCCGCCACCATGAGAGTGACCGCCCCCAGAACCCTGATCCTGCTG |
| | murine | CTGTCTGGCGCCCTGGCCCTGACAGAGACATGGGCCGGAAGCGGATCCATGTGGGAGCTGGAGAAAGACGTTTATGTTGTAGAGGTGG |
| | IL-12 | ACTGGACTCCCGATGCCCCTGGAGAAACAGTGAACCTCACCTGTGACACGCCTGAAGAAGATGACATCACCTGGACCTCAGACCAGAG |
| | (DNA: | ACATGGAGTCATAGGCTCTGGAAAGACCCTGACCATCACTGTCAAAGAGTTTCTAGATGCTGGCCAGTACACCTGCCACAAAGGAGGC |
| | 5′ UTR- | GAGACTCTGAGCCACTCACATCTGCTGCTCCACAAGAAGGAAAATGGAATTTGGTCCACTGAAATTTTAAAAAATTTCAAAAACAAGA |
| | CDS- | CTTTCCTGAAGTGTGAAGCACCAAATTACTCCGGACGGTTCACGTGCTCATGGCTGGTGCAAAGAAACATGGACTTGAAGTTCAACAT |
| | 3′ UTR) | CAAGAGCAGTAGCAGTTCCCCTGACTCTCGGGCAGTGACATGTGGAATGGCGTCTCTGTCTGCAGAGAAGGTCACACTGGACCAAAGG |
| | | GACTATGAGAAGTATTCAGTGTCCTGCCAGGAGGATGTCACCTGCCCAACTGCCGAGGAGACCCTGCCCATTGAACTGGCGTTGGAAG |
| | | CACGGCAGCAGAATAAATATGAGAACTACAGCACCAGCTTCTTCATCAGGGACATCATCAAACCAGACCCGCCCAAGAACTTGCAGAT |
| | | GAAGCCTTTGAAGAACTCACAGGTGGAGGTCAGCTGGGAGTACCCTGACTCCTGGAGCACTCCCCATTCCTACTTCTCCCTCAAGTTC |
| | | TTTGTTCGAATCCAGCGCAAGAAAGAAAAGATGAAGGAGACAGAGGAGGGGTGTAACCAGAAAGGTGCGTTCCTCGTAGAGAAGACAT |
| | | CTACCGAAGTCCAATGCAAAGGCGGGAATGTCTGCGTGCAAGCTCAGGATCGCTATTACAATTCCTCATGCAGCAAGTGGGCATGTGT |
| | | TCCCTGCAGAGTCCGATCGGTTCCTGGAGTAGGGGTACCTGGAGTGGGCAGGGTCATACCGGTCTCTGGACCTGCCAGGTGTCTTAGC |
| | | CAGTCCCGAAACCTGCTGAAGACCACAGATGACATGGTGAAGACGGCCAGAGAAAAGCTGAAACATTATTCCTGCACTGCTGAAGACA |
| | | TCGATCATGAAGACATCACACGGGACCAAACCAGCACATTGAAGACCTGTTTACCACTGGAACTACACAAGAACGAGAGTTGCCTGGC |
| | | TACTAGAGAGACTTCTTCCACAACAAGAGGGAGCTGCCTGCCCCCACAGAAGACGTCTTTGATGATGACCCTGTGCCTTGGTAGCATC |
| | | TATGAGGACTTGAAGATGTACCAGACAGAGTTCCAGGCCATCAACGCAGCACTTCAGAATCACAACCATCAGCAGATCATTCTAGACA |
| | | AGGGCATGCTGGTGGCCATCGATGAGCTGATGCAGTCTCTGAATCATAATGGCGAGACTCTGCGCCAGAAACCTCCTGTGGGAGAAGC |
| | | AGACCCTTACAGAGTGAAAATGAAGCTCTGCATCCTGCTTCACGCCTTCAGCACCCGCGTCGTGACCATCAACAGGGTGATGGGCTAT |
| | | CTGTCCAGCGCCTAATAGCTCGAGAGCTCGCTTTCTTGCTGTCCAATTTCTATTAAAGGTTCCTTTGTTCCCTAAGTCCAACTACTAA |
| | | ACTGGGGGATATTATGAAGGGCCTTGAGCATCTGGATTCTGCCTAATAAAAAACATTTATTTTCATTGCTGCGTCGAGAGCTCGCTTT |
| | | CTTGCTGTCCAATTTCTATTAAAGGTTCCTTTGTTCCCTAAGTCCAACTACTAAACTGGGGGATATTATGAAGGGCCTTGAGCATCTG |
| | | GATTCTGCCTAATAAAAAACATTTATTTTCATTGCTGCGTCGAGACCTGGTCCAGAGTCGCTAGCAAAAAAAAAAAAAAAAAAAAAAA |
| | | AAAAAAAGCATATGACTAAAAAAAAAAAAAAAAAAAAAAAAAAAAAAAAAAAAAAAAAAAAAAAAAAAAAAAAAAAAAAAAAAAAAA |
| 38 | ModA | GGGCGAACUAGUAUUCUUCUGGUCCCCACAGACUCAGAGAGAACCCGCCACCAUGAGAGUGACCGCCCCCAGAACCCUGAUCCUGCUG |
| | murine | CUGUCUGGCGCCCUGGCCCUGACAGAGACAUGGGCCGGAAGCGGAUCCAUGUGGGAGCUGGAGAAAGACGUUUAUGUUGUAGAGGUGG |
| | IL-12 | ACUGGACUCCCGAUGCCCCUGGAGAAACAGUGAACCUCACCUGUGACACGCCUGAAGAAGAUGACAUCACCUGGACCUCAGACCAGAG |
| | (RNA) | ACAUGGAGUCAUAGGCUCUGGAAAGACCCUGACCAUCACUGUCAAAGAGUUUCUAGAUGCUGGCCAGUACACCUGCCACAAAGGAGGC |
| | | GAGACUCUGAGCCACUCACAUCUGCUGCUCCACAAGAAGGAAAAUGGAAUUUGGUCCACUGAAAUUUUAAAAAAUUUCAAAAACAAGA |
| | | CUUUCCUGAAGUGUGAAGCACCAAAUUACUCCGGACGGUUCACGUGCUCAUGGCUGGUGCAAAGAAACAUGGACUUGAAGUUCAACAU |
| | | CAAGAGCAGUAGCAGUUCCCCUGACUCUCGGGCAGUGACAUGUGGAAUGGCGUCUCUGUCUGCAGAGAAGGUCACACUGGACCAAAGG |
| | | GACUAUGAGAAGUAUUCAGUGUCCUGCCAGGAGGAUGUCACCUGCCCAACUGCCGAGGAGACCCUGCCCAUUGAACUGGCGUUGGAAG |
| | | CACGGCAGCAGAAUAAAUAUGAGAACUACAGCACCAGCUUCUUCAUCAGGGACAUCAUCAAACCAGACCCGCCCAAGAACUUGCAGAU |
| | | GAAGCCUUUGAAGAACUCACAGGUGGAGGUCAGCUGGGAGUACCCUGACUCCUGGAGCACUCCCCAUUCCUACUUCUCCCUCAAGUUC |
| | | UUUGUUCGAAUCCAGCGCAAGAAAGAAAAGAUGAAGGAGACAGAGGAGGGGUGUAACCAGAAAGGUGCGUUCCUCGUAGAGAAGACAU |
| | | CUACCGAAGUCCAAUGCAAAGGCGGGAAUGUCUGCGUGCAAGCUCAGGAUCGCUAUUACAAUUCCUCAUGCAGCAAGUGGGCAUGUGU |
| | | UCCCUGCAGAGUCCGAUCGGUUCCUGGAGUAGGGGUACCUGGAGUGGGCAGGGUCAUACCGGUCUCUGGACCUGCCAGGUGUCUUAGC |
| | | CAGUCCCGAAACCUGCUGAAGACCACAGAUGACAUGGUGAAGACGGCCAGAGAAAAGCUGAAACAUUAUUCCUGCACUGCUGAAGACA |
| | | UCGAUCAUGAAGACAUCACACGGGACCAAACCAGCACAUUGAAGACCUGUUUACCACUGGAACUACACAAGAACGAGAGUUGCCUGGC |
| | | UACUAGAGAGACUUCUUCCACAACAAGAGGGAGCUGCCUGCCCCCACAGAAGACGUCUUUGAUGAUGACCCUGUGCCUUGGUAGCAUC |
| | | UAUGAGGACUUGAAGAUGUACCAGACAGAGUUCCAGGCCAUCAACGCAGCACUUCAGAAUCACAACCAUCAGCAGAUCAUUCUAGACA |
| | | AGGGCAUGCUGGUGGCCAUCGAUGAGCUGAUGCAGUCUCUGAAUCAUAAUGGCGAGACUCUGCGCCAGAAACCUCCUGUGGGAGAAGC |
| | | AGACCCUUACAGAGUGAAAAUGAAGCUCUGCAUCCUGCUUCACGCCUUCAGCACCCGCGUCGUGACCAUCAACAGGGUGAUGGGCUAU |
| | | CUGUCCAGCGCCUAAUAGCUCGAGAGCUCGCUUUCUUGCUGUCCAAUUUCUAUUAAAGGUUCCUUUGUUCCCUAAGUCCAACUACUAA |
| | | ACUGGGGGAUAUUAUGAAGGGCCUUGAGCAUCUGGAUUCUGCCUAAUAAAAAACAUUUAUUUUCAUUGCUGCGUCGAGAGCUCGCUUU |
| | | CUUGCUGUCCAAUUUCUAUUAAAGGUUCCUUUGUUCCCUAAGUCCAACUACUAAACUGGGGGAUAUUAUGAAGGGCCUUGAGCAUCUG |
| | | GAUUCUGCCUAAUAAAAAACAUUUAUUUUCAUUGCUGCGUCGAGACCUGGUCCAGAGUCGCUAGCAAAAAAAAAAAAAAAAAAAAAAA |
| | | AAAAAAAGCAUAUGACUAAAAAAAAAAAAAAAAAAAAAAAAAAAAAAAAAAAAAAAAAAAAAAAAAAAAAAAAAAAAAAAAAAAAAA |
| 39 | ModB | MGAMAPRTLLLLLAAALAPTQTRAGPGSMWELEKDVYVVEVDWTPDAPGETVNLTCDTPEEDDITWTSDQRHGVIGSGKTLTITVKEF |
| | murine | LDAGQYTCHKGGETLSHSHLLLHKKENGIWSTEILKNFKNKTFLKCEAPNYSGRFTCSWLVQRNMDLKFNIKSSSSSPDSRAVTCGMA |
| | IL-12 | SLSAEKVTLDQRDYEKYSVSCQEDVTCPTAEETLPIELALEARQQNKYENYSTSFFIRDIIKPDPPKNLQMKPLKNSQVEVSWEYPDS |
| | (amino | WSTPHSYFSLKFFVRIQRKKEKMKETEEGCNQKGAFLVEKTSTEVQCKGGNVCVQAQDRYYNSSCSKWACVPCRVRSVPGVGVPGVGR |
| | acid) | VIPVSGPARCLSQSRNLLKTTDDMVKTAREKLKHYSCTAEDIDHEDITRDQTSTLKTCLPLELHKNESCLATRETSSTTRGSCLPPQK |
| | | TSLMMTLCLGSIYEDLKMYQTEFQAINAALQNHNHQQIILDKGMLVAIDELMQSLNHNGETLRQKPPVGEADPYRVKMKLCILLHAFS |
| | | TRVVTINRVMGYLSSA |
| 40 | ModB | GGAATAAACTAGTCTCAACACAACATATACAAAACAAACGAATCTCAAGCAATCAAGCATTCTACTTCTATTGCAGCAATTTAAATCA |
| | murine | TTTCTTTTAAAGCAAAAGCAATTTTCTGAAAATTTTCACCATTTACGAACGATAGCCATGGGCGCCATGGCCCCTAGAACATTGCTCC |
| | IL-12 | TGCTGCTGGCCGCTGCCCTGGCCCCTACACAGACAAGAGCTGGACCTGGATCCATGTGGGAGCTGGAGAAAGACGTTTATGTTGTAGA |
| | (DNA: 5′ | GGTGGACTGGACTCCCGATGCCCCTGGAGAAACAGTGAACCTCACCTGTGACACGCCTGAAGAAGATGACATCACCTGGACCTCAGAC |
| | UTR- | CAGAGACATGGAGTCATAGGCTCTGGAAAGACCCTGACCATCACTGTCAAAGAGTTTCTAGATGCTGGCCAGTACACCTGCCACAAAG |
| | CDS-3′ | GAGGCGAGACTCTGAGCCACTCACATCTGCTGCTCCACAAGAAGGAAAATGGAATTTGGTCCACTGAAATTTTAAAAAATTTCAAAAA |
| | UTR) | CAAGACTTTCCTGAAGTGTGAAGCACCAAATTACTCCGGACGGTTCACGTGCTCATGGCTGGTGCAAAGAAACATGGACTTGAAGTTC |
| | | AACATCAAGAGCAGTAGCAGTTCCCCTGACTCTCGGGCAGTGACATGTGGAATGGCGTCTCTGTCTGCAGAGAAGGTCACACTGGACC |
| | | AAAGGGACTATGAGAAGTATTCAGTGTCCTGCCAGGAGGATGTCACCTGCCCAACTGCCGAGGAGACCCTGCCCATTGAACTGGCGTT |
| | | GGAAGCACGGCAGCAGAATAAATATGAGAACTACAGCACCAGCTTCTTCATCAGGGACATCATCAAACCAGACCCGCCCAAGAACTTG |
| | | CAGATGAAGCCTTTGAAGAACTCACAGGTGGAGGTCAGCTGGGAGTACCCTGACTCCTGGAGCACTCCCCATTCCTACTTCTCCCTCA |
| | | AGTTCTTTGTTCGAATCCAGCGCAAGAAAGAAAAGATGAAGGAGACAGAGGAGGGGTGTAACCAGAAAGGTGCGTTCCTCGTAGAGAA |
| | | GACATCTACCGAAGTCCAATGCAAAGGCGGGAATGTCTGCGTGCAAGCTCAGGATCGCTATTACAATTCCTCATGCAGCAAGTGGGCA |
| | | TGTGTTCCCTGCAGAGTCCGATCGGTTCCTGGAGTAGGGGTACCTGGAGTGGGCAGGGTCATACCGGTCTCTGGACCTGCCAGGTGTC |
| | | TTAGCCAGTCCCGAAACCTGCTGAAGACCACAGATGACATGGTGAAGACGGCCAGAGAAAAGCTGAAACATTATTCCTGCACTGCTGA |
| | | AGACATCGATCATGAAGACATCACACGGGACCAAACCAGCACATTGAAGACCTGTTTACCACTGGAACTACACAAGAACGAGAGTTGC |
| | | CTGGCTACTAGAGAGACTTCTTCCACAACAAGAGGGAGCTGCCTGCCCCCACAGAAGACGTCTTTGATGATGACCCTGTGCCTTGGTA |
| | | GCATCTATGAGGACTTGAAGATGTACCAGACAGAGTTCCAGGCCATCAACGCAGCACTTCAGAATCACAACCATCAGCAGATCATTCT |
| | | AGACAAGGGCATGCTGGTGGCCATCGATGAGCTGATGCAGTCTCTGAATCATAATGGCGAGACTCTGCGCCAGAAACCTCCTGTGGGA |
| | | GAAGCAGACCCTTACAGAGTGAAAATGAAGCTCTGCATCCTGCTTCACGCCTTCAGCACCCGCGTCGTGACCATCAACAGGGTGATGG |
| | | GCTATCTGTCCAGCGCCTAATAGCTCGACGTCCTGGTACTGCATGCACGCAATGCTAGCTGCCCCTTTCCCGTCCTGGGTACCCCGAG |
| | | TCTCCCCCGACCTCGGGTCCCAGGTATGCTCCCACCTCCACCTGCCCCACTCACCACCTCTGCTAGTTCCAGACACCTCCCAAGCACG |
| | | CAGCAATGCAGCTCAAAACGCTTAGCCTAGCCACACCCCCACGGGAAACAGCAGTGATTAACCTTTAGCAATAAACGAAAGTTTAACT |
| | | AAGCTATACTAACCCCAGGGTTGGTCAATTTCGTGCCAGCCACACCCTCGAGCTAGCAAAAAAAAAAAAAAAAAAAAAAAAAAAAAAG |
| | | CATATGACTAAAAAAAAAAAAAAAAAAAAAAAAAAAAAAAAAAAAAAAAAAAAAAAAAAAAAAAAAAAAAAAAAAAAAA |
| 41 | ModB | GGAAUAAACUAGUCUCAACACAACAUAUACAAAACAAACGAAUCUCAAGCAAUCAAGCAUUCUACUUCUAUUGCAGCAAUUUAAAUCA |
| | murine | UUUCUUUUAAAGCAAAAGCAAUUUUCUGAAAAUUUUCACCAUUUACGAACGAUAGCCAUGGGCGCCAUGGCCCCUAGAACAUUGCUCC |
| | IL-12 | UGCUGCUGGCCGCUGCCCUGGCCCCUACACAGACAAGAGCUGGACCUGGAUCCAUGUGGGAGCUGGAGAAAGACGUUUAUGUUGUAGA |
| | (RNA) | GGUGGACUGGACUCCCGAUGCCCCUGGAGAAACAGUGAACCUCACCUGUGACACGCCUGAAGAAGAUGACAUCACCUGGACCUCAGAC |
| | | CAGAGACAUGGAGUCAUAGGCUCUGGAAAGACCCUGACCAUCACUGUCAAAGAGUUUCUAGAUGCUGGCCAGUACACCUGCCACAAAG |
| | | CAAGACUUUCCUGAAGUGUGAAGCACCAAAUUACUCCGGACGGUUCACGUGCUCAUGGCUGGUGCAAAGAAACAUGGACUUGAAGUUC |
| | | AACAUCAAGAGCAGUAGCAGUUCCCCUGACUCUCGGGCAGUGACAUGUGGAAUGGCGUCUCUGUCUGCAGAGAAGGUCACACUGGACC |
| | | AAAGGGACUAUGAGAAGUAUUCAGUGUCCUGCCAGGAGGAUGUCACCUGCCCAACUGCCGAGGAGACCCUGCCCAUUGAACUGGCGUU |
| | | GGAAGCACGGCAGCAGAAUAAAUAUGAGAACUACAGCACCAGCUUCUUCAUCAGGGACAUCAUCAAACCAGACCCGCCCAAGAACUUG |
| | | CAGAUGAAGCCUUUGAAGAACUCACAGGUGGAGGUCAGCUGGGAGUACCCUGACUCCUGGAGCACUCCCCAUUCCUACUUCUCCCUCA |
| | | AGUUCUUUGUUCGAAUCCAGCGCAAGAAAGAAAAGAUGAAGGAGACAGAGGAGGGGUGUAACCAGAAAGGUGCGUUCCUCGUAGAGAA |
| | | GACAUCUACCGAAGUCCAAUGCAAAGGCGGGAAUGUCUGCGUGCAAGCUCAGGAUCGCUAUUACAAUUCCUCAUGCAGCAAGUGGGCA |
| | | UGUGUUCCCUGCAGAGUCCGAUCGGUUCCUGGAGUAGGGGUACCUGGAGUGGGCAGGGUCAUACCGGUCUCUGGACCUGCCAGGUGUC |
| | | UUAGCCAGUCCCGAAACCUGCUGAAGACCACAGAUGACAUGGUGAAGACGGCCAGAGAAAAGCUGAAACAUUAUUCCUGCACUGCUGA |
| | | AGACAUCGAUCAUGAAGACAUCACACGGGACCAAACCAGCACAUUGAAGACCUGUUUACCACUGGAACUACACAAGAACGAGAGUUGC |
| | | CUGGCUACUAGAGAGACUUCUUCCACAACAAGAGGGAGCUGCCUGCCCCCACAGAAGACGUCUUUGAUGAUGACCCUGUGCCUUGGUA |
| | | GCAUCUAUGAGGACUUGAAGAUGUACCAGACAGAGUUCCAGGCCAUCAACGCAGCACUUCAGAAUCACAACCAUCAGCAGAUCAUUCU |
| | | AGACAAGGGCAUGCUGGUGGCCAUCGAUGAGCUGAUGCAGUCUCUGAAUCAUAAUGGCGAGACUCUGCGCCAGAAACCUCCUGUGGGA |
| | | GAAGCAGACCCUUACAGAGUGAAAAUGAAGCUCUGCAUCCUGCUUCACGCCUUCAGCACCCGCGUCGUGACCAUCAACAGGGUGAUGG |
| | | GCUAUCUGUCCAGCGCCUAAUAGCUCGACGUCCUGGUACUGCAUGCACGCAAUGCUAGCUGCCCCUUUCCCGUCCUGGGUACCCCGAG |
| | | UCUCCCCCGACCUCGGGUCCCAGGUAUGCUCCCACCUCCACCUGCCCCACUCACCACCUCUGCUAGUUCCAGACACCUCCCAAGCACG |
| | | CAGCAAUGCAGCUCAAAACGCUUAGCCUAGCCACACCCCCACGGGAAACAGCAGUGAUUAACCUUUAGCAAUAAACGAAAGUUUAACU |
| | | AAGCUAUACUAACCCCAGGGUUGGUCAAUUUCGUGCCAGCCACACCCUCGAGCUAGCAAAAAAAAAAAAAAAAAAAAAAAAAAAAAAG |
| | | CAUAUGACUAAAAAAAAAAAAAAAAAAAAAAAAAAAAAAAAAAAAAAAAAAAAAAAAAAAAAAAAAAAAAAAAAAAAAA |

IFNα (IFNα4) mouse

| | | |
|---|---|---|
| 42 | ModA | MRVTAPRTLILLLSGALALTETWAGSGSCDLPHTYNLGNKRALTVLEEMRRLPPLSCLKDRKDFGFPLEKVDNQQIQKAQAILVLRDL |
| | murine | TQQILNLFTSKDLSATWNATLLDSFCNDLHQQLNDLKACVMQEPPLTQEDSLLAVRTYFHRITVYLRKKKHSLCAWEVIRAEVWRALS |
| | IFNα4 | SSTNLLARLSEEKE |
| | (amino | |
| | acid) | |
| 43 | ModA | GGGCGAACTAGTATTCTTCTGGTCCCCACAGACTCAGAGAGAACCCGCCACCATGAGAGTGACCGCCCCCAGAACCCTGATCCTGCTG |
| | murine | CTGTCTGGCGCCCTGGCCCTGACAGAGACATGGGCCGGAAGCGGATCCTGTGACCTGCCTCACACTTATAACCTCGGGAACAAGAGGG |
| | IFNα4 | CCTTGACAGTCCTGGAAGAAATGAGAAGACTCCCCCCTCTTTCCTGCCTGAAGGACAGGAAGGATTTTGGATTCCCCTTGGAGAAGGT |
| | (DNA: | GGATAACCAACAGATCCAGAAGGCTCAAGCCATCCTTGTGCTAAGAGATCTTACCCAGCAGATTTTGAACCTCTTCACATCAAAAGAC |
| | 5′ UTR- | TTGTCTGCTACTTGGAATGCAACTCTCCTAGACTCATTCTGCAATGACCTCCATCAGCAGCTCAATGATCTCAAAGCCTGTGTGATGC |
| | CDS-3′ | AGGAACCTCCTCTGACCCAGGAAGACTCCCTGCTGGCTGTGAGGACATACTTCCACAGGATCACTGTGTACCTGAGAAAGAAGAAACA |
| | UTR) | CAGCCTCTGTGCCTGGGAGGTGATCAGAGCAGAAGTCTGGAGAGCCCTCTCTTCCTCAACCAACTTGCTGGCAAGACTGAGTGAGGAG |
| | | AAGGAGTGATAACTCGAGAGCTCGCTTTCTTGCTGTCCAATTTCTATTAAAGGTTCCTTTGTTCCCTAAGTCCAACTACTAAACTGGG |
| | | GGATATTATGAAGGGCCTTGAGCATCTGGATTCTGCCTAATAAAAAACATTTATTTTCATTGCTGCGTCGAGAGCTCGCTTTCTTGCT |
| | | GTCCAATTTCTATTAAAGGTTCCTTTGTTCCCTAAGTCCAACTACTAAACTGGGGGATATTATGAAGGGCCTTGAGCATCTGGATTCT |
| | | GCCTAATAAAAAACATTTATTTTCATTGCTGCGTCGAGACCTGGTCCAGAGTCGCTAGCAAAAAAAAAAAAAAAAAAAAAAAAAAAAA |
| | | AGCATATGACTAAAAAAAAAAAAAAAAAAAAAAAAAAAAAAAAAAAAAAAAAAAAAAAAAAAAAAAAAAAAAAAAAAAAAA |
| 44 | ModA | GGGCGAACUAGUAUUCUUCUGGUCCCCACAGACUCAGAGAGAACCCGCCACCAUGAGAGUGACCGCCCCCAGAACCCUGAUCCUGCUG |
| | murine | CUGUCUGGCGCCCUGGCCCUGACAGAGACAUGGGCCGGAAGCGGAUCCUGUGACCUGCCUCACACUUAUAACCUCGGGAACAAGAGGG |
| | IFNα4 | CCUUGACAGUCCUGGAAGAAAUGAGAAGACUCCCCCCUCUUUCCUGCCUGAAGGACAGGAAGGAUUUUGGAUUCCCCUUGGAGAAGGU |
| | (RNA) | GGAUAACCAACAGAUCCAGAAGGCUCAAGCCAUCCUUGUGCUAAGAGAUCUUACCCAGCAGAUUUUGAACCUCUUCACAUCAAAAGAC |
| | | UUGUCUGCUACUUGGAAUGCAACUCUCCUAGACUCAUUCUGCAAUGACCUCCAUCAGCAGCUCAAUGAUCUCAAAGCCUGUGUGAUGC |
| | | AGGAACCUCCUCUGACCCAGGAAGACUCCCUGCUGGCUGUGAGGACAUACUUCCACAGGAUCACUGUGUACCUGAGAAAGAAGAAACA |
| | | CAGCCUCUGUGCCUGGGAGGUGAUCAGAGCAGAAGUCUGGAGAGCCCUCUCUUCCUCAACCAACUUGCUGGCAAGACUGAGUGAGGAG |
| | | AAGGAGUGAUAACUCGAGAGCUCGCUUUCUUGCUGUCCAAUUUCUAUUAAAGGUUCCUUUGUUCCCUAAGUCCAACUACUAAACUGGG |
| | | GGAUAUUAUGAAGGGCCUUGAGCAUCUGGAUUCUGCCUAAUAAAAAACAUUUAUUUUCAUUGCUGCGUCGAGAGCUCGCUUUCUUGCU |
| | | GUCCAAUUUCUAUUAAAGGUUCCUUUGUUCCCUAAGUCCAACUACUAAACUGGGGGAUAUUAUGAAGGGCCUUGAGCAUCUGGAUUCU |
| | | GCCUAAUAAAAAACAUUUAUUUUCAUUGCUGCGUCGAGACCUGGUCCAGAGUCGCUAGCAAAAAAAAAAAAAAAAAAAAAAAAAAAAA |
| | | AGCAUAUGACUAAAAAAAAAAAAAAAAAAAAAAAAAAAAAAAAAAAAAAAAAAAAAAAAAAAAAAAAAAAAAAAAAAAAAA |
| 45 | ModB | MGAMAPRTLLLLLAAALAPTQTRAGPGSCDLPHTYNLGNKRALTVLEEMRRLPPLSCLKDRKDFGFPLEKVDNQQIQKAQAILVLRDL |
| | murine | TQQILNLFTSKDLSATWNATLLDSFCNDLHQQLNDLKACVMQEPPLTQEDSLLAVRTYFHRITVYLRKKKHSLCAWEVIRAEVWRALS |
| | IFNα4 | SSTNLLARLSEEKE |
| | (amino | |
| | acid) | |
| 46 | ModB | GGAATAAACTAGTCTCAACACAACATATACAAAACAAACGAATCTCAAGCAATCAAGCATTCTACTTCTATTGCAGCAATTTAAATCA |
| | murine | TTTCTTTTAAAGCAAAAGCAATTTTCTGAAAATTTTCACCATTTACGAACGATAGCCATGGGCGCCATGGCCCCTAGAACATTGCTCC |
| | IFNα4 | TGCTGCTGGCCGCTGCCCTGGCCCCTACACAGACAAGAGCTGGACCTGGATCCTGTGACCTGCCTCACACTTATAACCTCGGGAACAA |
| | (DNA: | GAGGGCCTTGACAGTCCTGGAAGAAATGAGAAGACTCCCCCCTCTTTCCTGCCTGAAGGACAGGAAGGATTTTGGATTCCCCTTGGAG |
| | 5′ UTR- | AAGGTGGATAACCAACAGATCCAGAAGGCTCAAGCCATCCTTGTGCTAAGAGATCTTACCCAGCAGATTTTGAACCTCTTCACATCAA |
| | CDS-3′ | AAGACTTGTCTGCTACTTGGAATGCAACTCTCCTAGACTCATTCTGCAATGACCTCCATCAGCAGCTCAATGATCTCAAAGCCTGTGT |
| | UTR) | GATGCAGGAACCTCCTCTGACCCAGGAAGACTCCCTGCTGGCTGTGAGGACATACTTCCACAGGATCACTGTGTACCTGAGAAAGAAG |
| | | AAACACAGCCTCTGTGCCTGGGAGGTGATCAGAGCAGAAGTCTGGAGAGCCCTCTCTTCCTCAACCAACTTGCTGGCAAGACTGAGTG |
| | | AGGAGAAGGAGTGATAACTCGACGTCCTGGTACTGCATGCACGCAATGCTAGCTGCCCCTTTCCCGTCCTGGGTACCCCGAGTCTCCC |
| | | CCGACCTCGGGTCCCAGGTATGCTCCCACCTCCACCTGCCCCACTCACCACCTCTGCTAGTTCCAGACACCTCCCAAGCACGCAGCAA |
| | | TGCAGCTCAAAACGCTTAGCCTAGCCACACCCCCACGGGAAACAGCAGTGATTAACCTTTAGCAATAAACGAAAGTTTAACTAAGCTA |
| | | TACTAACCCCAGGGTTGGTCAATTTCGTGCCAGCCACACCCTCGAGCTAGCAAAAAAAAAAAAAAAAAAAAAAAAAAAAAAGCATATG |
| | | ACTAAAAAAAAAAAAAAAAAAAAAAAAAAAAAAAAAAAAAAAAAAAAAAAAAAAAAAAAAAAAAAAAAAAAAA |
| 47 | ModB | GGAAUAAACUAGUCUCAACACAACAUAUACAAAACAAACGAAUCUCAAGCAAUCAAGCAUUCUACUUCUAUUGCAGCAAUUUAAAUCA |
| | murine | UUUCUUUUAAAGCAAAAGCAAUUUUCUGAAAAUUUUCACCAUUUACGAACGAUAGCCAUGGGCGCCAUGGCCCCUAGAACAUUGCUCC |
| | IFNα4 | UGCUGCUGGCCGCUGCCCUGGCCCCUACACAGACAAGAGCUGGACCUGGAUCCUGUGACCUGCCUCACACUUAUAACCUCGGGAACAA |
| | (RNA) | GAGGGCCUUGACAGUCCUGGAAGAAAUGAGAAGACUCCCCCCUCUUUCCUGCCUGAAGGACAGGAAGGAUUUUGGAUUCCCCUUGGAG |
| | | AAGGUGGAUAACCAACAGAUCCAGAAGGCUCAAGCCAUCCUUGUGCUAAGAGAUCUUACCCAGCAGAUUUUGAACCUCUUCACAUCAA |
| | | AAGACUUGUCUGCUACUUGGAAUGCAACUCUCCUAGACUCAUUCUGCAAUGACCUCCAUCAGCAGCUCAAUGAUCUCAAAGCCUGUGU |
| | | GAUGCAGGAACCUCCUCUGACCCAGGAAGACUCCCUGCUGGCUGUGAGGACAUACUUCCACAGGAUCACUGUGUACCUGAGAAAGAAG |
| | | AAACACAGCCUCUGUGCCUGGGAGGUGAUCAGAGCAGAAGUCUGGAGAGCCCUCUCUUCCUCAACCAACUUGCUGGCAAGACUGAGUG |
| | | AGGAGAAGGAGUGAUAACUCGACGUCCUGGUACUGCAUGCACGCAAUGCUAGCUGCCCCUUUCCCGUCCUGGGUACCCCGAGUCUCCC |
| | | CCGACCUCGGGUCCCAGGUAUGCUCCCACCUCCACCUGCCCCACUCACCACCUCUGCUAGUUCCAGACACCUCCCAAGCACGCAGCAA |
| | | UGCAGCUCAAAACGCUUAGCCUAGCCACACCCCCACGGGAAACAGCAGUGAUUAACCUUUAGCAAUAAACGAAAGUUUAACUAAGCUA |
| | | UACUAACCCCAGGGUUGGUCAAUUUCGUGCCAGCCACACCCUCGAGCUAGCAAAAAAAAAAAAAAAAAAAAAAAAAAAAAAGCAUAUG |
| | | ACUAAAAAAAAAAAAAAAAAAAAAAAAAAAAAAAAAAAAAAAAAAAAAAAAAAAAAAAAAAAAAAAAAAAAAA |

IL-15 sushi mouse

| | | |
|---|---|---|
| 48 | ModA | MGAMAPRTLLLLLAAALAPTQTRAGPGSTTCPPPVSIEHADIRVKNYSVNSRERYVCNSGFKRKAGTSTLIECVINKNINVAHWTTPS |
| | murine | LKCIRDPSLAGGSGGSGGSGGSGGSGGSGGNWIDVRYDLEKIESLIQSIHIDTTLYTDSDFHPSCKVTAMNCFLLELQVILHEYSNMT |
| | IL-15 | LNETVRNVLYLANSTLSSNKNVAESGCKECEELEEKTFTEFLQSFIRIVQMFINTS |
| | sushi | |
| | (amino | |
| | acid) | |
| 49 | ModA | GGGCGAACTAGTATTCTTCTGGTCCCCACAGACTCAGAGAGAACCCGCCACCATGGGCGCCATGGCCCCTAGAACATTGCTCCTGCTG |
| | murine | CTGGCCGCTGCCCTGGCCCCTACACAGACAAGAGCTGGACCTGGATCCACCACGTGTCCACCTCCCGTATCTATTGAGCATGCTGACA |
| | IL-15 | TCCGGGTCAAGAATTACAGTGTGAACTCCAGGGAGAGGTATGTCTGTAACTCTGGCTTTAAGCGGAAAGCTGGAACATCCACCCTGAT |
| | sushi | TGAGTGTGTGATCAACAAGAACACAAATGTTGCCCACTGGACAACTCCCAGCCTCAAGTGCATCAGAGACCCCTCCCTAGCTGGAGGG |
| | (DNA: | AGCGGAGGCTCTGGCGGAAGCGGCGGGTCTGGAGGCTCCGGGGGAAGCGGCGGAAATTGGATCGACGTGCGCTACGACCTGGAAAAGA |
| | 5′ UTR- | TCGAGAGCCTGATCCAGAGCATCCACATCGACACCACCCTGTACACCGACAGCGACTTCCACCCCAGCTGCAAAGTGACCGCTATGAA |
| | CDS-3′ | CTGCTTCCTGCTGGAACTGCAAGTGATCCTGCACGAGTACAGCAACATGACCCTGAACGAGACAGTGCGGAACGTGCTGTACCTGGCC |
| | UTR) | AACAGCACCCTGAGCAGCAACAAGAACGTGGCCGAGAGCGGCTGCAAAGAGTGCGAGGAACTGGAAGAAAAGACCTTCACCGAGTTTC |
| | | TGCAGAGCTTCATCAGGATCGTGCAGATGTTCATCAACACCTCTTGATGAGTCGACGTCCTGGTACTGCATGCACGCAATGCTAGCTG |
| | | CCCCTTTCCCGTCCTGGGTACCCCGAGTCTCCCCCGACCTCGGGTCCCAGGTATGCTCCCACCTCCACCTGCCCCACTCACCACCTCT |
| | | GCTAGTTCCAGACACCTCCCAAGCACGCAGCAATGCAGCTCAAAACGCTTAGCCTAGCCACACCCCCACGGGAAACAGCAGTGATTAA |
| | | CCTTTAGCAATAAACGAAAGTTTAACTAAGCTATACTAACCCCAGGGTTGGTCAATTTCGTGCCAGCCACACCCTCGAGCTAGCAAAA |
| | | AAAAAAAAAAAAAAAAAAAAAAAAAAGCATATGACTAAAAAAAAAAAAAAAAAAAAAAAAAAAAAAAAAAAAAAAAAAAAAAAAAAAA |
| | | AAAAAAAAAAAAAAAAAA |
| 50 | ModA | GGGCGAACUAGUAUUCUUCUGGUCCCCACAGACUCAGAGAGAACCCGCCACCAUGGGCGCCAUGGCCCCUAGAACAUUGCUCCUGCUG |
| | murine | CUGGCCGCUGCCCUGGCCCCUACACAGACAAGAGCUGGACCUGGAUCCACCACGUGUCCACCUCCCGUAUCUAUUGAGCAUGCUGACA |
| | IL-15 | UCCGGGUCAAGAAUUACAGUGUGAACUCCAGGGAGAGGUAUGUCUGUAACUCUGGCUUUAAGCGGAAAGCUGGAACAUCCACCCUGAU |
| | sushi | UGAGUGUGUGAUCAACAAGAACACAAAUGUUGCCCACUGGACAACUCCCAGCCUCAAGUGCAUCAGAGACCCCUCCCUAGCUGGAGGG |
| | (RNA) | AGCGGAGGCUCUGGCGGAAGCGGCGGGUCUGGAGGCUCCGGGGGAAGCGGCGGAAAUUGGAUCGACGUGCGCUACGACCUGGAAAAGA |
| | | UCGAGAGCCUGAUCCAGAGCAUCCACAUCGACACCACCCUGUACACCGACAGCGACUUCCACCCCAGCUGCAAAGUGACCGCUAUGAA |
| | | CUGCUUCCUGCUGGAACUGCAAGUGAUCCUGCACGAGUACAGCAACAUGACCCUGAACGAGACAGUGCGGAACGUGCUGUACCUGGCC |
| | | AACAGCACCCUGAGCAGCAACAAGAACGUGGCCGAGAGCGGCUGCAAAGAGUGCGAGGAACUGGAAGAAAAGACCUUCACCGAGUUUC |
| | | UGCAGAGCUUCAUCAGGAUCGUGCAGAUGUUCAUCAACACCUCUUGAUGAGUCGACGUCCUGGUACUGCAUGCACGCAAUGCUAGCUG |
| | | CCCCUUUCCCGUCCUGGGUACCCCGAGUCUCCCCCGACCUCGGGUCCCAGGUAUGCUCCCACCUCCACCUGCCCCACUCACCACCUCU |
| | | GCUAGUUCCAGACACCUCCCAAGCACGCAGCAAUGCAGCUCAAAACGCUUAGCCUAGCCACACCCCCACGGGAAACAGCAGUGAUUAA |
| | | CCUUUAGCAAUAAACGAAAGUUUAACUAAGCUAUACUAACCCCAGGGUUGGUCAAUUUCGUGCCAGCCACACCCUCGAGCUAGCAAAA |
| | | AAAAAAAAAAAAAAAAAAAAAAAAAAGCAUAUGACUAAAAAAAAAAAAAAAAAAAAAAAAAAAAAAAAAAAAAAAAAAAAAAAAAAAA |
| | | AAAAAAAAAAAAAAAAAA |
| 51 | ModB | MGAMAPRTLLLLLAAALAPTQTRAGPGSTTCPPPVSIEHADIRVKNYSVNSRERYVCNSGFKRKAGTSTLIECVINKNTNVAHWTTPS |
| | murine | LKCIRDPSLAGGSGGSGGSGGSGGSGGSGGNWIDVRYDLEKIESLIQSIHIDTTLYTDSDFHPSCKVTAMNCFLLELQVILHEYSNMT |
| | IL-15 | LNETVRNVLYLANSTLSSNKNVAESGCKECEELEEKTFTEFLQSFIRIVQMFINTS |
| | sushi | |
| | (amino | |
| | acid) | |
| 52 | ModB | GGAATAAACTAGTCTCAACACAACATATACAAAACAAACGAATCTCAAGCAATCAAGCATTCTACTTCTATTGCAGCAATTTAAATCA |
| | murine | TTTCTTTTAAAGCAAAAGCAATTTTCTGAAAATTTTCACCATTTACGAACGATAGCCATGGGCGCCATGGCCCCTAGAACATTGCTCC |
| | IL-15 | TGCTGCTGGCCGCTGCCCTGGCCCCTACACAGACAAGAGCTGGACCTGGATCCACCACGTGTCCACCTCCCGTATCTATTGAGCATGC |
| | sushi | TGACATCCGGGTCAAGAATTACAGTGTGAACTCCAGGGAGAGGTATGTCTGTAACTCTGGCTTTAAGCGGAAAGCTGGAACATCCACC |
| | (DNA: | CTGATTGAGTGTGTGATCAACAAGAACACAAATGTTGCCCACTGGACAACTCCCAGCCTCAAGTGCATCAGAGACCCCTCCCTAGCTG |
| | 5′ UTR- | GAGGGAGCGGAGGCTCTGGCGGAAGCGGCGGGTCTGGAGGCTCCGGGGGAAGCGGCGGAAATTGGATCGACGTGCGCTACGACCTGGA |
| | CDS-3′ | AAAGATCGAGAGCCTGATCCAGAGCATCCACATCGACACCACCCTGTACACCGACAGCGACTTCCACCCCAGCTGCAAAGTGACCGCT |
| | UTR) | ATGAACTGCTTCCTGCTGGAACTGCAAGTGATCCTGCACGAGTACAGCAACATGACCCTGAACGAGACAGTGCGGAACGTGCTGTACC |
| | | TGGCCAACAGCACCCTGAGCAGCAACAAGAACGTGGCCGAGAGCGGCTGCAAAGAGTGCGAGGAACTGGAAGAAAAGACCTTCACCGA |
| | | GTTTCTGCAGAGCTTCATCAGGATCGTGCAGATGTTCATCAACACCTCTTGATGAGTCGACGTCCTGGTACTGCATGCACGCAATGCT |
| | | AGCTGCCCCTTTCCCGTCCTGGGTACCCCGAGTCTCCCCCGACCTCGGGTCCCAGGTATGCTCCCACCTCCACCTGCCCCACTCACCA |
| | | CCTCTGCTAGTTCCAGACACCTCCCAAGCACGCAGCAATGCAGCTCAAAACGCTTAGCCTAGCCACACCCCCACGGGAAACAGCAGTG |
| | | ATTAACCTTTAGCAATAAACGAAAGTTTAACTAAGCTATACTAACCCCAGGGTTGGTCAATTTCGTGCCAGCCACACCCTCGAGCTAG |
| | | CAAAAAAAAAAAAAAAAAAAAAAAAAAAAAAGCATATGACTAAAAAAAAAAAAAAAAAAAAAAAAAAAAAAAAAAAAAAAAAAAAAAA |
| | | AAAAAAAAAAAAAAAAAAAAAAA |
| 53 | ModB | GGAAUAAACUAGUCUCAACACAACAUAUACAAAACAAACGAAUCUCAAGCAAUCAAGCAUUCUACUUCUAUUGCAGCAAUUUAAAUCA |
| | murine | UUUCUUUUAAAGCAAAAGCAAUUUUCUGAAAAUUUUCACCAUUUACGAACGAUAGCCAUGGGCGCCAUGGCCCCUAGAACAUUGCUCC |
| | IL-15 | UGCUGCUGGCCGCUGCCCUGGCCCCUACACAGACAAGAGCUGGACCUGGAUCCACCACGUGUCCACCUCCCGUAUCUAUUGAGCAUGC |
| | sushi | UGACAUCCGGGUCAAGAAUUACAGUGUGAACUCCAGGGAGAGGUAUGUCUGUAACUCUGGCUUUAAGCGGAAAGCUGGAACAUCCACC |
| | (RNA) | CUGAUUGAGUGUGUGAUCAACAAGAACACAAAUGUUGCCCACUGGACAACUCCCAGCCUCAAGUGCAUCAGAGACCCCUCCCUAGCUG |
| | | GAGGGAGCGGAGGCUCUGGCGGAAGCGGCGGGUCUGGAGGCUCCGGGGGAAGCGGCGGAAAUUGGAUCGACGUGCGCUACGACCUGGA |
| | | AAAGAUCGAGAGCCUGAUCCAGAGCAUCCACAUCGACACCACCCUGUACACCGACAGCGACUUCCACCCCAGCUGCAAAGUGACCGCU |
| | | AUGAACUGCUUCCUGCUGGAACUGCAAGUGAUCCUGCACGAGUACAGCAACAUGACCCUGAACGAGACAGUGCGGAACGUGCUGUACC |
| | | UGGCCAACAGCACCCUGAGCAGCAACAAGAACGUGGCCGAGAGCGGCUGCAAAGAGUGCGAGGAACUGGAAGAAAAGACCUUCACCGA |
| | | GUUUCUGCAGAGCUUCAUCAGGAUCGUGCAGAUGUUCAUCAACACCUCUUGAUGAGUCGACGUCCUGGUACUGCAUGCACGCAAUGCU |
| | | AGCUGCCCCUUUCCCGUCCUGGGUACCCCGAGUCUCCCCCGACCUCGGGUCCCAGGUAUGCUCCCACCUCCACCUGCCCCACUCACCA |
| | | CCUCUGCUAGUUCCAGACACCUCCCAAGCACGCAGCAAUGCAGCUCAAAACGCUUAGCCUAGCCACACCCCCACGGGAAACAGCAGUG |
| | | AUUAACCUUUAGCAAUAAACGAAAGUUUAACUAAGCUAUACUAACCCCAGGGUUGGUCAAUUUCGUGCCAGCCACACCCUCGAGCUAG |
| | | AAAAAAAAAAAAAAAAAAAAAAA |

GM-CSF mouse

| | | |
|---|---|---|
| 54 | ModA | MWLQNLLFLGIVVYSLSAPTRSPITVTRPWKHVEAIKEALNLLDDMPVTLNEEVEVVSNEFSFKKLTCVQTRLKIFEQGLRGNFTKLK |
| | murine | GALNMTASYYQTYCPPTPETDCETQVTTYADFIDSLKTFLTDIPFECKKPGQK |
| | GM-CSF | |
| | (amino | |
| | acid) | |
| 55 | ModA | GGGCGAACTAGTATTCTTCTGGTCCCCACAGACTCAGAGAGAACCCGCCACCATGTGGCTGCAGAACCTGCTGTTCCTGGGCATCGTG |
| | murine | GTGTACAGCCTGAGCGCCCCCACCAGGAGCCCCATCACCGTGACCAGGCCCTGGAAGCACGTGGAGGCCATCAAGGAGGCCCTGAACC |
| | GM-CSF | TGCTGGACGACATGCCCGTGACCCTGAACGAGGAGGTGGAGGTGGTGAGCAACGAGTTCAGCTTCAAGAAGCTGACCTGCGTGCAGAC |
| | (DNA: | CAGGCTGAAGATCTTCGAGCAGGGCCTGAGGGGCAACTTCACCAAGCTGAAGGGCGCCCTGAACATGACCGCCAGCTACTACCAGACC |
| | 5′ UTR- | TACTGCCCCCCCACCCCCGAGACCGACTGCGAGACCCAGGTGACCACCTACGCCGACTTCATCGACAGCCTGAAGACCTTCCTGACCG |
| | CDS-3′ | ACATCCCCTTCGAGTGCAAGAAGCCCGGCCAGAAGTGATGACTCGAGCTGGTACTGCATGCACGCAATGCTAGCTGCCCCTTTCCCGT |
| | UTR) | CCTGGGTACCCCGAGTCTCCCCCGACCTCGGGTCCCAGGTATGCTCCCACCTCCACCTGCCCCACTCACCACCTCTGCTAGTTCCAGA |
| | | CACCTCCCAAGCACGCAGCAATGCAGCTCAAAACGCTTAGCCTAGCCACACCCCCACGGGAAACAGCAGTGATTAACCTTTAGCAATA |
| | | AACGAAAGTTTAACTAAGCTATACTAACCCCAGGGTTGGTCAATTTCGTGCCAGCCACACCGAGACCTGGTCCAGAGTCGCTAGCCGC |
| | | GTCGCTAAAAAAAAAAAAAAAAAAAAAAAAAAAAAAGCATATGACTAAAAAAAAAAAAAAAAAAAAAAAAAAAAAAAAAAAAAAAAAA |
| | | AAAAAAAAAAAAAAAAAAAAAAAAAAAA |
| 56 | ModA | GGGCGAACUAGUAUUCUUCUGGUCCCCACAGACUCAGAGAGAACCCGCCACCAUGUGGCUGCAGAACCUGCUGUUCCUGGGCAUCGUG |
| | murine | GUGUACAGCCUGAGCGCCCCCACCAGGAGCCCCAUCACCGUGACCAGGCCCUGGAAGCACGUGGAGGCCAUCAAGGAGGCCCUGAACC |
| | GM-CSF | UGCUGGACGACAUGCCCGUGACCCUGAACGAGGAGGUGGAGGUGGUGAGCAACGAGUUCAGCUUCAAGAAGCUGACCUGCGUGCAGAC |
| | (RNA) | CAGGCUGAAGAUCUUCGAGCAGGGCCUGAGGGGCAACUUCACCAAGCUGAAGGGCGCCCUGAACAUGACCGCCAGCUACUACCAGACC |
| | | UACUGCCCCCCCACCCCCGAGACCGACUGCGAGACCCAGGUGACCACCUACGCCGACUUCAUCGACAGCCUGAAGACCUUCCUGACCG |
| | | ACAUCCCCUUCGAGUGCAAGAAGCCCGGCCAGAAGUGAUGACUCGAGCUGGUACUGCAUGCACGCAAUGCUAGCUGCCCCUUUCCCGU |
| | | CCUGGGUACCCCGAGUCUCCCCCGACCUCGGGUCCCAGGUAUGCUCCCACCUCCACCUGCCCCACUCACCACCUCUGCUAGUUCCAGA |
| | | CACCUCCCAAGCACGCAGCAAUGCAGCUCAAAACGCUUAGCCUAGCCACACCCCCACGGGAAACAGCAGUGAUUAACCUUUAGCAAUA |
| | | AACGAAAGUUUAACUAAGCUAUACUAACCCCAGGGUUGGUCAAUUUCGUGCCAGCCACACCGAGACCUGGUCCAGAGUCGCUAGCCGC |
| | | GUCGCUAAAAAAAAAAAAAAAAAAAAAAAAAAAAAAGCAUAUGACUAAAAAAAAAAAAAAAAAAAAAAAAAAAAAAAAAAAAAAAAAA |
| | | AAAAAAAAAAAAAAAAAAAAAAAAAAAA |
| 57 | ModB | MWLQNLLFLGIVVYSLSAPTRSPITVTRPWKHVEAIKEALNLLDDMPVTLNEEVEVVSNEFSFKKLTCVQTRLKIFEQGLRGNFTKLK |
| | murine | GALNMTASYYQTYCPPTPETDCETQVTTYADFIDSLKTFLTDIPFECKKPGQK |
| | GM-CSF | |
| | (amino | |
| | acid) | |
| 58 | ModB | GGAATAAACTAGTCTCAACACAACATATACAAAACAAACGAATCTCAAGCAATCAAGCATTCTACTTCTATTGCAGCAATTTAAATCA |
| | murine | TTTCTTTTAAAGCAAAAGCAATTTTCTGAAAATTTTCACCATTTACGAACGATAGCCATGTGGCTGCAGAACCTGCTGTTCCTGGGCA |
| | GM-CSF | TCGTGGTGTACAGCCTGAGCGCCCCCACCAGGAGCCCCATCACCGTGACCAGGCCCTGGAAGCACGTGGAGGCCATCAAGGAGGCCCT |
| | (DNA: | GAACCTGCTGGACGACATGCCCGTGACCCTGAACGAGGAGGTGGAGGTGGTGAGCAACGAGTTCAGCTTCAAGAAGCTGACCTGCGTG |
| | 5′ UTR- | CAGACCAGGCTGAAGATCTTCGAGCAGGGCCTGAGGGGCAACTTCACCAAGCTGAAGGGCGCCCTGAACATGACCGCCAGCTACTACC |
| | CDS-3′ | AGACCTACTGCCCCCCCACCCCCGAGACCGACTGCGAGACCCAGGTGACCACCTACGCCGACTTCATCGACAGCCTGAAGACCTTCCT |
| | UTR) | GACCGACATCCCCTTCGAGTGCAAGAAGCCCGGCCAGAAGTGATGACTCGAGCTGGTACTGCATGCACGCAATGCTAGCTGCCCCTTT |
| | | CCCGTCCTGGGTACCCCGAGTCTCCCCCGACCTCGGGTCCCAGGTATGCTCCCACCTCCACCTGCCCCACTCACCACCTCTGCTAGTT |
| | | CCAGACACCTCCCAAGCACGCAGCAATGCAGCTCAAAACGCTTAGCCTAGCCACACCCCCACGGGAAACAGCAGTGATTAACCTTTAG |
| | | CAATAAACGAAAGTTTAACTAAGCTATACTAACCCCAGGGTTGGTCAATTTCGTGCCAGCCACACCGAGACCTGGTCCAGAGTCGCTA |
| | | GCCGCGTCGCTAAAAAAAAAAAAAAAAAAAAAAAAAAAAAAGCATATGACTAAAAAAAAAAAAAAAAAAAAAAAAAAAAAAAAAAAAA |
| | | AAAAAAAAAAAAAAAAAAAAAAAAAAAAAAAAA |
| 59 | ModB | GGAAUAAACUAGUCUCAACACAACAUAUACAAAACAAACGAAUCUCAAGCAAUCAAGCAUUCUACUUCUAUUGCAGCAAUUUAAAUCA |
| | murine | UCGUGGUGUACAGCCUGAGCGCCCCCACCAGGAGCCCCAUCACCGUGACCAGGCCCUGGAAGCACGUGGAGGCCAUCAAGGAGGCCCU |
| | GM-CSF | GAACCUGCUGGACGACAUGCCCGUGACCCUGAACGAGGAGGUGGAGGUGGUGAGCAACGAGUUCAGCUUCAAGAAGCUGACCUGCGUG |
| | (RNA) | CAGACCAGGCUGAAGAUCUUCGAGCAGGGCCUGAGGGGCAACUUCACCAAGCUGAAGGGCGCCCUGAACAUGACCGCCAGCUACUACC |
| | | AGACCUACUGCCCCCCCACCCCCGAGACCGACUGCGAGACCCAGGUGACCACCUACGCCGACUUCAUCGACAGCCUGAAGACCUUCCU |
| | | GACCGACAUCCCCUUCGAGUGCAAGAAGCCCGGCCAGAAGUGAUGACUCGAGCUGGUACUGCAUGCACGCAAUGCUAGCUGCCCCUUU |
| | | CCCGUCCUGGGUACCCCGAGUCUCCCCCGACCUCGGGUCCCAGGUAUGCUCCCACCUCCACCUGCCCCACUCACCACCUCUGCUAGUU |
| | | CCAGACACCUCCCAAGCACGCAGCAAUGCAGCUCAAAACGCUUAGCCUAGCCACACCCCCACGGGAAACAGCAGUGAUUAACCUUUAG |
| | | CAAUAAACGAAAGUUUAACUAAGCUAUACUAACCCCAGGGUUGGUCAAUUUCGUGCCAGCCACACCGAGACCUGGUCCAGAGUCGCUA |
| | | GCCGCGUCGCUAAAAAAAAAAAAAAAAAAAAAAAAAAAAAAGCAUAUGACUAAAAAAAAAAAAAAAAAAAAAAAAAAAAAAAAAAAAA |
| | | AAAAAAAAAAAAAAAAAAAAAAAAAAAAAAAAA |

FLT3L mouse

| | | |
|---|---|---|
| 60 | ModA | MGAMAPRTLLLLLAAALAPTQTRAGPGSTQDCSFQHSPISSDFAVKIRELSDYLLQDYPVTVASNLQDEELCGGLWRLVLAQRWMERL |
| | FLT3L | KTVAGSKMQGLLERVNTEIHFVTKCAFQPPPSCLRFVQTNISRLLQETSEQLVALKPWITRQNFSRCLELQCQPDSSTLPPPWSPRPL |
| | (amino | EATAPTAPQPP |
| | acid, | |
| | human | |
| | FLT3L in | |
| | combi- | |
| | nation | |
| | with a | |
| | mouse | |
| | opti- | |
| | mized | |
| | secre- | |
| | tion | |
| | se- | |
| | quence)) | |
| 61 | ModA | GGGCGAACTAGTATTCTTCTGGTCCCCACAGACTCAGAGAGAACCCGCCACCATGGGCGCCATGGCCCCTAGAACATTGCTCCTGCTG |
| | FLT3L | CTGGCCGCTGCCCTGGCCCCTACACAGACAAGAGCTGGACCTGGATCCACCCAGGACTGCAGCTTCCAGCACTCCCCTATCTCCTCCG |
| | (DNA: | ACTTCGCCGTGAAGATCCGGGAGCTGTCCGATTACCTGCTGCAGGACTACCCTGTGACCGTGGCCAGCAACCTGCAGGACGAAGAACT |
| | 5′ UTR- | GTGTGGCGGCCTGTGGCGGCTGGTGCTGGCCCAGCGGTGGATGGAACGGCTGAAAACCGTGGCCGGCTCCAAGATGCAGGGCCTGCTC |
| | CDS-3′ | GAGCGGGTGAACACCGAGATCCACTTCGTGACCAAGTGCGCCTTCCAGCCTCCTCCTTCCTGCCTGCGGTTCGTGCAGACCAACATCT |
| | UTR) | CCCGGCTGCTGCAGGAAACCTCCGAGCAGCTGGTCGCCCTGAAGCCTTGGATCACCCGGCAGAACTTCTCCCGGTGTCTGGAACTCCA |
| | | GTGTCAGCCCGACTCCTCCACCCTGCCTCCTCCCTGGTCCCCCAGGCCTCTGGAAGCCACCGCCCCTACCGCCCCACAGCCTCCTTGA |
| | | TAGGTCGACGTCCTGGTACTGCATGCACGCAATGCTAGCTGCCCCTTTCCCGTCCTGGGTACCCCGAGTCTCCCCCGACCTCGGGTCC |
| | | CAGGTATGCTCCCACCTCCACCTGCCCCACTCACCACCTCTGCTAGTTCCAGACACCTCCCAAGCACGCAGCAATGCAGCTCAAAACG |
| | | CTTAGCCTAGCCACACCCCCACGGGAAACAGCAGTGATTAACCTTTAGCAATAAACGAAAGTTTAACTAAGCTATACTAACCCCAGGG |
| | | TTGGTCAATTTCGTGCCAGCCACACCCTCGAGCTAGCAAAAAAAAAAAAAAAAAAAAAAAAAAAAAAGCATATGACTAAAAAAAAAAA |
| | | AAAAAAAAAAAAAAAAAAAAAAAAAAAAAAAAAAAAAAAAAAAAAAAAAAAAAAAAAAA |
| 62 | ModA | GGGCGAACUAGUAUUCUUCUGGUCCCCACAGACUCAGAGAGAACCCGCCACCAUGGGCGCCAUGGCCCCUAGAACAUUGCUCCUGCUG |
| | FLT3L | CUGGCCGCUGCCCUGGCCCCUACACAGACAAGAGCUGGACCUGGAUCCACCCAGGACUGCAGCUUCCAGCACUCCCCUAUCUCCUCCG |
| | (RNA) | ACUUCGCCGUGAAGAUCCGGGAGCUGUCCGAUUACCUGCUGCAGGACUACCCUGUGACCGUGGCCAGCAACCUGCAGGACGAAGAACU |
| | | GUGUGGCGGCCUGUGGCGGCUGGUGCUGGCCCAGCGGUGGAUGGAACGGCUGAAAACCGUGGCCGGCUCCAAGAUGCAGGGCCUGCUC |
| | | GAGCGGGUGAACACCGAGAUCCACUUCGUGACCAAGUGCGCCUUCCAGCCUCCUCCUUCCUGCCUGCGGUUCGUGCAGACCAACAUCU |
| | | CCCGGCUGCUGCAGGAAACCUCCGAGCAGCUGGUCGCCCUGAAGCCUUGGAUCACCCGGCAGAACUUCUCCCGGUGUCUGGAACUCCA |
| | | GUGUCAGCCCGACUCCUCCACCCUGCCUCCUCCCUGGUCCCCCAGGCCUCUGGAAGCCACCGCCCCUACCGCCCCACAGCCUCCUUGA |
| | | UAGGUCGACGUCCUGGUACUGCAUGCACGCAAUGCUAGCUGCCCCUUUCCCGUCCUGGGUACCCCGAGUCUCCCCCGACCUCGGGUCC |
| | | CAGGUAUGCUCCCACCUCCACCUGCCCCACUCACCACCUCUGCUAGUUCCAGACACCUCCCAAGCACGCAGCAAUGCAGCUCAAAACG |
| | | CUUAGCCUAGCCACACCCCCACGGGAAACAGCAGUGAUUAACCUUUAGCAAUAAACGAAAGUUUAACUAAGCUAUACUAACCCCAGGG |
| | | UUGGUCAAUUUCGUGCCAGCCACACCCUCGAGCUAGCAAAAAAAAAAAAAAAAAAAAAAAAAAAAAAGCAUAUGACUAAAAAAAAAAA |
| | | AAAAAAAAAAAAAAAAAAAAAAAAAAAAAAAAAAAAAAAAAAAAAAAAAAAAAAAAAAA |
| 63 | ModB | MGAMAPRTLLLLLAAALAPTQTRAGPGSTQDCSFQHSPISSDFAVKIRELSDYLLQDYPVTVASNLQDEELCGGLWRLVLAQRWMERL |
| | FLT3L | KTVAGSKMQGLLERVNTEIHFVTKCAFQPPPSCLRFVQTNISRLLQETSEQLVALKPWITRQNFSRCLELQCQPDSSTLPPPWSPRPL |
| | (amino | EATAPTAPQPP |
| | acid, | |
| | human | |
| | FLT3L in | |
| | combi- | |
| | nation | |
| | with a | |
| | mouse | |
| | opti- | |
| | mized | |
| | secre- | |
| | tion | |
| | se- | |
| | quence) | |
| 64 | ModB | GGAATAAACTAGTCTCAACACAACATATACAAAACAAACGAATCTCAAGCAATCAAGCATTCTACTTCTATTGCAGCAATTTAAATCA |
| | FLT3L | TTTCTTTTAAAGCAAAAGCAATTTTCTGAAAATTTTCACCATTTACGAACGATAGCCATGGGCGCCATGGCCCCTAGAACATTGCTCC |
| | (DNA: | TGCTGCTGGCCGCTGCCCTGGCCCCTACACAGACAAGAGCTGGACCTGGATCCACCCAGGACTGCAGCTTCCAGCACTCCCCTATCTC |
| | 5′ UTR- | CTCCGACTTCGCCGTGAAGATCCGGGAGCTGTCCGATTACCTGCTGCAGGACTACCCTGTGACCGTGGCCAGCAACCTGCAGGACGAA |
| | CDS-3′ | GAACTGTGTGGCGGCCTGTGGCGGCTGGTGCTGGCCCAGCGGTGGATGGAACGGCTGAAAACCGTGGCCGGCTCCAAGATGCAGGGCC |
| | UTR) | TGCTCGAGCGGGTGAACACCGAGATCCACTTCGTGACCAAGTGCGCCTTCCAGCCTCCTCCTTCCTGCCTGCGGTTCGTGCAGACCAA |
| | | CATCTCCCGGCTGCTGCAGGAAACCTCCGAGCAGCTGGTCGCCCTGAAGCCTTGGATCACCCGGCAGAACTTCTCCCGGTGTCTGGAA |
| | | CTCCAGTGTCAGCCCGACTCCTCCACCCTGCCTCCTCCCTGGTCCCCCAGGCCTCTGGAAGCCACCGCCCCTACCGCCCCACAGCCTC |
| | | CTTGATAGGTCGACGTCCTGGTACTGCATGCACGCAATGCTAGCTGCCCCTTTCCCGTCCTGGGTACCCCGAGTCTCCCCCGACCTCG |
| | | GGTCCCAGGTATGCTCCCACCTCCACCTGCCCCACTCACCACCTCTGCTAGTTCCAGACACCTCCCAAGCACGCAGCAATGCAGCTCA |
| | | AAACGCTTAGCCTAGCCACACCCCCACGGGAAACAGCAGTGATTAACCTTTAGCAATAAACGAAAGTTTAACTAAGCTATACTAACCC |
| | | CAGGGTTGGTCAATTTCGTGCCAGCCACACCCTCGAGCTAGCAAAAAAAAAAAAAAAAAAAAAAAAAAAAAAGCATATGACTAAAAAA |
| | | AAAAAAAAAAAAAAAAAAAAAAAAAAAAAAAAAAAAAAAAAAAAAAAAAAAAAAAAAAAAAAAA |
| 65 | ModB | GGAAUAAACUAGUCUCAACACAACAUAUACAAAACAAACGAAUCUCAAGCAAUCAAGCAUUCUACUUCUAUUGCAGCAAUUUAAAUCA |
| | murine | UGCUGCUGGCCGCUGCCCUGGCCCCUACACAGACAAGAGCUGGACCUGGAUCCACCCAGGACUGCAGCUUCCAGCACUCCCCUAUCUC |
| | FLT3L | CUCCGACUUCGCCGUGAAGAUCCGGGAGCUGUCCGAUUACCUGCUGCAGGACUACCCUGUGACCGUGGCCAGCAACCUGCAGGACGAA |
| | (RNA) | GAACUGUGUGGCGGCCUGUGGCGGCUGGUGCUGGCCCAGCGGUGGAUGGAACGGCUGAAAACCGUGGCCGGCUCCAAGAUGCAGGGCC |
| | | UGCUCGAGCGGGUGAACACCGAGAUCCACUUCGUGACCAAGUGCGCCUUCCAGCCUCCUCCUUCCUGCCUGCGGUUCGUGCAGACCAA |
| | | CAUCUCCCGGCUGCUGCAGGAAACCUCCGAGCAGCUGGUCGCCCUGAAGCCUUGGAUCACCCGGCAGAACUUCUCCCGGUGUCUGGAA |
| | | CUCCAGUGUCAGCCCGACUCCUCCACCCUGCCUCCUCCCUGGUCCCCCAGGCCUCUGGAAGCCACCGCCCCUACCGCCCCACAGCCUC |
| | | CUUGAUAGGUCGACGUCCUGGUACUGCAUGCACGCAAUGCUAGCUGCCCCUUUCCCGUCCUGGGUACCCCGAGUCUCCCCCGACCUCG |
| | | GGUCCCAGGUAUGCUCCCACCUCCACCUGCCCCACUCACCACCUCUGCUAGUUCCAGACACCUCCCAAGCACGCAGCAAUGCAGCUCA |
| | | AAACGCUUAGCCUAGCCACACCCCCACGGGAAACAGCAGUGAUUAACCUUUAGCAAUAAACGAAAGUUUAACUAAGCUAUACUAACCC |
| | | CAGGGUUGGUCAAUUUCGUGCCAGCCACACCCUCGAGCUAGCAAAAAAAAAAAAAAAAAAAAAAAAAAAAAAGCAUAUGACUAAAAAA |
| | | AAAAAAAAAAAAAAAAAAAAAAAAAAAAAAAAAAAAAAAAAAAAAAAAAAAAAAAAAAAAAAAA |

41BBL mouse

| | | |
|---|---|---|
| 66 | ModA | MDQHTLDVEDTADARHPAGTSCPSDAALLRDTGLLADAALLSDTVRPTNAALPTDAAYPAVNVRDREAAWPPALNFCSRHPKLYGLVA |
| | murine | LVLLLLIAACVPIFTRTEPRPALTITTSPNLGTRENNADQVTPVSHIGCPNTTQQGSPVFAKLLAKNQASLCNTTLNWHSQDGAGSSY |
| | 41BBL | LSQGLRYEEDKKELVVDSPGLYYVFLELKLSPTFTNTGHKVQGWVSLVLQAKPQVDDFDNLALTVELFPCSMENKLVDRSWSQLLLLK |
| | (amino | AGHRLSVGLRAYLHGAQDAYRDWELSYPNTTSFGLFLVKPDNPWE |
| | acid) | |
| 67 | ModA | GGGCGAACTAGTATTCTTCTGGTCCCCACAGACTCAGAGAGAACCCGCCACCATGGACCAGCACACACTTGATGTGGAGGATACCGCG |
| | murine | GATGCCAGACATCCAGCAGGTACTTCGTGCCCCTCGGATGCGGCGCTCCTCAGAGATACCGGGCTCCTCGCGGACGCTGCGCTCCTCT |
| | 41BBL | CAGATACTGTGCGCCCCACAAATGCCGCGCTCCCCACGGATGCTGCCTACCCTGCGGTTAATGTTCGGGATCGCGAGGCCGCGTGGCC |
| | (DNA: | GCCTGCACTGAACTTCTGTTCCCGCCACCCAAAGCTCTATGGCCTAGTCGCTTTGGTTTTGCTGCTTCTGATCGCCGCCTGTGTTCCT |
| | 5′ UTR- | ATCTTCACCCGCACCGAGCCTCGGCCAGCGCTCACAATCACCACCTCGCCCAACCTGGGTACCCGAGAGAATAATGCAGACCAGGTCA |
| | CDS-3′ | CCCCTGTTTCCCACATTGGCTGCCCCAACACTACACAACAGGGCTCTCCTGTGTTCGCCAAGCTACTGGCTAAAAACCAAGCATCGTT |
| | UTR) | GTGCAATACAACTCTGAACTGGCACAGCCAAGATGGAGCTGGGAGCTCATACCTATCTCAAGGTCTGAGGTACGAAGAAGACAAAAAG |
| | | GAGTTGGTGGTAGACAGTCCCGGGCTCTACTACGTATTTTTGGAACTGAAGCTCAGTCCAACATTCACAAACACAGGCCACAAGGTGC |
| | | AGGGCTGGGTCTCTCTTGTTTTGCAAGCAAAGCCTCAGGTAGATGACTTTGACAACTTGGCCCTGACAGTGGAACTGTTCCCTTGCTC |
| | | CATGGAGAACAAGTTAGTGGACCGTTCCTGGAGTCAACTGTTGCTCCTGAAGGCTGGCCACCGCCTCAGTGTGGGTCTGAGGGCTTAT |
| | | CTGCATGGAGCCCAGGATGCATACAGAGACTGGGAGCTGTCTTATCCCAACACCACCAGCTTTGGACTCTTTCTTGTGAAACCCGACA |
| | | ACCCATGGGAATGATAGGGATCCGATCTGGTACTGCATGCACGCAATGCTAGCTGCCCCTTTCCCGTCCTGGGTACCCCGAGTCTCCC |
| | | CCGACCTCGGGTCCCAGGTATGCTCCCACCTCCACCTGCCCCACTCACCACCTCTGCTAGTTCCAGACACCTCCCAAGCACGCAGCAA |
| | | TGCAGCTCAAAACGCTTAGCCTAGCCACACCCCCACGGGAAACAGCAGTGATTAACCTTTAGCAATAAACGAAAGTTTAACTAAGCTA |
| | | TACTAACCCCAGGGTTGGTCAATTTCGTGCCAGCCACACCCTCGAGCTAGCAAAAAAAAAAAAAAAAAAAAAAAAAAAAAAGCATATG |
| | | ACTAAAAAAAAAAAAAAAAAAAAAAAAAAAAAAAAAAAAAAAAAAAAAAAAAAAAAAAAAAAAAAAAAAAAAA |
| 68 | ModA | GGGCGAACUAGUAUUCUUCUGGUCCCCACAGACUCAGAGAGAACCCGCCACCAUGGACCAGCACACACUUGAUGUGGAGGAUACCGCG |
| | murine | GAUGCCAGACAUCCAGCAGGUACUUCGUGCCCCUCGGAUGCGGCGCUCCUCAGAGAUACCGGGCUCCUCGCGGACGCUGCGCUCCUCU |
| | 41BBL | CAGAUACUGUGCGCCCCACAAAUGCCGCGCUCCCCACGGAUGCUGCCUACCCUGCGGUUAAUGUUCGGGAUCGCGAGGCCGCGUGGCC |
| | (RNA) | GCCUGCACUGAACUUCUGUUCCCGCCACCCAAAGCUCUAUGGCCUAGUCGCUUUGGUUUUGCUGCUUCUGAUCGCCGCCUGUGUUCCU |
| | | AUCUUCACCCGCACCGAGCCUCGGCCAGCGCUCACAAUCACCACCUCGCCCAACCUGGGUACCCGAGAGAAUAAUGCAGACCAGGUCA |
| | | CCCCUGUUUCCCACAUUGGCUGCCCCAACACUACACAACAGGGCUCUCCUGUGUUCGCCAAGCUACUGGCUAAAAACCAAGCAUCGUU |
| | | GUGCAAUACAACUCUGAACUGGCACAGCCAAGAUGGAGCUGGGAGCUCAUACCUAUCUCAAGGUCUGAGGUACGAAGAAGACAAAAAG |
| | | GAGUUGGUGGUAGACAGUCCCGGGCUCUACUACGUAUUUUUGGAACUGAAGCUCAGUCCAACAUUCACAAACACAGGCCACAAGGUGC |
| | | AGGGCUGGGUCUCUCUUGUUUUGCAAGCAAAGCCUCAGGUAGAUGACUUUGACAACUUGGCCCUGACAGUGGAACUGUUCCCUUGCUC |
| | | CAUGGAGAACAAGUUAGUGGACCGUUCCUGGAGUCAACUGUUGCUCCUGAAGGCUGGCCACCGCCUCAGUGUGGGUCUGAGGGCUUAU |
| | | CUGCAUGGAGCCCAGGAUGCAUACAGAGACUGGGAGCUGUCUUAUCCCAACACCACCAGCUUUGGACUCUUUCUUGUGAAACCCGACA |
| | | ACCCAUGGGAAUGAUAGGGAUCCGAUCUGGUACUGCAUGCACGCAAUGCUAGCUGCCCCUUUCCCGUCCUGGGUACCCCGAGUCUCCC |
| | | CCGACCUCGGGUCCCAGGUAUGCUCCCACCUCCACCUGCCCCACUCACCACCUCUGCUAGUUCCAGACACCUCCCAAGCACGCAGCAA |
| | | UGCAGCUCAAAACGCUUAGCCUAGCCACACCCCCACGGGAAACAGCAGUGAUUAACCUUUAGCAAUAAACGAAAGUUUAACUAAGCUA |
| | | UACUAACCCCAGGGUUGGUCAAUUUCGUGCCAGCCACACCCUCGAGCUAGCAAAAAAAAAAAAAAAAAAAAAAAAAAAAAAGCAUAUG |
| | | ACUAAAAAAAAAAAAAAAAAAAAAAAAAAAAAAAAAAAAAAAAAAAAAAAAAAAAAAAAAAAAAAAAAAAAAA |
| 69 | ModB | MDQHTLDVEDTADARHPAGTSCPSDAALLRDTGLLADAALLSDTVRPTNAALPTDAAYPAVNVRDREAAWPPALNFCSRHPKLYGLVA |
| | murine | LVLLLLIAACVPIFTRTEPRPALTITTSPNLGTRENNADQVTPVSHIGCPNTTQQGSPVFAKLLAKNQASLCNTTLNWHSQDGAGSSY |
| | 41BBL | LSQGLRYEEDKKELVVDSPGLYYVFLELKLSPTFTNTGHKVQGWVSLVLQAKPQVDDFDNLALTVELFPCSMENKLVDRSWSQLLLLK |
| | (amino | AGHRLSVGLRAYLHGAQDAYRDWELSYPNTTSFGLFLVKPDNPWE |
| | acid) | |
| 70 | ModB | GGAATAAACTAGTCTCAACACAACATATACAAAACAAACGAATCTCAAGCAATCAAGCATTCTACTTCTATTGCAGCAATTTAAATCA |
| | murine | TTTCTTTTAAAGCAAAAGCAATTTTCTGAAAATTTTCACCATTTACGAACGATAGCCATGGACCAGCACACACTTGATGTGGAGGATA |
| | 41BBL | CCGCGGATGCCAGACATCCAGCAGGTACTTCGTGCCCCTCGGATGCGGCGCTCCTCAGAGATACCGGGCTCCTCGCGGACGCTGCGCT |
| | (DNA: | CCTCTCAGATACTGTGCGCCCCACAAATGCCGCGCTCCCCACGGATGCTGCCTACCCTGCGGTTAATGTTCGGGATCGCGAGGCCGCG |
| | 5′ UTR- | TGGCCGCCTGCACTGAACTTCTGTTCCCGCCACCCAAAGCTCTATGGCCTAGTCGCTTTGGTTTTGCTGCTTCTGATCGCCGCCTGTG |
| | CDS-3′ | TTCCTATCTTCACCCGCACCGAGCCTCGGCCAGCGCTCACAATCACCACCTCGCCCAACCTGGGTACCCGAGAGAATAATGCAGACCA |
| | UTR) | GGTCACCCCTGTTTCCCACATTGGCTGCCCCAACACTACACAACAGGGCTCTCCTGTGTTCGCCAAGCTACTGGCTAAAAACCAAGCA |
| | | TCGTTGTGCAATACAACTCTGAACTGGCACAGCCAAGATGGAGCTGGGAGCTCATACCTATCTCAAGGTCTGAGGTACGAAGAAGACA |
| | | AAAAGGAGTTGGTGGTAGACAGTCCCGGGCTCTACTACGTATTTTTGGAACTGAAGCTCAGTCCAACATTCACAAACACAGGCCACAA |
| | | GGTGCAGGGCTGGGTCTCTCTTGTTTTGCAAGCAAAGCCTCAGGTAGATGACTTTGACAACTTGGCCCTGACAGTGGAACTGTTCCCT |
| | | TGCTCCATGGAGAACAAGTTAGTGGACCGTTCCTGGAGTCAACTGTTGCTCCTGAAGGCTGGCCACCGCCTCAGTGTGGGTCTGAGGG |
| | | CTTATCTGCATGGAGCCCAGGATGCATACAGAGACTGGGAGCTGTCTTATCCCAACACCACCAGCTTTGGACTCTTTCTTGTGAAACC |
| | | CGACAACCCATGGGAATGATAGGGATCCGATCTGGTACTGCATGCACGCAATGCTAGCTGCCCCTTTCCCGTCCTGGGTACCCCGAGT |
| | | CTCCCCCGACCTCGGGTCCCAGGTATGCTCCCACCTCCACCTGCCCCACTCACCACCTCTGCTAGTTCCAGACACCTCCCAAGCACGC |
| | | AGCAATGCAGCTCAAAACGCTTAGCCTAGCCACACCCCCACGGGAAACAGCAGTGATTAACCTTTAGCAATAAACGAAAGTTTAACTA |
| | | AGCTATACTAACCCCAGGGTTGGTCAATTTCGTGCCAGCCACACCCTCGAGCTAGCAAAAAAAAAAAAAAAAAAAAAAAAAAAAAAGC |
| | | ATATGACTAAAAAAAAAAAAAAAAAAAAAAAAAAAAAAAAAAAAAAAAAAAAAAAAAAAAAAAAAAAAAAAAAAAAAA |
| 71 | ModB | GGAAUAAACUAGUCUCAACACAACAUAUACAAAACAAACGAAUCUCAAGCAAUCAAGCAUUCUACUUCUAUUGCAGCAAUUUAAAUCA |
| | murine | UUUCUUUUAAAGCAAAAGCAAUUUUCUGAAAAUUUUCACCAUUUACGAACGAUAGCCAUGGACCAGCACACACUUGAUGUGGAGGAUA |
| | 41BBL | CCGCGGAUGCCAGACAUCCAGCAGGUACUUCGUGCCCCUCGGAUGCGGCGCUCCUCAGAGAUACCGGGCUCCUCGCGGACGCUGCGCU |
| | (RNA) | CCUCUCAGAUACUGUGCGCCCCACAAAUGCCGCGCUCCCCACGGAUGCUGCCUACCCUGCGGUUAAUGUUCGGGAUCGCGAGGCCGCG |
| | | UGGCCGCCUGCACUGAACUUCUGUUCCCGCCACCCAAAGCUCUAUGGCCUAGUCGCUUUGGUUUUGCUGCUUCUGAUCGCCGCCUGUG |
| | | UUCCUAUCUUCACCCGCACCGAGCCUCGGCCAGCGCUCACAAUCACCACCUCGCCCAACCUGGGUACCCGAGAGAAUAAUGCAGACCA |
| | | GGUCACCCCUGUUUCCCACAUUGGCUGCCCCAACACUACACAACAGGGCUCUCCUGUGUUCGCCAAGCUACUGGCUAAAAACCAAGCA |
| | | UCGUUGUGCAAUACAACUCUGAACUGGCACAGCCAAGAUGGAGCUGGGAGCUCAUACCUAUCUCAAGGUCUGAGGUACGAAGAAGACA |
| | | AAAAGGAGUUGGUGGUAGACAGUCCCGGGCUCUACUACGUAUUUUUGGAACUGAAGCUCAGUCCAACAUUCACAAACACAGGCCACAA |
| | | GGUGCAGGGCUGGGUCUCUCUUGUUUUGCAAGCAAAGCCUCAGGUAGAUGACUUUGACAACUUGGCCCUGACAGUGGAACUGUUCCCU |
| | | UGCUCCAUGGAGAACAAGUUAGUGGACCGUUCCUGGAGUCAACUGUUGCUCCUGAAGGCUGGCCACCGCCUCAGUGUGGGUCUGAGGG |
| | | CUUAUCUGCAUGGAGCCCAGGAUGCAUACAGAGACUGGGAGCUGUCUUAUCCCAACACCACCAGCUUUGGACUCUUUCUUGUGAAACC |
| | | CGACAACCCAUGGGAAUGAUAGGGAUCCGAUCUGGUACUGCAUGCACGCAAUGCUAGCUGCCCCUUUCCCGUCCUGGGUACCCCGAGU |
| | | CUCCCCCGACCUCGGGUCCCAGGUAUGCUCCCACCUCCACCUGCCCCACUCACCACCUCUGCUAGUUCCAGACACCUCCCAAGCACGC |
| | | AGCAAUGCAGCUCAAAACGCUUAGCCUAGCCACACCCCCACGGGAAACAGCAGUGAUUAACCUUUAGCAAUAAACGAAAGUUUAACUA |
| | | AGCUAUACUAACCCCAGGGUUGGUCAAUUUCGUGCCAGCCACACCCUCGAGCUAGCAAAAAAAAAAAAAAAAAAAAAAAAAAAAAAGC |
| | | AUAUGACUAAAAAAAAAAAAAAAAAAAAAAAAAAAAAAAAAAAAAAAAAAAAAAAAAAAAAAAAAAAAAAAAAAAAAA |

CD27L-CD40L mouse

| | | |
|---|---|---|
| 72 | ModA | MRVTAPRTLILLLSGALALTETWAGSGSHPEPHTAELQLNLTVPRKDPTLRWGAGPALGRSFTHGPELEEGHLRIHQDGLYRLHIQVT |
| | murine | LANCSSPGSTLQHRATLAVGICSPAAHGISLLRGRFGQDCTVALQRLTYLVHGDVLCTNLTLPLLPSRNADETFFGVQWICPGGGSGG |
| | CD27L- | GHPEPHTAELQLNLTVPRKDPTLRWGAGPALGRSFTHGPELEEGHLRIHQDGLYRLHIQVTLANCSSPGSTLQHRATLAVGICSPAAH |
| | CD40L | GISLLRGRFGQDCTVALQRLTYLVHGDVLCTNLTLPLLPSRNADETFFGVQWICPGGGSGGGHPEPHTAELQLNLTVPRKDPTLRWGA |
| | (amino | GPALGRSFTHGPELEEGHLRIHQDGLYRLHIQVTLANCSSPGSTLQHRATLAVGICSPAAHGISLLRGRFGQDCTVALQRLTYLVHGD |
| | acid) | VLCTNLTLPLLPSRNADETFFGVQWICPGGGGSGGGGSGGGGSGDEDPQIAAHVVSEANSNAASVLQWAKKGYYTMKSNLVMLENGKQ |
| | Sequence | LTVKREGLYYVYTQVTFCSNREPSSQRPFIVGLWLKPSSGSERILLKAANTHSSSQLCEQQSVHLGGVFELQAGASVFVNVTEASQVI |
| | annota- | HRVGFSSFGLLKLGGGSGGGGDEDPQIAAHVVSEANSNAASVLQWAKKGYYTMKSNLVMLENGKQLTVKREGLYYVYTQVTFCSNREP |
| | tions | SSQRPFIVGLWLKPSSGSERILLKAANTHSSSQLCEQQSVHLGGVFELQAGASVFVNVTEASQVIHRVGFSSFGLLKLGGGSGGGGDE |
| | CAPS: | DPQIAAHVVSEANSNAASVLQWAKKGYYTMKSNLVMLENGKQLTVKREGLYYVYTQVTFCSNREPSSQRPFIVGLWLKPSSGSERILL |
| | CD27L; | KAANTHSSSQLCEQQSVHLGGVFELQAGASVFVNVTEASQVIHRVGFSSFGLLKL |
| | CAPS: | |
| | linker; | |
| | CAPS: | |
| | CD40L | |
| 73 | ModA | GGGCGAACTAGTATTCTTCTGGTCCCCACAGACTCAGAGAGAACCCGCCACCATGAGAGTGACCGCCCCCAGAACCCTGATCCTGCTG |
| | murine | CTGTCTGGCGCCCTGGCCCTGACAGAGACATGGGCCGGAAGCGGATCCCACCCCGAGCCCCACACCGCCGAACTGCAGCTGAACCTGA |
| | CD27L- | CCGTGCCCAGAAAGGACCCCACCCTGAGATGGGGAGCTGGCCCTGCTCTGGGCAGATCCTTTACACACGGCCCCGAGCTGGAAGAAGG |
| | CD40L | CCACCTGAGAATCCACCAGGACGGCCTGTACAGACTGCACATCCAAGTGACCCTGGCCAACTGCAGCAGCCCTGGCTCTACCCTGCAG |
| | (DNA: | CACAGAGCCACACTGGCCGTGGGCATCTGTAGCCCTGCTGCTCACGGAATCAGCCTGCTGAGAGGCAGATTCGGCCAGGACTGTACCG |
| | 5′ UTR- | TGGCCCTGCAGAGGCTGACCTATCTGGTGCATGGCGACGTGCTGTGCACCAACCTGACACTGCCTCTGCTGCCCAGCAGAAACGCCGA |
| | CDS-3′ | CGAAACATTCTTTGGAGTGCAGTGGATTTGTCCTGGCGGAGGGTCCGGGGGAGGACACCCAGAACCTCATACAGCTGAACTGCAGCTG |
| | UTR) | AACCTGACCGTGCCCAGAAAGGACCCCACCCTGAGATGGGGAGCTGGCCCTGCTCTGGGCAGATCCTTTACACACGGCCCCGAGCTGG |
| | | AAGAAGGCCACCTGAGAATCCACCAGGACGGCCTGTACAGACTGCACATCCAAGTGACCCTGGCCAACTGCAGCAGCCCTGGCTCTAC |
| | | CCTGCAGCACAGAGCCACACTGGCCGTGGGCATCTGTAGCCCTGCTGCTCACGGAATCAGCCTGCTGAGAGGCAGATTCGGCCAGGAC |
| | | TGTACCGTGGCCCTGCAGAGGCTGACCTATCTGGTGCATGGCGACGTGCTGTGCACCAACCTGACACTGCCTCTGCTGCCCAGCAGAA |
| | | ACGCCGACGAAACATTCTTTGGAGTGCAGTGGATTTGTCCTGGGGGAGGCTCCGGAGGCGGACACCCTGAACCTCATACAGCTGAACT |
| | | GCAGCTGAACCTGACCGTGCCCAGAAAGGACCCCACCCTGAGATGGGGAGCTGGCCCTGCTCTGGGCAGATCCTTTACACACGGCCCC |
| | | GAGCTGGAAGAAGGCCACCTGAGAATCCACCAGGACGGCCTGTACAGACTGCACATCCAAGTGACCCTGGCCAACTGCAGCAGCCCTG |
| | | GCTCTACCCTGCAGCACAGAGCCACACTGGCCGTGGGCATCTGTAGCCCTGCTGCTCACGGAATCAGCCTGCTGAGAGGCAGATTCGG |
| | | CCAGGACTGTACCGTGGCCCTGCAGAGGCTGACCTATCTGGTGCATGGCGACGTGCTGTGCACCAACCTGACACTGCCTCTGCTGCCC |
| | | AGCAGAAACGCCGACGAGACCTTCTTCGGCGTCCAGTGGATCTGCCCCGGAGGCGGTGGTAGTGGAGGTGGCGGGTCCGGTGGAGGTG |
| | | GAAGCGGCGACGAGGACCCCCAGATCGCCGCCCACGTGGTGTCTGAGGCCAACAGCAACGCCGCCTCTGTGCTGCAGTGGGCCAAGAA |
| | | AGGCTACTACACCATGAAGTCCAACCTCGTGATGCTGGAAAACGGCAAGCAGCTGACCGTGAAGCGCGAGGGCCTGTACTATGTGTAC |
| | | ACCCAAGTGACATTCTGCAGCAACCGCGAGCCCAGCAGCCAGAGGCCTTTTATCGTGGGCCTGTGGCTGAAGCCTAGCAGCGGCAGCG |
| | | AGAGAATCCTGCTGAAGGCCGCCAACACCCACAGCAGCTCTCAGCTGTGCGAGCAGCAGTCTGTGCACCTGGGAGGCGTGTTCGAGCT |
| | | GCAAGCTGGCGCTTCCGTGTTCGTGAACGTGACCGAGGCCAGCCAAGTGATCCACAGAGTGGGCTTCAGCAGCTTTGGACTGCTCAAA |
| | | CTGGGCGGAGGGTCCGGCGGAGGCGGAGATGAAGATCCTCAGATTGCTGCCCACGTGGTGTCTGAGGCCAACAGCAACGCCGCCTCTG |
| | | TGCTGCAGTGGGCCAAGAAAGGCTACTACACCATGAAGTCCAACCTCGTGATGCTGGAAAACGGCAAGCAGCTGACCGTGAAGCGCGA |
| | | GGGCCTGTACTATGTGTACACCCAAGTGACATTCTGCAGCAACCGCGAGCCCAGCAGCCAGAGGCCTTTTATCGTGGGCCTGTGGCTG |
| | | AAGCCTAGCAGCGGCAGCGAGAGAATCCTGCTGAAGGCCGCCAACACCCACAGCAGCTCTCAGCTGTGCGAGCAGCAGTCTGTGCACC |
| | | TGGGAGGCGTGTTCGAGCTGCAAGCTGGCGCTTCCGTGTTCGTGAACGTGACCGAGGCCAGCCAAGTGATCCACAGAGTGGGCTTCAG |
| | | CAGCTTTGGACTGCTCAAACTGGGAGGCGGCTCCGGAGGCGGAGGAGATGAAGATCCTCAGATTGCTGCCCACGTGGTGTCTGAGGCC |
| | | AACAGCAACGCCGCCTCTGTGCTGCAGTGGGCCAAGAAAGGCTACTACACCATGAAGTCCAACCTCGTGATGCTGGAAAACGGCAAGC |
| | | AGCTGACCGTGAAGCGCGAGGGCCTGTACTATGTGTACACCCAAGTGACATTCTGCAGCAACCGCGAGCCCAGCAGCCAGAGGCCTTT |
| | | TATCGTGGGCCTGTGGCTGAAGCCTAGCAGCGGCAGCGAGAGAATCCTGCTGAAGGCCGCCAACACCCACAGCAGCTCTCAGCTGTGC |
| | | GAGCAGCAGTCTGTGCACCTGGGAGGCGTGTTCGAGCTGCAAGCTGGCGCTTCCGTGTTCGTGAACGTGACCGAGGCCAGCCAAGTGA |
| | | TCCACAGAGTGGGCTTCTCCTCCTTCGGCCTCCTGAAGCTGTGACTCGAGAGCTCGCTTTCTTGCTGTCCAATTTCTATTAAAGGTTC |
| | | CTTTGTTCCCTAAGTCCAACTACTAAACTGGGGGATATTATGAAGGGCCTTGAGCATCTGGATTCTGCCTAATAAAAAACATTTATTT |
| | | TCATTGCTGCGTCGAGAGCTCGCTTTCTTGCTGTCCAATTTCTATTAAAGGTTCCTTTGTTCCCTAAGTCCAACTACTAAACTGGGGG |
| | | ATATTATGAAGGGCCTTGAGCATCTGGATTCTGCCTAATAAAAAACATTTATTTTCATTGCTGCGTCGAGACCTGGTCCAGAGTCGCT |
| | | AGCAAAAAAAAAAAAAAAAAAAAAAAAAAAAAAGCATATGACTAAAAAAAAAAAAAAAAAAAAAAAAAAAAAAAAAAAAAAAAAAAAA |
| | | AAAAAAAAAAAAAAAAAAAAAAAAA |
| 74 | ModA | GGGCGAACUAGUAUUCUUCUGGUCCCCACAGACUCAGAGAGAACCCGCCACCAUGAGAGUGACCGCCCCCAGAACCCUGAUCCUGCUG |
| | murine | CUGUCUGGCGCCCUGGCCCUGACAGAGACAUGGGCCGGAAGCGGAUCCCACCCCGAGCCCCACACCGCCGAACUGCAGCUGAACCUGA |
| | CD27L- | CCGUGCCCAGAAAGGACCCCACCCUGAGAUGGGGAGCUGGCCCUGCUCUGGGCAGAUCCUUUACACACGGCCCCGAGCUGGAAGAAGG |
| | CD40L | CCACCUGAGAAUCCACCAGGACGGCCUGUACAGACUGCACAUCCAAGUGACCCUGGCCAACUGCAGCAGCCCUGGCUCUACCCUGCAG |
| | (RNA) | CACAGAGCCACACUGGCCGUGGGCAUCUGUAGCCCUGCUGCUCACGGAAUCAGCCUGCUGAGAGGCAGAUUCGGCCAGGACUGUACCG |
| | | UGGCCCUGCAGAGGCUGACCUAUCUGGUGCAUGGCGACGUGCUGUGCACCAACCUGACACUGCCUCUGCUGCCCAGCAGAAACGCCGA |
| | | CGAAACAUUCUUUGGAGUGCAGUGGAUUUGUCCUGGCGGAGGGUCCGGGGGAGGACACCCAGAACCUCAUACAGCUGAACUGCAGCUG |
| | | AACCUGACCGUGCCCAGAAAGGACCCCACCCUGAGAUGGGGAGCUGGCCCUGCUCUGGGCAGAUCCUUUACACACGGCCCCGAGCUGG |
| | | AAGAAGGCCACCUGAGAAUCCACCAGGACGGCCUGUACAGACUGCACAUCCAAGUGACCCUGGCCAACUGCAGCAGCCCUGGCUCUAC |
| | | CCUGCAGCACAGAGCCACACUGGCCGUGGGCAUCUGUAGCCCUGCUGCUCACGGAAUCAGCCUGCUGAGAGGCAGAUUCGGCCAGGAC |
| | | UGUACCGUGGCCCUGCAGAGGCUGACCUAUCUGGUGCAUGGCGACGUGCUGUGCACCAACCUGACACUGCCUCUGCUGCCCAGCAGAA |
| | | ACGCCGACGAAACAUUCUUUGGAGUGCAGUGGAUUUGUCCUGGGGGAGGCUCCGGAGGCGGACACCCUGAACCUCAUACAGCUGAACU |
| | | GCAGCUGAACCUGACCGUGCCCAGAAAGGACCCCACCCUGAGAUGGGGAGCUGGCCCUGCUCUGGGCAGAUCCUUUACACACGGCCCC |
| | | GAGCUGGAAGAAGGCCACCUGAGAAUCCACCAGGACGGCCUGUACAGACUGCACAUCCAAGUGACCCUGGCCAACUGCAGCAGCCCUG |
| | | GCUCUACCCUGCAGCACAGAGCCACACUGGCCGUGGGCAUCUGUAGCCCUGCUGCUCACGGAAUCAGCCUGCUGAGAGGCAGAUUCGG |
| | | CCAGGACUGUACCGUGGCCCUGCAGAGGCUGACCUAUCUGGUGCAUGGCGACGUGCUGUGCACCAACCUGACACUGCCUCUGCUGCCC |
| | | AGCAGAAACGCCGACGAGACCUUCUUCGGCGUCCAGUGGAUCUGCCCCGGAGGCGGUGGUAGUGGAGGUGGCGGGUCCGGUGGAGGUG |
| | | GAAGCGGCGACGAGGACCCCCAGAUCGCCGCCCACGUGGUGUCUGAGGCCAACAGCAACGCCGCCUCUGUGCUGCAGUGGGCCAAGAA |
| | | AGGCUACUACACCAUGAAGUCCAACCUCGUGAUGCUGGAAAACGGCAAGCAGCUGACCGUGAAGCGCGAGGGCCUGUACUAUGUGUAC |
| | | ACCCAAGUGACAUUCUGCAGCAACCGCGAGCCCAGCAGCCAGAGGCCUUUUAUCGUGGGCCUGUGGCUGAAGCCUAGCAGCGGCAGCG |
| | | AGAGAAUCCUGCUGAAGGCCGCCAACACCCACAGCAGCUCUCAGCUGUGCGAGCAGCAGUCUGUGCACCUGGGAGGCGUGUUCGAGCU |
| | | GCAAGCUGGCGCUUCCGUGUUCGUGAACGUGACCGAGGCCAGCCAAGUGAUCCACAGAGUGGGCUUCAGCAGCUUUGGACUGCUCAAA |
| | | CUGGGCGGAGGGUCCGGCGGAGGCGGAGAUGAAGAUCCUCAGAUUGCUGCCCACGUGGUGUCUGAGGCCAACAGCAACGCCGCCUCUG |
| | | UGCUGCAGUGGGCCAAGAAAGGCUACUACACCAUGAAGUCCAACCUCGUGAUGCUGGAAAACGGCAAGCAGCUGACCGUGAAGCGCGA |
| | | GGGCCUGUACUAUGUGUACACCCAAGUGACAUUCUGCAGCAACCGCGAGCCCAGCAGCCAGAGGCCUUUUAUCGUGGGCCUGUGGCUG |
| | | AAGCCUAGCAGCGGCAGCGAGAGAAUCCUGCUGAAGGCCGCCAACACCCACAGCAGCUCUCAGCUGUGCGAGCAGCAGUCUGUGCACC |
| | | UGGGAGGCGUGUUCGAGCUGCAAGCUGGCGCUUCCGUGUUCGUGAACGUGACCGAGGCCAGCCAAGUGAUCCACAGAGUGGGCUUCAG |
| | | CAGCUUUGGACUGCUCAAACUGGGAGGCGGCUCCGGAGGCGGAGGAGAUGAAGAUCCUCAGAUUGCUGCCCACGUGGUGUCUGAGGCC |
| | | AACAGCAACGCCGCCUCUGUGCUGCAGUGGGCCAAGAAAGGCUACUACACCAUGAAGUCCAACCUCGUGAUGCUGGAAAACGGCAAGC |
| | | AGCUGACCGUGAAGCGCGAGGGCCUGUACUAUGUGUACACCCAAGUGACAUUCUGCAGCAACCGCGAGCCCAGCAGCCAGAGGCCUUU |
| | | UAUCGUGGGCCUGUGGCUGAAGCCUAGCAGCGGCAGCGAGAGAAUCCUGCUGAAGGCCGCCAACACCCACAGCAGCUCUCAGCUGUGC |
| | | GAGCAGCAGUCUGUGCACCUGGGAGGCGUGUUCGAGCUGCAAGCUGGCGCUUCCGUGUUCGUGAACGUGACCGAGGCCAGCCAAGUGA |
| | | UCCACAGAGUGGGCUUCUCCUCCUUCGGCCUCCUGAAGCUGUGACUCGAGAGCUCGCUUUCUUGCUGUCCAAUUUCUAUUAAAGGUUC |
| | | CUUUGUUCCCUAAGUCCAACUACUAAACUGGGGGAUAUUAUGAAGGGCCUUGAGCAUCUGGAUUCUGCCUAAUAAAAAACAUUUAUUU |
| | | UCAUUGCUGCGUCGAGAGCUCGCUUUCUUGCUGUCCAAUUUCUAUUAAAGGUUCCUUUGUUCCCUAAGUCCAACUACUAAACUGGGGG |
| | | AUAUUAUGAAGGGCCUUGAGCAUCUGGAUUCUGCCUAAUAAAAAACAUUUAUUUUCAUUGCUGCGUCGAGACCUGGUCCAGAGUCGCU |
| | | AGCAAAAAAAAAAAAAAAAAAAAAAAAAAAAAAGCAUAUGACUAAAAAAAAAAAAAAAAAAAAAAAAAAAAAAAAAAAAAAAAAAAAA |
| | | AAAAAAAAAAAAAAAAAAAAAAAAA |
| 75 | ModB | MGAMAPRTLLLLLAAALAPTQTRAGPGSHPEPHTAELQLNLTVPRKDPTLRWGAGPALGRSFTHGPELEEGHLRIHQDGLYRLHIQVT |
| | murine | LANCSSPGSTLQHRATLAVGICSPAAHGISLLRGRFGQDCTVALQRLTYLVHGDVLCTNLTLPLLPSRNADETFFGVQWICPGGGSGG |
| | CD27L- | GHPEPHTAELQLNLTVPRKDPTLRWGAGPALGRSFTHGPELEEGHLRIHQDGLYRLHIQVTLANCSSPGSTLQHRATLAVGICSPAAH |
| | CD40L | GISLLRGRFGQDCTVALQRLTYLVHGDVLCTNLTLPLLPSRNADETFFGVQWICPGGGSGGGHPEPHTAELQLNLTVPRKDPTLRWGA |
| | (amino | GPALGRSFTHGPELEEGHLRIHQDGLYRLHIQVTLANCSSPGSTLQHRATLAVGICSPAAHGISLLRGRFGQDCTVALQRLTYLVHGD |
| | acid) | VLCTNLTLPLLPSRNADETFFGVQWICPGGGGSGGGGSGGGGSGDEDPQIAAHVVSEANSNAASVLQWAKKGYYTMKSNLVMLENGKQ |
| | | LTVKREGLYYVYTQVTFCSNREPSSQRPFIVGLWLKPSSGSERILLKAANTHSSSQLCEQQSVHLGGVFELQAGASVFVNVTEASQVI |
| | | HRVGFSSFGLLKLGGGSGGGGDEDPQIAAHVVSEANSNAASVLQWAKKGYYTMKSNLVMLENGKQLTVKREGLYYVYTQVTFCSNREP |
| | | SSQRPFIVGLWLKPSSGSERILLKAANTHSSSQLCEQQSVHLGGVFELQAGASVFVNVTEASQVIHRVGFSSFGLLKLGGGSGGGGDE |
| | | DPQIAAHVVSEANSNAASVLQWAKKGYYTMKSNLVMLENGKQLTVKREGLYYVYTQVTFCSNREPSSQRPFIVGLWLKPSSGSERILL |
| | | KAANTHSSSQLCEQQSVHLGGVFELQAGASVFVNVTEASQVIHRVGFSSFGLLKL |
| 76 | ModB | GGAATAAACTAGTCTCAACACAACATATACAAAACAAACGAATCTCAAGCAATCAAGCATTCTACTTCTATTGCAGCAATTTAAATCA |
| | murine | TTTCTTTTAAAGCAAAAGCAATTTTCTGAAAATTTTCACCATTTACGAACGATAGCCATGGGCGCCATGGCCCCTAGAACATTGCTCC |
| | CD27L- | TGCTGCTGGCCGCTGCCCTGGCCCCTACACAGACAAGAGCTGGACCTGGATCCCACCCCGAGCCCCACACCGCCGAACTGCAGCTGAA |
| | CD40L | CCTGACCGTGCCCAGAAAGGACCCCACCCTGAGATGGGGAGCTGGCCCTGCTCTGGGCAGATCCTTTACACACGGCCCCGAGCTGGAA |
| | (DNA: | GAAGGCCACCTGAGAATCCACCAGGACGGCCTGTACAGACTGCACATCCAAGTGACCCTGGCCAACTGCAGCAGCCCTGGCTCTACCC |
| | 5′ UTR- | TGCAGCACAGAGCCACACTGGCCGTGGGCATCTGTAGCCCTGCTGCTCACGGAATCAGCCTGCTGAGAGGCAGATTCGGCCAGGACTG |
| | CDS-3′ | TACCGTGGCCCTGCAGAGGCTGACCTATCTGGTGCATGGCGACGTGCTGTGCACCAACCTGACACTGCCTCTGCTGCCCAGCAGAAAC |
| | UTR) | GCCGACGAAACATTCTTTGGAGTGCAGTGGATTTGTCCTGGCGGAGGGTCCGGGGGAGGACACCCAGAACCTCATACAGCTGAACTGC |
| | | AGCTGAACCTGACCGTGCCCAGAAAGGACCCCACCCTGAGATGGGGAGCTGGCCCTGCTCTGGGCAGATCCTTTACACACGGCCCCGA |
| | | GCTGGAAGAAGGCCACCTGAGAATCCACCAGGACGGCCTGTACAGACTGCACATCCAAGTGACCCTGGCCAACTGCAGCAGCCCTGGC |
| | | TCTACCCTGCAGCACAGAGCCACACTGGCCGTGGGCATCTGTAGCCCTGCTGCTCACGGAATCAGCCTGCTGAGAGGCAGATTCGGCC |
| | | AGGACTGTACCGTGGCCCTGCAGAGGCTGACCTATCTGGTGCATGGCGACGTGCTGTGCACCAACCTGACACTGCCTCTGCTGCCCAG |
| | | CAGAAACGCCGACGAAACATTCTTTGGAGTGCAGTGGATTTGTCCTGGGGGAGGCTCCGGAGGCGGACACCCTGAACCTCATACAGCT |
| | | GAACTGCAGCTGAACCTGACCGTGCCCAGAAAGGACCCCACCCTGAGATGGGGAGCTGGCCCTGCTCTGGGCAGATCCTTTACACACG |
| | | GCCCCGAGCTGGAAGAAGGCCACCTGAGAATCCACCAGGACGGCCTGTACAGACTGCACATCCAAGTGACCCTGGCCAACTGCAGCAG |
| | | CCCTGGCTCTACCCTGCAGCACAGAGCCACACTGGCCGTGGGCATCTGTAGCCCTGCTGCTCACGGAATCAGCCTGCTGAGAGGCAGA |
| | | TTCGGCCAGGACTGTACCGTGGCCCTGCAGAGGCTGACCTATCTGGTGCATGGCGACGTGCTGTGCACCAACCTGACACTGCCTCTGC |
| | | TGCCCAGCAGAAACGCCGACGAGACCTTCTTCGGCGTCCAGTGGATCTGCCCCGGAGGCGGTGGTAGTGGAGGTGGCGGGTCCGGTGG |
| | | AGGTGGAAGCGGCGACGAGGACCCCCAGATCGCCGCCCACGTGGTGTCTGAGGCCAACAGCAACGCCGCCTCTGTGCTGCAGTGGGCC |
| | | AAGAAAGGCTACTACACCATGAAGTCCAACCTCGTGATGCTGGAAAACGGCAAGCAGCTGACCGTGAAGCGCGAGGGCCTGTACTATG |
| | | TGTACACCCAAGTGACATTCTGCAGCAACCGCGAGCCCAGCAGCCAGAGGCCTTTTATCGTGGGCCTGTGGCTGAAGCCTAGCAGCGG |
| | | CAGCGAGAGAATCCTGCTGAAGGCCGCCAACACCCACAGCAGCTCTCAGCTGTGCGAGCAGCAGTCTGTGCACCTGGGAGGCGTGTTC |
| | | GAGCTGCAAGCTGGCGCTTCCGTGTTCGTGAACGTGACCGAGGCCAGCCAAGTGATCCACAGAGTGGGCTTCAGCAGCTTTGGACTGC |
| | | TCAAACTGGGCGGAGGGTCCGGCGGAGGCGGAGATGAAGATCCTCAGATTGCTGCCCACGTGGTGTCTGAGGCCAACAGCAACGCCGC |
| | | CTCTGTGCTGCAGTGGGCCAAGAAAGGCTACTACACCATGAAGTCCAACCTCGTGATGCTGGAAAACGGCAAGCAGCTGACCGTGAAG |
| | | CGCGAGGGCCTGTACTATGTGTACACCCAAGTGACATTCTGCAGCAACCGCGAGCCCAGCAGCCAGAGGCCTTTTATCGTGGGCCTGT |
| | | GGCTGAAGCCTAGCAGCGGCAGCGAGAGAATCCTGCTGAAGGCCGCCAACACCCACAGCAGCTCTCAGCTGTGCGAGCAGCAGTCTGT |
| | | GCACCTGGGAGGCGTGTTCGAGCTGCAAGCTGGCGCTTCCGTGTTCGTGAACGTGACCGAGGCCAGCCAAGTGATCCACAGAGTGGGC |
| | | TTCAGCAGCTTTGGACTGCTCAAACTGGGAGGCGGCTCCGGAGGCGGAGGAGATGAAGATCCTCAGATTGCTGCCCACGTGGTGTCTG |
| | | AGGCCAACAGCAACGCCGCCTCTGTGCTGCAGTGGGCCAAGAAAGGCTACTACACCATGAAGTCCAACCTCGTGATGCTGGAAAACGG |
| | | CAAGCAGCTGACCGTGAAGCGCGAGGGCCTGTACTATGTGTACACCCAAGTGACATTCTGCAGCAACCGCGAGCCCAGCAGCCAGAGG |
| | | CCTTTTATCGTGGGCCTGTGGCTGAAGCCTAGCAGCGGCAGCGAGAGAATCCTGCTGAAGGCCGCCAACACCCACAGCAGCTCTCAGC |
| | | TGTGCGAGCAGCAGTCTGTGCACCTGGGAGGCGTGTTCGAGCTGCAAGCTGGCGCTTCCGTGTTCGTGAACGTGACCGAGGCCAGCCA |
| | | AGTGATCCACAGAGTGGGCTTCTCCTCCTTCGGCCTCCTGAAGCTGTGACTCGACGTCCTGGTACTGCATGCACGCAATGCTAGCTGC |
| | | CCCTTTCCCGTCCTGGGTACCCCGAGTCTCCCCCGACCTCGGGTCCCAGGTATGCTCCCACCTCCACCTGCCCCACTCACCACCTCTG |
| | | CTAGTTCCAGACACCTCCCAAGCACGCAGCAATGCAGCTCAAAACGCTTAGCCTAGCCACACCCCCACGGGAAACAGCAGTGATTAAC |
| | | CTTTAGCAATAAACGAAAGTTTAACTAAGCTATACTAACCCCAGGGTTGGTCAATTTCGTGCCAGCCACACCCTCGAGCTAGCAAAAA |
| | | AAAAAAAAAAAAAAAAAAAAAAAAAGCATATGACTAAAAAAAAAAAAAAAAAAAAAAAAAAAAAAAAAAAAAAAAAAAAAAAAAAAAA |
| | | AAAAAAAAAAAAAAAAA |
| 77 | ModB | GGAAUAAACUAGUCUCAACACAACAUAUACAAAACAAACGAAUCUCAAGCAAUCAAGCAUUCUACUUCUAUUGCAGCAAUUUAAAUCA |
| | murine | UUUCUUUUAAAGCAAAAGCAAUUUUCUGAAAAUUUUCACCAUUUACGAACGAUAGCCAUGGGCGCCAUGGCCCCUAGAACAUUGCUCC |
| | CD27L- | UGCUGCUGGCCGCUGCCCUGGCCCCUACACAGACAAGAGCUGGACCUGGAUCCCACCCCGAGCCCCACACCGCCGAACUGCAGCUGAA |
| | CD40L | CCUGACCGUGCCCAGAAAGGACCCCACCCUGAGAUGGGGAGCUGGCCCUGCUCUGGGCAGAUCCUUUACACACGGCCCCGAGCUGGAA |
| | (RNA) | GAAGGCCACCUGAGAAUCCACCAGGACGGCCUGUACAGACUGCACAUCCAAGUGACCCUGGCCAACUGCAGCAGCCCUGGCUCUACCC |
| | | UGCAGCACAGAGCCACACUGGCCGUGGGCAUCUGUAGCCCUGCUGCUCACGGAAUCAGCCUGCUGAGAGGCAGAUUCGGCCAGGACUG |
| | | UACCGUGGCCCUGCAGAGGCUGACCUAUCUGGUGCAUGGCGACGUGCUGUGCACCAACCUGACACUGCCUCUGCUGCCCAGCAGAAAC |
| | | GCAGACGAAACAUUCUUUGGAGUGCAGUGGAUUUGUCCUGGCGGAGGGUCCGGGGGAGGACACCCAGAACCUCAUACAGCUGAACUGC |
| | | AGCUGAACCUGACCGUGCCCAGAAAGGACCCCACCCUGAGAUGGGGAGCUGGCCCUGCUCUGGGCAGAUCCUUUACACACGGCCCCGA |
| | | GCUGGAAGAAGGCCACCUGAGAAUCCACCAGGACGGCCUGUACAGACUGCACAUCCAAGUGACCCUGGCCAACUGCAGCAGCCCUGGC |
| | | UCUACCCUGCAGCACAGAGCCACACUGGCCGUGGGCAUCUGUAGCCCUGCUGCUCACGGAAUCAGCCUGCUGAGAGGCAGAUUCGGCC |
| | | AGGACUGUACCGUGGCCCUGCAGAGGCUGACCUAUCUGGUGCAUGGCGACGUGCUGUGCACCAACCUGACACUGCCUCUGCUGCCCAG |
| | | CAGAAACGCCGACGAAACAUUCUUUGGAGUGCAGUGGAUUUGUCCUGGGGGAGGCUCCGGAGGCGGACACCCUGAACCUCAUACAGCU |
| | | GAACUGCAGCUGAACCUGACCGUGCCCAGAAAGGACCCCACCCUGAGAUGGGGAGCUGGCCCUGCUCUGGGCAGAUCCUUUACACACG |
| | | GCCCCGAGCUGGAAGAAGGCCACCUGAGAAUCCACCAGGACGGCCUGUACAGACUGCACAUCCAAGUGACCCUGGCCAACUGCAGCAG |
| | | CCCUGGCUCUACCCUGCAGCACAGAGCCACACUGGCCGUGGGCAUCUGUAGCCCUGCUGCUCACGGAAUCAGCCUGCUGAGAGGCAGA |
| | | UUCGGCCAGGACUGUACCGUGGCCCUGCAGAGGCUGACCUAUCUGGUGCAUGGCGACGUGCUGUGCACCAACCUGACACUGCCUCUGC |
| | | UGCCCAGCAGAAACGCCGACGAGACCUUCUUCGGCGUCCAGUGGAUCUGCCCCGGAGGCGGUGGUAGUGGAGGUGGCGGGUCCGGUGG |
| | | AGGUGGAAGCGGCGACGAGGACCCCCAGAUCGCCGCCCACGUGGUGUCUGAGGCCAACAGCAACGCCGCCUCUGUGCUGCAGUGGGCC |
| | | AAGAAAGGCUACUACACCAUGAAGUCCAACCUCGUGAUGCUGGAAAACGGCAAGCAGCUGACCGUGAAGCGCGAGGGCCUGUACUAUG |
| | | UGUACACCCAAGUGACAUUCUGCAGCAACCGCGAGCCCAGCAGCCAGAGGCCUUUUAUCGUGGGCCUGUGGCUGAAGCCUAGCAGCGG |
| | | CAGCGAGAGAAUCCUGCUGAAGGCCGCCAACACCCACAGCAGCUCUCAGCUGUGCGAGCAGCAGUCUGUGCACCUGGGAGGCGUGUUC |
| | | GAGCUGCAAGCUGGCGCUUCCGUGUUCGUGAACGUGACCGAGGCCAGCCAAGUGAUCCACAGACUGGGCUUCAGCAGCUUUGGACUGC |
| | | UCAAACUGGGCGGAGGGUCCGGCGGAGGCGGAGAUGAAGAUCCUCAGAUUGCUGCCCACGUGGUGUCUGAGGCCAACAGCAACGCCGC |
| | | CUCUGUGCUGCAGUGGGCCAAGAAAGGCUACUACACCAUGAAGUCCAACCUCGUGAUGCUGGAAAACGGCAAGCAGCUGACCGUGAAG |
| | | CGCGAGGGCCUGUACUAUGUGUACACCCAAGUGACAUUCUGCAGCAACCGCGAGCCCAGCAGCCAGAGGCCUUUUAUCGUGGGCCUGU |
| | | GGCUGAAGCCUAGCAGCGGCAGCGAGAGAAUCCUGCUGAAGGCCGCCAACACCCACAGCAGCUCUCAGCUGUGCGAGCAGCAGUCUGU |
| | | GCACCUGGGAGGCGUGUUCGAGCUGCAAGCUGGCGCUUCCGUGUUCGUGAACGUGACCGAGGCCAGCCAAGUGAUCCACAGAGUGGGC |
| | | UUCAGCAGCUUUGGACUGCUCAAACUGGGAGGCGGCUCCGGAGGCGGAGGAGAUGAAGAUCCUCAGAUUGCUGCCCACGUGGUGUCUG |
| | | AGGCCAACAGCACGCCGCCUCUGUGCUGCAGUGGGCCAAGAAAGGCUACUACACCAUGAAGUCCAACCUCGUGAUGCCYGGAAAACGG |
| | | CAAGCAGCUGACCGUGAAGCGCGAGGGCCUGUACUAUGUGUACACCCAAGUGACAUUCUGCAGCAACCGCGAGCCCAGCAGCCAGAGG |
| | | CCUUUUAUCGUGGGCCUGUGGCUGAAGCCUAGCAGCGGCAGCGAGAGAAUCCUGCUGAAGGCCGCCAACACCCACAGCAGCUCUCAGC |
| | | UGUGCGAGCAGCAGUCUGUGCACCUGGGAGGCGUGUUCGAGCUGCAAGCUGGCGCUUCCGUGUUCGUGAACGUGACCGAGGCCAGCCA |
| | | AGUGAUCCACAGAGUGGGCUUCUCCUCCUUCGGCCUCCUGAAGCUGUGACUCGACGUCCUGGUACUGCAUGCACGCAAUGCUAGCUGC |
| | | CCCUUUCCCGUCCUGGGUACCCCGAGUCUCCCCCGACCUCGGGUCCCAGGUAUGCUCCCACCUCCACCUGCCCCACUCACCACCUCUG |
| | | CUAGUUCCAGACACCUCCCAAGCACGCAGCAAUGCAGCUCAAAACGCUUAGCCUAGCCACACCCCCACGGGAAACAGCAGUGAUUAAC |
| | | CUUUAGCAAUAAACGAAAGUUUAACUAAGCUAUACUAACCCCAGGGUUGGUCAAUUUCGUGCCAGCCACACCCUCGAGCUAGCAAAAA |
| | | AAAAAAAAAAAAAAAAAAAAAAAAAGCAUAUGACUAAAAAAAAAAAAAAAAAAAAAAAAAAAAAAAAAAAAAAAAAAAAAAAAAAAAA |
| | | AAAAAAAAAAAAAAAAA |

Other sequences of the invention

| | | |
|---|---|---|
| 78 | Poly-A | AAAAAAAAAAAAAAAAAAAAAAAAAAAAAAGCAUAUGACUAAAAAAAAAAAAAAAAAAAAAAAAAAAAAAAAAAAAAAAAAAAAAAAA |
| | | AAAAAAAAAAAAAAAAAAAAAA |
| 79 | Anti- | EVQLLESGGV LVQPGGSLRL SCAASGFTFS NFGMTWVRQA PGKGLEWVSG ISGGGRDTYF ADSVKGRFTI SRDNSKNTLY |
| | PD1 | LQMNSLKGED TAVYYCVKWG NIYFDYWGQG TLVTVSSAST KGPSVFPLAP CSRSTSESTA ALGCLVKDYF PEPVTVSWNS |
| | Mab | GALTSGVHTF PAVLQSSGLY SLSSVVTVPS SSLGTKTYTC NVDHKPSNTK VDKRVESKYG PPCPPCPAPE FLGGPSVFLF |
| | heavy | PPKPKDTLMI SRTPEVTCVV VDVSQEDPEV QFNWYVDGVE VHNAKTKPRE EQFNSTYRVV SVLTVLHQDW LNGKEYKCKV |
| | chain | SNKGLPSSIE KTISKAKGQP REPQVYTLPP SQEEMTKNQV SLTCLVKGFY PSDIAVEWES NGQPENNYKT TPPVLDSDGS |
| | | FFLYSRLTVD KSRWQEGNVF SCSVMHEALH NHYTQKSLSL SLGK |
| 80 | Anti- | DIQMTQSPSS LSASVGDSIT ITCRASLSIN TFLNWYQQKP GKAPNLLIYA ASSLHGGVPS RFSGSGSGTD FTLTIRTLQP |
| | PD1 | EDFATYYCQQ SSNTPFTFGP GTVVDFRRTV AAPSVFIFPP SDEQLKSGTA SVVCLLNNFY PREAKVQWKV DNALQSGNSQ |
| | Mab | ESVTEQDSKD STYSLSSTLT LSKADYEKHK VYACEVTHQG LSSPVTKSEN RGEC |
| | light | |
| | chain | |
| 81 | HCDR1 | GFTFSNFG |
| 82 | HCDR2 | ISGGGRDT |
| 83 | HCDR3 | VKWGNIYFDY |
| 84 | LCDR1 | LSINTF |
| 85 | LCDR2 | AAS |
| 86 | LCDR3 | QQSSNTPFT |
| 87 | Anti- | EVQLLESGGV LVQPGGSLRL SCAASGFTFS NFGMTWVRQA PGKGLEWVSG ISGGGRDTYF ADSVKGRFTI SRDNSKNTLY |
| | PD1 | LQMNSLKGED TAVYYCVKWG NIYFDYWGQG TLVTVSS |
| | Mab VH | |
| 88 | Anti- | DIQMTQSPSS LSASVGDSIT ITCRASLSIN TFLNWYQQKP GKAPNLLIYA ASSLHGGVPS RFSGSGSGTD FTLTIRTLQP |
| | PD1 | EDFATYYCQQ SSNTPFTFGP GTVVDFR |
| | Mab VL | |

### DETAILED DESCRIPTION

### I. Definitions

The term “ModB” describes RNA comprising a modified nucleobase in place of at least one (e.g., every) uridine and further comprising a Cap1 structure at the 5′ end of the RNA. In some embodiments, the 5′ UTR of a ModB RNA comprises SEQ ID NOs: 4 or 6. ModB RNA has been processed to reduce double-stranded RNA (dsRNA). The “Cap1” structure may be generated after in-vitro translation by enzymatic capping or during in-vitro translation (co-transcriptional capping).

In some embodiments, the building block cap for ModB modified RNA is as follows, which is used when co-transcriptionally capping:

m₂^{7,3-O}Gppp(m₁^{2′-O})ApG (also sometimes referred to as m₂^{7,3′O}G(5′)ppp(5′)m^{2′-O}ApG), which has the following structure:

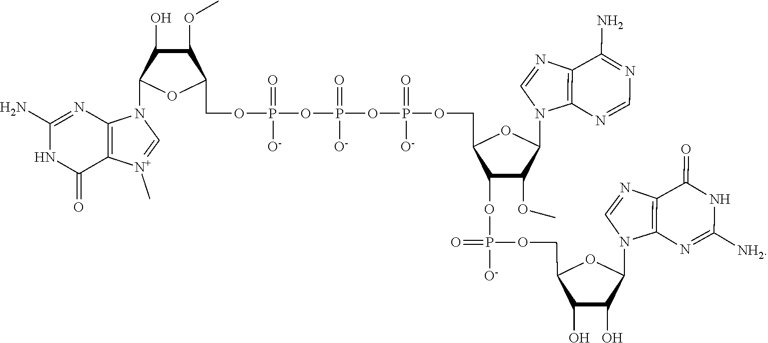

Below is an exemplary Cap1 RNA after co-transcriptional capping, which comprises RNA and m₂^{7,3′G}(5′)ppp(5′)m^{2′-O}ApG:

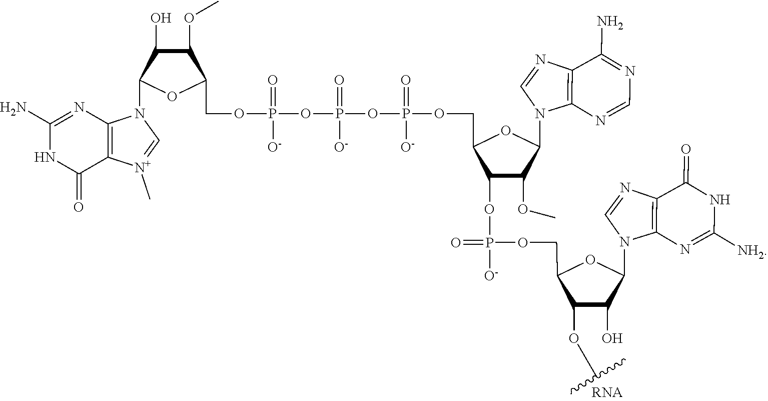

Below is another exemplary Cap1 RNA after enzymatic capping (no cap analog):

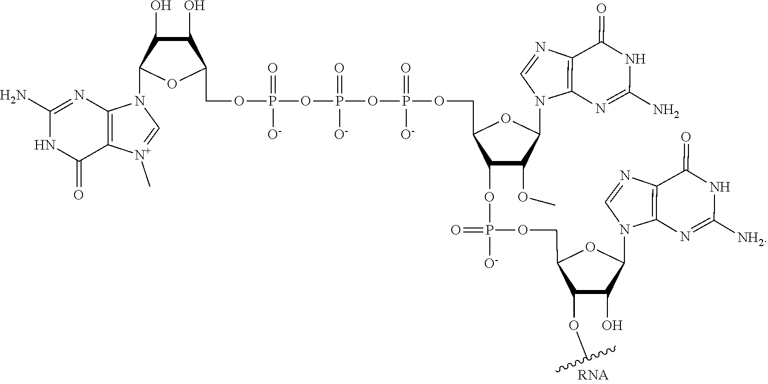

The term “ModA” describes RNA without dsRNA reduction that does not comprise a modified nucleobase in place of at least one uridine. ModA RNA comprises a Cap0 structure at the 5′ end of the RNA. The 5′ UTR of a ModA RNA may comprise SEQ ID NO: 2. “Cap0” structures are generated during in-vitro translation (co-transcriptional capping) using, in one embodiment, the cap analog anti-reverse cap (ARCA Cap (m₂^{7,3′O}G(5′)ppp(5′)G)) with the structure:

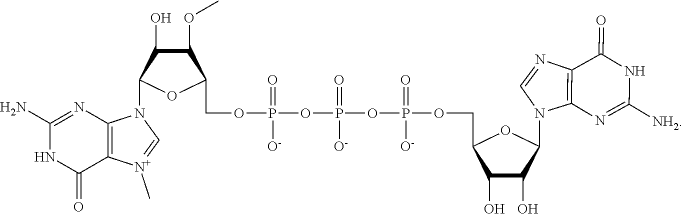

Below is an exemplary Cap0 RNA comprising RNA and m₂^{7,3′O}G(5′)ppp(5′)G:

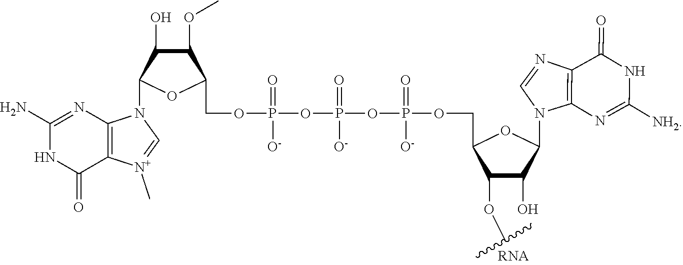

In some embodiments, the “Cap0” structures are generated during in-vitro translation (co-transcriptional capping) using the cap analog Beta-S-ARCA (m₂^{7,2′O}G(5′)ppSp(5′)G) with the structure:

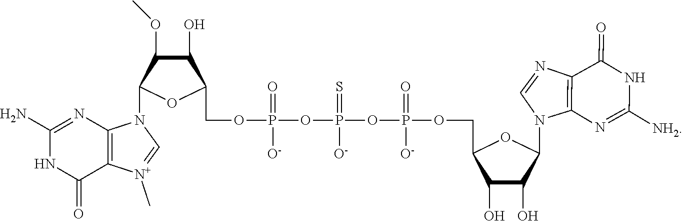

Below is an exemplary Cap0 RNA comprising Beta-S-ARCA (m₂^{7,2′O}G(5′)ppSp(5′)G) and RNA.

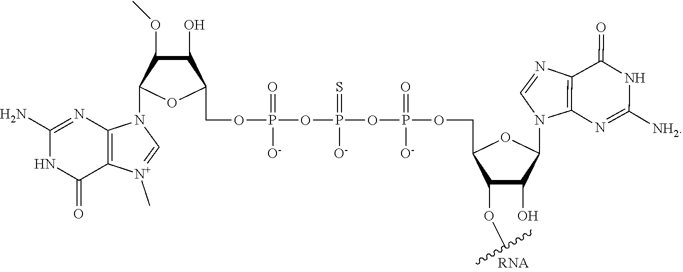

The term “uracil,” as used herein, describes one of the nucleobases that can occur in the nucleic acid of RNA. The structure of uracil is:

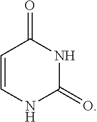

The term “uridine,” as used herein, describes one of the nucleosides that can occur in RNA. The structure of uridine is:

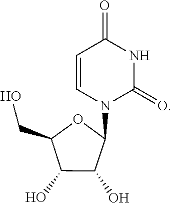 [SMILES: O=c1ccn([C@@H]2O[C@H](CO)[C@@H](O)[C@H]2O)c(=O)[nH]1]

UTP (uridine 5′-triphosphate) has the following structure:

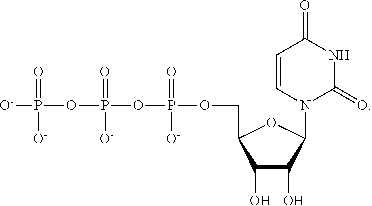

Pseudo-UTP (pseudouridine 5′-triphosphate) has the following structure:

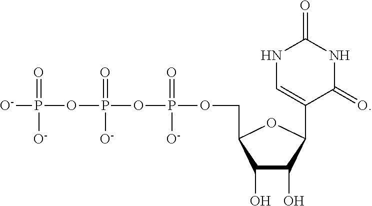

“Pseudouridine” is one example of a modified nucleoside that is an isomer of uridine, where the uracil is attached to the pentose ring via a carbon-carbon bond instead of a nitrogen-carbon glycosidic bond. Pseudouridine is described, for example, in Charette and Gray, Life; 49:341-351 (2000).

Another exemplary modified nucleoside is N1-methylpseudouridine (m1Ψ), which has the structure:

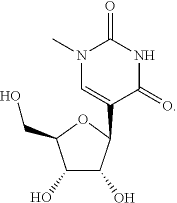 [SMILES: Cn1cc([C@@H]2O[C@H](CO)[C@@H](O)[C@H]2O)c(=O)[nH]c1=O]

N1-Methylpseudo-UTP has the following structure:

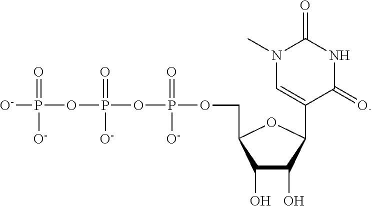

As used herein, the term “poly-A tail” or “poly-A sequence” refers to an uninterrupted or interrupted sequence of adenylate residues which is typically located at the 3′ end of an RNA molecule. Poly-A tails or poly-A sequences are known to those of skill in the art, and may follow the 3′ UTR in the RNAs described herein. An uninterrupted poly-A tail is characterized by consecutive adenylate residues. In nature, an uninterrupted poly-A tail is typical. RNAs disclosed herein can have a poly-A tail attached to the free 3′ end of the RNA by a template-independent RNA polymerase after transcription or a poly-A tail encoded by DNA and transcribed by a template-dependent RNA polymerase.

It has been demonstrated that a poly-A tail of about 120 A nucleotides has a beneficial influence on the levels of RNA in transfected eukaryotic cells, as well as on the levels of protein that is translated from an open reading frame that is present upstream (5′) of the poly-A tail (Holtkamp et al., 2006, Blood, vol. 108, pp. 4009-4017).

The poly-A tail may be of any length. In one embodiment, a poly-A tail comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context “essentially consists of” means that most nucleotides in the poly-A tail, typically at least 75%, at least 80%, at least 85%, at least 90%, at least 95%, at least 96%, at least 97%, at least 98%, or at least 99% by number of nucleotides in the poly-A tail are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly-A tail, i.e., 100% by number of nucleotides in the poly-A tail, are A nucleotides. The term “A nucleotide” or “A” refers to adenylate.

In some embodiments, a poly-A tail is attached during RNA transcription, e.g., during preparation of in vitro transcribed RNA, based on a DNA template comprising repeated dT nucleotides (deoxythymidylate) in the strand complementary to the coding strand. The DNA sequence encoding a poly-A tail (coding strand) is referred to as poly(A) cassette.

In one embodiment of the present invention, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present invention. A poly(A) cassette that essentially consists of dA nucleotides, but is interrupted by a random sequence having an equal distribution of the four nucleotides (dA, dC, dG, dT) and having a length of e.g. 5 to 50 nucleotides shows, on DNA level, constant propagation of plasmid DNA in E. coli and is still associated, on RNA level, with the beneficial properties with respect to supporting RNA stability and translational efficiency. Consequently, in one embodiment of the present invention, the poly-A tail contained in an RNA molecule described herein essentially consists of A nucleotides, but is interrupted by a random sequence of the four nucleotides (A, C, G, U). Such random sequence may be 5 to 50, 10 to 30, or 10 to 20 nucleotides in length.

In one embodiment of the invention, no nucleotides other than A nucleotides flank a poly-A tail at its 3′ end, i.e., the poly-A tail is not masked or followed at its 3′ end by a nucleotide other than A.

In some embodiments, a poly-A tail comprises the sequence:

(SEQ ID NO: 78)
AAAAAAAAAAAAAAAAAAAAAAAAAAAAAAGCAUAUGACUAAAAAAAAAA
AAAAAAAAAAAAAAAAAAAAAAAAAAAAAAAAAAAAAAAAAAAAAAAAAA
AAAAAAAAAA,

which is also shown in Table 2 following the 3′ UTR sequence.

“RNA” and “mRNA” are used interchangeably herein.

“IFNα” is used generically herein to describe any interferon alpha Type I cytokine, including IFNα2b and IFNα4. In the experiments described in the Example section, human IFNα2b and mouse IFNα4 were utilized. Any IFNα may be incorporated into the compositions and used in the methods described herein.

The term “treatment,” as used herein, covers any administration or application of a therapeutic for disease in a subject, and includes inhibiting the disease, arresting its development, relieving one or more symptoms of the disease, curing the disease, or preventing reoccurrence of the disease. For example, treatment of a solid tumor may comprise alleviating symptoms of the solid tumor, decreasing the size of the solid tumor, eliminating the solid tumor, reducing further growth of the tumor, or reducing or eliminating recurrence of a solid tumor after treatment. Treatment may also be measured as a change in a biomarker of effectiveness or in an imaging or radiographic measure.

The term “prevention,” as used herein, means inhibiting or arresting development of cancer, including solid tumors, in a subject deemed to be cancer free.

“Metastasis” means the process by which cancer spreads from the place at which it first arose as a primary tumor to other locations in the body.

The term “intra-tumorally,” as used herein, means into the tumor. For example, intra-tumoral injection means injecting the therapeutic at any location that touches the tumor.

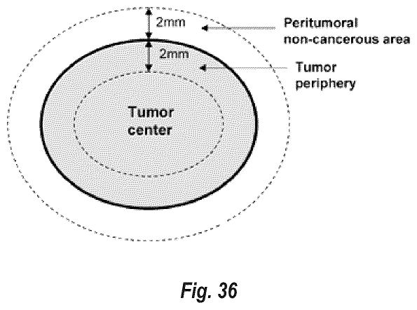

The term “peri-tumorally,” or “peri-tumoral,” as used herein, is an area that is about 2-mm wide and is adjacent to the invasive front of the tumor periphery. The peri-tumoral area comprises host tissue. See, for example, FIG. 36.

“Administering” means providing a pharmaceutical agent or composition to a subject, and includes, but is not limited to, administering by a medical professional and self-administering.

The disclosure describes nucleic acid sequences and amino acid sequences having a certain degree of identity to a given nucleic acid sequence or amino acid sequence, respectively (a reference sequence).

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. One important property includes the ability to act as a cytokine, in particular when administered to a subject. In some embodiments, a nucleic acid sequence or amino acid sequence having a particular degree of identity to a given nucleic acid sequence or amino acid sequence is functionally equivalent to said given sequence.

### II. Compositions and Medical Preparations

A. Interleukin-2 (IL-2)

In some embodiments, the composition comprises a DNA sequence encoding interleukin-2 (IL-2) (SEQ ID NO: 9). In some embodiments, the DNA sequence encoding IL-2 is provided in SEQ ID NO: 10.

In some embodiments, the composition comprises a codon-optimized DNA sequence encoding IL-2. In some embodiments, the codon-optimized DNA sequence comprises or consists of the nucleotides of SEQ ID NOs: 11. In some embodiments, the DNA sequence comprises a codon-optimized DNA sequence with 83%, 84%, 85%, 90%, 95%, 96%, 97%, 98%, or 99% identity to SEQ ID NO: 11.

The alignment of codon optimized IL-2 to native IL-2 is shown below, where the “Q” is native IL-2 (NM_000586.3; SEQ ID NO: 10) and the “S” is codon optimized IL-2 (SEQ ID NO: 11). The percent identity is 82.79%.

| | | | |
|---|---|---|---|
| Q: | 1 | ATGTACAGGATGCAACTCCTGTCTTGCATTGCACTAAGTCTTGCACTTGTCACAAACAGT | 60 |
| | | \|\|\|\|\|\|\|\| \|\|\|\|\| \|\| \|\|\|\|\|\|\|\|\|\|\|\|\|\| \|\| \|\|\|\|\|\| \|\|\|\|\| \|\|\|\|\| \| | |
| S: | 1 | ATGTACAGAATGCAGCTGCTGTCTTGCATTGCTCTTTCTCTTGCTCTTGTGACAAATTCT | 60 |
| Q: | 61 | GCACCTACTTCAAGTTCTACAAAGAAAACACAGCTACAACTGGAGCATTTACTGCTGGAT | 120 |
| | | \|\| \|\| \|\| \|\| \|\|\| \|\|\|\|\|\|\|\|\|\|\|\|\|\|\|\|\| \|\| \|\| \|\| \|\| \| \|\| \|\| \|\|\| | |
| S: | 61 | GCTCCAACATCTTCTTCAACAAAGAAAACACAGCTTCAGCTTGAACACCTTCTTCTTGAT | 120 |
| Q: | 121 | TTACAGATGATTTTGAATGGAATTAATAATTACAAGAATCCCAAACTCACCAGGATGCTC | 180 |
| | | \| \|\|\|\|\|\|\|\|\| \|\|\|\|\|\|\|\|\| \|\| \|\|\|\|\|\|\|\| \|\|\|\|\| \|\|\|\|\| \|\| \|\| \|\|\|\|\| | |
| S: | 121 | CTTCAGATGATTCTGAATGGAATCAACAATTACAAAAATCCAAAACTGACAAGAATGCTG | 180 |
| Q: | 181 | ACATTTAAGTTTTACATGCCCAAGAAGGCCACAGAACTGAAACATCTTCAGTGTCTAGAA | 240 |
| | | \|\|\|\|\|\|\|\| \|\|\|\|\|\|\|\|\|\|\| \|\|\|\|\| \|\| \|\|\|\|\|\|\|\|\|\|\|\|\|\| \|\|\|\|\|\|\|\| \|\| \|\|\| | |
| S: | 181 | ACATTTAAATTTTACATGCCAAAGAAAGCAACAGAACTGAAACACCTTCAGTGCCTTGAA | 240 |
| Q: | 241 | GAAGAACTCAAACCTCTGGAGGAAGTGCTAAATTTAGCTCAAAGCAAAAACTTTCACTTA | 300 |
| | | \|\|\|\|\|\|\|\| \|\|\|\|\|\|\|\|\|\|\| \|\|\|\|\|\|\|\| \|\|\| \| \|\|\|\|\| \|\|\|\|\|\|\|\| \|\|\|\|\|\| \| | |
| S: | 241 | GAAGAACTGAAACCTCTGGAAGAAGTGCTGAATCTGGCTCAGAGCAAAAATTTTCACCTG | 300 |
| Q: | 301 | AGACCCAGGGACTTAATCAGCAATATCAACGTAATAGTTCTGGAACTAAAGGGATCTGAA | 360 |
| | | \|\|\|\|\| \|\| \|\| \| \|\|\|\|\|\|\|\| \|\|\|\|\| \|\| \|\| \|\| \|\|\|\|\|\|\|\| \|\| \|\|\|\|\|\|\|\|\| | |
| S: | 301 | AGACCAAGAGATCTGATCAGCAACATCAATGTGATTGTGCTGGAACTGAAAGGATCTGAA | 360 |
| Q: | 361 | ACAACATTCATGTGTGAATATGCTGATGAGACAGCAACCATTGTAGAATTTCTGAACAGA | 420 |
| | | \|\|\|\|\|\|\|\|\|\|\|\|\|\|\|\|\|\|\|\|\|\|\|\|\|\|\|\|\| \|\|\|\|\|\|\|\| \|\|\|\|\| \|\|\|\|\|\|\|\|\|\|\|\|\|\|\| | |
| S: | 361 | ACAACATTCATGTGTGAATATGCTGATGAAACAGCAACAATTGTGGAATTTCTGAACAGA | 420 |
| Q: | 421 | TGGATTACCTTTTGTCAAAGCATCATCTCAACACTGACT | 459 |
| | | \|\|\|\|\| \|\| \|\|\|\|\| \|\| \|\|\|\|\| \|\|\|\|\|\|\|\|\|\|\| | |
| S: | 421 | TGGATCACATTTTGCCAGTCAATCATTTCAACACTGACA | 459 |

In some embodiments, the composition comprises an RNA sequence transcribed from a DNA sequence encoding IL-2. In some embodiments, the RNA sequence is transcribed from a nucleotide sequence comprising SEQ ID NO: 10 or 11. In some embodiments, the RNA sequence comprises or consists of SEQ ID NOs: 12 or 13. In some embodiments, the RNA sequence comprises or consists of an RNA sequence with 70%, 75%, 80%, 85%, 90%, 95%, 96%, 97%, 98%, or 99% identity to SEQ ID NOs: 12 or 13.

In some embodiments, one or more uridine in the IL-2 RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U).

In some embodiments, the IL-2 RNA comprises an altered nucleotide at the 5′ end. In some embodiments, the IL-2 RNA comprises a 5′ cap. Any 5′ cap known in the art may be used. In some embodiments, the 5′ cap comprises a 5′ to 5′ triphosphate linkage. In some embodiments, the 5′ cap comprises a 5′ to 5′ triphosphate linkage including thiophosphate modification. In some embodiments, the 5′ cap comprises a 2′-O or 3′-O-ribose-methylated nucleotide. In some embodiments, the 5′ cap comprises a modified guanosine nucleotide or modified adenosine nucleotide. In some embodiments, the 5′ cap comprises 7-methylguanylate. In some embodiments, the 5′ cap is Cap0 or Cap1. Exemplary cap structures include m7G(5′)ppp(5′)G, m7,2′ O-mG(5′)ppsp(5′)G, m7G(5′)ppp(5′)2′O-mG, and m7,3′ O-mG(5′)ppp(5′)2′ O-mA.

In some embodiments, the IL-2 RNA comprises a 5′ untranslated region (UTR). In some embodiments, the 5′ UTR is upstream of the initiation codon. In some embodiments, the 5′ UTR regulates translation of the RNA. In some embodiments, the 5′ UTR is a stabilizing sequence. In some embodiments, the 5′ UTR increases the half-life of RNA. Any 5′ UTR known in the art may be used. In some embodiments, the 5′ UTR RNA sequence is transcribed from a nucleotide sequence comprising SEQ ID NOs: 1, 3, or 5. In some embodiments, the 5′ UTR RNA sequence comprises or consists of SEQ ID NOs: 2, 4, or 6. In some embodiments, the 5′ UTR RNA sequence is at least 70%, 75%, 80%, 85%, 90%, 95%, 96%, 97%, 98%, or 99% identical to SEQ ID NOs: 2, 4, or 6.

In some embodiments, the IL-2 RNA comprises a 3′ UTR. In some embodiments, the 3′ UTR follows the translation termination codon. In some embodiments, the 3′ UTR regulates polyadenylation, translation efficiency, localization, or stability of the RNA. In some embodiments, the 3′ UTR RNA sequence is transcribed from a nucleotide sequence comprising SEQ ID NO: 7. In some embodiments, the 3′ UTR RNA sequence comprises or consists of SEQ ID NO: 8. In some embodiments, the 3′ UTR RNA sequence is at least 70%, 75%, 80%, 85%, 90%, 95%, 96%, 97%, 98%, or 99% identical to SEQ ID NO: 8.

In some embodiments, the IL-2 composition comprises both a 5′ UTR and a 3′ UTR. In some embodiments, the composition comprises only a 5′ UTR. In some embodiments, the composition comprises only a 3′ UTR.

In some embodiments, the IL-2 RNA comprises a poly-A tail. In some embodiments, the RNA comprises a poly-A tail of at least about 25, at least about 30, at least about 50, at least about 70, or at least about 100 nucleotides. In some embodiments, the poly-A tail comprises 200 or more nucleotides. In some embodiments, the poly-A tail comprises or consists of SEQ ID NO: 78.

In some embodiments, the RNA comprises a 5′ cap, a 5′ UTR, a nucleic acid encoding IL-2, a 3′ UTR, and a poly-A tail, in that order.

In some embodiments, the composition comprises a DNA sequence comprising or consisting of a nucleic acid sequence that is at least 70%, 75%, 80%, 85%, 90%, 95%, 96%, 97%, 98%, 99%, or 100% identical to SEQ ID NOs: 10 or 11 and at least 70%, 75%, 80%, 85%, 90%, 95%, 96%, 97%, 98%, 99%, or 100% identical to SEQ ID NOs: 1, 3, or 5.

In some embodiments, the composition comprises an RNA sequence, that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NOs: 10 or 11 and at least 70%, 75%, 80%, 85%, 90%, 95%, 96%, 97%, 98%, 99%, or 100% identical to SEQ ID NOs: 1, 3, or 5. The RNA may also be recombinantly produced. In some embodiments, one or more uridine in the IL-2 RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the composition comprises a DNA sequence comprising or consisting of a nucleic acid sequence at least 70%, 75%, 80%, 85%, 90%, 95%, 96%, 97%, 98%, 99%, or 100% identical to SEQ ID NOs: 10 or 11 and at least 70%, 75%, 80%, 85%, 90%, 95%, 96%, 97%, 98%, 99%, or 100% identical to SEQ ID NO: 7.

In some embodiments, the composition comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NOs: 10 or 11 and at least 70%, 75%, 80%, 85%, 90%, 95%, 96%, 97%, 98%, 99%, or 100% identical to SEQ ID NO: 7. In some embodiments, one or more uridine in the IL-2 RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the composition comprises a DNA sequence comprising or consisting of a nucleic acid sequence at least 70%, 75%, 80%, 85%, 90%, 95%, 96%, 97%, 98%, 99%, or 100% identical to SEQ ID NOs: 10 or 11; at least 70%, 75%, 80%, 85%, 90%, 95%, 96%, 97%, 98%, 99%, or 100% identical to SEQ ID NOs: 1, 3, or 5; and at least 70%, 75%, 80%, 85%, 90%, 95%, 96%, 97%, 98%, 99%, or 100% identical to SEQ ID NO: 7.

In some embodiments, the composition comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NOs: 10 or 11; at least 70%, 75%, 80%, 85%, 90%, 95%, 96%, 97%, 98%, 99%, or 100% identical to SEQ ID NOs: 1, 3, or 5; and at least 70%, 75%, 80%, 85%, 90%, 95%, 96%, 97%, 98%, 99%, or 100% identical to SEQ ID NO: 7. The RNA may also be recombinantly produced.

In some embodiments, one or more uridine in the IL-2 RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the composition comprises an RNA sequence comprising or consisting of a nucleic acid sequence at least 70%, 75%, 80%, 85%, 90%, 95%, 96%, 97%, 98%, 99%, or 100% identical to SEQ ID NOs: 12 or 13; at least 70%, 75%, 80%, 85%, 90%, 95%, 96%, 97%, 98%, 99%, or 100% identical to SEQ ID NOs: 2, 4, or 6; and at least 70%, 75%, 80%, 85%, 90%, 95%, 96%, 97%, 98%, 99%, or 100% identical to SEQ ID NO: 8. In some embodiments, one or more uridine in the IL-2 RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

B. Interleukin-12 single-chain (IL-12sc)

In some embodiments, the composition comprises a DNA sequence encoding interleukin-12 single-chain (IL-12sc) (e.g., SEQ ID NO: 14), which comprises IL-12 p40 (sometimes referred to as IL-12B), a linker, such as a GS linker, and IL-12 p35 (sometimes referred to as IL-12A). In some embodiments, the IL-12p40, linker, and IL-12p35 are consecutive with no intervening nucleotides. An exemplary DNA sequence encoding IL-12sc is provided in SEQ ID NO: 15.

The alignment of codon optimized IL-12 p40 to native IL-12 p40 is shown below, where the “S” is native IL-12 p40 (NM_002187.2; nucleotides 1-984 of SEQ ID NO: 15) and the “Q” is codon optimized IL-12 p40 (nucleotides 1-984 of SEQ ID NO: 16). The percent identity is 77%.

| | | | |
|---|---|---|---|
| Q: | 1 | ATGTGTCACCAGCAGCTGGTGATCTCATGGTTCTCCCTGGTATTTCTGGCATCTCCTCTT | 60 |
| | | \|\|\|\|\|\|\|\|\|\|\|\|\|\|\| \|\|\|\| \|\|\|\|\| \|\|\|\|\| \|\|\|\|\|\|\|\| \|\|\|\|\|\|\|\|\|\|\|\|\|\| \|\| | |
| S: | 1 | ATGTGTCACCAGCAGTTGGTCATCTCTTGGTTTTCCCTGGTTTTTCTGGCATCTCCCCTC | 60 |
| Q: | 61 | GTCGCAATCTGGGAACTGAAGAAAGACGTGTATGTCGTTGAGCTCGACTGGTATCCGGAT | 120 |
| | | \|\| \|\| \|\| \|\|\|\|\|\|\|\|\|\|\|\|\|\|\|\|\| \|\| \|\|\|\|\|\|\|\| \|\| \| \|\| \|\|\|\|\|\|\|\|\|\|\|\| | |
| S: | 61 | GTGGCCATATGGGAACTGAAGAAAGATGTTTATGTCGTAGAATTGGATTGGTATCCGGAT | 120 |
| Q: | 121 | GCGCCTGGCGAGATGGTGGTGCTGACCTGTGACACCCCAGAGGAGGATGGGATCACTTGG | 180 |
| | | \|\| \|\|\|\|\| \|\| \|\|\|\|\|\|\|\| \|\| \|\|\|\|\|\|\|\|\|\|\|\|\|\| \|\| \|\| \|\|\|\|\| \|\|\|\|\| \|\|\| | |
| S: | 121 | GCCCCTGGAGAAATGGTGGTCCTCACCTGTGACACCCCTGAAGAAGATGGTATCACCTGG | 180 |
| Q: | 181 | ACCCTTGATCAATCCTCCGAAGTGCTCGGGTCTGGCAAGACTCTGACCATACAAGTGAAA | 240 |
| | | \|\|\| \| \|\| \|\| \| \|\| \|\| \| \|\| \|\|\|\|\|\|\|\| \|\| \|\|\|\|\|\|\|\| \|\|\|\|\| \|\|\| | |
| S: | 181 | ACCTTGGACCAGAGCAGTGAGGTCTTAGGCTCTGGCAAAACCCTGACCATCCAAGTCAAA | 240 |
| Q: | 241 | GAGTTTGGCGATGCCGGGCAGTACACTTGCCATAAGGGCGGAGAAGTTCTGTCCCACTCA | 300 |
| | | \|\|\|\|\|\|\|\| \|\|\|\|\| \|\| \|\|\|\|\|\|\|\| \|\| \|\| \|\| \|\| \|\| \|\| \|\|\|\|\| \|\|\| \|\| | |
| S: | 241 | GAGTTTGGAGATGCTGGCCAGTACACCTGTCACAAAGGAGGCGAGGTTCTAAGCCATTCG | 300 |
| Q: | 301 | CTGCTGCTGCTGCACAAGAAAGAGGACGGAATTTGGAGTACCGATATCCTGAAAGATCAG | 360 |
| | | \|\| \|\|\|\|\|\|\|\| \|\|\|\|\| \|\| \|\| \|\| \|\|\|\|\|\|\|\|\| \|\| \|\|\|\|\| \| \|\| \|\| \|\|\| | |
| S: | 301 | CTCCTGCTGCTTCACAAAAAGGAAGATGGAATTTGGTCCACTGATATTTTAAAGGACCAG | 360 |
| Q: | 361 | AAAGAGCCCAAGAACAAAACCTTCTTGCGGTGCGAAGCCAAGAACTACTCAGGGAGATTT | 420 |
| | | \|\|\|\|\| \|\|\|\|\| \|\| \|\| \|\|\|\|\| \| \| \|\|\|\|\| \|\|\|\|\|\|\|\| \|\| \|\| \|\| \| \|\| | |
| S: | 361 | AAAGAACCCAAAAATAAGACCTTTCTAAGATGCGAGGCCAAGAATTATTCTGGACGTTTC | 420 |
| Q: | 421 | ACTTGTTGGTGGCTGACGACGATCAGCACCGATCTGACTTTCTCCGTGAAATCAAGTAGG | 480 |
| | | \|\| \|\| \|\|\|\|\|\|\|\|\|\|\|\|\|\| \|\|\|\|\| \|\| \|\|\| \|\|\|\| \|\|\| \|\| \|\|\| \|\| \|\| | |
| S: | 421 | ACCTGCTGGTGGCTGACGACAATCAGTACTGATTTGACATTCAGTGTCAAAAGCAGCAGA | 480 |
| Q: | 481 | GGATCATCTGACCCTCAAGGAGTCACATGTGGAGCGGCTACTCTGAGCGCTGAACGCGTA | 540 |
| | | \|\| \|\| \|\|\|\|\|\|\|\| \|\|\|\|\| \|\| \|\| \|\| \|\|\|\|\| \|\|\|\|\| \|\| \|\| \|\| \| \|\| | |
| S: | 481 | GGCTCTTCTGACCCCCAAGGGGTGACGTGCGGAGCTGCTACACTCTCTGCAGAGAGAGTC | 540 |
| Q: | 541 | AGAGGGGACAATAAGGAGTACGAGTATAGCGTTGAGTGCCAAGAGGATAGCGCATGCCCC | 600 |
| | | \|\|\|\|\|\|\|\|\|\|\| \|\|\|\|\|\|\|\| \|\|\|\|\| \|\| \|\|\|\|\|\|\|\| \|\|\|\|\| \|\| \|\| \|\|\|\|\| | |
| S: | 541 | AGAGGGGACAACAAGGAGTATGAGTACTCAGTGGAGTGCCAGGAGGACAGTGCCTGCCCA | 600 |
| Q: | 601 | GCCGCCGA--AGAATCATTGCCCATTGAAGTGATGGTGGATGCTGTACACAAGCTGAAGT | 658 |
| | | \|\| \|\| \|\| \|\|\| \|\| \| \|\|\|\|\|\|\|\|\| \|\| \|\|\|\|\|\|\|\|\|\|\| \|\| \|\|\|\|\|\|\|\| \|\|\|\| | |
| S: | 601 | GCTGCTGAGGAGAGTC-T-GCCCATTGAGGTCATGGTGGATGCCGTTCACAAGCTCAAGT | 658 |
| Q: | 659 | ATGAGAACTACACAAGCTCCTTCTTCATCCGTGACATCATCAAACCAGATCCTCCTAAGA | 718 |
| | | \|\|\|\| \|\|\|\|\|\|\|\| \|\|\| \|\|\|\|\|\|\|\|\|\| \| \|\|\|\|\|\|\|\|\|\|\|\|\|\| \|\| \|\| \|\| \|\|\|\| | |
| S: | 659 | ATGAAAACTACACCAGCAGCTTCTTCATCAGGGACATCATCAAACCTGACCCACCCAAGA | 718 |
| Q: | 719 | ACCTCCAGCTTAAACCTCTGAAGAACTCTAGACAGGTGGAAGTGTCTTGGGAGTATCCCG | 778 |
| | | \|\| \| \|\|\|\|\| \|\| \|\| \| \|\|\|\|\| \|\|\| \| \|\|\|\|\|\|\|\| \|\| \|\|\|\|\|\|\|\| \|\| \| | |
| S: | 719 | ACTTGCAGCTGAAGCCATTAAAGAATTCTCGGCAGGTGGAGGTCAGCTGGGAGTACCCTG | 778 |
| Q: | 779 | ACACCTGGTCTACACCACATTCCTACTTCAGTCTCACATTCTGCGTTCAGGTACAGGGCA | 838 |
| | | \|\|\|\|\|\|\|\| \|\|\| \|\|\|\|\|\|\|\|\|\|\|\|\|\|\| \|\| \|\|\|\|\|\|\|\|\|\|\|\|\|\|\|\|\| \|\|\|\|\|\|\| | |
| S: | 779 | ACACCTGGAGTACTCCACATTCCTACTTCTCCCTGACATTCTGCGTTCAGGTCCAGGGCA | 838 |
| Q: | 839 | AGTCCAAAAGGGAGAAGAAGGATCGGGTCTTTACAGATAAAACAAGTGCCACCGTTATAT | 898 |
| | | \|\| \|\|\| \|\| \|\| \|\|\|\|\| \|\|\| \| \|\|\|\|\| \|\| \|\| \|\| \|\| \|\|\|\|\| \|\| \|\| \| | |
| S: | 839 | AGAGCAAGNGAGAAAAGAAAGATAGAGTCTTCACGGACAAGACCTCAGCCACGGTCATCT | 898 |
| Q: | 899 | GCCGGAAGAATGCCTCTATTTCTGTGCGTGCGCAGGACAGATACTATAGCAGCTCTTGGA | 958 |
| | | \|\|\|\| \|\| \|\|\|\|\|\| \|\|\| \|\|\|\|\| \|\| \|\|\|\|\|\| \| \|\|\|\|\|\|\|\|\| \|\|\|\|\|\|\| | |
| S: | 899 | GCCGCAAAAATGCCAGCATTAGCGTGCGGGCCCAGGACCGCTACTATAGCTCATCTTGGA | 958 |
| Q: | 959 | GTGAATGGGC--CAGTGTCCCATGTTCA | 984 |
| | | \| \|\|\|\|\|\|\|\| \| \|\|\| \|\|\| \|\| \|\| | |
| S: | 959 | GCGAATGGGCATCTGTG-CCC-TG--CA | 982 |

The alignment of codon optimized IL-12 p35 to native IL-12 p35 is shown below, where the “S” is native IL-12 p35 (NM_00882.3; nucleotides 1027-1623 of SEQ ID NO: 15) and the “Q” is codon optimized IL-12 p35 (nucleotides 1027-1623 of SEQ ID NO: 16). The percent identity is 80%.

| | | | |
|---|---|---|---|
| Q: | 1 | AGAAATCTCCCTGTGGCTACACCTGATCCAGGCATGTTTCCCTGTTTGCACCATAGCCAA | 60 |
| | | \|\|\|\|\| \|\|\|\|\| \|\|\|\|\| \|\| \|\| \|\| \|\|\|\|\| \|\|\|\|\| \|\| \|\| \| \|\|\|\|\| \|\|\|\| | |
| S: | 1 | AGAAACCTCCCCGTGGCCACTCCAGACCCAGGAATGTTCCCATGCCTTCACCACTCCCAA | 60 |
| Q: | 61 | AACCTCCTGAGAGCAGTCAGCAACATGCTCCAGAAAGCTAGACAAACACTGGAATTCTAC | 120 |
| | | \|\|\|\|\| \|\|\|\|\| \|\| \|\|\|\|\|\|\|\|\|\|\|\|\|\|\|\|\|\|\|\| \|\| \|\|\|\|\|\|\|\| \|\| \|\|\|\|\| \|\|\| | |
| S: | 61 | AACCTGCTGAGGGCCGTCAGCAACATGCTCCAGAAGGCCAGACAAACTCTAGAATTTTAC | 120 |
| Q: | 121 | CCATGCACCTCCGAGGAAATAGATCACGAGGATATCACTAAGGACAAAACAAGCACTGTC | 180 |
| | | \|\| \|\|\|\|\| \|\| \|\| \|\| \|\| \|\|\|\|\| \|\| \|\|\|\|\|\|\|\| \|\| \|\| \|\|\|\|\| \|\|\|\|\| \|\|\| | |
| S: | 121 | CCTTGCACTTCTGAAGAGATTGATCATGAAGATATCACAAAAGATAAAACCAGCACAGTG | 180 |
| Q: | 181 | GAAGCATGCCTTCCCTTGGAACTGACAAAGAACGAGAGTTGCCTTAATTCAAGAGAAACA | 240 |
| | | \|\| \|\| \|\| \| \|\| \|\|\|\|\|\| \| \|\| \|\|\|\|\| \|\|\|\|\|\|\|\|\|\|\| \|\|\|\|\| \|\|\|\|\| \|\| | |
| S: | 181 | GAGGCCTGTTTACCATTGGAATTAACCAAGAATGAGAGTTGCCTAAATTCCAGAGAGACC | 240 |
| Q: | 241 | TCTTTCATTACAAACGGTAGCTGCTTGGCAAGCAGAAAAACATCTTTTATGATGGCCCTT | 300 |
| | | \|\|\|\|\|\|\|\| \|\| \|\| \|\| \|\| \|\|\| \|\|\|\| \|\|\|\|\|\| \|\| \|\|\|\|\|\|\|\|\|\|\|\|\|\|\|\|\| | |
| S: | 241 | TCTTTCATAACTAATGGGAGTTGCCTGGCCTCCAGAAAGACCTCTTTTATGATGGCCCTG | 300 |
| Q: | 301 | TGTCTGAGCAGTATTTATGAGGATCTCAAAATGTACCAGGTGGAGTTTAAGACCATGAAT | 360 |
| | | \|\| \|\| \|\| \|\|\|\|\|\|\|\|\|\|\| \|\| \| \|\| \|\|\|\|\|\|\|\|\|\|\|\|\|\|\|\|\| \|\|\|\|\|\|\|\|\|\|\|\| | |
| S: | 301 | TGCCTTAGTAGTATTTATGAAGACTTGAAGATGTACCAGGTGGAGTTCAAGACCATGAAT | 360 |
| Q: | 361 | GCCAAGCTGCTGATGGACCCAAAGAGACAGATTTTCCTCGATCAGAATATGCTGGCTGTG | 420 |
| | | \|\| \|\|\|\|\| \|\|\|\|\|\|\|\| \|\| \|\|\|\|\| \|\|\|\|\| \|\| \|\| \|\|\|\|\| \|\| \|\|\|\|\|\|\|\| \|\| | |
| S: | 361 | GCAAAGCTTCTGATGGATCCTAAGAGGCAGATCTTTCTAGATCAAAACATGCTGGCAGTT | 420 |
| Q: | 421 | ATTGATGAACTGATGCAGGCCTTGAATTTCAACAGCGAAACCGTTCCCCAGAAAAGCAGT | 480 |
| | | \|\|\|\|\|\|\|\| \|\|\|\|\|\|\|\|\|\|\|\| \|\|\|\|\|\|\|\|\|\|\|\|\| \|\| \|\| \|\| \|\| \|\| \|\|\| \| | |
| S: | 421 | ATTGATGAGCTGATGCAGGCCCTGAATTTCAACAGTGAGACTGTGCCACAAAAATCCTCC | 480 |
| Q: | 481 | CTTGAAGAACCTGACTTTTATAAGACCAAGATCAAACTGTGTATTCTCCTGCATGCCTTT | 540 |
| | | \|\|\|\|\|\|\|\|\|\|\| \|\| \|\|\|\|\|\|\|\| \|\| \|\| \|\|\|\|\| \|\| \|\| \|\| \|\| \|\| \|\|\|\|\| \|\| | |
| S: | 481 | CTTGAAGAACCGGATTTTTATAAAACTAAAATCAAGCTCTGCATACTTCTTCATGCTTTC | 540 |
| Q: | 541 | AGAATCAGAGCAGTCACTATAGATAGAGTGATGTCCTACCTGAATGCTTCC | 591 |
| | | \|\|\|\|\| \| \|\|\|\|\| \|\|\|\|\| \|\|\|\|\|\|\|\|\|\|\|\| \|\|\| \|\|\|\|\|\|\|\|\|\|\|\| | |
| S: | 541 | AGAATTCGGGCAGTGACTATTGATAGAGTGATGAGCTATCTGAATGCTTCC | 591 |

In some embodiments, the composition comprises a codon-optimized DNA sequence encoding IL-12sc. In some embodiments, the composition comprises a codon-optimized DNA sequence encoding IL-12 p40. In some embodiments, the composition comprises a codon-optimized DNA sequence encoding IL-12 p35. In some embodiments, the codon-optimized DNA sequence comprises or consists of SEQ ID NO: 16. In some embodiments, the DNA sequence comprises a codon-optimized DNA sequence with 70%, 75%, 80%, 85%, 90%, 95%, 96%, 97%, 98%, or 99% identity to SEQ ID NO: 16. In some embodiments, the codon-optimized DNA sequence encoding IL-12 p40 comprises the nucleotides encoding the IL-12sc-p40 (nucleotides 1-984 of SEQ ID NO: 16). In some embodiments, the codon-optimized DNA sequence encoding IL-12 p35 comprises the nucleotides encoding the IL-12sc-p35 (nucleotides 1027-1623 of SEQ ID NO: 16). In some embodiments, the codon-optimized DNA sequence encoding IL-12sc comprises the nucleotides encoding the IL-12sc-p40 (nucleotides 1-984 of SEQ ID NO: 16) and -p35 (nucleotides 1027-1623 of SEQ ID NO: 16) portions of SEQ ID NO: 16 and further comprises nucleotides between the p40 and p35 portions (e.g., nucleotides 985-1026 of SEQ ID NO: 16) encoding a linker polypeptide connecting the p40 and p35 portions. Any linker known to those of skill in the art may be used. The p40 portion may be 5′ or 3′ to the p35 portion.

In some embodiments, the composition comprises an RNA sequence that is, for example, transcribed from a DNA sequence encoding IL-12sc. The RNA may also be recombinantly produced. In some embodiments, the RNA sequence is transcribed from a nucleotide sequence comprising SEQ ID NOs: 15 or 16. In some embodiments, the RNA sequence comprises or consists of SEQ ID NOs: 17 or 18. In some embodiments, the RNA sequence comprises or consists of an RNA sequence with 70%, 75%, 80%, 85%, 90%, 95%, 96%, 97%, 98%, or 99% identity to SEQ ID NOs: 17 or 18. In some embodiments, the RNA sequence comprises the nucleotides encoding the IL-12sc-p40 (nucleotides 1-984 of SEQ ID NOs: 17 or 18) and -p35 (nucleotides 1027-1623 of SEQ ID NOs: 17 or 18) portions of SEQ ID NOs: 17 or 18. In some embodiments, the codon-optimized RNA sequence encoding IL-12sc comprises the nucleotides encoding the IL-12sc-p40 (nucleotides 1-984 of SEQ ID NO: 18) and -p35 (nucleotides 1027-1623 of SEQ ID NO: 18) portions of SEQ ID NO: 18 and further comprises nucleotides between the p40 and p35 portions encoding a linker polypeptide connecting the p40 and p35 portions. Any linker known to those of skill in the art may be used.

In some embodiments, one or more uridine in the IL-12sc RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the IL-12sc RNA comprises an altered nucleotide at the 5′ end. In some embodiments, the RNA comprises a 5′ cap. Any 5′ cap known in the art may be used. In some embodiments, the 5′ cap comprises a 5′ to 5′ triphosphate linkage. In some embodiments, the 5′ cap comprises a 5′ to 5′ triphosphate linkage including thiophosphate modification. In some embodiments, the 5′ cap comprises a 2′-O or 3′-O-ribose-methylated nucleotide. In some embodiments, the 5′ cap comprises a modified guanosine nucleotide or modified adenosine nucleotide. In some embodiments, the 5′ cap comprises 7-methylguanylate. In some embodiments, the 5′ cap is Cap0 or Cap1. Exemplary cap structures include m7G(5′)ppp(5′)G, m7,2′ O-mG(5′)ppsp(5′)G, m7G(5′)ppp(5′)2′O-mG, and m7,3′ O-mG(5′)ppp(5′)2′ O-mA.

In some embodiments, the IL-12sc RNA comprises a 5′ untranslated region (UTR). In some embodiments, the 5′ UTR is upstream of the initiation codon. In some embodiments, the 5′ UTR regulates translation of the RNA. In some embodiments, the 5′ UTR is a stabilizing sequence. In some embodiments, the 5′ UTR increases the half-life of RNA. Any 5′ UTR known in the art may be used. In some embodiments, the 5′ UTR RNA sequence is transcribed from SEQ ID NOs: 1, 3, or 5. In some embodiments, the 5′ UTR RNA sequence comprises or consists of SEQ ID NOs: 2, 4, or 6. In some embodiments, the 5′ UTR RNA sequence is at least 70%, 75%, 80%, 85%, 90%, 95%, 96%, 97%, 98%, or 99% identical to SEQ ID NOs: 2, 4, or 6.

In some embodiments, the IL-12sc RNA comprises a 3′ UTR. In some embodiments, the 3′ UTR follows the translation termination codon. In some embodiments, the 3′ UTR regulates polyadenylation, translation efficiency, localization, or stability of the RNA. In some embodiments, the 3′ UTR RNA sequence is transcribed from SEQ ID NO: 7. In some embodiments, the 3′ UTR RNA sequence comprises or consists of SEQ ID NO: 8. In some embodiments, the 3′ UTR RNA sequence is at least 70%, 75%, 80%, 85%, 90%, 95%, 96%, 97%, 98%, or 99% identical to SEQ ID NO: 8.

In some embodiments, the IL-12sc composition comprises both a 5′ UTR and a 3′ UTR. In some embodiments, the IL-12sc composition comprises only a 5′ UTR. In some embodiments, the IL-12sc composition comprises only a 3′ UTR.

In some embodiments, the IL-12sc RNA comprises a poly-A tail. In some embodiments, the RNA comprises a poly-A tail of at least about 25, at least about 30, at least about 50 nucleotides, at least about 70 nucleotides, or at least about 100 nucleotides. In some embodiments, the poly-A tail comprises 200 or more nucleotides. In some embodiments, the poly-A tail comprises or consists of SEQ ID NO: 78.

In some embodiments, the RNA comprises a 5′ cap, a 5′ UTR, a nucleic acid encoding IL-12sc, a 3′ UTR, and a poly-A tail, in that order.

In some embodiments, the composition comprises a DNA sequence comprising or consisting of a nucleic acid sequence that is at least 70%, 75%, 80%, 85%, 90%, 95%, 96%, 97%, 98%, 99%, or 100% identical to SEQ ID NOs: 15 or 16 and at least 70%, 75%, 80%, 85%, 90%, 95%, 96%, 97%, 98%, 99%, or 100% identical to SEQ ID NOs: 1, 3, or 5.

In some embodiments, the composition comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NOs: 15 or 16 and at least 70%, 75%, 80%, 85%, 90%, 95%, 96%, 97%, 98%, 99%, or 100% identical to SEQ ID NOs: 1, 3, or 5. The RNA may also be recombinantly produced. In some embodiments, one or more uridine in the IL-12sc RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the composition comprises a DNA sequence comprising or consisting of a nucleic acid sequence at least 70%, 75%, 80%, 85%, 90%, 95%, 96%, 97%, 98%, 99%, or 100% identical to SEQ ID NOs: 15 or 16 and at least 70%, 75%, 80%, 85%, 90%, 95%, 96%, 97%, 98%, 99%, or 100% identical to SEQ ID NO: 7.

In some embodiments, the composition comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NOs: 15 or 16 and at least 70%, 75%, 80%, 85%, 90%, 95%, 96%, 97%, 98%, 99%, or 100% identical to SEQ ID NO: 7. The RNA may also be recombinantly produced. In some embodiments, one or more uridine in the IL-12sc RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the composition comprises a DNA sequence comprising or consisting of a nucleic acid sequence at least 70%, 75%, 80%, 85%, 90%, 95%, 96%, 97%, 98%, 99%, or 100% identical to SEQ ID NOs: 15 or 16; at least 70%, 75%, 80%, 85%, 90%, 95%, 96%, 97%, 98%, 99%, or 100% identical to SEQ ID NOs: 1, 3, or 5; and at least 70%, 75%, 80%, 85%, 90%, 95%, 96%, 97%, 98%, 99%, or 100% identical to SEQ ID NO: 7.

In some embodiments, the composition comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NOs: 15 or 16; at least 70%, 75%, 80%, 85%, 90%, 95%, 96%, 97%, 98%, 99%, or 100% identical to SEQ ID NOs: 1, 3, or 5; and at least 70%, 75%, 80%, 85%, 90%, 95%, 96%, 97%, 98%, 99%, or 100% identical to SEQ ID NO: 7. The RNA may also be recombinantly produced. In some embodiments, one or more uridine in the IL-12sc RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the composition comprises an RNA sequence comprising or consisting of a nucleic acid sequence at least 70%, 75%, 80%, 85%, 90%, 95%, 96%, 97%, 98%, 99%, or 100% identical to SEQ ID NOs: 17 or 18; at least 70%, 75%, 80%, 85%, 90%, 95%, 96%, 97%, 98%, 99%, or 100% identical to SEQ ID NOs: 2, 4, or 6; and at least 70%, 75%, 80%, 85%, 90%, 95%, 96%, 97%, 98%, 99%, or 100% identical to SEQ ID NO: 8. In some embodiments, one or more uridine in the IL-12sc RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U).

C. Interferon Alpha (IFNα)

In some embodiments, the composition comprises a DNA sequence encoding interferon alpha (IFNα) (e.g., SEQ ID NO: 19). An exemplary DNA sequence encoding this IFNα is provided in SEQ ID NO: 20.

The alignment of codon optimized IFNα to native IFNα is shown below, where the “S” is native IFNα (NM_000605.3; SEQ ID NO: 20) and the “Q” is codon optimized IFNα (SEQ ID NO: 21). The percent identity is 79%.

| | | | |
|---|---|---|---|
| Q: | 1 | ATGGCCCTGACTTTTGCCCTTCTCGTGGCTTTGTTGGTGCTGAGTTGCAAATCTTCCTGT | 60 |
| | | \|\|\|\|\|\| \|\|\|\| \|\|\|\|\| \| \|\| \|\|\|\|\| \| \|\|\|\|\|\|\| \|\| \|\|\|\|\| \|\| \|\|\| | |
| S: | 1 | ATGGCCTTGACCTTTGCTTTACTGGTGGCCCTCCTGGTGCTCAGCTGCAAGTCAAGCTGC | 60 |
| Q: | 61 | AGTGTCGGATGTGATCTGCCTCAAACCCACAGTCTGGG-ATCTAGGAGAACACTGATGCT | 119 |
| | | \|\|\| \|\| \|\|\|\|\|\|\|\|\|\|\|\|\|\|\|\|\|\|\|\|\|\|\| \|\|\|\|\| \| \| \|\|\|\|\| \|\| \|\|\|\|\|\|\| | |
| S: | 61 | TCTGTGGGCTGTGATCTGCCTCAAACCCACAGCCTGGGTAGC-AGGAGGACCTTGATGCT | 119 |
| Q: | 120 | CTTGGCACAGATGAGGAGAAT-TAGC-CTCTTTTCCTGCCTGAAGGATAGACATGACTTC | 177 |
| | | \|\|\|\|\|\|\|\|\|\|\|\|\|\|\|\|\|\|\| \| \| \|\| \|\| \|\|\|\|\|\| \|\|\|\|\|\|\| \|\|\|\|\|\|\|\|\|\|\| | |
| S: | 120 | CCTGGCACAGATGAGGAGAATCT--CTCTTTTCTCCTGCTTGAAGGACAGACATGACTTT | 177 |
| Q: | 178 | GGCTTTCCCCAAGAGGAGTTTGGCAATCAGTTCCAGAAAGCGGAAACGATTCCCGTTCTG | 237 |
| | | \|\| \|\|\|\|\|\|\|\| \|\|\|\|\|\|\|\|\|\|\|\|\|\| \|\|\|\|\|\|\|\| \|\| \|\| \|\|\|\|\| \|\| \|\| \|\| \|\| | |
| S: | 178 | GGATTTCCCCAGGAGGAGTTTGGCAACCAGTTCCAAAAGGCTGAAACCATCCCTGTCCTC | 237 |
| Q: | 238 | CACGAGATGATCCAGCAGATCTTCAACCTCTTTTCAAC-CAAAG-ACAGCTCAGCAGCCT | 295 |
| | | \|\| \|\|\|\|\|\|\|\|\|\|\|\|\|\|\|\|\|\|\|\|\|\|\| \|\|\|\|\| \|\| \| \|\|\|\|\| \|\| \|\| \|\| \|\| \| | |
| S: | 238 | CATGAGATGATCCAGCAGATCTTCAATCTCTT--CAGCACAAAGGACTCATCTGCTGCTT | 295 |
| Q: | 296 | GGGATGAGACACTGCTGGACAAATTCTACACAGAACTGTATCAGCAGCTTAACGATCTGG | 355 |
| | | \|\|\|\|\|\|\|\|\|\| \|\| \|\| \|\|\|\|\|\|\|\|\|\|\|\|\|\| \|\|\|\|\| \|\| \|\|\|\|\|\|\|\| \|\| \|\| \|\|\|\| | |
| S: | 296 | GGGATGAGACCCTCCTAGACAAATTCTACACTGAACTCTACCAGCAGCTGAATGACCTGG | 355 |
| Q: | 356 | AGGCATGCGTGATCCAAGGGGTTGGTGTGACTGAAACTCCGCTTATGAAGGAGGACTCCA | 415 |
| | | \| \|\| \|\| \|\|\|\|\| \|\| \|\|\|\|\| \|\| \|\|\|\|\| \|\| \|\|\|\|\| \|\| \|\|\|\|\|\|\|\|\|\|\|\|\|\|\|\|\|\|\| | |
| S: | 356 | AAGCCTGTGTGATACAGGGGGTGGGGGTGACAGAGACTCCCCTGATGAAGGAGGACTCCA | 415 |
| Q: | 416 | TTCTGGCTGTACGGAAGTACTTCCAGAGAATAACCCTCTATCTGAAGGAGAAGAAGTACT | 475 |
| | | \|\|\|\|\|\|\|\|\|\| \|\|\|\| \|\|\|\|\|\|\|\| \|\|\|\|\| \|\| \|\|\|\|\|\|\|\|\|\|\| \|\|\|\|\|\|\|\| \|\|\| | |
| S: | 416 | TTCTGGCTGTGAGGAAATACTTCCAAAGAATCACTCTCTATCTGAAAGAGAAGAAATACA | 475 |
| Q: | 476 | CACCATGTGCTTGGGAAGTCGTGAGAGCCGAAATCATGAGATCCTTCAGCCTTAG-CACC | 534 |
| | | \|\| \|\|\|\|\| \|\|\|\|\| \|\| \|\| \|\|\|\|\| \|\|\|\|\|\|\|\|\|\|\|\|\|\| \|\| \| \|\| \| \|\| \| | |
| S: | 476 | GCCCTTGTGCCTGGGAGGTTGTCAGAGCAGAAATCATGAGATC-TTTTTC-TTTGTCAAC | 533 |
| Q: | 535 | AATC-TCCAGGAATCTCTGAGAAGCAAAGAG | 564 |
| | | \|\| \| \| \|\| \|\|\| \| \| \|\|\|\|\| \|\| \|\| | |
| S: | 534 | AAACTTGCAAGAAAGTTTAAGAAGTAAGGAA | 564 |

In some embodiments, the composition comprises a codon-optimized DNA sequence encoding IFNα. In some embodiments, the codon-optimized DNA sequence comprises or consists of the nucleotides of SEQ ID NO: 21. In some embodiments, the DNA sequence comprises or consists of a codon-optimized DNA sequence with 70%, 75%, 80%, 85%, 90%, 95%, 96%, 97%, 98%, or 99% identity to SEQ ID NO: 21.

In some embodiments, the composition comprises an RNA sequence that is, for example, transcribed from a DNA sequence encoding IFNα. The RNA may also be recombinantly produced. In some embodiments, the RNA sequence is transcribed from a nucleotide sequence comprising SEQ ID NOs: 20 or 21. In some embodiments, the RNA sequence comprises or consists of SEQ ID NOs: 22 or 23. In some embodiments, the RNA sequence comprises or consists of an RNA sequence with 70%, 75%, 80%, 85%, 90%, 95%, 96%, 97%, 98%, or 99% identity to SEQ ID NOs: 22 or 23.

In some embodiments, one or more uridine in the IFNα RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, each uridine in the RNA is modified. In some embodiments, each uridine in the RNA is modified with N1-methyl-pseudouridine (m¹ψ).

In some embodiments, the IFNα RNA comprises an altered nucleotide at the 5′ end. In some embodiments, the IFNα RNA comprises a 5′ cap. Any 5′ cap known in the art may be used. In some embodiments, the 5′ cap comprises a 5′ to 5′ triphosphate linkage. In some embodiments, the 5′ cap comprises a 5′ to 5′ triphosphate linkage including thiophosphate modification. In some embodiments, the 5′ cap comprises a 2′-O or 3′-O-ribose-methylated nucleotide. In some embodiments, the 5′ cap comprises a modified guanosine nucleotide or modified adenosine nucleotide. In some embodiments, the 5′ cap comprises 7-methylguanylate. In some embodiments, the 5′ cap is Cap0 or Cap1. Exemplary cap structures include m7G(5′)ppp(5′)G, m7,2′O-mG(5′)ppsp(5′)G, m7G(5′)ppp(5′)2′O-mG and m7,3′ O-mG(5′)ppp(5′)2′ O-mA.

In some embodiments, the IFNα RNA comprises a 5′ untranslated region (UTR). In some embodiments, the 5′ UTR is upstream of the initiation codon. In some embodiments, the 5′ UTR regulates translation of the RNA. In some embodiments, the 5′ UTR is a stabilizing sequence. In some embodiments, the 5′ UTR increases the half-life of RNA. Any 5′ UTR known in the art may be used. In some embodiments, the 5′ UTR RNA sequence is transcribed from a nucleotide sequence comprising SEQ ID NOs: 1, 3, or 5. In some embodiments, the 5′ UTR RNA sequence comprises or consists of SEQ ID NOs: 2, 4, or 6. In some embodiments, the 5′ UTR RNA sequence is at least 70%, 75%, 80%, 85%, 90%, 95%, 96%, 97%, 98%, or 99% identical to SEQ ID NOs: 2, 4, or 6.

In some embodiments, the IFNα RNA comprises a 3′ UTR. In some embodiments, the 3′ UTR follows the translation termination codon. In some embodiments, the 3′ UTR regulates polyadenylation, translation efficiency, localization, or stability of the RNA. In some embodiments, the 3′ UTR RNA sequence is transcribed from a nucleotide sequence comprising SEQ ID NO: 7. In some embodiments, the 3′ UTR RNA sequence comprises or consists of SEQ ID NO: 8. In some embodiments, the 3′ UTR RNA sequence is at least 70%, 75%, 80%, 85%, 90%, 95%, 96%, 97%, 98%, or 99% identical to SEQ ID NO: 8.

In some embodiments, the IFNα composition comprises both a 5′ UTR and a 3′ UTR. In some embodiments, the composition comprises only a 5′ UTR. In some embodiments, the composition comprises only a 3′ UTR.

In some embodiments, the IFNα RNA comprises a poly-A tail. In some embodiments, the IFNα RNA comprises a poly-A tail of at least about 25, at least about 30, at least about 50 nucleotides, at least about 70 nucleotides, or at least about 100 nucleotides. In some embodiments, the poly-A tail comprises 200 or more nucleotides. In some embodiments, the poly-A tail comprises or consists of SEQ ID NO: 78.

In some embodiments, the RNA comprises a 5′ cap, a 5′ UTR, a nucleic acid encoding IFNα, a 3′ UTR, and a poly-A tail, in that order.

In some embodiments, the composition comprises a DNA sequence comprising or consisting of a nucleic acid sequence that is at least 70%, 75%, 80%, 85%, 90%, 95%, 96%, 97%, 98%, 99%, or 100% identical to SEQ ID NOs: 20 or 21 and at least 70%, 75%, 80%, 85%, 90%, 95%, 96%, 97%, 98%, 99%, or 100% identical to SEQ ID NOs: 1, 3, or 5.

In some embodiments, the composition comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NOs: 20 or 21 and at least 70%, 75%, 80%, 85%, 90%, 95%, 96%, 97%, 98%, 99%, or 100% identical to SEQ ID NOs: 1, 3, or 5. The RNA may also be recombinantly produced. In some embodiments, one or more uridine in the IFNα RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the composition comprises a DNA sequence comprising or consisting of a nucleic acid sequence at least 70%, 75%, 80%, 85%, 90%, 95%, 96%, 97%, 98%, 99%, or 100% identical to SEQ ID NOs: 20 or 21 and at least 70%, 75%, 80%, 85%, 90%, 95%, 96%, 97%, 98%, 99%, or 100% identical to SEQ ID NO: 7.

In some embodiments, the composition comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NOs: 20 or 21 and at least 70%, 75%, 80%, 85%, 90%, 95%, 96%, 97%, 98%, 99%, or 100% identical to SEQ ID NO: 7. In some embodiments, one or more uridine in the IFNα RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the composition comprises a DNA sequence comprising or consisting of a nucleic acid sequence at least 70%, 75%, 80%, 85%, 90%, 95%, 96%, 97%, 98%, 99%, or 100% identical to SEQ ID NOs: 20 or 21; at least 70%, 75%, 80%, 85%, 90%, 95%, 96%, 97%, 98%, 99%, or 100% identical to SEQ ID NOs: 1, 3, or 5; and at least 70%, 75%, 80%, 85%, 90%, 95%, 96%, 97%, 98%, 99%, or 100% identical to SEQ ID NO: 7.

In some embodiments, the composition comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NOs: 20 or 21; at least 70%, 75%, 80%, 85%, 90%, 95%, 96%, 97%, 98%, 99%, or 100% identical to SEQ ID NOs: 1, 3, or 5; and at least 70%, 75%, 80%, 85%, 90%, 95%, 96%, 97%, 98%, 99%, or 100% identical to SEQ ID NO: 7. The RNA may also be recombinantly produced. In some embodiments, one or more uridine in the IFNα RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, the RNA comprises a modified nucleoside in place of each uridine. In some embodiments, the modified nucleoside is N1-methyl-pseudouridine (m1ψ). In some embodiments, the composition comprises an RNA sequence comprising or consisting of a nucleic acid sequence at least 70%, 75%, 80%, 85%, 90%, 95%, 96%, 97%, 98%, 99%, or 100% identical to SEQ ID NOs: 22 or 23; at least 70%, 75%, 80%, 85%, 90%, 95%, 96%, 97%, 98%, 99%, or 100% identical to SEQ ID NOs: 2, 4, or 6; and at least 70%, 75%, 80%, 85%, 90%, 95%, 96%, 97%, 98%, 99%, or 100% identical to SEQ ID NO: 8. In some embodiments, one or more uridine in the IFNα RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U).

D. IL-15 Sushi

As used herein, the term “IL-15 sushi” describes a construct comprising the soluble interleukin 15 (IL-15) receptor alpha sushi domain and mature interleukin alpha (IL-15) as a fusion protein. In some embodiments, the composition comprises a DNA sequence encoding IL-15 sushi (SEQ ID NO: 24), which comprises the soluble IL-15 receptor alpha chain (sushi) followed by a glycine-serine (GS) linker followed by the mature sequence of IL-15. The DNA sequence encoding this IL-15 sushi is provided in SEQ ID NO: 25.

In some embodiments, the composition comprises an RNA sequence that is, for example, transcribed from a DNA sequence encoding IL-15 sushi. The RNA may also be recombinantly produced. In some embodiments, the RNA sequence is transcribed from a nucleotide sequence comprising SEQ ID NO: 25. In some embodiments, the nucleotides encoding the linker may be completely absent or replaced in part or in whole with any nucleotides encoding a suitable linker. In some embodiments, the RNA sequence comprises or consists of SEQ ID NO: 26. In some embodiments, the RNA sequence comprises an RNA sequence with 70%, 75%, 80%, 85%, 90%, 95%, 96%, 97%, 98%, or 99% identity to SEQ ID NO: 26. In some embodiments, the DNA or RNA sequence encoding IL-15 sushi comprises the nucleotides encoding the sushi domain of IL-15 receptor alpha (e.g., nucleotide 1-321 of SEQ ID NOs: 25 or 26) and mature IL-15 (e.g., nucleotide 382-729 of SEQ ID NO: 25 or 26). In some embodiments, the DNA or RNA sequence encoding IL-15 sushi comprises the nucleotides encoding the sushi domain of IL-15 receptor alpha (e.g., nucleotide 1-321 of SEQ ID NOs: 25 or 26) and mature IL-15 (e.g., nucleotide 382-729 of SEQ ID NOs: 25 or 26) and further comprises nucleotides between these portions encoding a linker polypeptide connecting the portions. In some embodiments, the linker comprises nucleotides 322-381 of SEQ ID Nos: 25 or 26. Any linker known to those of skill in the art may be used.

In some embodiments, one or more uridine in the IL-15 sushi RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the IL-15 sushi RNA comprises an altered nucleotide at the 5′ end. In some embodiments, the IL-15 sushi RNA comprises a 5′ cap. Any 5′ cap known in the art may be used. In some embodiments, the 5′ cap comprises a 5′ to 5′ triphosphate linkage. In some embodiments, the 5′ cap comprises a 5′ to 5′ triphosphate linkage including thiophosphate modification. In some embodiments, the 5′ cap comprises a 2′-O or 3′-O-ribose-methylated nucleotide. In some embodiments, the 5′ cap comprises a modified guanosine nucleotide or modified adenosine nucleotide. In some embodiments, the 5′ cap comprises 7-methylguanylate. In some embodiments, the 5′ cap is Cap0 or Cap1. Exemplary cap structures include m7G(5′)ppp(5′)G, m7,2′ O-mG(5′)ppsp(5′)G, m7G(5′)ppp(5′)2′ O-mG and m7,3′ O-mG(5′)ppp(5′)2′ O-mA.

In some embodiments, the IL-15 sushi RNA comprises a 5′ untranslated region (UTR). In some embodiments, the 5′ UTR is upstream of the initiation codon. In some embodiments, the 5′ UTR regulates translation of the RNA. In some embodiments, the 5′ UTR is a stabilizing sequence. In some embodiments, the 5′ UTR increases the half-life of RNA. Any 5′ UTR known in the art may be used. In some embodiments, the 5′ UTR RNA sequence is transcribed from SEQ ID NOs: 1, 3, or 5. In some embodiments, the 5′ UTR RNA sequence comprises or consists of SEQ ID NOs: 2, 4, or 6. In some embodiments, the 5′ UTR RNA sequence is at least 70%, 75%, 80%, 85%, 90%, 95%, 96%, 97%, 98%, or 99% identical to SEQ ID NOs: 2, 4, or 6.

In some embodiments, the IL-15 sushi RNA comprises a 3′ UTR. In some embodiments, the 3′ UTR follows the translation termination codon. In some embodiments, the 3′ UTR regulates polyadenylation, translation efficiency, localization, or stability of the RNA. In some embodiments, the 3′ UTR RNA sequence is transcribed from SEQ ID NO: 7. In some embodiments, the 3′ UTR RNA sequence comprises or consists of SEQ ID NO: 8. In some embodiments, the 3′ UTR RNA sequence is at least 70%, 75%, 80%, 85%, 90%, 95%, 96%, 97%, 98%, or 99% identical to SEQ ID NO: 8.

In some embodiments, the IL-15 sushi composition comprises both a 5′ UTR and a 3′ UTR. In some embodiments, the IL-15 sushi composition comprises only a 5′ UTR. In some embodiments, the IL-15 sushi composition comprises only a 3′ UTR.

In some embodiments, the IL-15 sushi RNA comprises a poly-A tail. In some embodiments, the RNA comprises a poly-A tail of at least about 25, at least about 30, at least about 50 nucleotides, at least about 70 nucleotides, or at least about 100 nucleotides. In some embodiments, the poly-A tail comprises 200 or more nucleotides. In some embodiments, the poly-A tail comprises or consists of SEQ ID NO: 78.

In some embodiments, the RNA comprises a 5′ cap, a 5′ UTR, a nucleic acid encoding IL-15 sushi, a 3′ UTR, and a poly-A tail, in that order.

In some embodiments, the IL-15 sushi composition comprises a DNA sequence comprising or consisting of a nucleic acid sequence that is at least 70%, 75%, 80%, 85%, 90%, 95%, 96%, 97%, 98%, 99%, or 100% identical to SEQ ID NO: 25 and at least 70%, 75%, 80%, 85%, 90%, 95%, 96%, 97%, 98%, 99%, or 100% identical to SEQ ID NOs: 1, 3, or 5.

In some embodiments, the IL-15 sushi composition comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NO: 25 and at least 70%, 75%, 80%, 85%, 90%, 95%, 96%, 97%, 98%, 99%, or 100% identical to SEQ ID NOs: 1, 3, or 5. The RNA may also be recombinantly produced. In some embodiments, one or more uridine in the IFNα RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the IL-15 sushi composition comprises a DNA sequence comprising or consisting of a nucleic acid sequence at least 70%, 75%, 80%, 85%, 90%, 95%, 96%, 97%, 98%, 99%, or 100% identical to SEQ ID NO: 25 and at least 70%, 75%, 80%, 85%, 90%, 95%, 96%, 97%, 98%, 99%, or 100% identical to SEQ ID NO: 7.

In some embodiments, the IL-15 sushi composition comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NO: 25 and at least 70%, 75%, 80%, 85%, 90%, 95%, 96%, 97%, 98%, 99%, or 100% identical to SEQ ID NO: 7. The RNA may also be recombinantly produced. In some embodiments, one or more uridine in the IFNα RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the IL-15 sushi composition comprises a DNA sequence comprising or consisting of a nucleic acid sequence at least 70%, 75%, 80%, 85%, 90%, 95%, 96%, 97%, 98%, 99%, or 100% identical to SEQ ID NO: 25; at least 70%, 75%, 80%, 85%, 90%, 95%, 96%, 97%, 98%, 99%, or 100% identical to SEQ ID NOs: 1, 3, or 5; and at least 70%, 75%, 80%, 85%, 90%, 95%, 96%, 97%, 98%, 99%, or 100% identical to SEQ ID NO: 7.

In some embodiments, the IL-15 sushi composition comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NO: 25; at least 70%, 75%, 80%, 85%, 90%, 95%, 96%, 97%, 98%, 99%, or 100% identical to SEQ ID NOs: 1, 3, or 5; and at least 70%, 75%, 80%, 85%, 90%, 95%, 96%, 97%, 98%, 99%, or 100% identical to SEQ ID NO: 7. In some embodiments, one or more uridine in the IFNα RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the IL-15 sushi composition comprises an RNA sequence comprising or consisting of a nucleic acid sequence at least 70%, 75%, 80%, 85%, 90%, 95%, 96%, 97%, 98%, 99%, or 100% identical to SEQ ID NO: 26; at least 70%, 75%, 80%, 85%, 90%, 95%, 96%, 97%, 98%, 99%, or 100% identical to SEQ ID NOs: 2, 4, or 6; and at least 70%, 75%, 80%, 85%, 90%, 95%, 96%, 97%, 98%, 99%, or 100% identical to SEQ ID NO: 8. In some embodiments, one or more uridine in the IFNα RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U).

E. Granulocyte-Macrophage Colony-Stimulating Factor (GM-CSF)

In some embodiments, the composition comprises a DNA sequence encoding granulocyte-macrophage colony-stimulating factor (GM-CSF) (e.g., SEQ ID NO: 27). In some embodiments, the DNA sequence encoding GM-CSF is provided in SEQ ID NO: 28.

In some embodiments, the GM-CSF composition comprises an RNA sequence that is, for example, transcribed from a DNA sequence encoding GM-CSF. In some embodiments, the RNA sequence is transcribed from SEQ ID NO: 28. The RNA may also be recombinantly produced. In some embodiments, the RNA sequence comprises or consists of SEQ ID NO: 29. In some embodiments, the RNA sequence comprises an RNA sequence with 70%, 75%, 80%, 85%, 90%, 95%, 96%, 97%, 98%, or 99% identity to SEQ ID NOs: 29.

In some embodiments, one or more uridine in the GM-CSF RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, the RNA comprises a modified nucleoside in place of each uridine. In some embodiments, the modified nucleoside is N1-methyl-pseudouridine (m¹ψ). In some embodiments, the GM-CSF RNA comprises an altered nucleotide at the 5′ end. In some embodiments, the RNA comprises a 5′ cap. Any 5′ cap known in the art may be used. In some embodiments, the 5′ cap comprises a 5′ to 5′ triphosphate linkage. In some embodiments, the 5′ cap comprises a 5′ to 5′ triphosphate linkage including thiophosphate modification. In some embodiments, the 5′ cap comprises a 2′-O or 3′-O-ribose-methylated nucleotide. In some embodiments, the 5′ cap comprises a modified guanosine nucleotide or modified adenosine nucleotide. In some embodiments, the 5′ cap comprises 7-methylguanylate. In some embodiments, the 5′ cap is Cap0 or Cap1. Exemplary cap structures include m7G(5′)ppp(5′)G, m7,2′ O-mG(5′)ppsp(5′)G, m7G(5′)ppp(5′)2′O-mG and m7,3′ O-mG(5′)ppp(5′)2′ O-mA.

In some embodiments, the GM-CSF RNA comprises a 5′ untranslated region (UTR). In some embodiments, the 5′ UTR is upstream of the initiation codon. In some embodiments, the 5′ UTR regulates translation of the RNA. In some embodiments, the 5′ UTR is a stabilizing sequence. In some embodiments, the 5′ UTR increases the half-life of RNA. Any 5′ UTR known in the art may be used. In some embodiments, the 5′ UTR RNA sequence is transcribed from SEQ ID NOs: 1, 3, or 5. In some embodiments, the 5′ UTR RNA sequence comprises or consists of SEQ ID NOs: 2, 4, or 6. In some embodiments, the 5′ UTR RNA sequence is at least 70%, 75%, 80%, 85%, 90%, 95%, 96%, 97%, 98%, or 99% identical to SEQ ID NOs: 2, 4, or 6.

In some embodiments, the GM-CSF RNA comprises a 3′ UTR. In some embodiments, the 3′ UTR follows the translation termination codon. In some embodiments, the 3′ UTR regulates polyadenylation, translation efficiency, localization, or stability of the RNA. In some embodiments, the 3′ UTR RNA sequence is transcribed from SEQ ID NO: 7. In some embodiments, the 3′ UTR RNA sequence comprises or consists of SEQ ID NO: 8. In some embodiments, the 3′ UTR RNA sequence is at least 70%, 75%, 80%, 85%, 90%, 95%, 96%, 97%, 98%, or 99% identical to SEQ ID NO: 8.

In some embodiments, the GM-CSF composition comprises both a 5′ UTR and a 3′ UTR. In some embodiments, the composition comprises only a 5′ UTR. In some embodiments, the composition comprises only a 3′ UTR.

In some embodiments, the GM-CSF RNA comprises a poly-A tail. In some embodiments, the RNA comprises a poly-A tail of at least about 25, at least about 30, at least about 50 nucleotides, at least about 70 nucleotides, or at least about 100 nucleotides. In some embodiments, the poly-A tail comprises 200 or more nucleotides. In some embodiments, the poly-A tail comprises or consists of SEQ ID NO: 78.

In some embodiments, the GM-CSF RNA comprises a 5′ cap, a 5′ UTR, nucleotides encoding GM-CSF, a 3′ UTR, and a poly-A tail, in that order.

In some embodiments, the GM-CSF composition comprises a DNA sequence comprising or consisting of a nucleic acid sequence that is at least 70%, 75%, 80%, 85%, 90%, 95%, 96%, 97%, 98%, 99%, or 100% identical to SEQ ID NO: 28 and at least 70%, 75%, 80%, 85%, 90%, 95%, 96%, 97%, 98%, 99%, or 100% identical to SEQ ID NOs: 1, 3, or 5.

In some embodiments, the GM-CSF composition comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NO: 28 and at least 70%, 75%, 80%, 85%, 90%, 95%, 96%, 97%, 98%, 99%, or 100% identical to SEQ ID NOs: 1, 3, or 5. The RNA may also be recombinantly produced. In some embodiments, one or more uridine in the GM-CSF RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the GM-CSF composition comprises a DNA sequence comprising or consisting of a nucleic acid sequence at least 70%, 75%, 80%, 85%, 90%, 95%, 96%, 97%, 98%, 99%, or 100% identical to SEQ ID NO: 28 and at least 70%, 75%, 80%, 85%, 90%, 95%, 96%, 97%, 98%, 99%, or 100% identical to SEQ ID NO: 7.

In some embodiments, the GM-CSF composition comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NO: 28 and at least 70%, 75%, 80%, 85%, 90%, 95%, 96%, 97%, 98%, 99%, or 100% identical to SEQ ID NO: 7. The RNA may also be recombinantly produced. In some embodiments, one or more uridine in the GM-CSF RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the GM-CSF composition comprises a DNA sequence comprising or consisting of a nucleic acid sequence at least 70%, 75%, 80%, 85%, 90%, 95%, 96%, 97%, 98%, 99%, or 100% identical to SEQ ID NO: 28; at least 70%, 75%, 80%, 85%, 90%, 95%, 96%, 97%, 98%, 99%, or 100% identical to SEQ ID NOs: 1, 3, or 5; and at least 70%, 75%, 80%, 85%, 90%, 95%, 96%, 97%, 98%, 99%, or 100% identical to SEQ ID NO: 7.

In some embodiments, the composition comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NO: 28; at least 70%, 75%, 80%, 85%, 90%, 95%, 96%, 97%, 98%, 99%, or 100% identical to SEQ ID NOs: 1, 3, or 5; and at least 70%, 75%, 80%, 85%, 90%, 95%, 96%, 97%, 98%, 99%, or 100% identical to SEQ ID NO: 7. The RNA may also be recombinantly produced. In some embodiments, one or more uridine in the GM-CSF RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the GM-CSF composition comprises an RNA sequence comprising or consisting of a nucleic acid sequence at least 70%, 75%, 80%, 85%, 90%, 95%, 96%, 97%, 98%, 99%, or 100% identical to SEQ ID NO: 29; at least 70%, 75%, 80%, 85%, 90%, 95%, 96%, 97%, 98%, 99%, or 100% identical to SEQ ID NOs: 2, 4, or 6; and at least 70%, 75%, 80%, 85%, 90%, 95%, 96%, 97%, 98%, 99%, or 100% identical to SEQ ID NO: 8. In some embodiments, one or more uridine in the GM-CSF RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U).

F. Modifications

Each of the RNAs and compositions described herein may be modified in any way known to those of skill in the art. In some embodiments, the modifications are “ModA” or “ModB” modified as described herein.

In some embodiments, one or more uridine in the RNA is replaced by a modified nucleoside. In some embodiments, the modified nucleoside is a modified uridine.

In some embodiments, the modified uridine replacing uridine is pseudouridine (W), N1-methyl-pseudouridine (m¹ψ), or 5-methyl-uridine (m5U).

In some embodiments, one or more cytosine, adenine or guanine in the RNA is replaced by modified nucleobase(s). In one embodiment, the modified nucleobase replacing cytosine is 5-methylcytosine (m⁵C). In another embodiment, the modified nucleobase replacing adenine is N⁶-methyladenine (m⁶A). In another embodiment, any other modified nucleobase known in the art for reducing the immunogenicity of the molecule can be used.

The modified nucleoside replacing one or more uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmoSU), uridine 5-oxyacetic acid methyl ester (mcmoSU), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (win), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

G. Combination Compositions

In some embodiments, the invention comprises a composition comprising more than one RNA as described herein. In some embodiments, the composition comprises two RNAs. In some embodiments, the composition comprises three RNAs. In some embodiments, the composition comprises four RNAs. In some embodiments, the composition comprises five RNAs. In some embodiments, any or all of the RNAs encoding IL-2, IL12sc, IL-15 sushi, GM-CSF, or IFNα may be replaced by IL-2, IL12sc, IL-15 sushi, GM-CSF, and/or IFNα polypeptides, e.g., in any of the compositions and formulations comprising these RNAs described herein.

In some embodiments, the modified or unmodified RNAs encoding IL-2, IL12sc, IL-15 sushi, GM-CSF, and/or IFNα may be replaced by modified or unmodified polycistronic RNAs encoding two or more polypeptides selected from IL-2, IL12sc, IL15 sushi, GM-CSF and IFNα polypeptides, e.g., in any of the compositions and formulations comprising these RNAs described herein.

Any of the combination compositions may further comprise an excipient or diluent. The excipient or diluent may be pharmaceutically acceptable for administration to a subject.

In some embodiments, a combination composition comprises RNAs with the same modifications. In some embodiments, a combination composition comprises RNAs with different modifications. In some embodiments, a combination composition comprises RNAs with ModA modification. In some embodiments, a combination composition comprises RNAs with ModB modification. In some embodiments, a combination composition comprises RNAs with ModA and ModB modifications.

In some embodiments, a composition comprising DNA or RNA encoding IL-2 and one or more of a DNA or RNA encoding IL-12sc, IFNα, IL-15 sushi, and GM-CSF is encompassed. In some embodiments, the composition comprises a DNA or RNA encoding IL-2 or codon-optimized IL-2 (SEQ ID NOs: 10-13) and one or more of a DNA or RNA encoding IL-12sc or optimized IL-12sc (SEQ ID Nos: 15-18), IFNα or optimized IFNα (SEQ ID Nos: 20-23), IL-15 sushi (SEQ ID NOs: 25-26), and GM-CSF (SEQ ID NOs: 28-29), as described herein. In some embodiments, one or more uridine in the RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, one or more of the RNAs in the composition further comprises a 5′ cap, a 5′ UTR, a 3′ UTR, and a poly-A tail as described herein in the composition section.

In some embodiments, a composition comprising DNA or RNA encoding IL-12sc and one or more of a DNA or RNA encoding IL-2, IFNα, IL-15 sushi, and GM-CSF is encompassed. In some embodiments, the composition comprises a DNA or RNA encoding IL-12sc or codon-optimized IL-12sc (SEQ ID NOs: 15-18) and one or more of a DNA or RNA encoding IL-2 or optimized IL-2 (SEQ ID NOs: 10-13), IFNα or optimized IFNα (SEQ ID NOs: 20-23), IL-15 sushi (SEQ ID NOs: 25-26), and GM-CSF (SEQ ID NOs: 28-29), as described herein. In some embodiments, one or more uridine in the RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U).

In some embodiments, one or more of the RNAs in the composition further comprises a 5′ cap, a 5′ UTR, a 3′ UTR, and a poly-A tail, as described herein in the composition section.

In some embodiments, a composition comprising DNA or RNA encoding IFNα and one or more of a DNA or RNA encoding IL-2, IL-12sc, IL-15 sushi, and GM-CSF is encompassed. In some embodiments, the composition comprises a DNA or RNA encoding IFNα or codon-optimized IFNα (SEQ ID NOs: 20-23) and one or more of a DNA or RNA encoding IL-12sc or optimized IL-12sc (SEQ ID NOs: 15-18), IL-2 or optimized IL-2 (SEQ ID NOs: 10-13), IL-15 sushi (SEQ ID NOs: 25-26), and GM-CSF (SEQ ID NOs: 28-29), as described herein. In some embodiments, one or more uridine in the RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, one or more of the RNAs in the composition further comprises a 5′ cap, a 5′ UTR, a 3′ UTR, and a poly-A tail as described herein in the composition section.

In some embodiments, a composition comprising DNA or RNA encoding IL-15 sushi and one or more of a DNA or RNA encoding IL-2, IL-12sc, IFNα, and GM-CSF is encompassed. In some embodiments, the composition comprises a DNA or RNA encoding IL-15 sushi (SEQ ID NOs: 25-26) and one or more of a DNA or RNA encoding IL-12sc or optimized IL-12sc (SEQ ID NOs: 15-18), IFNα or optimized IFNα (SEQ ID NOs: 20-23), IL-2 or optimized IL-2 (SEQ ID NOs: 10-13), and GM-CSF (SEQ ID NOs: 28-29), as described herein. In some embodiments, one or more uridine in the RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, one or more of the RNAs in the composition further comprises a 5′ cap, a 5′ UTR, a 3′ UTR, and a poly-A tail as described herein in the composition section.

In some embodiments, a composition comprising DNA or RNA encoding GM-CSF and one or more of a DNA or RNA encoding IL-2, IL-12sc, IFNα, and IL-15 sushi is encompassed. In some embodiments, the composition comprises a DNA or RNA encoding GM-CSF (SEQ ID NOs: 28-29) and one or more of a DNA or RNA encoding IL-12sc or optimized IL-12sc (SEQ ID NOs: 15-18), IFNα or optimized IFNα (SEQ ID NOs: 20-23), IL-2 or optimized IL-2 (SEQ ID NOs: 10-13), and IL-15 sushi (SEQ ID NOs: 25-26), as described herein. In some embodiments, one or more uridine in the RNA is replaced by a modified nucleoside as described herein. In some embodiments, the modified nucleoside replacing uridine is pseudouridine (ψ), N1-methyl-pseudouridine (m1ψ) or 5-methyl-uridine (m⁵U). In some embodiments, one or more of the RNAs in the composition further comprises a 5′ cap, a 5′ UTR, a 3′ UTR, and a poly-A tail as described herein in the composition section.

In some embodiments, the composition comprises GM-CSF, IL-2, and IL-12sc RNA. In some embodiments, the composition is modified, for example, as ModA or ModB. In some embodiments, the IL-12sc RNA is optimized as shown in SEQ ID NO: 18.

In some embodiments, the composition comprises GM-CSF, IL-15 sushi, and IL-12sc RNA. In some embodiments, the composition is modified, for example, as ModA or ModB. In some embodiments, the IL-12sc RNA is optimized as shown in SEQ ID NO: 18.

In some embodiments, the composition comprises GM-CSF, IL-2, IL-12sc, and IFNα RNA. In some embodiments, the composition is modified, for example, as ModA or ModB. In some embodiments, the IL-12sc RNA and IFNα RNA is optimized as shown in SEQ ID NOs: 18 and 23, respectively.

In some embodiments, the composition comprises GM-CSF, IL-15 sushi, IL-12sc, and IFNα RNA. In some embodiments, the composition is modified, for example, as ModA or ModB. In some embodiments, the IL-12sc RNA and IFNα RNA is optimized as shown in SEQ ID NOs: 18 and 23, respectively.

In some embodiments, the composition comprises GM-CSF, IL-15 sushi, IL-12sc, and IFNα RNA, wherein the RNAs comprise or consist of the nucleotides shown in SEQ ID Nos: 18 (IL-12sc), 23 (IFNα), 26 (IL-15 sushi), or 29 (GM-CSF). In some embodiments, the composition is modified, for example, as ModA or ModB.

In some embodiments, combinations of RNA are administered as a 1:1, 1:1:1, or 1:1:1:1 ratio based on equal RNA mass. For example, 20 μg of IL15-sushi, 20 μg of IL-12sc, 20 μg of IFNα2b and 20 μg GM-CSF. In some embodiments, the ratio is adjusted so that different ratios by mass are administered, for example, 1:10:1:10 ratio (20 μg, 200 μg, 20 μg, 200 μg). Likewise, in some embodiments, for example, a ratio of 1:2:3:4 (20 μg, 40 μg, 60 μg, 80 μg) is used. Alternatively, rather than basing the ratio on the mass of the RNA, the ratio may be based on the molarity of the RNA.

In some embodiments, a mixture of RNAs is administered with an equal ratio of each RNA of the mixture.

In some embodiments, a mixture of RNAs is administered with an unequal ratio of each RNA of the mixture. In some embodiments, one or more RNAs are administered at a ratio that is 1, 2, 3, 4, 5, 6, 7, 8, 9, or 10 times greater than another RNA in the mixture. In some embodiments, one or more RNAs are administered at a ratio that is 1, 2, 3, 4, 5, 6, 7, 8, 9, or 10 times less than another RNA in the mixture.

In some embodiments, the compositions described herein may be a medical preparation. In some embodiments, the medical preparation comprises a kit, wherein the included RNAs may be in the same or separate vials. In some embodiments, the medical preparation further comprising instructions for use of the composition for treating or preventing a solid tumor.

In some embodiments, a kit comprising the compositions described herein is provided, wherein the included RNAs may be in the same or separate vials. In some embodiments, the kit further comprising instructions for use of the composition for treating or preventing a solid tumor.

H. Effect of IFNα Addition to Modified mRNA Treatment

RNA can activate the immune system through stimulating various pattern recognition receptors (PRR) leading to production of Type I interferons (like IFNα). The incorporation of various modified nucleotides, or other alterations like reducing the amount of dsRNA administered, can reduce the immune stimulatory effects of RNA. Unexpectedly, as described in the Examples, inclusion of nucleotide-modified and dsRNA-reduced mRNA encoding interferon alpha improved anti-tumor activity relative to that of mRNA that was not nucleotide modified and dsRNA-reduced. The addition of mRNA encoding interferon alpha restored a portion of the immune stimulatory effects removed by the inclusion of modified nucleotides and the dsRNA purification.

In some embodiments, RNA encoding IFN (in any form or subtype) is provided, wherein the IFN RNA is altered to have reduced immunogenicity compared to un-altered RNA. In certain embodiments, the administration of this IFN improves the anti-tumor response of non-IFN encoding RNA. In some embodiments, RNA encoding IFNα improves the anti-tumor response of other RNAs, so long as the other RNAs have been altered to reduce immunogenicity. In one embodiment, the alteration to reduce immunogenicity is a reduction in the amount of dsRNA. In some aspects, the alteration to reduce immunogenicity is the replacement of one or more uridines with a modified nucleoside. In some aspects, the alteration to reduce immunogenicity is both a reduction in the amount of dsRNA and the replacement of one or more uridines with modified nucleoside. In some embodiments, the IFN is IFNα.

In some embodiments, IFN RNA improves the anti-tumor response of modified RNAs. In some embodiments, IFN RNA improves the anti-tumor response of RNAs comprising modified nucleotides. In some embodiments, IFN RNA improves the anti-tumor response of mRNAs comprising pseudouridine. In some embodiments, IFN RNA improves the anti-tumor response of RNAs with ModB modifications.

In some embodiments, IFN RNA improves the anti-tumor response of RNAs of IL-2 (SEQ ID NO: 12 or 13), IL-12sc (SEQ ID NO: 17 or 18), IL-15 sushi (SEQ ID NO: 26) or GM-CSF (SEQ ID NO: 29). In some embodiments, the RNAs comprise ModB modifications.

In some embodiments, the IFN is IFNα.

In some embodiments, the IFN RNA construct is SEQ ID NO: 22 or 23.

### III. Methods and Uses

Any of the RNAs, compositions, medical preparations and combination compositions described herein may be administered to a subject to treat cancer or a solid tumor. In some embodiments, the cancer is a solid tumor. In some embodiments, the solid tumor is an abnormal mass of tissue that does not contain cysts or liquid areas. In some embodiments, the solid tumor may be benign or malignant. In some embodiments, the solid tumor is a pre-cancerous lesion. In some embodiments, the solid tumor occurs in lung, colon, ovary, cervix, uterus, peritoneum, testicles, penis, tongue, lymph node, pancreas, bone, breast, prostate, soft tissue, connective tissue, kidney, liver, brain, thyroid, or skin.

In some embodiments, the solid tumor is a sarcoma, carcinoma, or lymphoma. In some embodiments, the solid tumor is an epithelial tumor, Hodgkin lymphoma (HL), non-Hodgkin lymphoma, prostate tumor, ovarian tumor, renal cell tumor, gastrointestinal tract tumor, hepatic tumor, colorectal tumor, tumor with vasculature, mesothelioma tumor, pancreatic tumor, breast tumor, sarcoma tumor, lung tumor, colon tumor, brain tumor, melanoma tumor, basal cell carcinoma, squamous cell carcinoma, small cell lung tumor, neuroblastoma tumor, testicular tumor, carcinoma tumor, adenocarcinoma tumor, glioma tumor, seminoma tumor, retinoblastoma, or osteosarcoma tumor. In some embodiments, the solid tumor is a precancerous lesion such as actinic keratosis.

In some embodiments, the RNA compositions may be delivered via injection into (e.g., intra-tumorally) or near (peri-tumorally) the tumor. In some embodiments, the RNA compositions may be delivered at or near the site of a tumor removal.

In some embodiments, the RNA compositions may be delivered via a topical solution, ointment, or cream.

In some embodiments, the RNA compositions may be delivered via a virus. In some embodiments, the RNA compositions may be delivered by infection with a virus encoding the RNA compositions, such as an oncolytic virus. In some embodiments, the RNA compositions may be delivered by an oncolytic virus.

In some embodiments, more than one administration is delivered. In some embodiments, a catheter is placed into or near the site of the tumor for multiple administrations. In some embodiments, a catheter is placed at the site of removal of a tumor for multiple administrations.

In some embodiments, the subject is human. In some embodiments, the subject is a non-human mammal such as a dog, cat, mouse, rat, rabbit, sheep, cattle, horse and pig.

In some embodiments, RNA compositions are combined with another therapy. In some embodiments, RNA compositions are combined with more than one other therapy. In some embodiments, RNA compositions are combined in a multi-modal therapy.

In some embodiments, the other therapy is surgery to excise, resect, or debulk the tumor. In some embodiments, therapeutic RNA compositions are administered during a surgery to excise, resect, or debulk the tumor.

In some embodiments, the other therapy is radiotherapy. In some embodiments, the radiotherapy is external beam radiation therapy or particle beam radiation. In some embodiments, the radiotherapy is brachytherapy involving temporary or permanent implantation of radioactive isotopes directly into the tumor via catheter or large bore needle. In some embodiments, the radioactive isotope is 137Cesium, 192Iridium, or radioactive iodine. In some embodiments, the radiotherapy is radioisotope preparations administered intravenously. In some embodiments, the radioisotope preparations are radioactive iodine (131I), Strontium (89Sr), or Samarium (153Sm).

In some embodiments, the other therapy is chemotherapy. In some embodiments, the chemotherapy is an alkylating agent, an antimetabolite, an anti-microtubule agent, a topoisomerase inhibitor, or a cytotoxic antibody.

In some embodiments, the chemotherapy comprises anti-invasion agents (e.g., metalloproteinase inhibitors like marimastat and inhibitors of urokinase plasminogen activator receptor function). In some embodiments, the chemotherapy comprises inhibitors of growth factor function (e.g., platelet derived growth factor and hepatocyte growth factor), growth factor antibodies, or growth factor receptor antibodies, (e.g., anti-erbb2 antibody trastuzumab [Herceptin™] and the anti-erbb1 antibody Cetuximab™). In some embodiments, the chemotherapy is a farnesyl transferase inhibitor. In some embodiments, the chemotherapy is a tyrosine kinase inhibitor such as inhibitors of the epidermal growth factor family (e.g., EGFR family tyrosine kinase inhibitors such as gefitinib (Iressa™), erlotinib (Tarceva™), and Canertinib (CI 1033), or a serine/threonine kinase inhibitor).

In some embodiments, the chemotherapy comprises antiproliferative/antineoplastic drugs such as antimetabolites (e.g., antifolates like methotrexate, fluoropyrimidines like 5-fluorouracil, tegafur, purine and adenosine analogues, cytosine arabinoside); antitumour antibiotics (e.g., anthracyclines like adriamycin, bleomycin, doxorubicin, daunomycin, epirubicin and idarubicin, mitomycin-C, dactinomycin, mithramycin); platinum derivatives (e.g., cisplatin, carboplatin); alkylating agents (e.g., nitrogen mustard, melphalan, chlorambucil, busulphan, cyclophosphamide, ifosfamide, nitrosoureas, thiotepa); antimitotic agents (e.g., vinca alkaloids like vincristine, vinblastine, vindesine, vinorelbine, and taxoids like taxol, taxotere); topoisomerase inhibitors (e.g., epipodophyllotoxins like etoposide and teniposide, amsacrine, topotecan, camptothecin and also irinotecan); or thymidylate synthase inhibitors (e.g., raltitrexed).

In some embodiments, the chemotherapy is an antibody-drug conjugate (ADC). In some embodiments, the ADC is an antibody linked to a cytotoxic (anticancer) drug. In some embodiments, the ADC allows targeted delivery of cytotoxic drugs to tumor cells. In some embodiments, the ADC allows preferential deliver of cytotoxic drugs to tumor cells versus normal tissue.

In some embodiments, the chemotherapy is combination chemotherapy with a combination of different agents. In some embodiments, the combination comprises different agents that have different mechanisms of action and/or different, non-overlapping toxicities.

In some embodiments, the other therapy is an immune stimulator or immunotherapy, such as, for example, a checkpoint modulator/inhibitor. Checkpoint modulators/inhibitors are well known in the art to prevent the host immune system from attacking itself, and include, for example, CTLA-4, PD1, PDL1, GITR, OX40, LAG-3, and TIM-3. In some embodiments, the immune stimulator, immunotherapy, or checkpoint modulator/inhibitor is a monoclonal antibody. In some embodiments, the monoclonal antibody is an antibody against PD1, PDL1, CTLA-4, LAG3, OX40, CD40, CD40L, 41BB, 41BBL, GITR, CD3, CD28, CD38, or TGFbeta. In some embodiments, the monoclonal antibody is a bispecific antibody. In some embodiments, the immune stimulator is a cell-based immunotherapy. In some embodiments, the immune stimulator is a cytokine or chemokine. In some embodiments, the immune stimulator is a cancer vaccine. As a wide range of immune stimulators would be known to scientists and clinicians skilled in the art, the invention is not limited to a specific combination with a particular immune stimulator.

In some instances, any of the RNAs, RNA compositions, medical preparations, and RNA combination compositions described herein may be administered in combination with an immune stimulator, immunotherapy, or checkpoint modulator. In some instances, the RNAs, RNA compositions, and RNA combination compositions described herein are administered in combination with an antibody to a subject to treat cancer, including solid tumors. In some embodiments, the antibody is an anti-PD1 antibody, an anti-CTLA4 antibody, or a combination of an anti-PD1 antibody and anti-CTLA4 antibody. In some embodiments, the antibody is a multi-specific antibody such as, for example, a tri-specific or bi-specific antibody.

In some embodiments, the anti-PD1 antibody is a chimeric, humanized or human antibody. In some embodiments, the anti-PD-1 antibody is isolated and/or recombinant. Examples of anti-PD-1 antibodies are nivolumab, pembrolizumab, cemiplimab, MEDI0608 (formerly AMP-514; see, e.g., WO 2012/145493 and U.S. Pat. No. 9,205,148), PDR001 (see, e.g., WO 2015/112900), PF-06801591 (see, e.g., WO 2016/092419), BGB-A317 (see, e.g., WO 2015/035606).

In some embodiments, the anti-PD-1 antibody is one of those disclosed in WO 2015/112800 (such as those referred to as H1M7789N, H1M7799N, H1M7800N, H2M7780N, H2M7788N, H2M7790N, H2M7791N, H2M7794N, H2M7795N, H2M7796N, H2M7798N, H4H9019P, H4xH9034P2, H4xH9035P2, H4xH9037P2, H4xH9045P2, H4xH9048P2, H4H9057P2, H4H9068P2, H4xH9119P2, H4xH9120P2, H4xH9128P2, H4xH9135P2, H4xH9145P2, H4xH8992P, H4xH8999P and H4xH9008P in Table 1 of the PCT publication, and those referred to as H4H7798N, H4H7795N2, H4H9008P and H4H9048P2 in Table 3 of the PCT publication). The disclosure of WO 2015/112800 is incorporated by reference herein in its entirety. For example, the antibodies disclosed in WO 2015/112800 and related antibodies, including antibodies and antigen-binding fragments having the CDRs, VH and VL sequences, or heavy and light chain sequences disclosed in that PCT publication, as well as antibodies and antigen-binding fragments binding to the same PD-1 epitope as the antibodies disclosed in that PCT publication, can be used in conjunction with the RNA compositions of the present invention to treat and/or prevent cancer.

In related embodiments, the anti-PD-1 antibody may comprise the heavy and light chain amino acid sequences shown below as SEQ ID NOs: 79 and 80, respectively; the VH and VL sequences in SEQ ID NOs: 87 and 88 (shown in italics), or one or more (e.g., all six) CDRs in SEQ ID NOs: 79 and 80 (shown in bold boxes). In some embodiments, an antibody comprising the following CDRs is encompassed:

HCDR1 =
(SEQ ID NO: 81)
GFTFSNFG
HCDR2 =
(SEQ ID NO: 82)
ISGGGRDT
HCDR3 =
(SEQ ID NO: 83)
VKWGNIYFDY
LCDR1 =
(SEQ ID NO: 84)
LSINTF
LCDR2 =
(SEQ ID NO: 85)
AAS
LCDR3 =
(SEQ ID NO: 86)
QQSSNTPFT.

An exemplary antibody comprising a heavy chain comprising the VH and VL sequences in SEQ ID NOs: 87 and 88 (shown in italics) is the fully human anti-PD-1 antibody known as REGN2810 (cemiplimab).

Anti-PD-1 Mab heavy chain

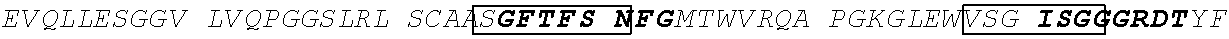

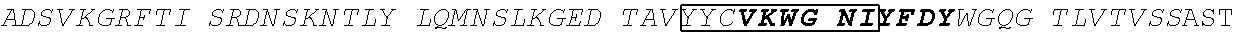

SLSSVVTVPS SSLGTKTYTC NVDHKPSNTK VDKRVESKYG PPCPPCPAPE FLGGPSVFLF
PPKPKDTLMI SRTPEVTCVV VDVSQEDPEV QFNWYVDGVE VHNAKTKPRE EQFNSTYRVV
SVLTVLHQDW LNGKEYKCKV SNKGLPSSIE KTISKAKGQP REPQVYTLPP SQEEMTKNQV
SLTCLVKGFY PSDIAVEWES NGQPENNYKT TPPVLDSDGS FFLYSRLTVD KSRWQEGNVF
SCSVMHEALH NHYTQKSLSL SLGK (SEQ ID NO: 79)
HCDR1 = GFTFSNFG (SEQ ID NO: 81)
HCDR2 = ISGGGRDT (SEQ ID NO: 82)
HCDR3 = VKWGNIYFDY (SEQ ID NO: 83)
Anti-PD-1 Mab light chain

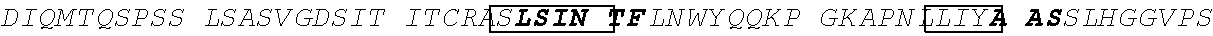

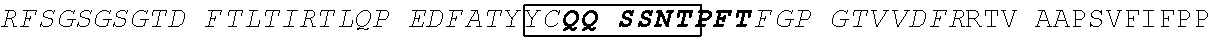

SDEQLKSGTA SVVCLLNNFY PREAKVQWKV DNALQSGNSQ ESVTEQDSKD STYSLSSTLT
LSKADYEKHK VYACEVTHQG LSSPVTKSEN RGEC (SEQ ID NO: 80)
LCDR1 = LSINTF (SEQ ID NO: 84)
LCDR2 = AAS (SEQ ID NO: 85)
LCDR3 = QQ SSNTPFT (SEQ ID NO: 86)

In some embodiments, the RNAs, RNA compositions, and RNA combination compositions may be delivered via injection into the tumor (e.g., intratumorally), near the tumor (peri-tumorally), or near the site of a tumor removal, and the antibody may be delivered in the same manner or systemically, such as, for example, enteral or parenteral, including, via injection, infusion, and implantation. “Administered in combination” includes simultaneous or sequential administration. If sequential, administration can be in any order and at any appropriate time interval known to those of skill in the art.

In some embodiments, the other therapy is hormonal therapy. In some embodiments, the hormonal therapy is antiestrogen drugs for treatment of breast cancer or anti-androgen drugs for treating prostate cancer. Example agents include antiestrogens (e.g., tamoxifen, toremifene, raloxifene, droloxifene, iodoxyfene), estrogen receptor down regulators (e.g., fulvestrant), progestogens (e.g., megestrol acetate), aromatase inhibitors (e.g., anastrozole, letrazole, vorazole, exemestane), antiprogestogens, antiandrogens (e.g., flutamide, nilutamide, bicalutamide, cyproterone acetate), LHRH agonists and antagonists (e.g., goserelin acetate, luprolide, buserelin), and inhibitors of 5-alpha-reductase (e.g., finasteride).

In some embodiments, the other therapy is a targeted therapy. In some embodiments, the targeted therapy is a kinase inhibitor. In some embodiments, the targeted therapy is one that inhibits activity of a gene product of a proto-oncogene. In some embodiments, the targeted therapy is an anti-angiogenic agent. In some embodiments, the targeted therapy is one directed to modulate activity of VEGF, BCR-ABL, BRAF, EGFR, c-Met, MEK, ERK, mTOR, or ALK.

In some embodiments, the other therapy is stem cell transplantation.

In some embodiments, therapeutic RNA compositions are delivered at the same time as another therapy.

In some embodiments, therapeutic RNA compositions are delivered before another therapy.

In some embodiments, therapeutic RNA compositions are delivered after another therapy.

In some embodiments, therapeutic RNA compositions are delivered directly into the tumor, or near the tumor or the site of tumor removal together with another therapy. In some embodiments, therapeutic RNA compositions are delivered directly into the tumor, or near the tumor or site of tumor removal while another agent is delivered systemically.

### IV. Pharmaceutical Formulations

In some embodiments, any of the DNAs, RNAs, and compositions described herein are pharmaceutical formulations. In some embodiments, the pharmaceutical formulations comprise a diluent, excipient, or other pharmaceutically acceptable carrier. Thus, provided herein are pharmaceutical compositions comprising the DNA, RNA, compositions, or combinations thereof provided herein, and a pharmaceutically acceptable excipient. In certain embodiments, the pharmaceutically acceptable excipient is an aqueous solution. In certain embodiments, the aqueous solution is a saline solution. As used herein, pharmaceutically acceptable excipients are understood to be sterile. In some embodiments, a pharmaceutical composition is administered in the form of a dosage unit. For example, in certain embodiments, a dosage unit is in the form of a tablet, capsule, implantable device, or a bolus injection.

In some embodiments, the pharmaceutical compositions provided herein may additionally contain other adjunct components conventionally found in pharmaceutical compositions. For example, the compositions may contain additional, compatible, pharmaceutically-active materials such as, for example, antipruritics, astringents, local anesthetics or anti-inflammatory agents. The compositions may also contain additional, compatible, pharmaceutically-inactive materials such as excipients, diluents, and carriers.

Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

Lipid moieties may be used to deliver the RNAs provided herein. In one method, the RNA is introduced into preformed liposomes or lipoplexes made of mixtures of cationic lipids and neutral lipids. In another method, RNA complexes with mono- or poly-cationic lipids are formed without the presence of a neutral lipid. In certain embodiments, a lipid moiety is selected to increase distribution of a pharmaceutical agent to a particular cell or tissue.

In some embodiments, a pharmaceutical composition provided herein comprises a polyamine compound or a lipid moiety complexed with the DNA or RNA provided herein.

In some embodiments, a pharmaceutical composition provided herein comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

Certain pharmaceutical compositions for injection are presented in unit dosage form, e.g., in ampoules or in multi-dose containers. In certain embodiments, a pharmaceutical composition provided herein comprises a RNA or combination of RNAs in a therapeutically effective amount. In certain embodiments, the therapeutically effective amount is sufficient to treat or prevent cancer in the subject being treated.

The following clauses provide numerous embodiments and are non-limiting:
Clause 1. A composition comprising RNA encoding an IL-12sc protein, RNA encoding an IL-15 sushi protein, RNA encoding an IFNα protein, and RNA encoding a GM-CSF protein.
Clause 2. The composition of clause 1, wherein the IFNα protein is an IFNα2b protein.
Clause 3. The composition of clause 1, wherein (i) the RNA encoding an IL-12sc protein comprises the nucleotide sequence of SEQ ID NO: 17 or 18, or a nucleotide sequence having at least 99%, 98%, 97%, 96%, 95%, 90%, 85%, or 80% identity to the nucleotide sequence of SEQ ID NO: 17 or 18 and/or (ii) the IL-12sc protein comprises the amino acid sequence of SEQ ID NO: 14, or an amino acid sequence having at least 99%, 98%, 97%, 96%, 95%, 90%, 85%, or 80% identity to the amino acid sequence of SEQ ID NO: 14.
Clause 4. The composition of clause 1, wherein (i) the RNA encoding an IL-15 sushi protein comprises the nucleotide sequence of SEQ ID NO: 26, or a nucleotide sequence having at least 99%, 98%, 97%, 96%, 95%, 90%, 85%, or 80% identity to the nucleotide sequence of SEQ ID NO: 26 and/or (ii) the IL-15 sushi protein comprises the amino acid sequence of SEQ ID NO: 24, or an amino acid sequence having at least 99%, 98%, 97%, 96%, 95%, 90%, 85%, or 80% identity to the amino acid sequence of SEQ ID NO: 24.
Clause 5. The composition of clause 1, wherein (i) the RNA encoding an IFNα protein comprises the nucleotide sequence of SEQ ID NO: 22 or 23, or a nucleotide sequence having at least 99%, 98%, 97%, 96%, 95%, 90%, 85%, or 80% identity to the nucleotide sequence of SEQ ID NO: 22 or 23 and/or (ii) the IFNα protein comprises the amino acid sequence of SEQ ID NO: 19, or an amino acid sequence having at least 99%, 98%, 97%, 96%, 95%, 90%, 85%, or 80% identity to the amino acid sequence of SEQ ID NO: 19.
Clause 6. The composition of clause 1, wherein (i) the RNA encoding a GM-CSF protein comprises the nucleotide sequence of SEQ ID NO: 29, or a nucleotide sequence having at least 99%, 98%, 97%, 96%, 95%, 90%, 85%, or 80% identity to the nucleotide sequence of SEQ ID NO: 29 and/or (ii) the GM-CSF protein comprises the amino acid sequence of SEQ ID NO: 27, or an amino acid sequence having at least 99%, 98%, 97%, 96%, 95%, 90%, 85%, or 80% identity to the amino acid sequence of SEQ ID NO: 27.
Clause 7. The composition of clause 1, wherein at least one RNA comprises a modified nucleoside in place of at least one uridine, wherein the modified nucleoside is pseudouridine (ψ), N1-methyl-pseudouridine (m¹ψ), 5-methyl-uridine (m5U), or a combination thereof.
Clause 8. The composition of clause 1, wherein each RNA comprises a modified nucleoside in place of at least one uridine, wherein the modified nucleoside is pseudouridine (ψ), N1-methyl-pseudouridine (m¹ψ), 5-methyl-uridine (m5U), or a combination thereof.
Clause 9. The composition of clause 1, wherein at least one RNA comprises the 5′ cap m₂^{7,3′-O}Gppp(m₁^{2′-O})ApG, or 3′-O-Me-m⁷G(5′)ppp(5′)G, or m₂^{7,3′-O}Gppp(m₁^{2′-O})ApG, or 3′-O-Me-m⁷G(5′)ppp(5′)G.
Clause 10. The composition of clause 1, wherein at least one RNA comprises a 5′ UTR comprising (i) a nucleotide sequence selected from the group consisting of SEQ ID NOs: 2, 4, and 6, or (ii) a nucleotide sequence having at least 99%, 98%, 97%, 96%, 95%, 90%, 85%, or 80% identity to a nucleotide sequence selected from the group consisting of SEQ ID NOs: 2, 4, and 6 and/or a 3′ UTR comprising (i) the nucleotide sequence of SEQ ID NO: 8, or (ii) a nucleotide sequence having at least 99%, 98%, 97%, 96%, 95%, 90%, 85%, or 80% identity to the nucleotide sequence of SEQ ID NO: 8.
Clause 11. The composition of clause 1, wherein at least one RNA comprises a poly-A tail of at least 100 nucleotides.
Clause 12. The composition of clause 11, wherein the poly-A tail comprises the poly-A tail shown in SEQ ID NO: 78.
Clause 13. The composition of clause 1, wherein one or more RNA comprises:
   a 5′ cap comprising m₂^{7,3′-O}Gppp(m₁^{2′-O})ApG or 3′-O-Me-m⁷G(5′)ppp(5′)G;
   a 5′ UTR comprising (i) a nucleotide sequence selected from the group consisting of SEQ ID NOs: 2, 4, and 6, or (ii) a nucleotide sequence having at least 99%, 98%, 97%, 96%, 95%, 90%, 85%, or 80% identity to a nucleotide sequence selected from the group consisting of SEQ ID NOs: 2, 4, and 6;
   a 3′ UTR comprising (i) the nucleotide sequence of SEQ ID NO: 8, or (ii) a nucleotide sequence having at least 99%, 98%, 97%, 96%, 95%, 90%, 85%, or 80% identity to the nucleotide sequence of SEQ ID NO:8; and
   a poly-A tail comprising at least 100 nucleotides.
Clause 14. The composition of clause 13, wherein the poly-A tail comprises SEQ ID NO: 78.
Clause 15. A pharmaceutical composition comprising the composition of clause 1 and a pharmaceutically acceptable carrier, diluent and/or excipient.
Clause 16. A method for treating or reducing the likelihood of a solid tumor comprising administering to a subject in need thereof the composition of clause 1.
Clause 17. A method for treating or reducing the likelihood of a solid tumor comprising administering to a subject in need thereof a first RNA, wherein the first RNA encodes an IL-12sc protein, an IL-15 sushi protein, an IFNα protein, or a GM-CSF protein, and additional RNA, wherein:
   if the first RNA encodes an IL-12sc protein, then the additional RNA encodes an IL-15 sushi protein, an IFNα protein, and a GM-CSF protein;
   if the first RNA encodes an IL-15 sushi protein, then the additional RNA encodes an IL-12sc protein, an IFNα protein, and a GM-CSF protein;
   if the first RNA encodes an IFNα protein, then the additional RNA encodes an IL-15 sushi protein, an IL-12sc protein, and a GM-CSF protein; and
   if the first RNA encodes a GM-CSF protein, then the additional RNA encodes an IL-15 sushi protein, an IFNα protein, and an IL-12sc protein.
Clause 18. The method of clause 17, wherein the first RNA is administered to the subject at the same time as the additional RNA.
Clause 19. The method of clause 17, wherein the RNA is administered intra-tumorally or peri-tumorally.
Clause 20. The method of clause 17, wherein the subject is further treated with an additional therapy comprising (i) surgery to excise, resect, or debulk a tumor, (ii) immunotherapy, (iii) radiotherapy, or (iv) chemotherapy.
Clause 21. The method of clause 17, wherein the subject is further treated with a checkpoint modulator.
Clause 22. The method of clause 21, wherein the checkpoint modulator is an anti-PD1 antibody, an anti-CTLA-4 antibody, or a combination of an anti-PD1 antibody and an anti-CTLA-4 antibody.
Clause 23. The method of clause 21, wherein the RNA is administered intra-tumorally or peri-tumorally via injection, and the checkpoint modulator is administered systemically.
Clause 24. The method of clause 17, wherein the solid tumor is a sarcoma, carcinoma, or lymphoma.
Clause 25. The method of clause 17, wherein the solid tumor is in the lung, colon, ovary, cervix, uterus, peritoneum, testicles, penis, tongue, lymph node, pancreas, bone, breast, prostate, soft tissue, connective tissue, kidney, liver, brain, thyroid, or skin.
Clause 26. The method of clause 17, wherein the solid tumor is an epithelial tumor, Hodgkin lymphoma (HL), non-Hodgkin lymphoma, prostate tumor, ovarian tumor, renal cell tumor, gastrointestinal tract tumor, hepatic tumor, colorectal tumor, tumor with vasculature, mesothelioma tumor, pancreatic tumor, breast tumor, sarcoma tumor, lung tumor, colon tumor, brain tumor, melanoma tumor, small cell lung tumor, neuroblastoma tumor, testicular tumor, carcinoma tumor, adenocarcinoma tumor, glioma tumor, seminoma tumor, retinoblastoma, or osteosarcoma tumor.
Clause 27. The method of clause 17, wherein treating or reducing the likelihood of the solid tumor comprises reducing the size of a tumor, reducing the likelihood of a reoccurrence of cancer in remission, or reducing the likelihood of cancer metastasis in the subject.
Clause 28. A combination therapy method for treating or reducing the likelihood of a solid tumor, comprising administering to a subject in need thereof RNAs and a further therapy, wherein the RNAs encode an IL-12sc protein, an IL-15 sushi protein, an IFNα protein, and a GM-CSF protein, and the further therapy comprises immunotherapy, chemotherapy, or a checkpoint modulator.
Clause 29. The combination therapy method of clause 28, wherein the further therapy comprises an anti-PD1 antibody, an anti-CTLA-4 antibody, or a combination of an anti-PD1 antibody and an anti-CTLA-4 antibody.
Clause 30. An isolated nucleic acid comprising a sequence encoding an IL-12sc protein, wherein:
   the sequence encoding the IL-12sc protein comprises a) contiguous nucleotides having at least 99%, 98%, 97%, 96%, 95%, 90%, 85%, 80%, or 78% identity to nucleotides 1-984 of SEQ ID NO: 16 or 18, b) contiguous nucleotides having at least 99%, 98%, 97%, 96%, 95%, 90%, 85%, or 81% identity to nucleotides 1027-1623 of SEQ ID NO: 16 or 18, and c) nucleotides encoding a linker between the nucleotides of a) and b).
Clause 31. The nucleic acid of clause 30, which is a DNA.
Clause 32. The nucleic acid of clause 30, which is an RNA.
Clause 33. An isolated nucleic acid comprising a sequence encoding an IFNα protein, wherein:
   the sequence encoding the IFNα protein has at least 99%, 98%, 97%, 96%, 95%, 90%, 85%, or 80% identity to the nucleotide sequence of SEQ ID NO: 21 or 23.
Clause 34. The nucleic acid of clause 33, which is a DNA.
Clause 35. The nucleic acid of clause 33, which is an RNA.
Clause 36. A method for treating or reducing the likelihood of a solid tumor comprising administering to a subject in need thereof the RNA of clause 34, wherein: the subject is further treated with additional RNA encoding an IL-15 sushi protein, an IFNα protein, and a GM-CSF protein.
Clause 37. A method for treating or reducing the likelihood of a solid tumor comprising administering to a subject in need thereof the RNA of clause 35, wherein: the subject is further treated with additional RNA encoding an IL-15 sushi protein, an IL-12sc protein, and a GM-CSF protein.
Clause 38. A method of producing an RNA encoding IL-12sc, comprising contacting an expression construct comprising the nucleic acid of clause 32 operably linked to a promoter with an RNA polymerase under conditions permissive for transcription.
Clause 39. A method of producing an RNA encoding IFNα, comprising contacting an expression construct comprising the nucleic acid of clause 35 operably linked to a promoter with an RNA polymerase under conditions permissive for transcription.
Clause 40. A kit comprising the composition of clause 1.
Clause 41. A kit comprising RNA encoding an IL-12sc protein, RNA encoding an IL-15 sushi protein, RNA encoding an IFNα protein, and RNA encoding a GM-CSF protein, wherein the RNAs are not in the same container.
Clause 42. The kit of clause 41, wherein each RNA is in a separate container.
Clause 43. The kit of any one of clause 40-42, further comprising instructions for use of the composition or RNAs for treating or reducing the likelihood of a solid tumor.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

### EXAMPLES

The following examples are provided to illustrate certain disclosed embodiments and are not to be construed as limiting the scope of this disclosure in any way.

### Example 1—Materials and Methods

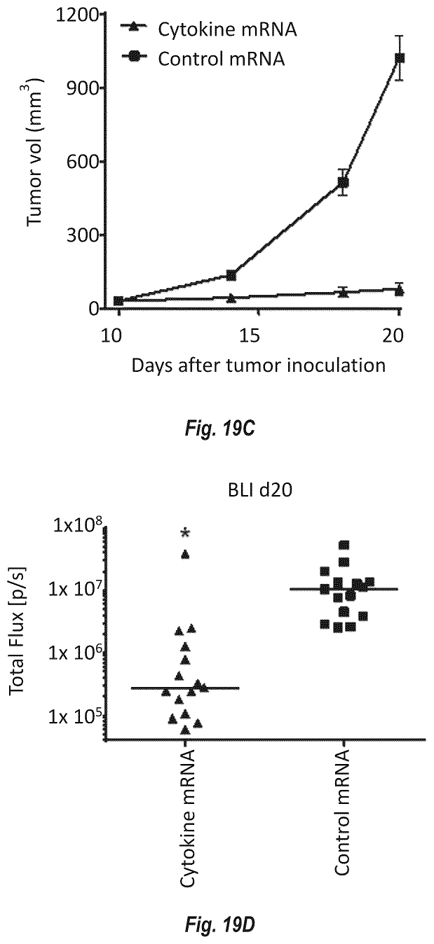

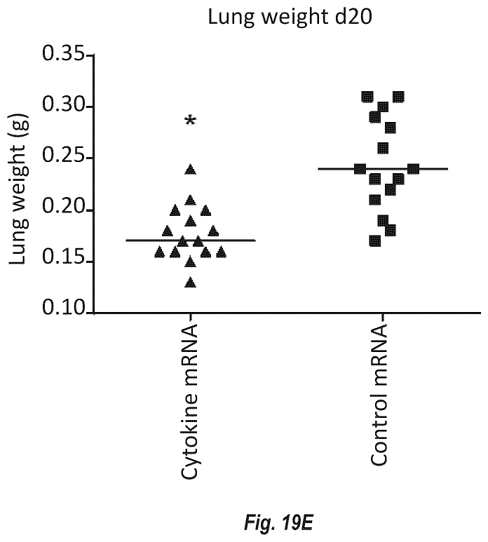

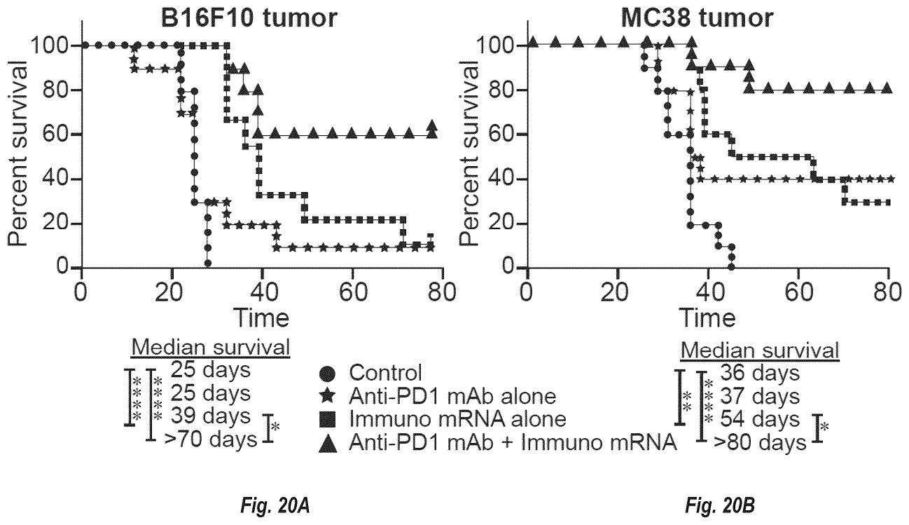

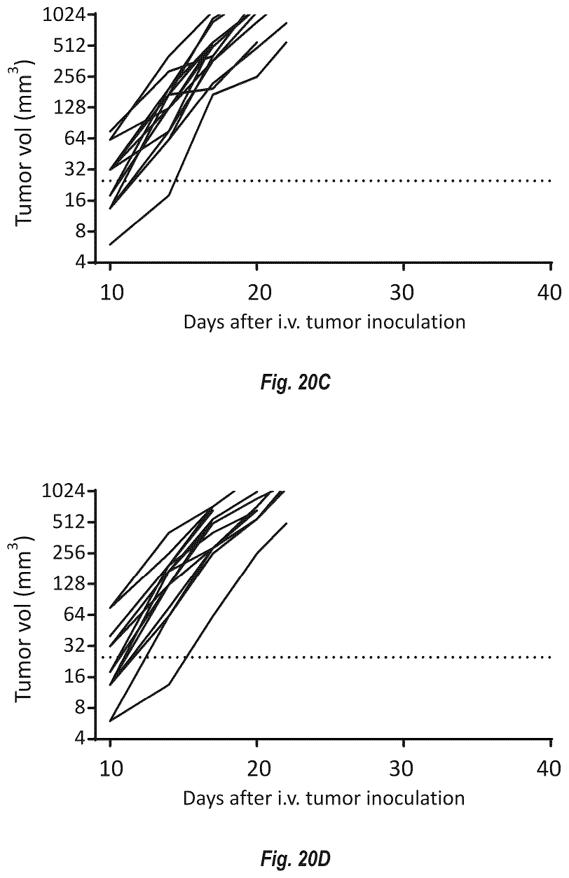

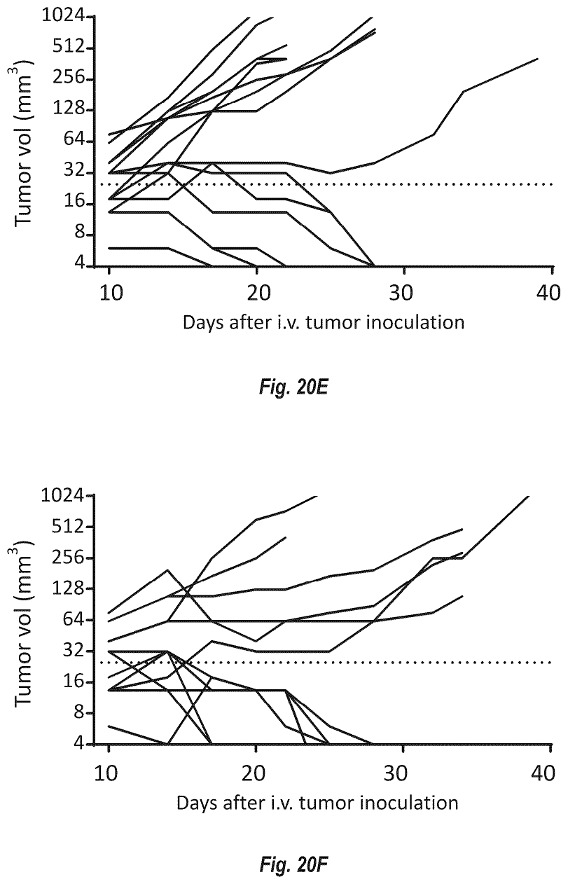

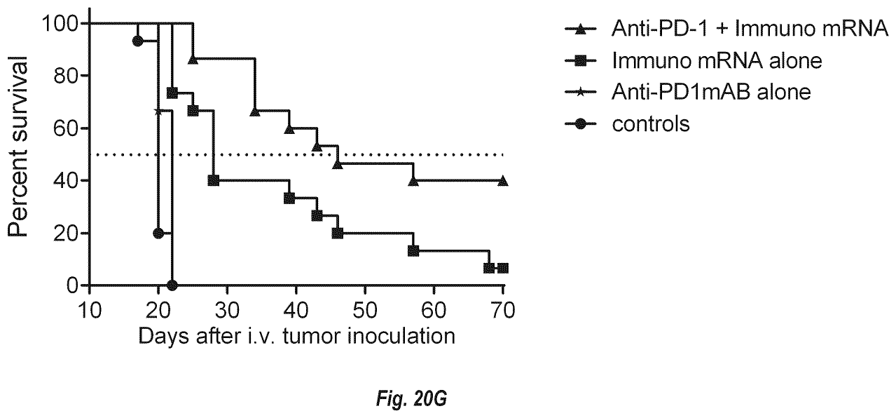

B16F10 Tumor Model: Female C57BL/6J mice (Jackson Laboratory; Bar Harbor, ME), 6-8 weeks-old and weighing between 17.0 and 20.9 g were acclimated for at least three days prior to study enrollment. Mice had free access to food (Harlan 2916 rodent diet, Massachusetts, USA) and sterile water and housed on 12 hours light/dark cycle at 22° C.±2° C. with a relative humidity of 55%±15%. B16F10 cells were obtained from the American Type Culture Collection (ATCC) (Manassas, Virginia USA) (Cat No. CRL-6475) and cultured in Dulbecco's Modified Eagle's Medium (DMEM) (Life technologies, Cat No. 11995) supplemented with 10% heat inactivated Fetal Bovine Serum (HI FBS) (Life technologies, Cat No. 10082-147) in 5% CO2 at 37° C. The cells were harvested using 0.25% Trypsin-EDTA (Life technologies, Cat No. 25200-056), resuspended in Dulbecco's phosphate-buffered saline (DPBS) (Life technologies, Cat No. 14190-144), and 0.5×10\hat{ }6 cells/200 μl per mouse subcutaneously (SC) implanted into the right flank of female C57BL/6J mice. For dual flank tumor models on day 0, 0.5×10\hat{ }6 B16F10 cells/200 μl per mouse were subcutaneously (SC) implanted into the right flank and 0.25×10\hat{ }6 B16F10 cells/200 μl per mouse were SC implanted into the left flank. To test whether local administration of cytokine mRNA can have distant effects (see FIG. 19), female C57BL/6JOlaHsd mice (Envigo, Rossdorf, Germany), 6-8 weeks of age, with a weight between 17 and 24 g, were acclimated for at least six days prior to study enrollment. Mice had free access to food (ssniff M-Z autoclavable Soest, Germany) and sterile water and housed on 12 hours light/dark cycle at 22° C.±2° C. with a relative humidity of 55%±10%. B16F10 cells were obtained from the American Type Culture Collection (ATCC® CRL-6475™) and cultured in DMEM, high glucose, GlutaMAX™ (Life technologies, Cat No. 31966047) supplemented with 10% Fetal Bovine Serum (FBS) (Biochrom, Cat No. S 0115) in 7.5% CO2 at 37° C. The cells were harvested using StemPro® Accutase® Cell Dissociation Reagent (Life technologies, Cat No. A1110501), re-suspended in Dulbecco's phosphate-buffered saline (DPBS) (Life technologies, Cat No. 14190-169), and 0.3×10\hat{ }6 cells/100 μl per mouse subcutaneously (SC) were implanted into the right shaven flank of female C57BL/6J mice. For seeding of distant lung tumors, B16F10_luc-gfp cells were used. These cells were derived from B16F10 by stable transfection with a plasmid coding for Luciferase and GFP. B16F10_luc-gfp cells were cultured with the same conditions as the B16F10; only 0.5 μg/mL Puromycin was added to the culture medium. One day after SC implantation 0.3×10\hat{ }6 cells/200 μl of B16F10_luc-gfp cells were injected per mouse intravenously (IV) into the tail vein. Intratumoral mRNA injections were initiated 10-14 days after SC inoculation of the tumors. Tumor growth was assessed by caliper measurements every 2-3 days and is expressed as the product of the perpendicular diameters using the following formula: a2*b/2, with a<b. Engraftment of luciferase-positive tumor cells in the lung was analyzed by in vivo bioluminescence imaging using the Xenogen IVIS Spectrum imaging system (Caliper Life Sciences). An aqueous solution of L-luciferin (250 μl, 1.6 mg; BD Biosciences) was injected intraperitoneally. Emitted photons from live animals quantified 10 min later with an exposure time of 1 min. Regions of interest (ROT) were quantified as average radiance (photons s⁻¹ cm⁻²sr⁻¹, represented by as color-scaled images superimposed on grayscale photos of mice using the Living Image software from Caliper Life Sciences). For absolute quantification, the bioluminescence signal was blotted as total flux (photons s⁻¹). For studies performed in FIGS. 20C-G and 21E-I the above described model of C57BL/6JOlaHsd mice (Envigo, Rossdorf, Germany) without seeding of lung metastasis was used as well.

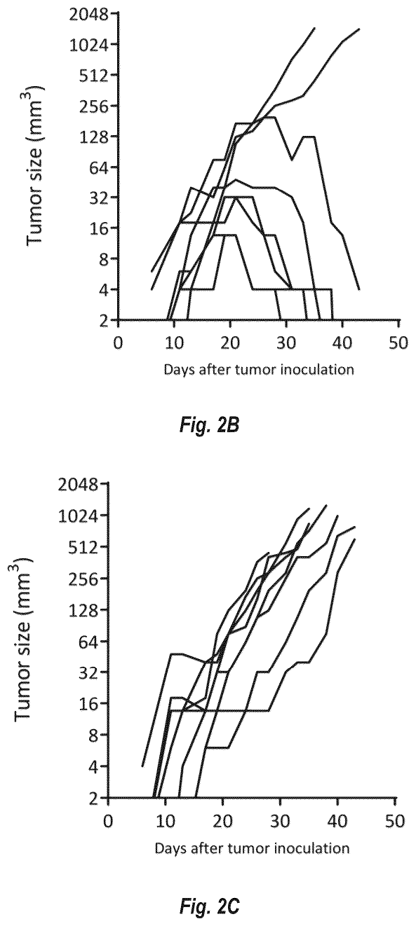

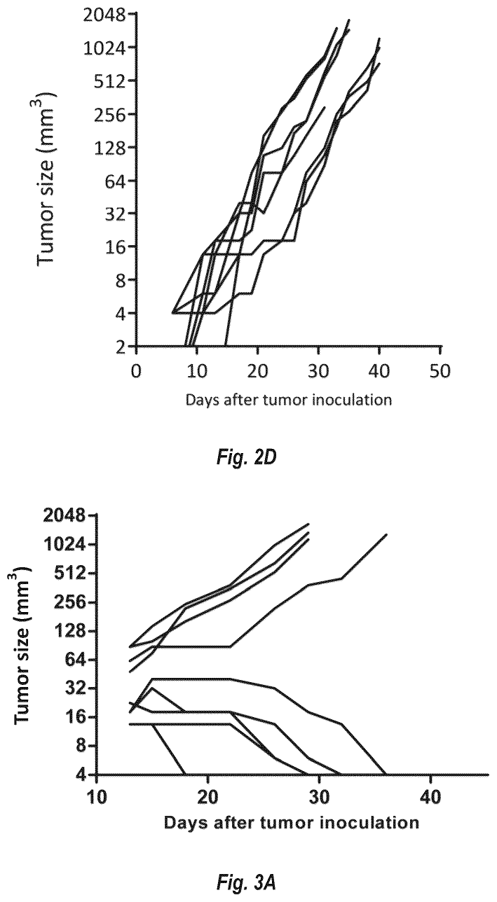

CT26 tumor model: For studies performed in FIGS. 2, 3, 7D-F, 8, 9, 12D and 21, female Balb/c Rj mice (Janvier, Genest-St.-Îsle, France), 6-8 weeks of age, with a weight between 17 and 24 g, were acclimated for at least six days prior to study enrollment. Mice had free access to food (ssniff M-Z autoclavable Soest, Germany) and sterile water and housed on 12 hours light/dark cycle at 22° C.±2° C. with a relative humidity of 55%±10%. CT26 cells were obtained from the (ATCC® CRL-2638™) and cultured in Roswell Park Memorial Institute medium (RPMI) 1640 Medium, GlutaMAX™ (Life technologies, Cat No. 61870-044) supplemented with 10% Fetal Bovine Serum (FBS) (Biochrom, Cat No. S 0115) in 5% CO2 at 37° C. The cells were harvested using StemPro® Accutase® Cell Dissociation Reagent (Life technologies, Cat No. A1110501), resuspended in DPBS (Life technologies, Cat No. 14190-169), and 0.5×10\hat{ }6 cells/100 μl per mouse SC implanted into the right shaven flank of female Balb/c Rj mice. Intratumoral RNA injections were initiated 13-19 days after inoculation of the tumors. Tumor growth was assessed by caliper measurements every 2-3 days and is expressed as the product of the perpendicular diameters using the following formula: a2*b/2 where b is the longer of the two diameters (a<b).

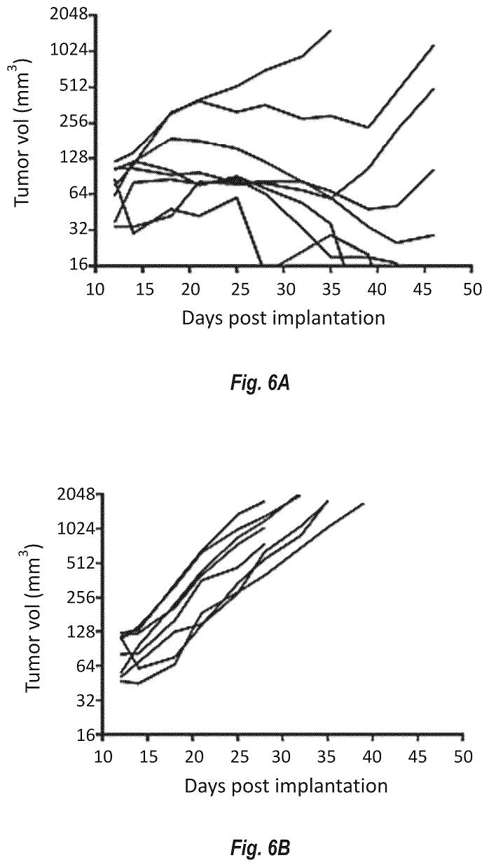

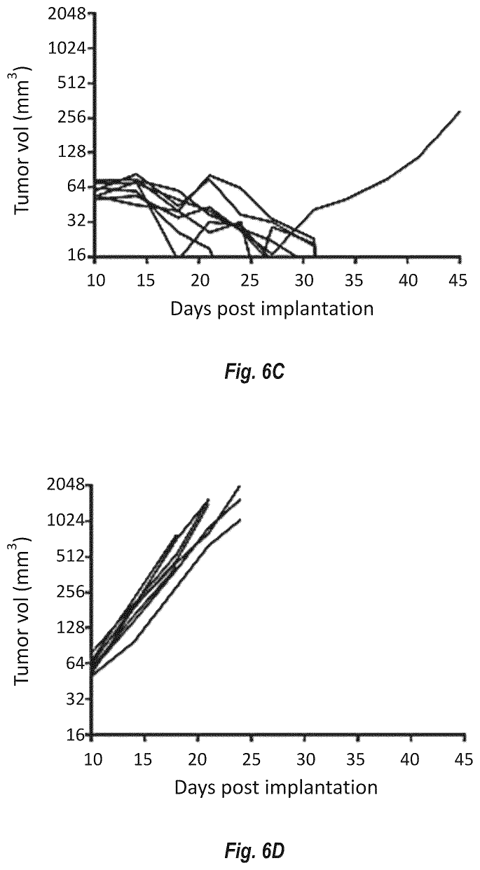

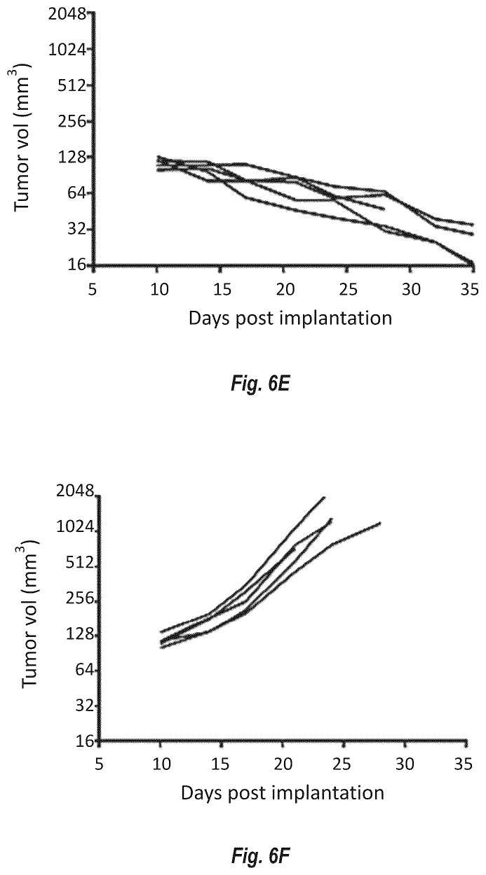

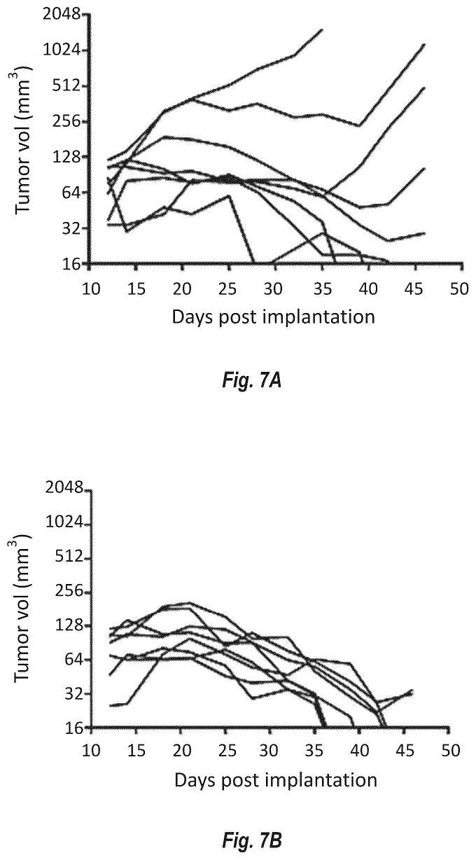

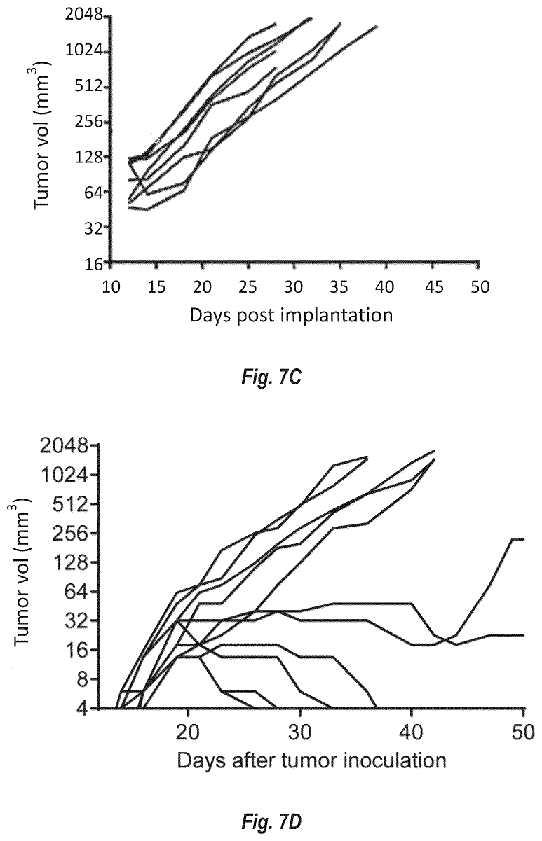

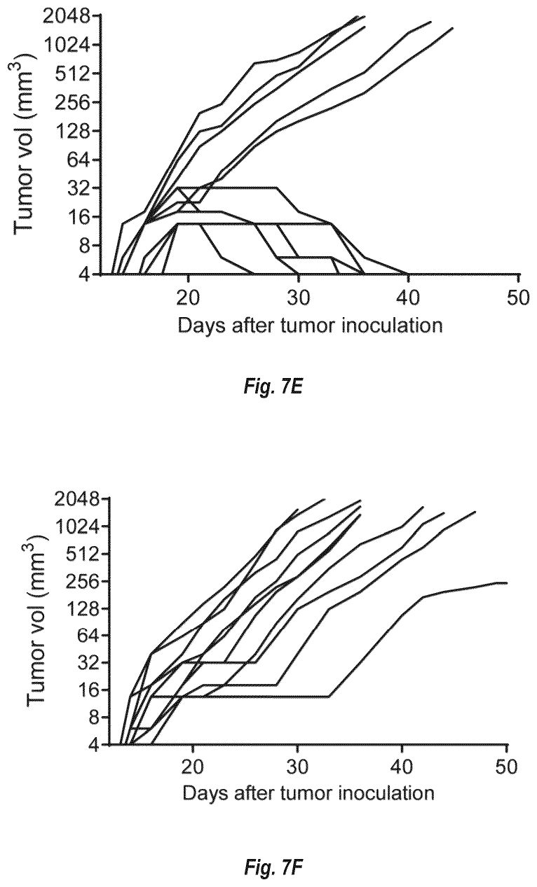

For studies performed in FIGS. 6, and 7A-C female BALB/c mice (Jackson Laboratory; Bar Harbor, ME), 6-8 weeks-old and weighing between 17.0 and 20.9 g were acclimated for at least three days prior to study enrollment. Mice had free access to food (Harlan 2916 rodent diet, Massachusetts, USA) and sterile water and housed on 12 hours light/dark cycle at a temperature (22° C.±2° C.), relative humidity (55%±15%). CT26 cells were obtained from the ATCC (Manassas, Virginia USA) (Cat No. CRL-2638) and cultured in RPMI-1640 (Life technologies, Cat No. 11875-093) supplemented with 10% HI FBS (Life technologies, Cat No. 10082-147) in 5% CO2 at 37° C. The cells were harvested using 0.25% Trypsin-EDTA (Life technologies, Cat No. 25200-056), re-suspended in DPBS (Life technologies, Cat No. 14190-144), and 0.5×10\hat{ }6 cells/200 μl per mouse SC implanted into the right flank of BALB/c female mice.

In CT26 tumor model in addition to tumor growth gp70-reactive CD8+ T-cells were measured in blood where indicated. Blood samples were taken using EDTA-coated tubes. 100 μL of blood was transferred to FACS tubes and antibody mixture was added containing T-Select H-2Ld MuLV gp70 Tetramer-SPSYVYHQF-APC (MBL (TS-M-521-2), 4 μL for 100 μL blood), anti-CD8a FITC (life technologies (MCD801), 1 μL for 100 μL blood)) and anti-CD45 V500 (BD (561487), 1 μL for 100 μL blood)). After 20 min incubation at room temperature Blood Lysis Buffer (BD (349202), 300 μL per tube) was added and incubated for further 6 min. Then samples were washed twice with PBS-EDTA buffer. FACS samples were analyzed on a FACS Canto II flow cytometer.

MC38 tumor model: Female C57BL/6J mice (Jackson Laboratory; Bar Harbor, ME), 6-8 weeks-old and weighing between 17.0 and 20.9 g were acclimated for at least three days prior to study enrollment. Mice had free access to food (Harlan 2916 rodent diet, Massachusetts, USA) and sterile water and housed on 12 hours light/dark cycle at 22° C.±2° C. with a relative humidity of 55%±15%. MC38 cells were generous gifts from Dr S. A. Rosenberg (National Institute of Health, Bethesda, MD, USA). The cell line was cultured in RPMI-1640 with L-glutamine (Gibco, Cat No. 11875) supplemented with 10% HI FBS (Gibco, Cat No. 100082) in 5% CO2 at 37° C. The cells were harvested, re-suspended in DPBS (Gibco, Cat No. 14190), and 1×10\hat{ }6 cells/200 μl per mouse SC implanted into the right flank of female C57BL/6J mice.

A375 tumor model: Female severe combined immune deficiency (SCID) mice (Jackson Laboratory; Bar Harbor, ME), 6-8 weeks-old and weighing between 17.0 and 20.9 g were acclimated for at least three days prior to study enrollment. Mice had free access to food (Harlan 2916 rodent diet, Massachusetts, USA), sterile water and housed on 12 hours light/dark cycle at 22° C.±2° C. with a relative humidity of 55%±15%. A375 cells were obtained from the ATCC (Manassas, Virginia USA) (Cat No. CRL-1619). The cell line was cultured in DMEM (Life technologies, Cat No. 11995) supplemented with 10% HI FBS (Life technologies, Cat No. 10082-147) in 5% CO2 at 37° C. The cells were harvested using 0.25% Trypsin-EDTA (Life technologies, Cat No. 25200-056), re-suspended in DPBS (Life technologies, Cat No. 14190-144), and 3.0×10⁶/100 μl PBS were mixed with 100 ul BD Matrigel Matrix (BD, Cat No. 354234) and implanted SC into the right flank of female SCID mice.

KM12 (CRC) Xenograft Model: Female NOD.CB17-Prkdcscid/SCID mice (Jackson Laboratory, Bar Harbor, ME), 10-weeks-old and weighing between 17.3 g and 21.9 g were acclimated for at least three days prior to study enrollment. Mice had free access to food (Harlan 2916 rodent diet, Massachusetts, USA), sterile water and were housed on 12 hours light/dark cycle at (22±2° C.) with a relative humidity (55±15%). KM-12 cells were obtained from the American National Cancer Institute (NCI) (Cat No. 507345). The cells were grown in RPMI medium 1640 with L-glutamine (Gibco, Cat No. 11875) supplemented with 10% HI FBS (Gibco, Cat No. 10082), and incubated at 37° C. with 5% CO2. The cells were harvested using 0.25% Trypsin-EDTA (Gibco, Cat No. 25200), re-suspended in DPBS (Gibco, Cat No. 14190), and for each mouse 5.0×106 cells in 200 μl DPBS with 50% matrigel (BD, Cat No. 356234) were SC implanted into the right flank of female SCID mice.

RPMI8226 (Myeloma) Xenograft Model: Female NSG mice (Jackson Laboratory, Bar Harbor, ME), 12-weeks-old and weighing between 19.8 g and 26.6 g were acclimated for at least three days prior to study enrollment. Mice had free access to food (Harlan 2916 rodent diet, Massachusetts, USA), sterile water and were housed on 12 hours light/dark cycle at (22±2° C.) with a relative humidity (55±15%). RPMI8226 cells were obtained from the ATCC (Cat No. CCL-155). The cells were grown in RPMI medium 1640 with L-glutamine (Gibco, Cat No. 11875) supplemented with 10% HI FBS (Gibco, Cat No. 10082), and incubated at 37° C. with 5% CO2. The cells were harvested using 0.25% Trypsin-EDTA (Gibco, Cat No. 25200), re-suspended in DPBS (Gibco, Cat No. 14190), and for each mouse 5.0×106 cells in 200 μl DPBS with 50% matrigel (BD, Cat No. 356234) were SC implanted into the right flank of female NSG mice.

NCI-N87 (Gastric) Xenograft Model: Female NOD.CB17-Prkdcscid/SCID (Jackson Laboratory, Bar Harbor, ME), 11-weeks-old and weighing between 18.3 and 22.7 g were acclimated for at least three days before the study enrollment. Mice had free access to food (Harlan 2916 rodent diet, Massachusetts, USA), sterile water and were housed on 12 hours light/dark cycle at (22±2° C.) with a relative humidity (55±15%). NCI-N87 cells were obtained from the ATCC (Cat No. CRL-5822). The cells were grown in RPMI medium 1640 with L-glutamine (Gibco, Cat No. 11875) supplemented with 10% HI FBS (Gibco, Cat No. 10082), and incubated at 37° C. with 5% CO2. The cells were harvested using 0.25% Trypsin-EDTA (Gibco, Cat No. 25200), re-suspended in DPBS (Gibco, Cat No. 14190), and for each mouse 3.0×106 cells in 200 μl DPBS with 50% matrigel (BD, Cat No. 356234) were SC implanted into the right flank of female SCID mice.

NCI-H1975 (NSCLC) Xenograft Model: Female NSG mice (Jackson Laboratory, Bar Harbor, ME), 10-weeks-old and weighed between 18.8 g and 26.0 g were allowed to acclimate for at least three days before study enrollment. Mice had free access to food (Harlan 2916 rodent diet, Massachusetts, USA), sterile water and were housed on 12 hours light/dark cycle at (22±2° C.) with a relative humidity (55±15%). NCI-H1975 cells were obtained from the ATCC (Cat No. CRL-5908) and cultured in RPMI medium 1640 with L-glutamine (Gibco, Cat No. 11875) supplemented with 10% HI FBS (Gibco, Cat No. 10082), and incubated at 37° C. with 5% CO2. The cells were harvested using 0.25% Trypsin-EDTA (Gibco, Cat No. 25200), re-suspended in DPBS (Gibco, Cat No. 14190), and for each mouse 5.0×106 cells in 200 μl DPBS with 50% matrigel (BD, Cat No. 356234) were SC implanted into the right flank of female NSG mice.

Tumor re-challenge: Female C57BL/6J mice were implanted with B16F10 cells as described above. Mice were treated with 4 intratumoral injections (80 μg mRNA/20 μg per target) on days 11, 13, 15, and 17 with ModB cytokine mRNA mixture (IL-15sushi, IL-12sc, GM-CSF, IFNα). After cytokine mRNA treatment 8 mice were tumor free. Four weeks after the last cytokine mRNA treatment tumor free mice were re-challenged with 0.5×10\hat{ }6 B16F10 cells/200 μl per mouse by SC injection and tumor growth was monitored.

Tumor monitoring: Tumors were measured with a caliper twice weekly until final sacrifice. When a tumor size reached approximately 2000 mm³ or there are animal health issues (20% area of a tumor ulcerated), animals were euthanized. Tumor regression was defined as i) tumor volume <20 mm3 at the end of the study or ii) T^{F}/T⁰<1, where the T^{F} equals the final tumor volume and T⁰ equals tumor volume on the day of the first intratumoral mRNA injection.

mRNA modification A (ModA): Synthetic DNA fragments coding for the gene of interest were cloned into a common starting vector, comprising a 5′-UTR (corresponding in some cases to SEQ ID NO: 1), a 3′ UTR consisting of two elements called F and I (corresponding in some cases to SEQ ID NO: 7), and a poly(A)-tail of 110 nucleotides in total (A30-linker-A70 structure; corresponding in some cases to SEQ ID NO: 78). Linearization of plasmid DNA was performed downstream of the poly(dA:dT) with a classIIS restriction enzyme to generate a template with no additional nucleotide beyond the poly(dA:dT) (see Holtkamp et al., Blood 108(13):4009-172006 (2006)). Linearized plasmid DNA was subjected to in vitro transcription with T7 RNA polymerase (Thermo Fisher) as previously described (see Grudzien-Nogalska E et al., Methods Mol Biol. 969:55-72 (2013)) in the presence of 7.5 mM ATP, CTP, UTP, GTP and 6 mM D1, a beta-S-antireverse cap analogue (beta-S-ARCA, Cap0) (see Kuhn A N et al, Gene Ther. 17(8):961-71 (2010)). RNA was purified using magnetic particles (see Berensmeier S. Appl Microbiol Biotechnol. 73(3):495-504 (2006)), and RNA concentration and quality were assessed spectrophotometry and analyzed by capillary gel electrophoresis systems, respectively.

mRNA modification B (ModB): Synthetic DNA fragments coding for the gene of interest were cloned into a common starting vector, comprising a 5′-UTR (corresponding in some cases to the Tobacco Etch Viral leader sequences TEV, SEQ ID NO: 3), a 3′ UTR consisting of two elements called F and I (corresponding is some cases to SEQ ID NO: 7), and a poly(A)-tail of 110 nucleotides in total (A30-Linker-A70 structure). Upon linearization of plasmid DNA as described above, in vitro transcription with T7 RNA polymerase (Thermo Fisher) was performed. This was carried out as described for ModA, but no cap structure was added to the reaction and UTP was substituted for N1-methyl-pseudouridinetriphosphate. RNA was then purified using magnetic particles (Berensmeier 2006), and subsequently Cap1 structure was enzymatically introduced using a commercially available system based on the Vaccinia virus capping enzyme (NEB) and addition of mRNA Cap 2′-O-methyltransferase (NEB). Afterwards, the RNA was subjected to a further purification procedure by Cellulose-based chromatography to remove double-stranded RNA impurities (see Day P R et al, Phytopathology 67:1393 (1977); Morris T J et al., Phytopathology 69:854-858 (1979); and Castillo A et al., Virol J. 8:38 (2011)). RNA concentration and quality were assessed spectrophotometry and analyzed by capillary gel electrophoresis systems, respectively. Presence of dsRNA was assessed in a Northwestern dot-blot assay using dsRNA-specific J2 mAb (English & Scientific Consulting) as described in Karikó et al. Nucleic Acids Res. 39(21):e142 (2011).

mRNA codon optimization: The coding sequence of a protein may influence the efficiency as well as the accuracy of protein translation (see Bossi L et al., Nature. 286(5769):123-7 (1980) and Irwin et al., J Biol Chem. 270(39):22801-6 (1995)).

Therefore, different codon variants of each target were designed and tested. The design of the different codon variants for each target utilized publicly available software from Life Technologies GmbH GeneArt® (Regensburg, Germany) (see Raab D et al., Syst Synth Biol. 4(3):215-25 (2010)) and Eurofins MWG Operon (Ebersberg, Germany). In addition, codon optimization was performed manually editing each codon separately. A GC-content comparable to the wild type sequence was maintained during the optimization process.

Evaluation and selection of constructs was determined by in vitro expression performed using: (i) mRNA lipofection of HEK293T/17 cells and (ii) mRNA electroporation K562 cells.

Forty thousand (40,000) HEK293T/17 cells (ATCC® CRL-11268™) were seeded in flat bottom 96-well plates (VWR International, Cat No. 734-1794) in DMEM, high glucose, GlutaMAX™ (Life technologies, Cat No. 31966047) containing 0.5% FBS (Biochrom, Cat No. S 0115). Seeded cells in 96-well plates were incubated at 37° C., 7.5% CO₂ for 16-18 hours. Adherent HEK293T/17 cells were transfected with RNA using Lipofectamine™ Messenger MAX Reagent (Invitrogen, Cat No. LMRNA) according to the manufacturer's protocol by adding 1.2 μl of the transfection reagent to 20 μl of OptiMEM (Thermo Fisher, Cat No. 31985070) in an RNAse-free 1.5 ml Safe-Lock tube biopur (Eppendorf, Germany, Cat No. 0030121589); in a second tube the indicated RNA was added to 20 μl of OptiMEM. After 10 minutes of incubation the tube containing the RNA was diluted into the tube containing the Lipofectamine™ Messenger MAX and incubated an additional 5 minutes prior to adding 10 μl of the RNA-lipid-complex drop wise to one well of the 96-well plate containing the HEK293T/17 cell layer in 100 μl medium. The 10 μl RNA-lipid-complex contained 5 ng, 25 ng and 100 ng of target RNA respectively. The plates were placed into the incubator for 3 h before an additional 140 μl of fresh medium (DMEM+0.5% FBS) was added. The transfected cells were incubated for 15-18 hours and the supernatants were collected and analyzed for protein content by ELISA as described herein.

K562, a human cell line derived from chronic myeloid leukemia (ATCC® CCL-243™) was cultivated in RPMI 1640 Medium, GlutaMAX™ (Life technologies, Cat No. 61870-044) supplemented with 5% FBS. K562 were electroporated in a 96-well plate system as follows. Cells were washed once in X-VIVO15 medium (Lonza, Cat No. BE02-060Q) and suspended to a final concentration of two hundred fifty thousand (250,000) cells/150 μl in X-VIVO15. A 150 μl of cell suspension was added per well of a 96-well plate containing 5 ng, 25 ng, or 100 ng of RNA. Cells and RNA were mixed and electroporation was performed in a 96 well Gene Pulser MX cell electroporation system from Biorad (250 V, 1×30 ms pulse). Immediately following electroporation, cells were transferred into a new culture plate with fresh medium without antibiotics and rested for 1 hour in the incubator at 37° C. The medium was exchanged for fresh RPMI 1640 GlutaMAX supplemented with 0.5% FCS and incubated 15-18 hours. Supernatants were harvested and analyzed for protein content by ELISA as described herein.

Protein concentrations were determined by ELISAs specific for the RNA encoded cytokine according to the manufacturer's protocol. (i) Human IL-15 sushi/IL-15 sushi R alpha Complex DuoSet ELISA (ii) Mouse IL-12sc Duo Set Development System (DY419-05) (iii) Mouse GM-CSF DuoSet ELISA Development Systems (DY415), all obtained from RnD systems, and mouse IFNα ELISA Kit (TCM) (PBL assay science, 42120-2).

For each mRNA target, the protein expression was evaluated for the wt-sequence and the different codon-optimized variants. Both data sets from lipofection of HEK293T/17 and electroporation of K562 each tested with the three different amounts of modified RNA were considered for selection of the protein coding sequence. A codon-optimized sequence was selected if an at least 1.5-fold increase of protein expression compared to WT sequence was measured. If this was not the case, the WT sequence was selected. For all constructs EarI-restriction sequences were eliminated by mutating the DNA recognition sequence (5′-CTCTTC-3′), while preserving the WT amino acid.

Cell lines: HEK293 (ATCC CRL-1573) cell line and human melanoma cell lines, A101D (ATCC CRL-7798), A375 (ATCC CRL-1619), A2058 (ATCC CRL-11147), and Hs294T (ATCC HTB-140), were obtained from ATCC and cultured in DMEM (ThermoFisher Scientific, Cat 11885-084) supplemented with 10% FBS HI FBS Life Technologies, Cat. 10082) in a humidified atmosphere of 5% CO2 at 37° C.

mRNA transfection: Cells were transfected using Lipofectamine™ MessengerMAX Reagent (Invitrogen, Cat #LMRNA001) according to the manufacturer protocol. Briefly, for each well 0.3 μl of the transfection reagent was diluted with 5 μl of the Opti-MEM™ media (Life Technologies, Cat. 31985062) and incubated for 10 min at room temperature; mRNA mixtures were diluted with Opti-MEM™ media (5 μl per well) and mixed with diluted MessengerMax reagent, incubated for 5 min at room temperature and aliquoted to the 96-well plate. Cells were diluted in complete growth media and 40,000 cells per well were added to the transfection mixtures. Cells were incubated for 24 hours at 37° C. in a CO₂ incubator then media was collected and cytokine concentration was determined by Meso Scale Discovery (MSD) assay.

Meso Scale Discovery Assay: Cytokine concentration was determine using MSD assays: Proinflammatory Panel 1 (human) MSD kit (catalog N05049A-1) for IL12p70, Cytokine Panel 1 (human) MSD kit (catalog N05050A-1) for GM-CSF and IL-15 sushi, and Human IFN-α 2a Ultra-sensitive Kit (catalog N05050A-1) for IFNα. Data were analyzed using MSD Discovery Workbench V. 4.0.12 software and GraphPad Prism V.6.00 software.

PBMC isolation and treatment: Peripheral Blood Mononuclear Cells (PBMCs) were isolated by density gradient media (Ficoll-Paque) from a leukopak (Research Blood Components). 600,000 PBMCs were added per well of a 96-well plate. Cells were treated with a cytokine mixture for 24 hrs and IFNγ production was measured in the cell culture media using Human IFNγ 384-Tissue Culture MSD Assay.

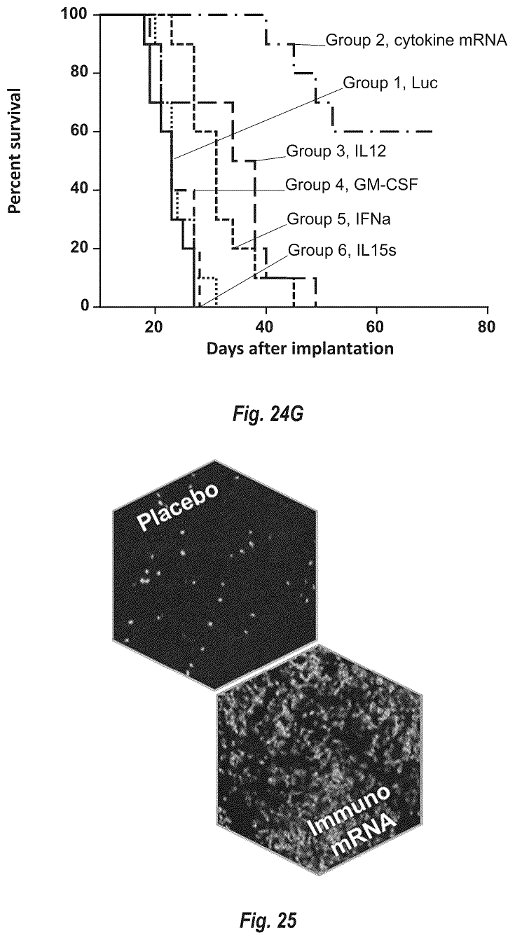

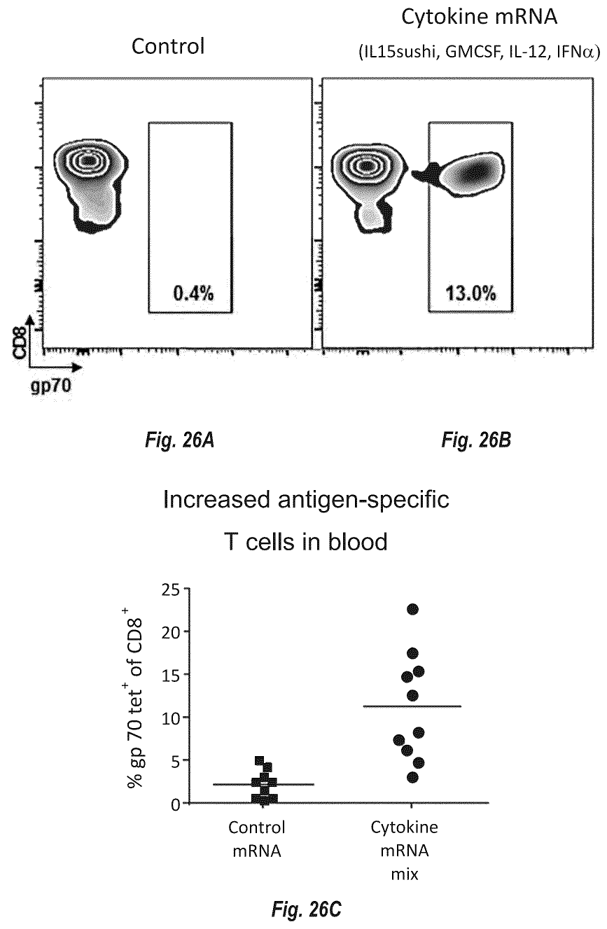

Measuring CD8 response: B16F10 tumor bearing mice received a single intratumoral injection of Immuno mRNA (IFNα, IL-15 sushi, GM-CSF, and IL-12sc, ModB) or control luciferase mRNA (Placebo). Seven days after intratumoral mRNA injection tumors were excised, processed for immunofluorescence and stained with an antibody for CD8 (gray). As shown in FIG. 25, CD8 cells were present after cytokine mRNA intratumoral injection.

Preparation of mRNA for in vivo studies: The respective mRNA mixtures were prepared for in vivo studies by mixing equal quantities (micrograms) of mRNA in water at 2× the intended dose. The mRNA mixture was frozen at −80 C until the day of intratumoral injection. On the day of injection, mRNA was thawed and mixed with an equal volume of 2× sterile Ringer's solution. The resulting 1× mRNA/Ringer solution was used for intratumoral injection.

Gene expression analysis: A375 tumors were homogenized in the RLT Buffer (Qiagen) using Precellys 24 homogenizer (Bertin Instruments). Total RNA was isolated with the RNeasy-96 Kit (Qiagen), following the spin protocol with the DNase treatment. RNA was eluted with nuclease-free water and quantified by ultraviolet absorbance using a NanoDrop 8000 (Thermo Scientific). cDNA synthesis was performed with the High Capacity RNA to cDNA Kit (Applied Biosystems) according to the manufacturer's recommendations. Real-time PCR were performed on a ViiA™7 (Applied Biosystems) according to standard protocol. Amplification was performed using the TaqMan Gene Expression Master Mix (Applied Biosystems) and predesigned Taqman Assays (Applied Biosystems). Gene expression was normalized to the endogenous control GAPDH. Comparative ddCT method was used to evaluate gene expression.

TABLE 3

| | | Lot | Fluorescent |
|---|---|---|---|
| Gene | Assay ID | Number | dye |
| ISG15 | Hs01921425_s1 | 1548867 | FAM |
| ISG54 | Hs01922738_s1 | 1532771 | FAM |
| Mx1 | Hs00895608_m1 | 1539223 | FAM |
| hGAPDH | 4326317E | 1311049 | VIC |

### Example 2—Combinations of Three mRNAs Reduce Tumor Volume In Vivo

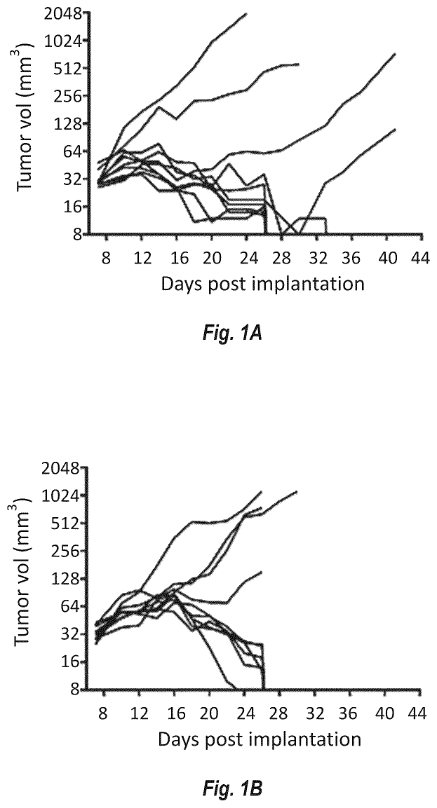

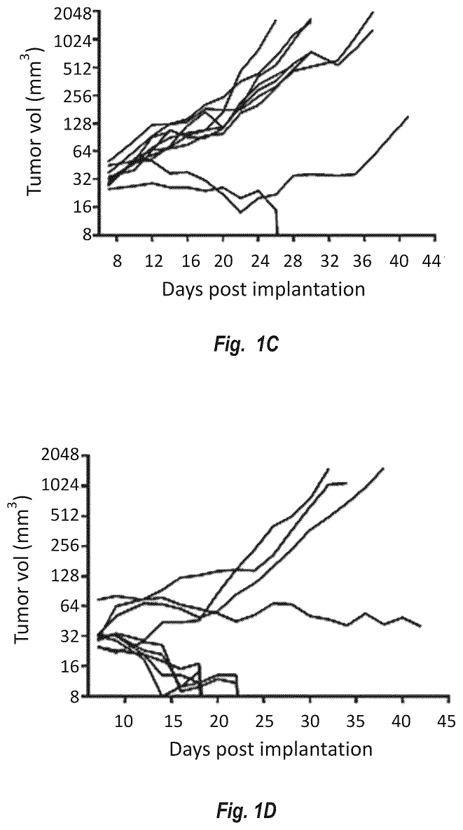

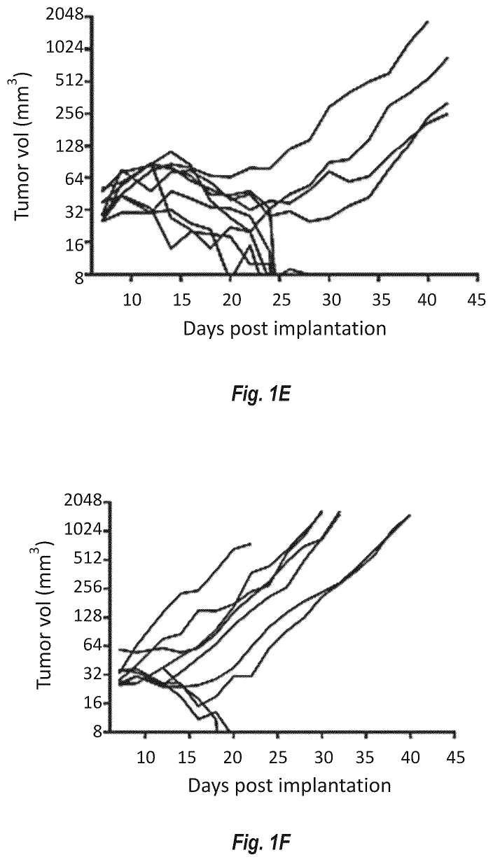

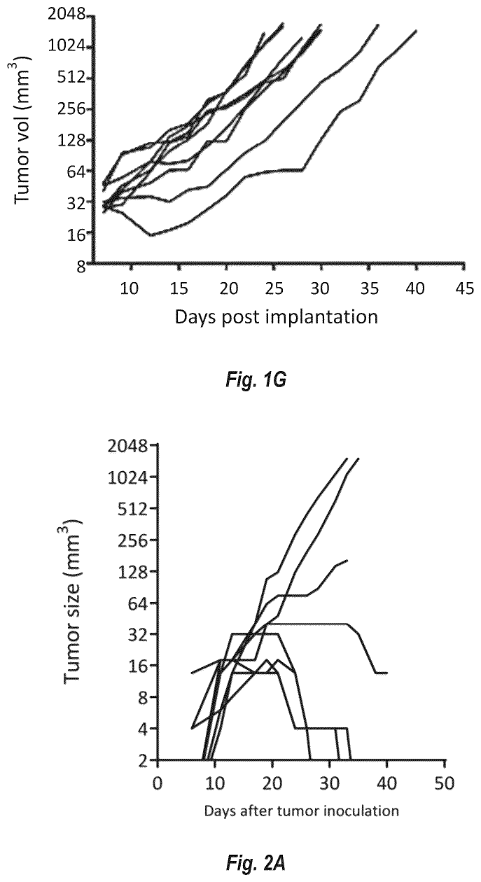

A mixture of modified mRNAs encoding GM-CSF, IL-2, and IL-12sc was injected into B16F10 tumor bearing mice and tumor growth was monitored to day 41. As shown in FIG. 1, intratumoral injection of a combination of three mRNAs encoding GM-CSF, IL-2, and IL-12sc having ModA (SEQ ID NOs: 32, 38, and 56; FIG. 1A), or a combination of three mRNAs encoding GM-CSF, IL-2, and IL-12sc having ModB (SEQ ID NOs: 35, 41, and 59; FIG. 1B) induced regression in 6 out of the 10 mice, while mice treated with a control mRNA encoding luciferase (ModA) displayed tumor regression in 1 of 10 animals (FIG. 1C). These data were confirmed in a repeat study of similar design (FIG. 1D-1G). In the repeat experiment, a mixture of cytokine mRNA (GM-CSF, IL-2, IL-12sc) with ModA or ModB lead to tumor regression in 5 of 9 mice, while treatment with control mRNA in ModA and ModB displayed 2 of 9 and 0 of 9 tumor regressions, respectively.

The effects of cytokine mRNA treatment were evaluated in CT26 tumors. Mice with established CT26 tumors were injected with a cytokine mRNA mixture encoding GM-CSF, IL-2, and IL-12sc in ModA and ModB formats, respectively. Two control groups were included: i) mRNA Ringer's diluent and ii) ModA mRNA encoding firefly luciferase. A total of 6 intratumoral injections were administered on days 19, 21, 24, 26, 28 and 31. As shown in FIG. 2, both GM-CSF, IL-2, and IL-12sc mRNA ModA (SEQ ID NOs: 56, 32, and 38; FIG. 2A) and ModB (SEQ ID NOs: 59, 35, and 41; FIG. 2B) resulted in tumor regression in 5 and 6 out of 8 mice, respectively, while no tumors treated with control mRNA in ModA (FIG. 2C) or Ringer's solution (FIG. 2D) displayed tumor regression.

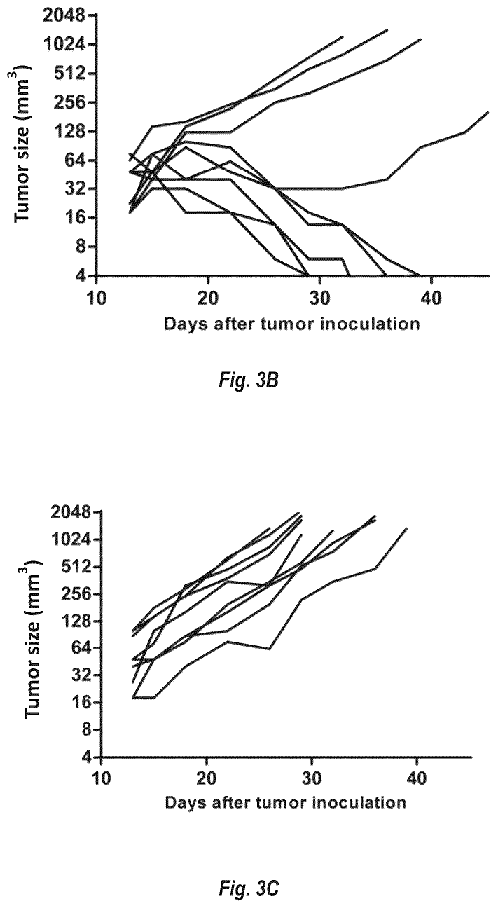

The cytokine mRNA mixture encoding IL-15 sushi, GM-CSF and IL-12sc (ModB; SEQ ID NOs: 53, 59, and 41) and IL-2, GM-CSF and IL-12sc (ModB; SEQ ID NOs: 35, 59, and 41) were evaluated for anti-tumor activity in the CT26 tumor model. Tumors received intratumoral mRNA injections on days 13, 15, 18, 20 and 22 after tumor inoculation. As shown in FIG. 3, intratumoral injection of either the IL-2 mixture (FIG. 3A) or IL-15 sushi mixture (FIG. 3B) resulted in tumor regression in 5 out of 10 or 11 tumor-bearing animals, respectively (FIGS. 3A and B), whereas in the control group injected with luciferase mRNA (ModB) no tumor regression was observed (FIG. 3C).

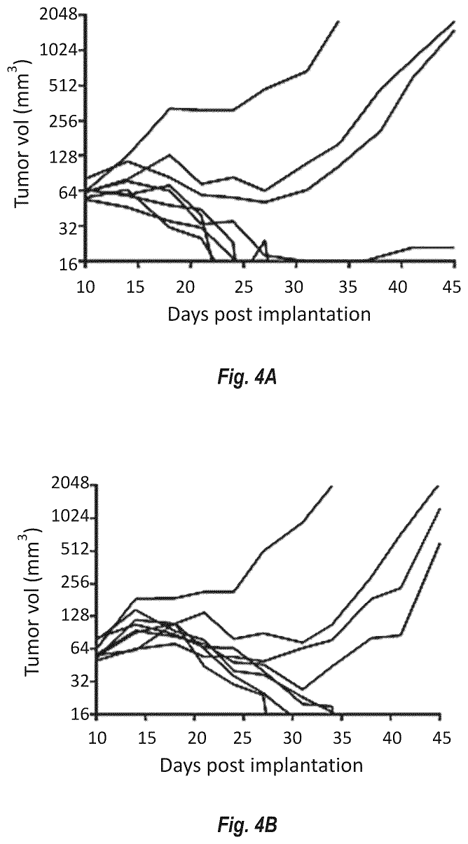

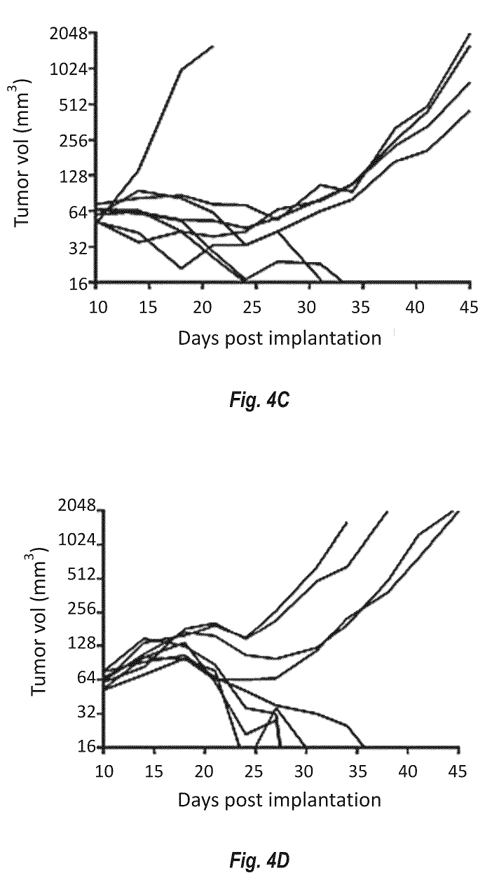

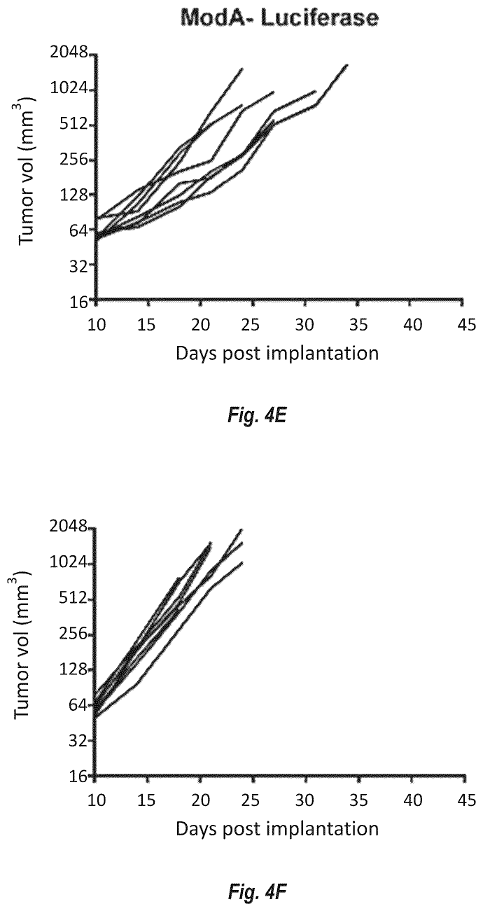

Anti-tumor activity of the mRNA mixture of IL-2, IL-12sc, and GM-CSF (FIG. 4A-ModA [SEQ ID NOs: 32, 38, and 56], 4B-ModB [SEQ ID NOs: 35, 41, and 59]), or IL-15 sushi, IL-12sc, and GM-CSF or (FIG. 4C-ModA [SEQ ID NOs: 50, 38, and 56], 4D-ModB [SEQ ID NOs: 53, 41, and 59) was further evaluated in the B16F10 tumor model. Mice with B16F10 tumors were injected intratumorally with mRNA on days 11, 13, 15, and 17. Treatment of B16F10 tumors with the cytokine mRNA mixture that encoded IL-15 sushi, IL-12sc, GM-CSF resulted in tumor regression in 3 and 4 of 8 mice in ModA and ModB, respectively. Tumors treated with an mRNA mixture of IL-2, IL-12sc, and GM-CSF (ModA or ModB) had tumor regression in 4 out of 8 mice, while no tumor regression was noted for mice treated with ModA or ModB mRNA encoding luciferase (FIG. 4E—ModA, FIG. 4F—ModB).

### Example 3—Combinations of Four mRNAs Reduce Tumor Volume In Vivo

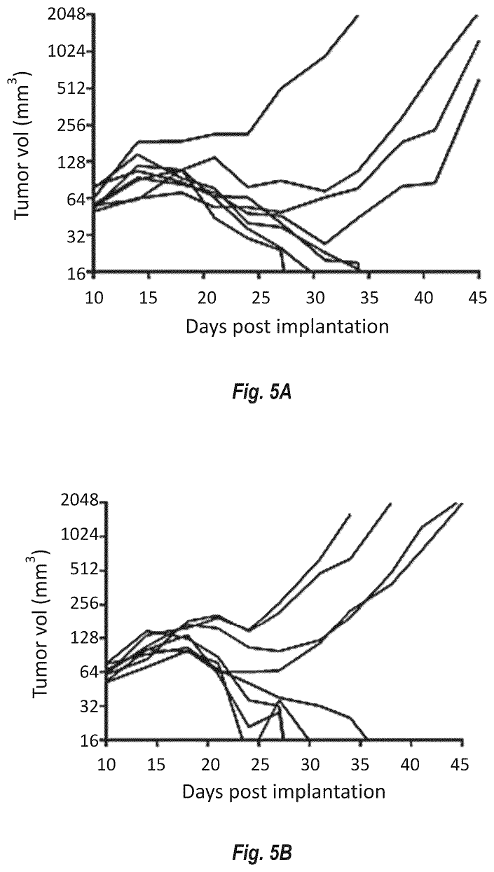

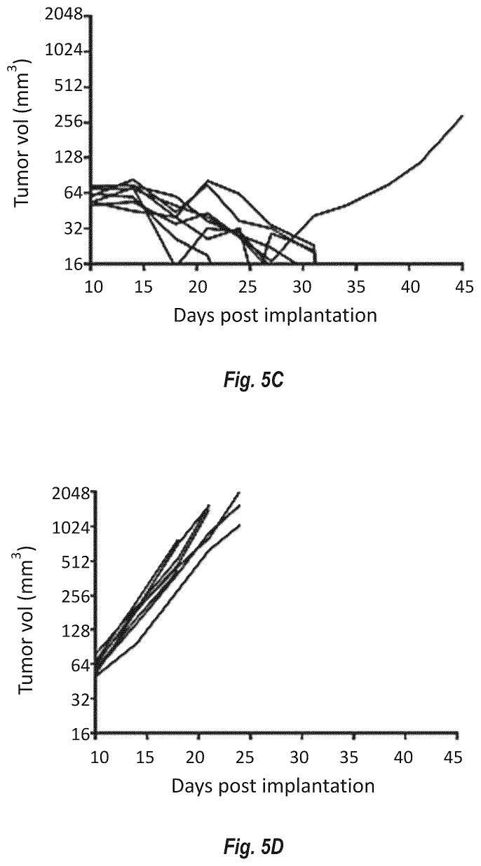

We next tested the effect of adding a fourth mRNA to the cytokine mRNA mixture. B16F10 tumor bearing mice received four intratumoral injections of ModB cytokine mRNA mixture encoding: i) GM-CSF, IL-2, IL-12sc (SEQ ID NOs: 59, 35, and 41; FIG. 5A), ii) GM-CSF, IL-15 sushi, IL-12sc (SEQ ID NOs: 59, 53, and 41; FIG. 5B) and iii) GM-CSF, IL-15 sushi, IL-12sc, IFNα (SEQ ID NOs: 59, 53, 41, and 47; FIG. 5C), and tumor growth was monitored to day 45. Each of the cytokine mRNA mixtures had an anti-tumor effect with 4 out of 8 tumors regressing following intratumoral injection of cytokine mRNA mixtures of GM-CSF, IL-2, and IL-12sc or GM-CSF, IL-15 sushi, and IL-12sc, and 7 out of 8 tumors regressed upon treatment with GM-CSF, IL-15 sushi, IL-12sc, and IFNα. Mice treated with control mRNA (ModB) exhibited no tumor regression (FIG. 5D).

The anti-tumor activity of GM-CSF, IL-2, IL-12sc, and IFNα was examined in three different murine in vivo tumor models, CT26, B16F10 and MC38. Tumor bearing mice received 4-6 intratumoral injections of ModB cytokine mRNA encoding IL-2, IL-12sc, GM-CSF and IFNα (SEQ ID NOs: 35, 41, 59, and 47) or a control ModB mRNA encoding firefly luciferase. Anti-tumor activity was assessed in each tumor model. Mice treated with this combination of cytokine mRNA had 4/8, 7/8 and 5/5 regressing tumors in the CT26 (FIG. 6A), B16F10 (FIG. 6C) and MC38 (FIG. 6E) models, respectively. No tumor regression was observed in the comparator tumor bearing mice treated with luciferase mRNA (FIGS. 6B [CT26], 6D [B16F10], and 6F [MC38]).

The anti-tumor activity of a four-component cytokine mRNA mixture encoding each of IL-2, IL-12sc, GM-CSF and IFNα (ModB, SEQ ID NOs: 35, 41, 59, and 47) or IL-15 sushi, IL-12sc, GM-CSF and IFNα (ModB, SEQ ID NOs: 53, 41, 59, and 47) was evaluated in the CT26 tumor model. Tumor bearing mice were treated with 6 intratumoral injections and tumor growth was monitored. Treatment with both IL-2, IL-12sc, GM-CSF and IFNα (FIG. 7A) and IL-15 sushi, IL-12sc, GM-CSF and IFNα (FIG. 7B) effectively induced tumor regression in 4/8 and 8/8 mice, respectively, while no tumor regressions were observed for mice treated with the control mRNA (FIG. 7C). These data were confirmed in a repeat study of similar design (FIG. 7D-F).

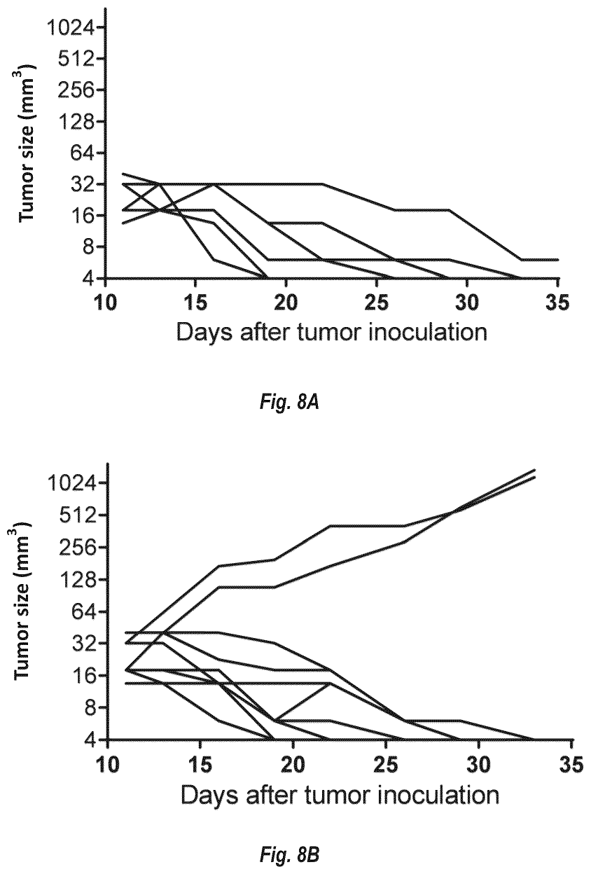

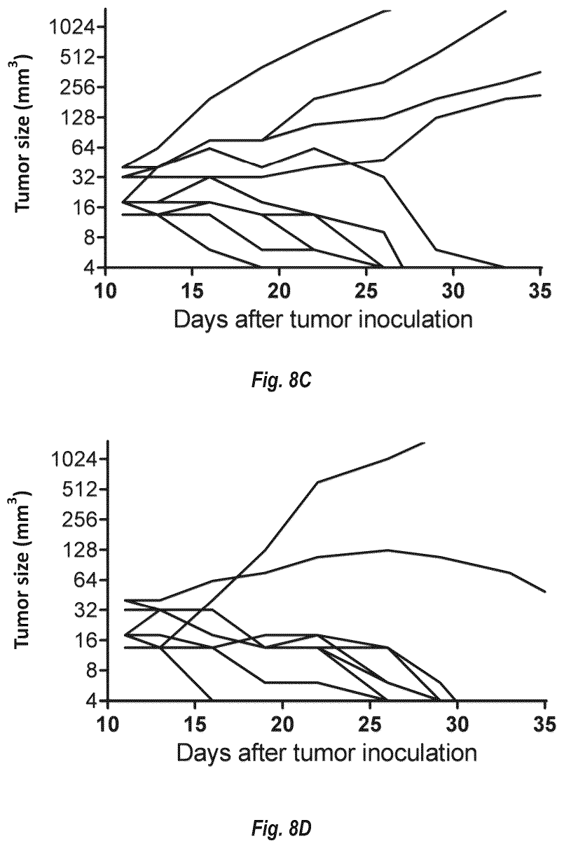

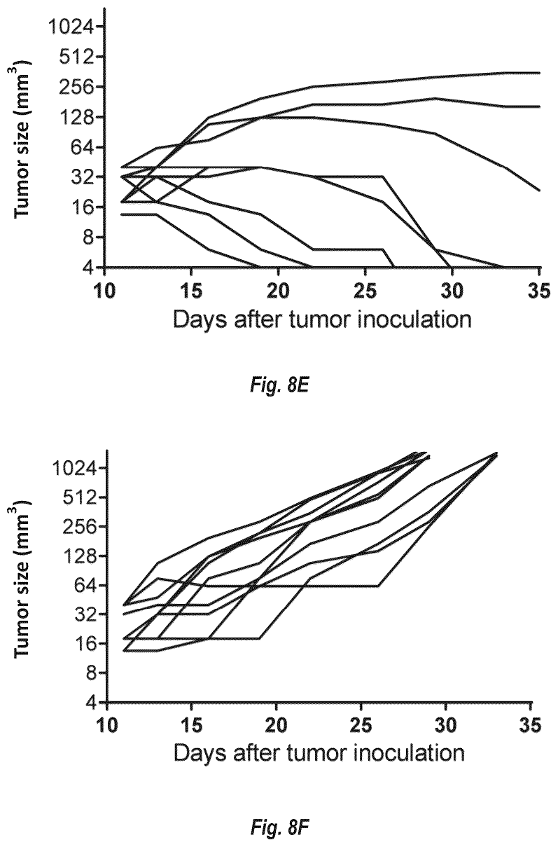

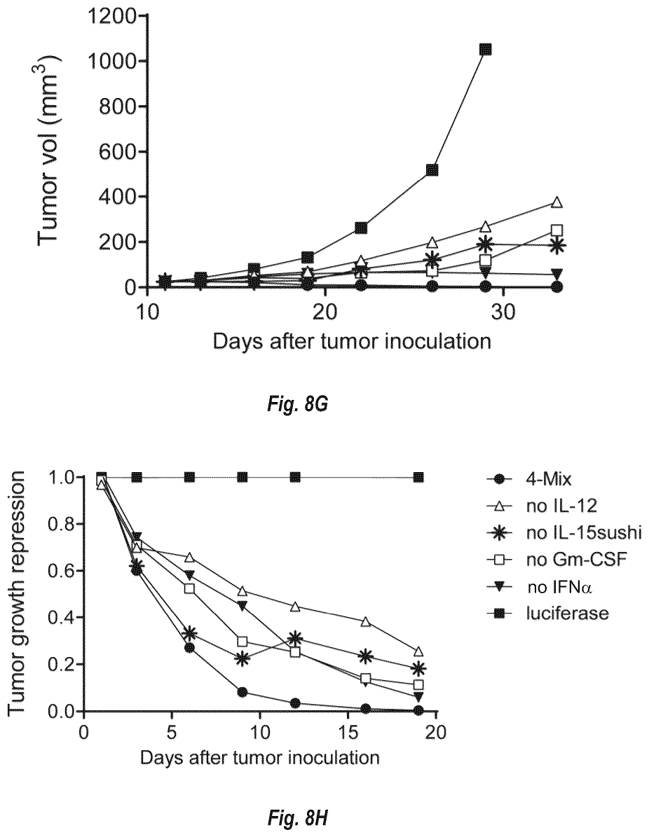

A study in CT26 tumor model was conducted in which individual components were systematically removed from the mRNA mixture of IL-15 sushi, IL-12sc, GM-CSF and IFNα (ModB, SEQ ID NOs: 53, 41, 59, and 47). CT26 tumors were injected with cytokine mRNA on days 12, 15, 19 and 22 after inoculation. Tumors treated with four injections of mRNA encoding IL-15 sushi, IL-12sc, GM-CSF and IFNα induced regression in all 10 treated tumors (FIG. 8A). Tumors treated with the ModB mRNA mixtures of i) IL-15 sushi, IL-12sc and IFNα, ii) IL-15 sushi, GM-CSF and IFNα, iii) IL-12sc, GM-CSF and IFNα, iv) IL-15 sushi, IL-12sc, and GM-CSF resulted in regression of 8, 6, 8, 7 out of 10 tumors, respectively (FIG. 8B-E). No tumors treated with control mRNA (ModB) displayed tumor regression (FIG. 8F). To analyze tumor growth kinetics, mean tumor volumes were calculated for each treatment group up to day 33 (FIG. 8G). The smallest mean tumor volume was observed for mice treated with the mixture of IL-15 sushi, IL-12sc, GM-CSF and IFNα, while the largest mean tumor volume was observed in the luciferase treated animals. Tumor growth repression T/C (Tumor/Control based on mean tumor volume) was plotted to day 19 (FIG. 8H) for each of the treatment groups. The four-component mixture of IL-15 sushi, IL-12sc, GM-CSF and IFNα exhibited the largest T/C.

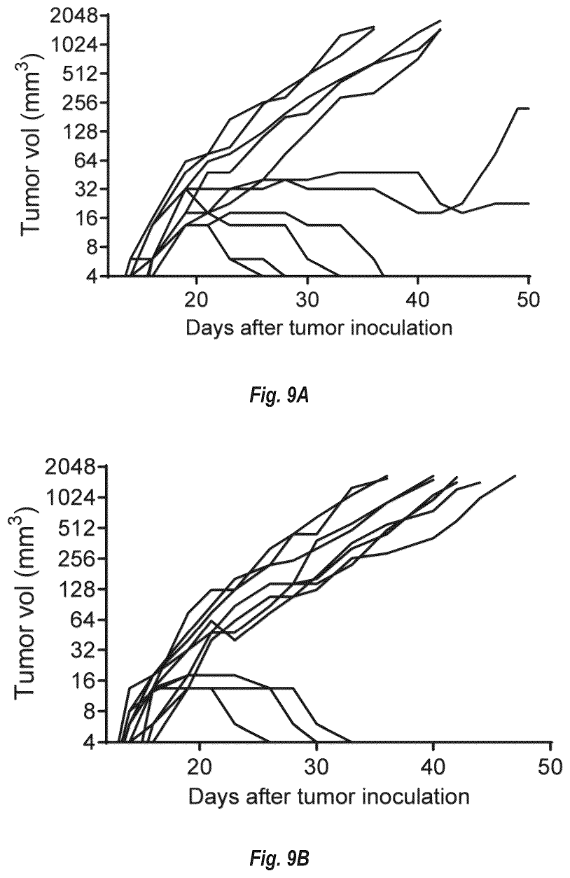

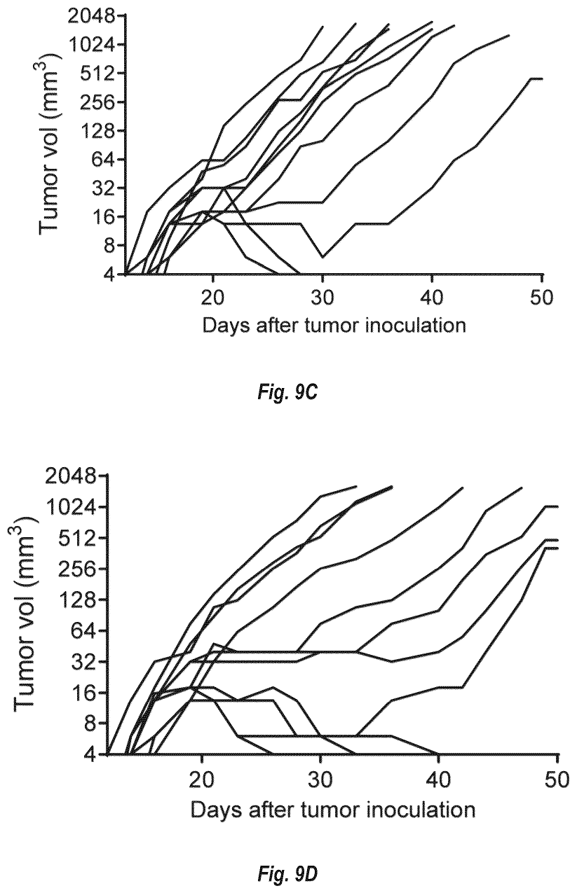

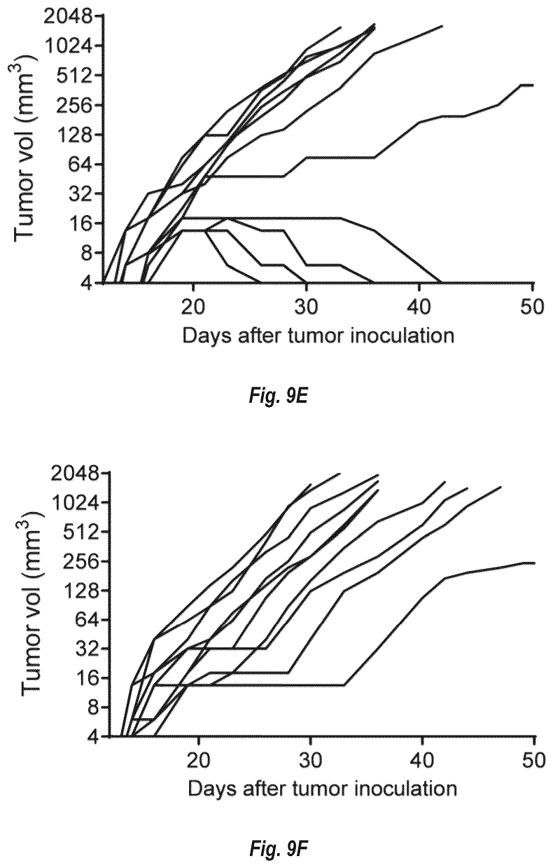

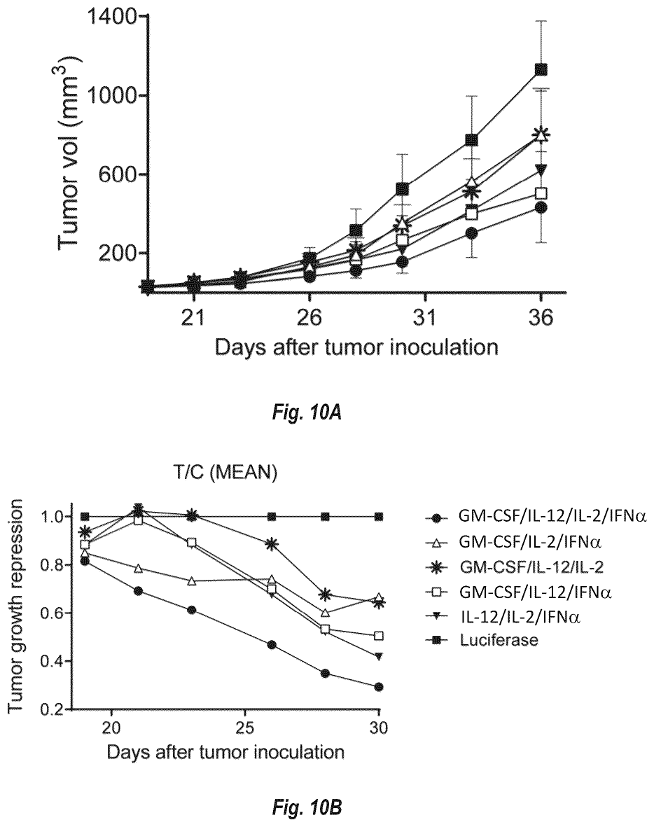

Analogous to the study shown in FIG. 8, a study was conducted in which individual components were systematically removed from the mRNA mixture of IL-2, IL-12sc, GM-CSF and IFNα (ModB; SEQ ID NOs: 35, 41, 59, and 47). CT26 tumors received 6 intratumoral mRNA injections on days 19, 21, 23, 26, 28 and 30. Treatment with the cytokine mRNA mixture of IL-2, IL-12sc, GM-CSF and IFNα resulted in tumor regression in 4 of 10 animals led to complete tumor regression in 4 animals and stable disease beyond day 40 in two individuals (FIG. 9A). Tumors treated with the ModB 3-component mRNA mixtures of i) IL-2, IL-12sc, and IFNα, ii) IL-2, GM-CSF and IFNα, iii) IL-12sc, GM-CSF and IFNα, and iv) IL-12sc, GM-CSF and IL-2 resulted in regression of 2, 3, 3 and 4 tumors, respectively (FIG. 9B-E) and CT26 tumors treated with a control luciferase mRNA displayed no tumor regression (FIG. 9F).

To analyze tumor growth kinetics, mean tumor volumes were calculated for each treatment group up to day 36. The smallest mean tumor volume was observed for mice treated with the mixture of IL-2, IL-12sc, GM-CSF and IFNα, while the largest mean tumor volume was observed in the luciferase treated animals (FIG. 10 A). Tumor growth repression T/C (Tumor/Control based on mean tumor volume) was plotted to day 30 (FIG. 10B) for each of the treatment groups. The four-component mixture of IL-2, IL-12sc, GM-CSF and IFNα exhibited the largest T/C.

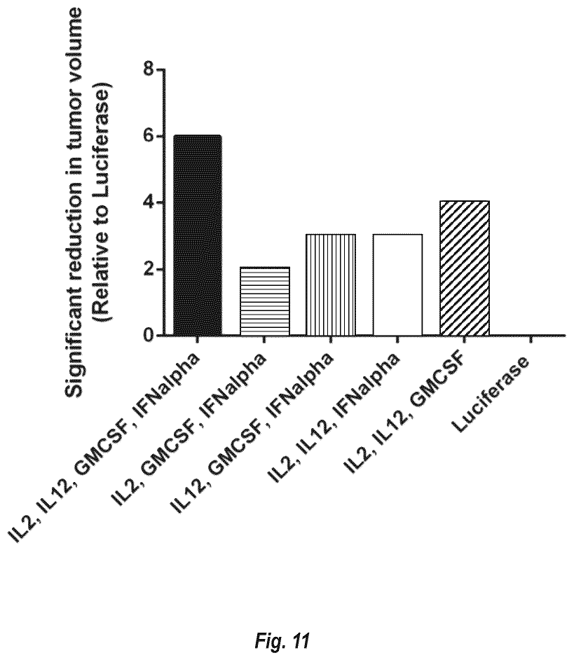

The anti-tumor response from the different cytokine mRNA mixtures in FIG. 9 were further analyzed as follows: The tumor volume change (ΔV) as defined by the difference in tumor volume between the measurement at the end of experiment or termination (Vₜ) and the measurement at the beginning of the experiment (V_{b}) (ΔV=Vₜ−V_{b}), was calculated for all mice in each of the six treatment groups, including mice treated with 4 component cytokine mRNA mixture (IL-2, IL-12sc, GM-CSF and IFNα), mice treated with 3 component mixture missing one of the four cytokines and mice treated with control luciferase mRNA. Mean (μ) and standard deviation (σ) of a trimmed data (removing top and bottom 10% of the original data) on tumor volume change was calculated for control luciferase group and the Shapiro-Wilk normality test of this trimmed data showed it approximately followed a normal distribution. A Z score (Z=(ΔV−μ)/σ) was then calculated for tumor volume change data in each individual mouse from all treatment groups. A ratio (R) between each tumor volume change value and the trimmed mean of control group was calculated (R=ΔV/μ). Given the definitions, we consider the Z score as “significance of tumor volume reduction” and R as “extent of tumor volume reduction”. To identify a mouse that shows a significantly smaller tumor volume increase than that in control luciferase group (in other words “significant tumor reduction” as compared with control group), a cutoff of Z<=−1.645 (p(Z<=−1.645)=0.05) and R<=0.15 was applied. The results showed that the number of mice in each treatment group that has significantly smaller tumor volume increase than control group as follows: i) 6 out of 8 mice for the cytokine mRNA mixture of IL-2, IL-12sc, GM-CSF and IFNα (ModB). ii) 2 of 8 mice for IL-2, GM-CSF and IFNα (ModB), iii) 3 of 8 for IL-12sc, GM-CSF and IFNα and IL-2, IL-12sc, and IFNα and iv) 4 of 8 mice treated with the cytokine mRNA mixture of IL-2, IL-12sc, and GM-CSF (ModB) (FIG. 11).

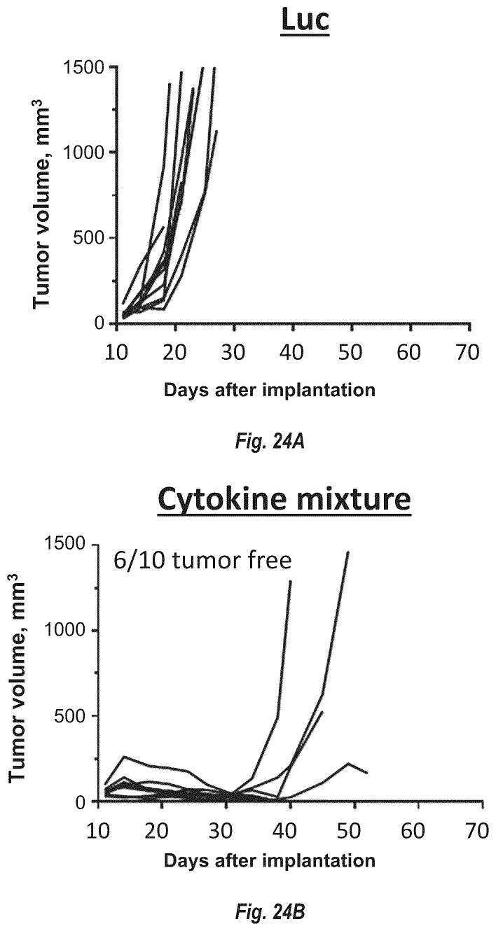

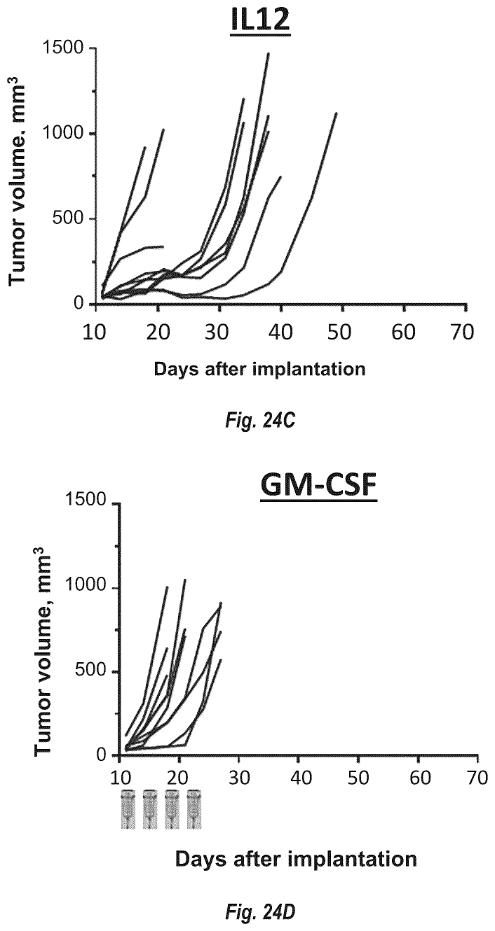

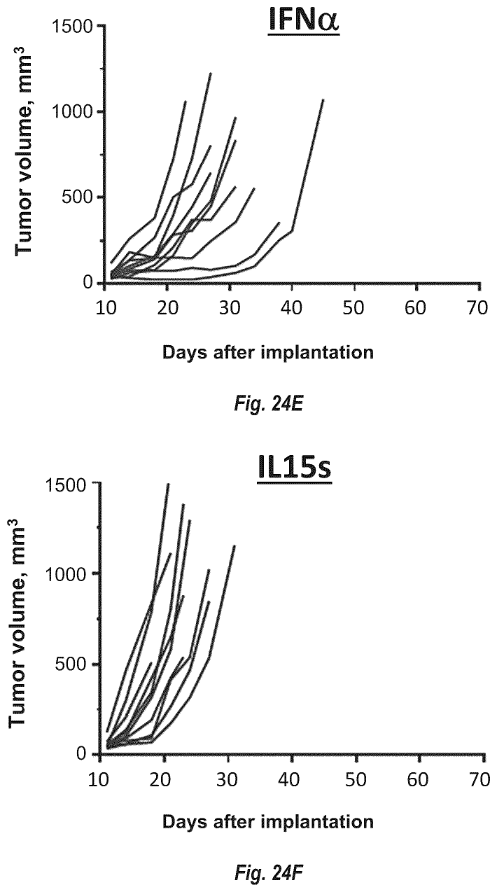

Female C57BL/6J mice were implanted with B16F10 cells as described above. Mice were treated with 4 intratumoral injection (8 μg mRNA/2 μg per target) on days 11, 15, 19, and 23 with ModB cytokine mRNA mixture (IL-15 sushi, IL-12sc, GM-CSF, IFNα) or control luciferase mRNA. Treatment with the 4-component mixture of cytokine mRNA resulted in tumor rejection in 6/10 treated mice. See, FIG. 24B. In comparison, no tumor free mice were observed in any of the groups treated with a single mRNA (FIGS. 24C-F). The combination of IL-15 sushi, IL-12sc, GM-CSF, IFNα led to increased overall survival with 60% of the mice tumor free at Day 70, while all tumors of mice treated with a single cytokine mRNA progressing to the stage where the animals needed to be euthanized (FIG. 24G). Luciferase control is shown in FIG. 24A.

### Example 4—Cytokine mRNA Protects Against Tumor Re-Challenge

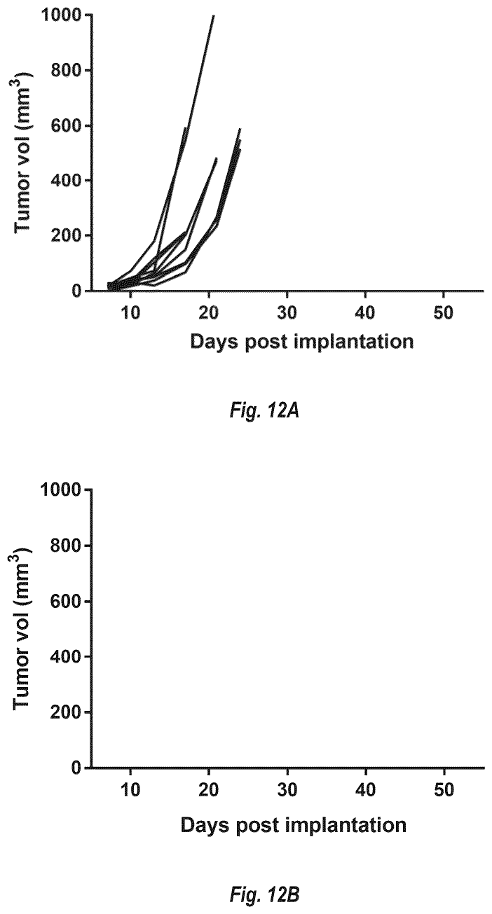

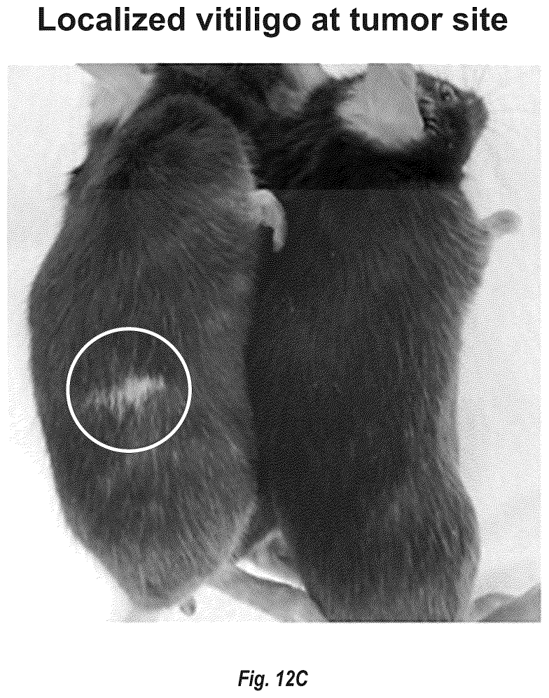

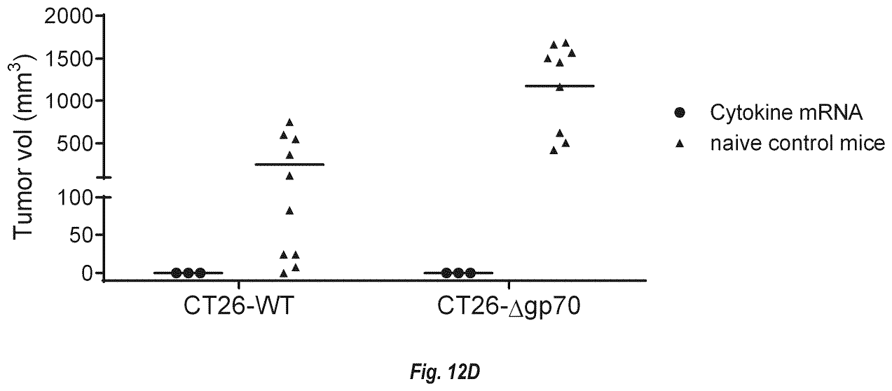

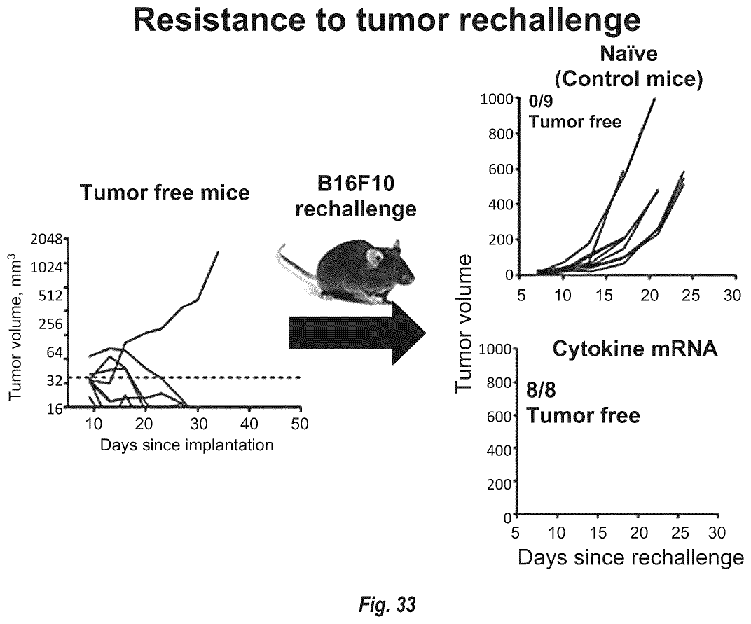

To evaluate the effect of the cytokine mRNA mixture on the development of immunological memory, re-challenge experiments were performed. Briefly, B16F10 tumor bearing mice were treated with a cytokine mRNA mixture of IL-15sushi, IL-12sc, GM-CSF, and IFNα (Mod B; SEQ ID NOs: 53, 41, 59, and 47). A portion of the cytokine mRNA treatment B16F10 tumors completely regressed leading to tumor free animals. These tumor free animals were then re-challenged with B16F10 cells as a way to assess adaptive immune memory and 9 naïve mice were implanted with B16F10 tumor cells as a positive control for tumor engraftment (FIG. 12A). All 9 naïve mice engrafted with B16F10 cells developed tumors, whereas all eight tumor-free mice rejected the B16F10 cells and did not exhibit growth of B16F10 tumors (FIG. 12B). A portion of mice previously treated with cytokine mRNA develop localized vitiligo at the original site of the tumor (FIG. 12C). This experiment was essentially repeated and results are shown in FIG. 33.

To evaluate the effect of the cytokine mRNA mixture on the development of immunological memory, a re-challenge experiment was performed using the CT26 tumor model. Therefore, CT26 tumor bearing mice were treated with a cytokine mRNA mixture of IL-15sushi, IL-12sc, GM-CSF, and IFNα (Mod B; SEQ ID NOs: 53, 41, 59, and 47). A portion of the cytokine mRNA treatment CT26 tumors completely regressed leading to tumor free animals. Three tumor free animals were then re-challenged with CT26 tumor cells and three tumor free animals were then re-challenged with CT26 tumor cells, in which the gp70 epitope (CT26-Δgp70) was knocked out. 9 and 10, respectively, naïve mice were implanted with CT26 tumor cells and CT26-Δgp70 as a positive control for tumor engraftment. On day 21 after tumor inoculation 8 out of 9 naïve mice had engrafted with CT26 tumor cells developed tumors and all 10 naïve mice engrafted with CT26-Δgp70 tumor cells developed tumors whereas all three tumor-free mice in each group rejected the CT26 and CT26-Δgp70 cells and did not exhibit growth of CT26 tumors and CT26-Δgp70, respectively (FIG. 12D). This experiment shows that immunological memory upon cytokine mRNA injection in the CT26 tumor model is not restricted to T-cells specific for the immunodominant epitope gp70.

### Example 5—Systemic Anti-Tumor Activity of Cytokine mRNA

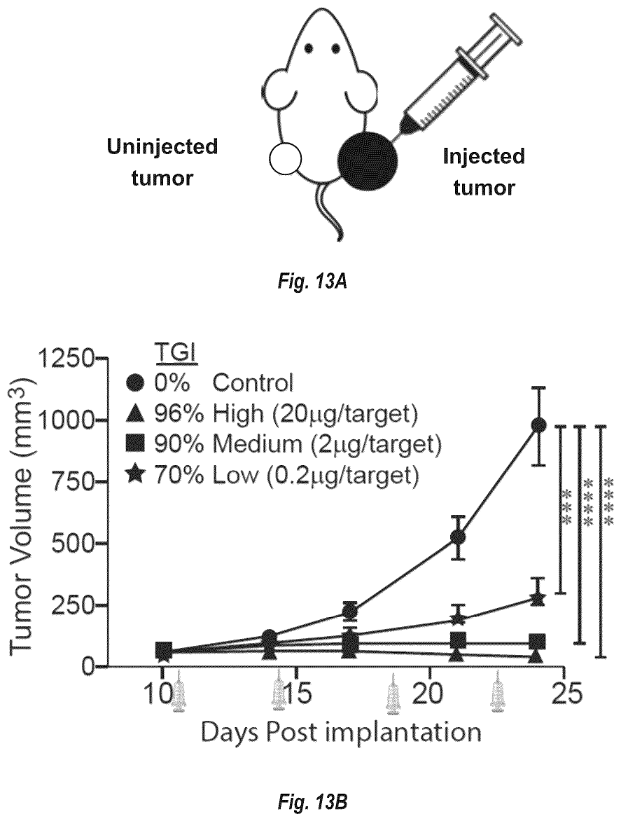

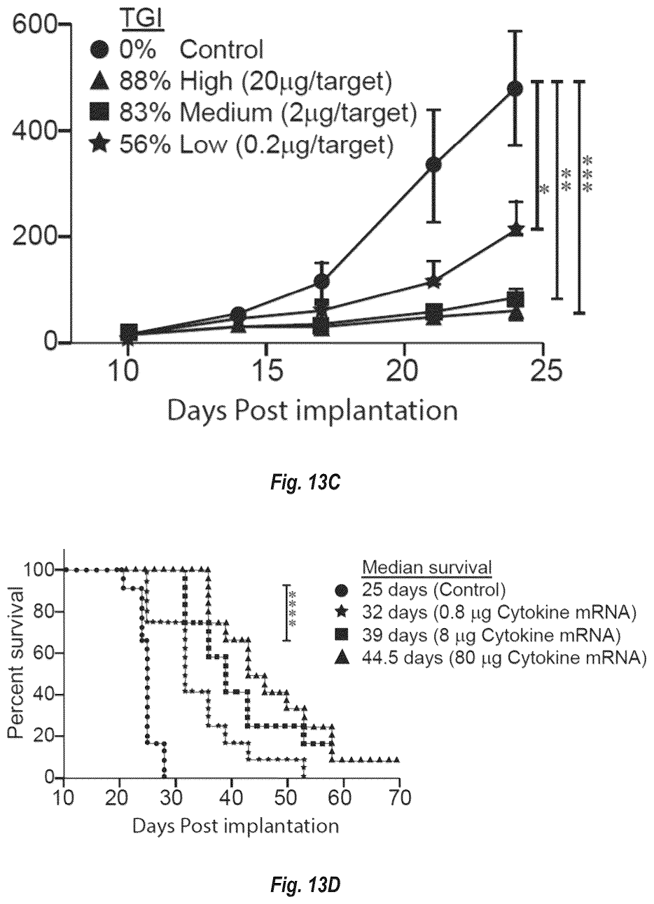

To evaluate the ability of local intratumoral cytokine mRNA to exert a systemic anti-tumor response, mice were engrafted with B16F10 tumor cells on both the left and right flanks (FIG. 13A). Mice bearing bilateral B16F10 tumors received four intratumoral injections with control mRNA encoding luciferase or a cytokine mRNA mixture encoding IL-15 sushi, IL-12sc, GM-CSF and IFNα (ModB; SEQ ID NOs: 53, 41, 59, and 47). The right tumor was injected with mRNA at three different dose levels (80 μg, 8 μg, and 0.8 μg mRNA corresponding to 20 μg, 2 μg and 0.2 μg mRNA/target), while tumors on the left flank were untreated. Dose dependent anti-tumor activity was observed in both the injected (FIG. 13B) and uninjected (FIG. 13C) tumors with tumor growth inhibition ranging from 88% in the uninjected tumor to 96% in the injected tumor. Groups treated with cytokine mRNA treatment had increased median survival compared to groups treated with the Luciferase control mRNA (FIG. 13D).

To further evaluate the ability of local intratumoral cytokine mRNA to exert a systemic anti-tumor response, mice were engrafted with B16F10 tumor cells on the right flank and received an IV injection of Luciferase-expressing B16F10 cells for induction of tumors in the lung (FIG. 19A). On day 11, 14 and 18 after SC tumor implantation mice bearing B16F10 tumors received in total three intratumoral injections with cytokine mRNA mixture of IL-15 sushi, IL-12sc, GM-CSF and IFNα (ModB; SEQ ID NOs: 53, 41, 59, and 47) into the flank tumor only, while tumors in the lung were untreated. The control group received intratumoral injection of equal amounts of control mRNA without any coding sequence. Mice were sacrificed for endpoint analysis on day 20, at which time lungs were taken out and weighed. FIG. 19B shows exemplarily bioluminescence measurements of four animals and pictures of the according lungs taken out in order to visualize the dark tumor nodes. Tumor growth of SC tumors was strongly suppressed by injection of cytokine mRNA mixture, whereas tumors injected with control mRNA grew progressively as depicted in FIG. 19C showing mean tumor volume of 15 mice in each group. Lung tumor growth was suppressed in animals which received intratumoral cytokine mRNA injection in SC tumors when compared to animals treated with control mRNA; FIG. 19D shows total flux analysis of bioluminescence measurements of all 15 animals per group on day 20, which is a correlate for tumor burden due to Luciferase-expressing tumor cells; line indicates median and asterisk indicates p<0.05 analyzed by T-test. Additionally, lungs of animals treated with cytokine mRNA had significantly less weight (FIG. 19E, line indicates median). Higher weight of lungs of animals treated with control RNA resulted from higher tumor burden.

### Example 6—Human Cytokine mRNA

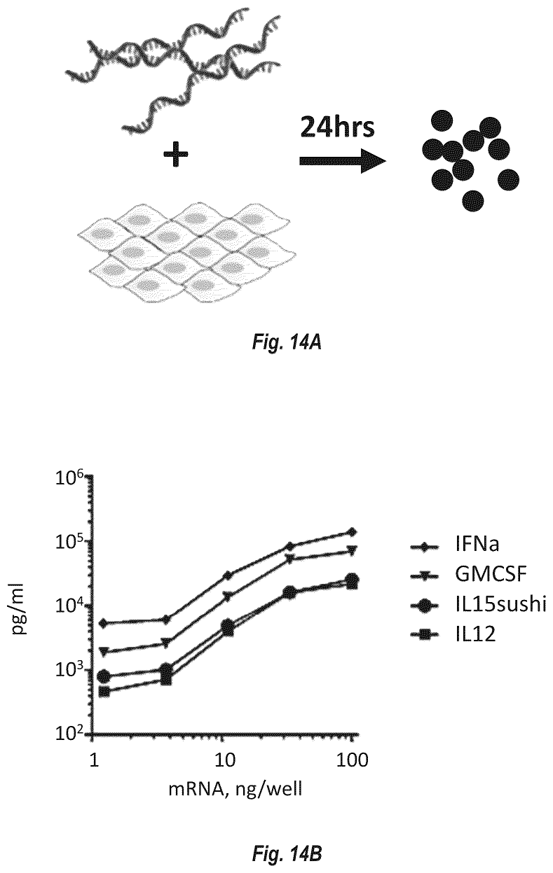

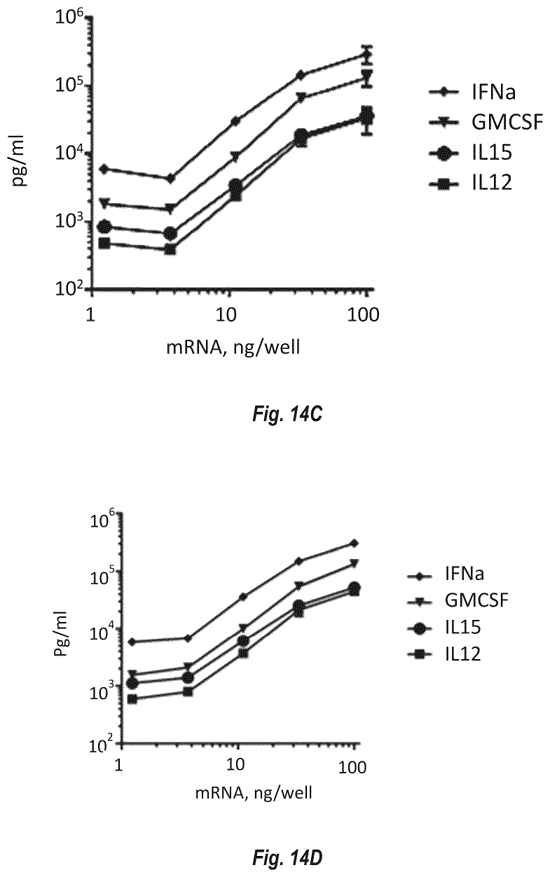

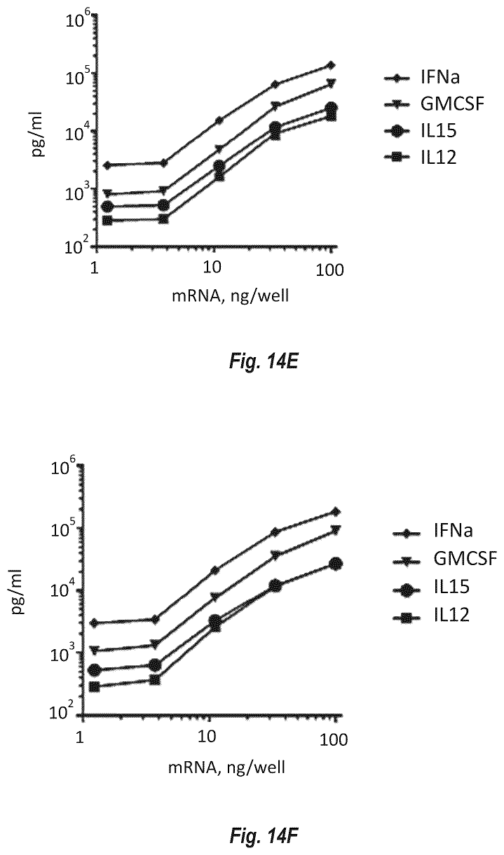

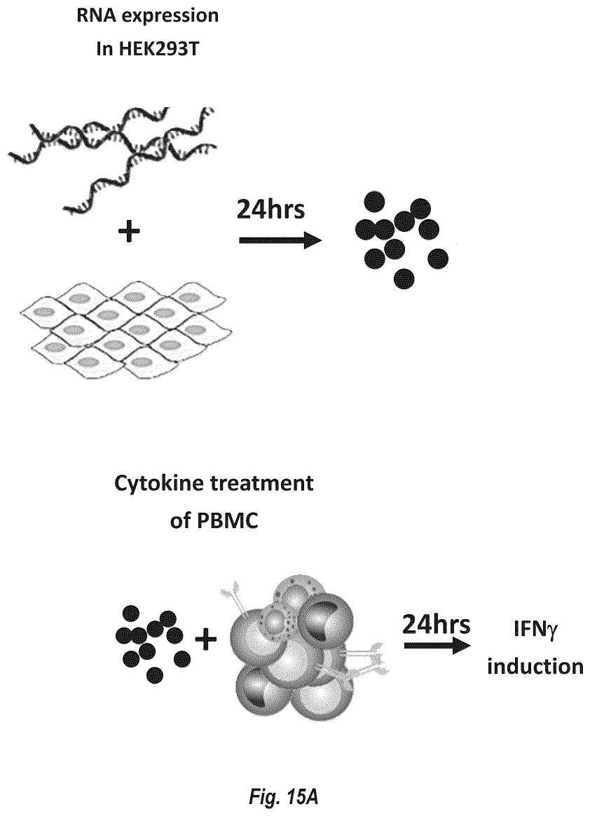

To evaluate in vitro expression of the human cytokine mRNA, an mRNA mixture encoding the human cytokines IL-15 sushi, IL-12sc, GM-CSF, and IFNα2b (SEQ ID Nos: 26, 18, 29, and 23) (ModB) were transfected into the HEK293 cell line along with four melanoma tumor cell lines (A375, A101D, A2058 and Hs294T) (FIG. 14A). The cytokine mRNA mixture exhibited dose dependent expression and secretion across a panel of five human cell lines (FIG. 14B-F).

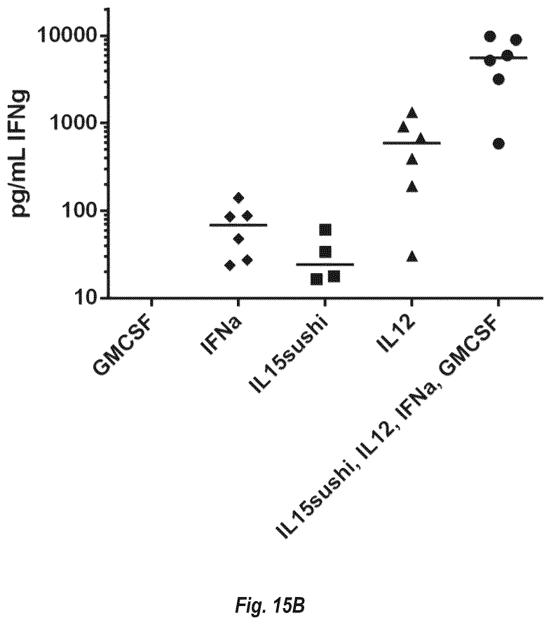

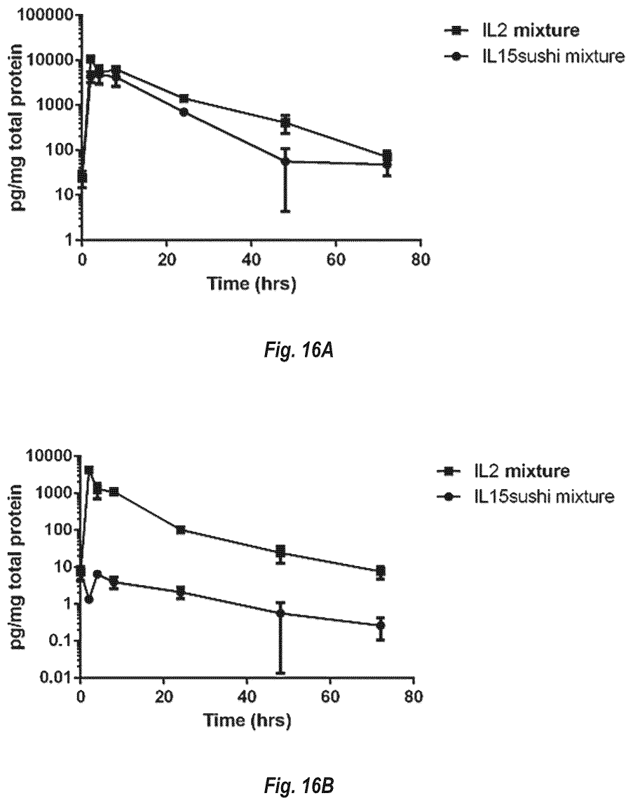

The pharmacodynamic effects of the human mRNA mixture of IL-15 sushi, IL-12sc, GM-CSF and IFNα2b were evaluated in vitro with human peripheral blood mononuclear cells (PBMC). In short, human cytokine mRNA mixture of IL-15 sushi, IL-12sc, GM-CSF and IFNα2b (ModB) or the individual cytokine mRNAs encoding IL-12sc, IFNα2b, IL-15 sushi or GM-CSF (ModB) were transfected in HEK293 cells and the conditioned media was collected at 24 hrs, diluted and added to human PBMC (FIG. 15A). The median IFNγ levels from 6 donors treated with the cytokine mRNA mixture of IL-15 sushi, IL-12sc, GM-CSF and IFNα2b was 5623 μg/mL, while treatment with the individual cytokine mRNA for IL-12sc, IFNα2b, IL-15 sushi or GM-CSF induced median IFNγ levels of 534, 67, 17, and 4 μg/mL, respectively (FIG. 15B).

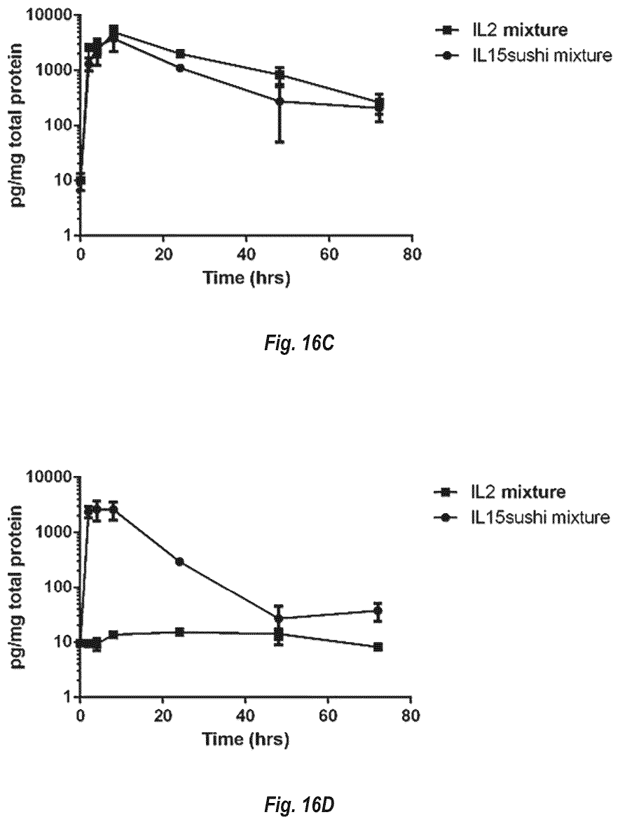

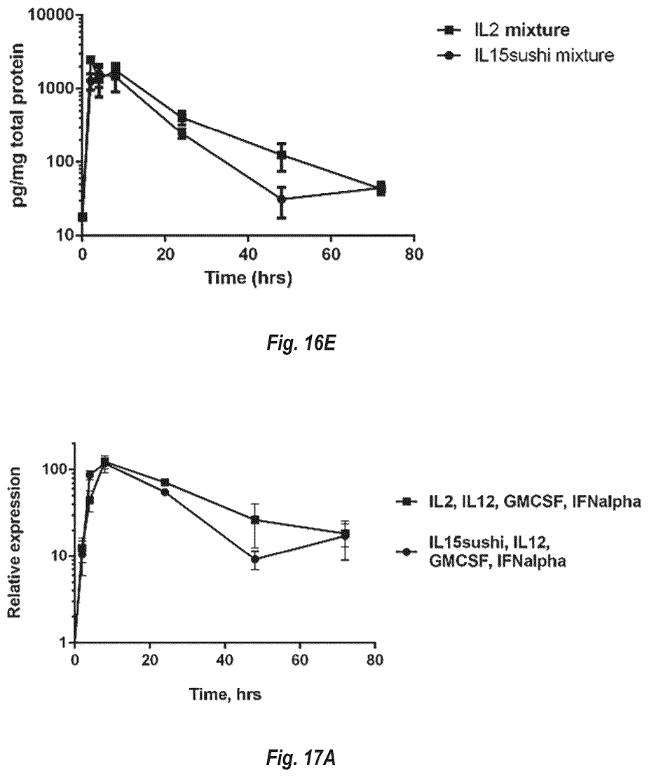

In vivo expression of the human cytokine mRNA mixture of IL-15 sushi, IL-12sc, GM-CSF and IFNα2b (ModB) and IL-2, IL-12sc, GM-CSF and IFNα2b (ModB) was monitored in the A375 human melanoma xenograft. Tumor bearing mice received a single injection of cytokine mRNA and tumor samples were collected at 2 hrs, 4 hrs, 8 hrs, 24 hrs, 48 hrs and 72 hrs after mRNA injection. Tumor lysates were prepared and expression of the individual human cytokines IFNα2b (FIG. 16A), IL-2 (FIG. 16B), IL-12sc (FIG. 16C), IL-15 sushi (FIG. 16D), GM-CSF (FIG. 16E) were evaluated.

Time dependent expression was observed for each of the individual cytokines with the maximal concentration (Cmax) occurring between 2-8 hrs for the mixtures of IL-15 sushi, IL-12sc, GM-CSF and IFNα2b (Table 4) and IL-2, IL-12sc, GM-CSF and IFNα2b (Table 5).

TABLE 4
Pharmacokinetic results for the IL-15 sushi mixture

| Tumor | GM-CSF | IFNα2b | IL-12sc | IL-15sushi |
|---|---|---|---|---|
| t_{1/2} (hrs) | 12.4 | 9.59 | 15.2 | 10.2 |
| Tmax (hrs) | 4 | 4 | 8 | 4 |
| Cmax (pg/mg) | 1591 | 4862 | 3767 | 2639 |

TABLE 5
Pharmacokinetic results for the IL-2 mixture

| Tumor | GM-CSF | IFNα2b | IL-12sc | IL-2 |
|---|---|---|---|---|
| t_{1/2} (hrs) | 14.8 | 10.2 | 15.5 | 12.8 |
| Tmax (hrs) | 2 | 2 | 8 | 2 |
| Cmax (pg/mg) | 2487 | 10685 | 4961 | 4271 |

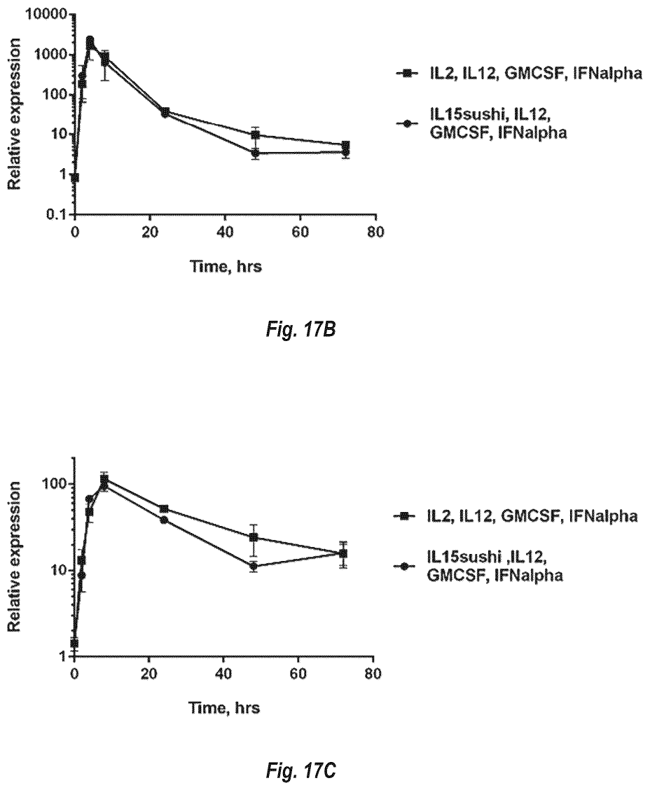

Induction of the human interferon alpha genes ISG15, ISG54 and MX1 were monitored in the A375 tumors as a pharmacodynamics marker at 2 h, 4 h, 8 h, 24 h, 48 h and 72h following mRNA injection of the cytokine mRNA mixtures of IL-15 sushi, IL-12sc, GM-CSF and IFNα2b (ModB) and IL-2, IL-12sc, GM-CSF and IFNα2b (ModB). Compared to control treated tumors, A375 tumors treated with cytokine mRNA displayed greater than 100-fold induction of ISG15 (FIG. 17A), ISG54 (FIG. 17B) and MX1 (FIG. 17C) with peak induction occurring by 8 hrs after intratumoral mRNA injection.

### Example 7—Interferon Effect

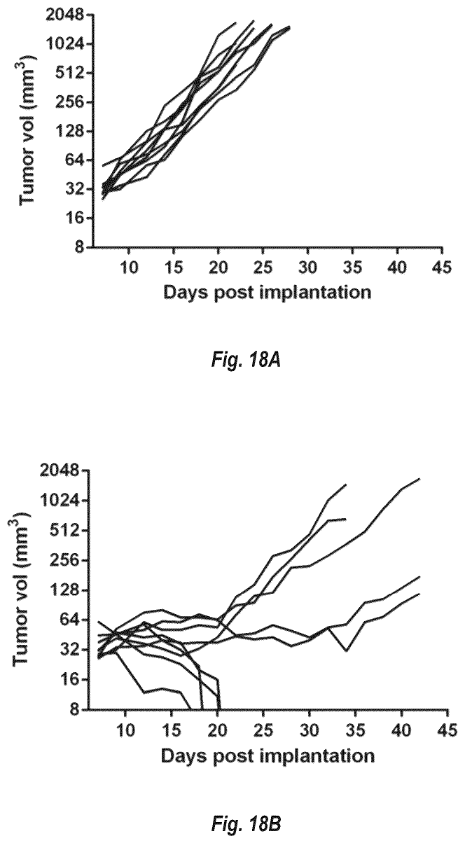

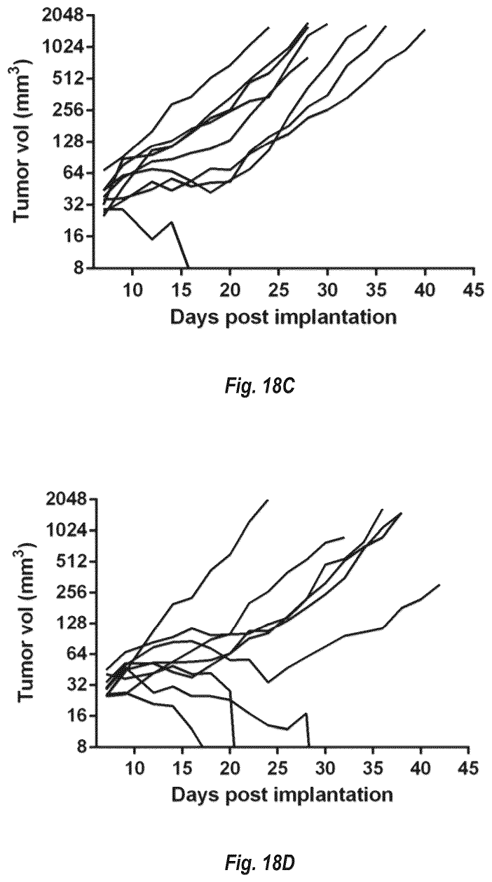

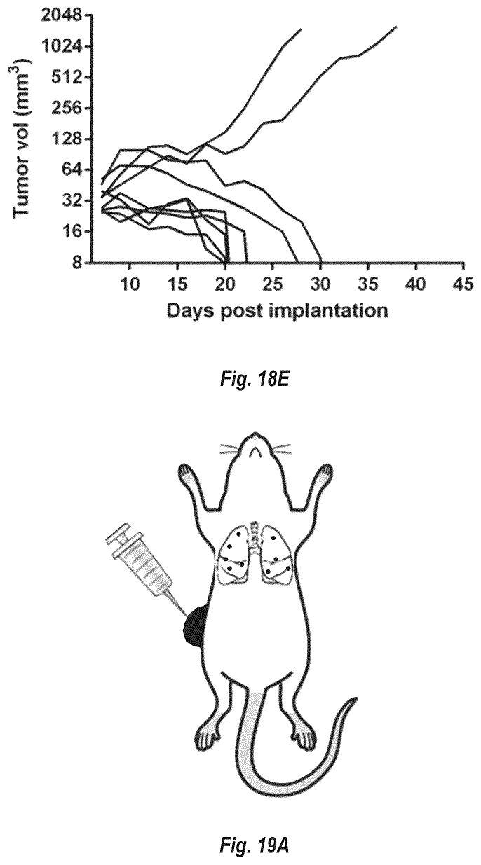

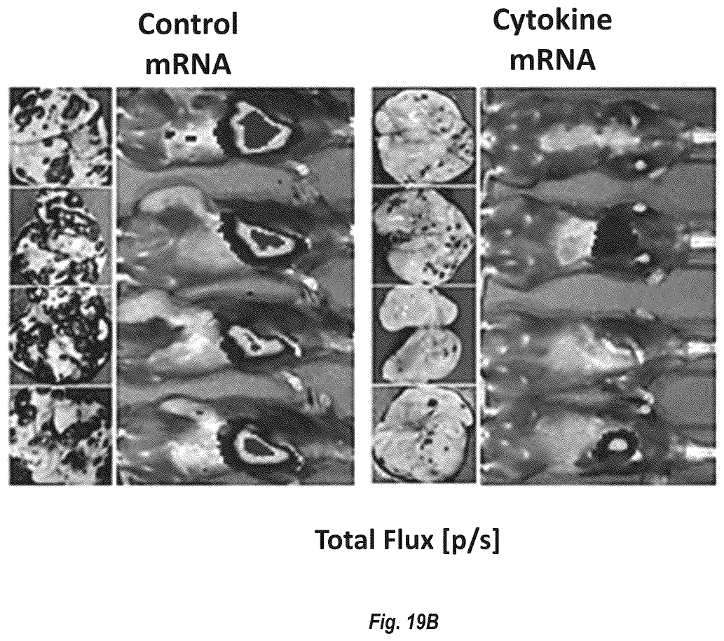

B16F10-tumor-bearing mice received intratumoral injections of ModA (“standard”) cytokine mRNA encoding IL-2, Flt3 ligand (FLT3L), 41BBL (also known as CD137L or tumor necrosis factor superfamily member 9), and CD27L-CD40L (this comprises a fusion protein of the soluble domain of CD27L also known as CD70, and CD40L; both the CD27L and the CD40L is comprised of three soluble domains of either CD27L or CD40L, all separated by GS-Linker sequences (FIG. 18A, SEQ ID NOs: 32, 62, 68, and 74) or ModB (“modified”) cytokine mRNA encoding IL-2, FLT3L, 41BBL, and CD27L-CD40L (FIG. 18B, SEQ ID NOs: 35, 65, 71, and 77). In addition, either ModA mRNA encoding IFNα (SEQ ID NO: 44) or ModB mRNA encoding IFNα (SEQ ID No: 47) was added to the ModA or ModB mRNA mixes, respectively (FIGS. 18D and 18E). Anti-tumor activity was assessed.

Mice treated with this combination of ModA mRNA had 4/9 mice tumor-free without IFNα (FIG. 18B) and 3/9 mice tumor-free with IFNα (FIG. 18D). Therefore, treatment with IFNα mRNA did not appear to increase the response to the cytokines when mRNA was dosed in the ModA form.

In contrast, mice treated with the combination of ModB mRNA had 1/9 mice tumor-free without IFNα (FIG. 18C) and 7/9 mice tumor-free with IFNα (FIG. 18E). Thus, treatment with IFNα mRNA increased the response to the mixture of cytokines when mRNA was dosed in the ModB form.

### Example 8—Cytokine mRNA in Combination with Antibodies

To evaluate the effect of intratumoral injection of cytokine mRNA in combination with systemic administration of antibodies, mice were engrafted with B16F10 or MC38 tumor cells on both the left and right flanks. Mice received four intratumoral injections with cytokine mRNA mixture of IL-15 sushi, IL-12sc, GM-CSF and IFNα (ModB; SEQ ID NOs: 53, 41, 59, and 47) into only one of the flank tumors on Days 11, 15, 19, 23, while the other flank tumor was left untreated. Mice also received intraperitoneal injection anti-PD1 antibody (Sanofi murinized version of rat IgG2a anti-mouse PD-1 clone RMP1-14 at 5 mg/kg) on Days 10, 13, 16, 19, 22, 25. Groups were as follows: 1) control mRNA (80 μg total mRNA; 50 μL intratumoral injection at 1.6 mg/mL plus control isotype antibody (clone MOPC-21 (BioLegend); 5 mg/kg): 2) control mRNA plus anti-PD1 antibody; 3) cytokine mRNA plus control isotype antibody; and 4) cytokine mRNA plus anti-PD1. Overall survival was monitored in both the B16F10 (FIG. 20A) and MC38 (FIG. 20B) tumor models. In both tumor models the highest overall survival was observed with the combination of cytokine mRNA and anti-PD-1 treatment with 60% of mice bearing B16F10 and 80% of MC38 bearing mice tumor free at the end of the study. In the B16F10 tumor model 10% of mice treated with anti-PD-1 or cytokine mRNA alone were tumor free, while in the MC38 model 40% of mice treated with anti-PD-1 and 30% of mice treated with cytokine alone were tumor free. The results indicate strong antitumor activity associated with cytokine mRNA and PD-1 combination.

To further evaluate the ability of local intratumoral cytokine mRNA in combination with the PD-1 antibody to exert a systemic anti-tumor response, mice were engrafted with B16F10 tumor cells on the right flank and received one day later an IV injection of Luciferase-expressing B16F10 cells for induction of lung metastasis. On day 11, 14 and 17 after IV tumor implantation mice bearing B16F10 tumors received in total three intratumoral injections with cytokine mRNA mixture of IL-15 sushi, IL-12sc, GM-CSF and IFNα (ModB; SEQ ID NOs: 53, 41, 59, and 47) into the flank tumor only, while tumors in the lung were untreated. On the same day mice also received intraperitoneal (IP) injections of PD-1 antibody (Sanofi murinized version of rat IgG2a anti-mouse PD-1 clone RMP1-14 at 10 mg/kg). Groups were as follows: 1) control mRNA (40 μg total mRNA; 50 μL intratumoral injection of control isotype antibody (clone MOPC-21 (BioLegend); 10 mg/kg) (FIG. 20C); 2) control mRNA plus anti-PD1 antibody (FIG. 20D); 3) cytokine mRNA plus control isotype antibody (FIG. 20E); and 4) cytokine mRNA plus anti-PD1 (FIG. 20F). Tumor growth of SC tumors was monitored (FIGS. 20C-F) as well as survival (FIG. 20G). Overall survival in this model was determined by tumor burden due to SC tumors as well as lung pseudometastasis tumor (not shown in this Figure); in some mice the SC tumor was rejected, while lung metastasis grew progressively. The highest overall survival was observed with the combination of cytokine mRNA and anti-PD-1 treatment. 6-7% of mice treated with cytokine mRNA alone were tumor free, while mice that had received anti-PD-1 alone or control mRNA+isotype antibody were all sacrificed at day 22 due to high tumor burden. The results indicate strong antitumor activity associated with cytokine mRNA and PD-1 combination in this B16F10 tumor model with lung pseudo-metastasis, while anti-PD-1 antibody alone did not show any anti-tumor activity.

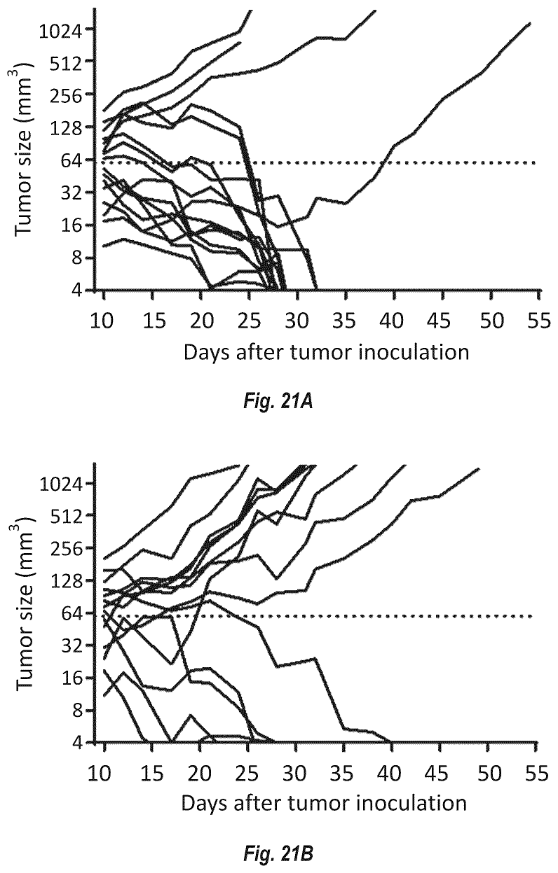

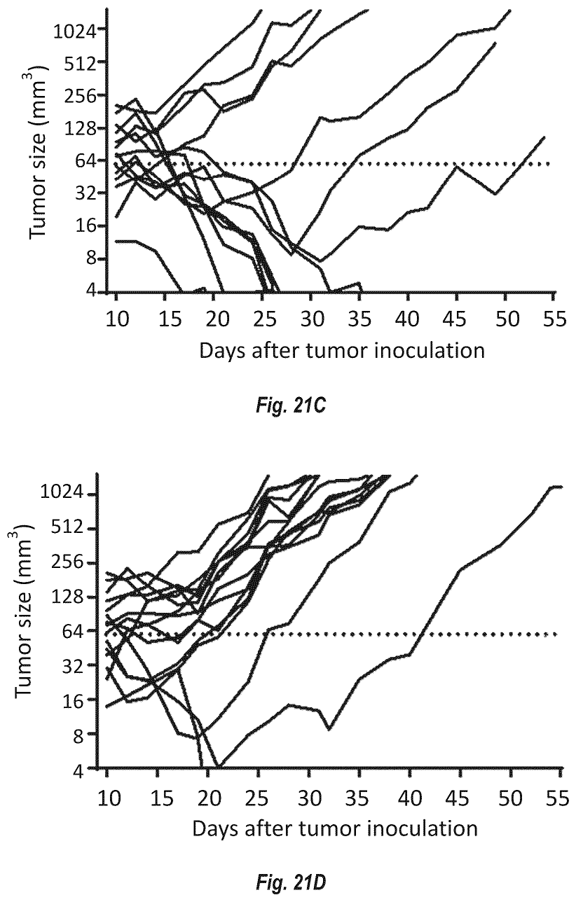

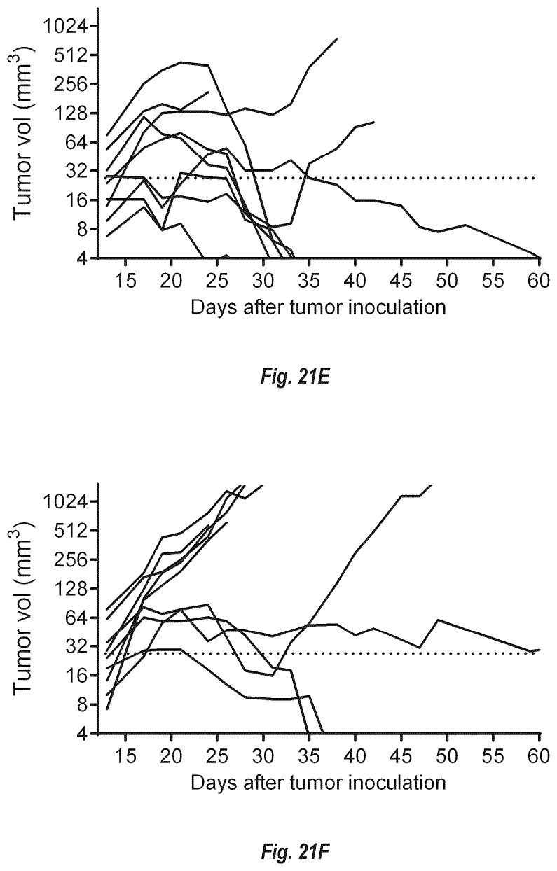

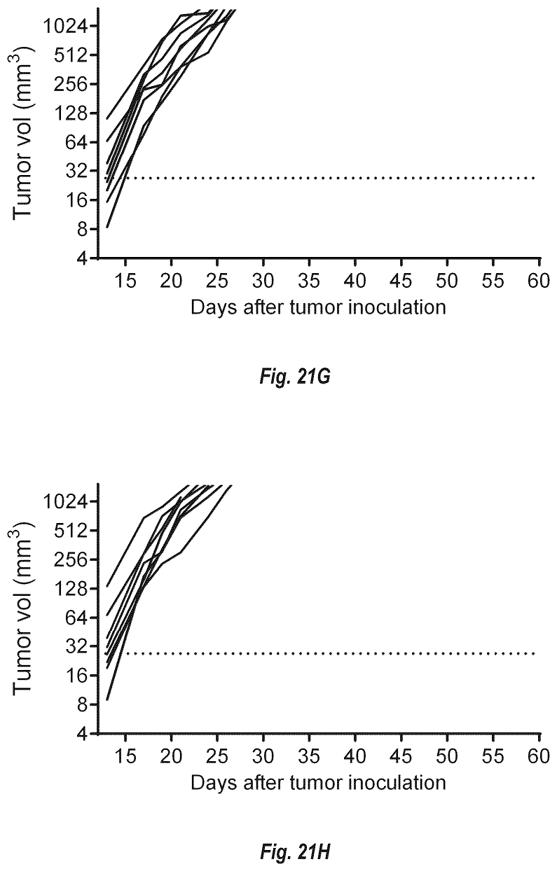

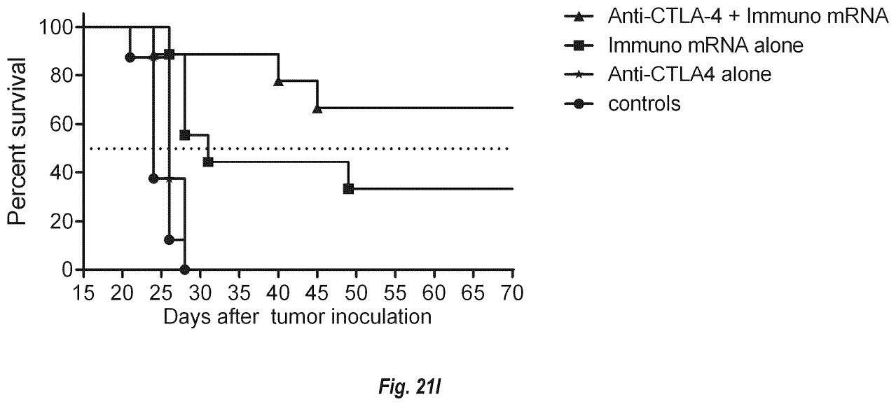

To further evaluate the effect of intratumoral injection of cytokine mRNA in combination with systemic administration of antibodies, mice were engrafted with CT26 tumor cells on right flanks. Mice received four intratumoral injections with cytokine mRNA mixture of IL-15 sushi, IL-12sc, GM-CSF and IFNα (ModB; SEQ ID NOs: 53, 41, 59, and 47) on day 11, 14, 18 and 21 after tumor inoculation. On the same day mice also received intraperitoneal (IP) injections of an anti-CTLA-4 antibody (100 μg/200 μL per mouse; clone 9H10 from InVivoMAb) or the isotype control antibody (100 μg/200 μL per mouse; Armenian hamster IgG from BioXCell). Groups were as follows: 1) cytokine mRNA plus anti-CTLA-4 antibody (FIG. 21A); 2) cytokine mRNA plus isotype control antibody (FIG. 21B); 3) control mRNA plus anti-CTLA-4 antibody (FIG. 21C) and 4) control mRNA plus isotype control antibody (FIG. 21D). Combination therapy of intratumoral cytokine mRNA and IP-injected anti-CTLA-4 resulted in strongest anti-tumoral activity with 12 tumor-free mice out of 16 mice on day 55 after tumor inoculation (FIG. 21A). Treatment with either cytokine mRNA plus isotype control antibody (FIG. 21B) or control mRNA plus anti-CTLA-4 antibody (FIG. 21C) induced less anti-tumoral activity with 5 and 7 tumor-free mice at the end of the study, respectively. In comparison, in the group that received control mRNA plus isotype control antibody (FIG. 21D), only one tumor-free mouse remained at the conclusion of the study.

To further evaluate the effect of intratumoral injection of cytokine mRNA in combination with an anti-CTLA-4 antibody, mice were engrafted with B16F10 tumor cells on right flanks. Mice received three intratumoral injections with cytokine mRNA mixture of IL-15 sushi, IL-12sc, GM-CSF and IFNα (ModB; SEQ ID NOs: 53, 41, 59, and 47) on day 13, 17 and 20 after tumor inoculation. On day 13, 17, 20 and 24 after tumor inoculation mice also received intraperitoneal (IP) injections of an anti-CTLA-4 antibody (100 μg/200 μL per mouse; clone 9H10 from InVivoMAb) or the isotype control antibody (100 μg/200 μL per mouse; Armenian hamster IgG from BioXCell). Tumor growth of SC tumors as well as survival was monitored. Groups were as follows: 1) cytokine mRNA plus anti-CTLA-4 antibody (FIG. 21 E); 2) cytokine mRNA plus isotype control antibody (FIG. 21F); 3) control mRNA plus anti-CTLA-4 antibody (FIG. 21G) and 4) control mRNA plus isotype control antibody (FIG. 21H). Combination therapy of intratumoral cytokine mRNA and IP-injected anti-CTLA-4 resulted in strongest anti-tumoral activity with 6 tumor-free mice out of 9 mice on day 60 after tumor inoculation (FIG. 21E). Treatment with cytokine mRNA plus isotype control antibody (FIG. 21F) induced less anti-tumoral activity with 2 tumor-free mice out of 9 mice. In comparison, in the two groups that either received control mRNA plus anti-CTLA-4 antibody (FIG. 21G) or control mRNA plus isotype control antibody (FIG. 21H), no tumor-free mouse remained at the conclusion of the study. Percent survival is depicted in FIG. 21I, showing the highest overall survival in the combination of cytokine mRNA and anti-CTLA-4 treatment with 67% of mice bearing tumors at the end of the study (day 70), while 33% of mice treated with cytokine mRNA alone were tumor free. The results indicate strong antitumor activity associated with cytokine mRNA alone and cytokine mRNA and anti-CTLA-4 antibody in this B16F10 tumor model, in which anti-CTLA-4 antibody alone did not show any anti-tumor activity.

### Example 9—mRNA Cytokine Injection in Multiple Cancer Types

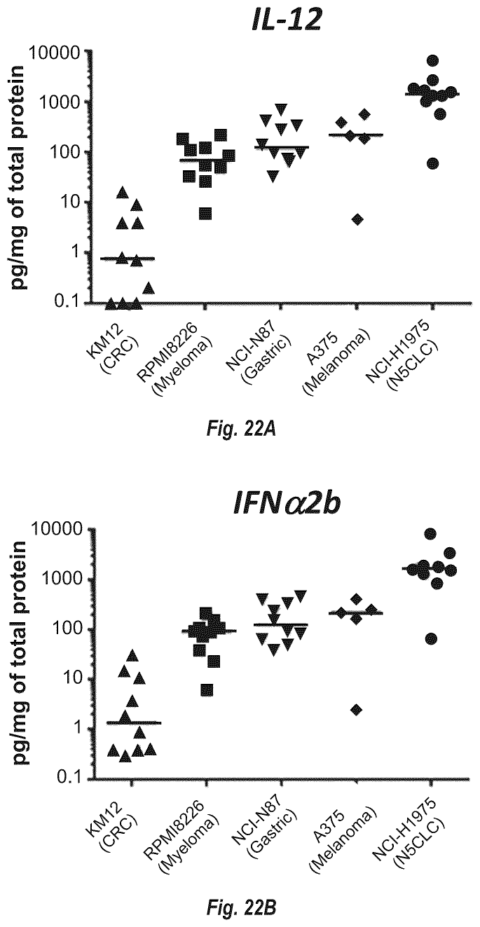

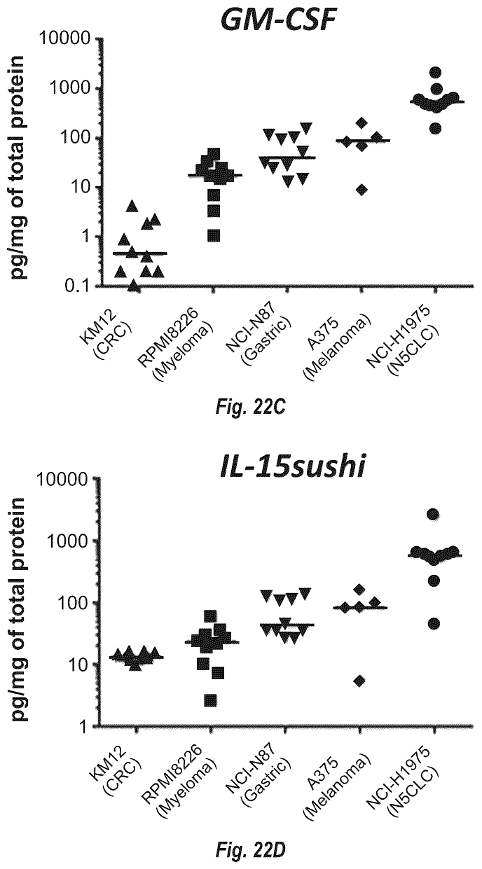

To evaluate the effect of intratumoral injection of cytokine mRNA in various types of cancer, five xenograft mouse models—KM12 (CRC), RPMI8226 (Myeloma), NCI-N87 (Gastric), A375 (Melanoma), and NCI-H1975 (NSCLC)—were tested as described in Example 1. Mice bearing KM12 (CRC), RPMI8226 (Myeloma), NCI-N87 (Gastric), A375 (Melanoma), and NCI-H1975 (NSCLC) tumors received a single intratumoral injection with 8 μg (2 μg/target) human cytokine mRNA mixture of IL-15 sushi, IL-12sc, GM-CSF and IFNα (ModB; SEQ ID NOs: 26, 18, 29, and 23) and the encoded cytokines were assessed in the tumor at 24 hours. Expression of each of the 4 cytokines of IL-15 sushi (FIG. 22D), IL-12sc (FIG. 22A), GM-CSF (FIG. 22C) and IFNα (FIG. 22B) was detected in all five of the xenograft models with the highest cytokines levels observed in NCI-H1975, followed by A375, NCI-N87, RPMI8226, and KM12.

### Example 10—Dose Dependent Serum Expression of Cytokines after Intratumoral mRNA Cytokine Injection

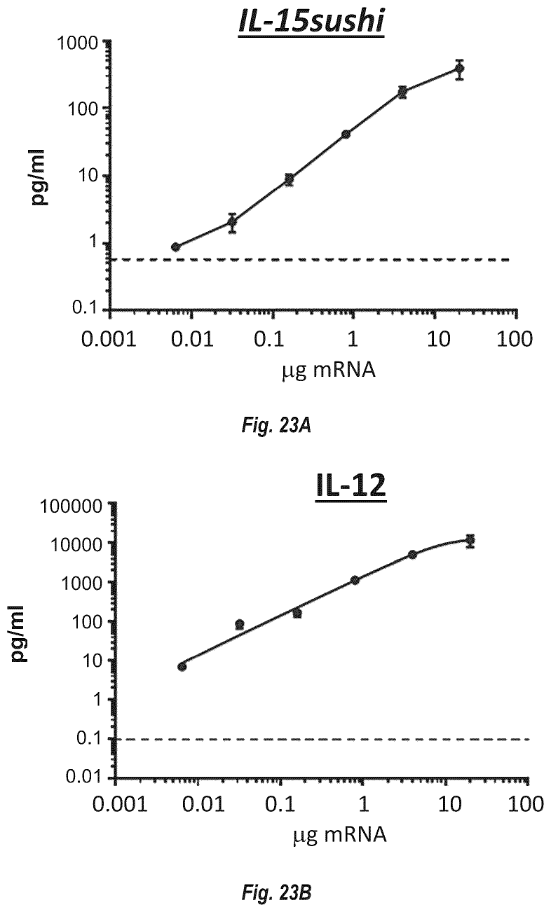

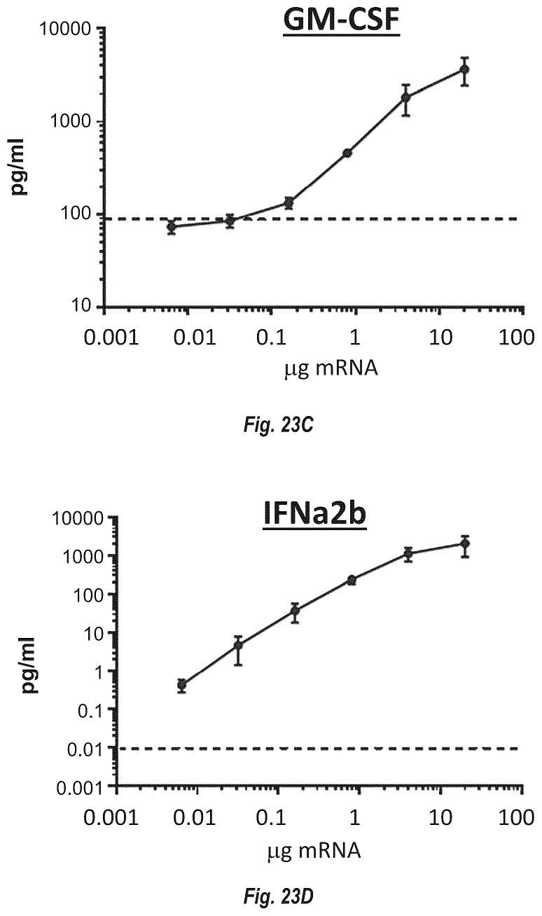

The effect of different intratumoral mRNA doses on the expression of the encoded cytokines was examined in the serum of mice engrafted with a single A375 tumor on the right flank. Mice received a single intratumoral injection of a cytokine mRNA mixture of human IL-15 sushi, IL-12sc, GM-CSF and IFNα (ModB; SEQ ID NOs: 26, 18, 29, and 23). At 6 hours after intratumoral mRNA injection, serum was collected and cytokine expression was analyzed by Meso Scale Discovery assay. Dose dependent expression of each of the mRNA encoded cytokines was observed in the serum from the highest dose of 80 μg (20 μg) to the lowest dose of 0.0256 μg (0.0064 μg). Results are shown in FIGS. 23A-D.

### Example 11—Cytokine mRNA Leads to Expansion of Gp70+ CD8 T Cells

Mice bearing a single CT26 tumor on one flank received a four intratumoral injections of a cytokine mRNA mixture of IL-15 sushi, GM-CSF, IFNα, and IL-12sc (ModB; SEQ ID NOs: 53, 41, 59, and 47). Blood was collected 13 days after first intratumoral mRNA administration and T cells specific for the gp70 tumor antigen were quantified by flow cytometry. Frequency of T cells specific for the gp70 tumor antigen in blood were strongly increased in mice upon intratumoral injection of mRNA cytokines compared to mice that had received control RNA.

### Example 12—Cytokine mRNA Induces Multiple Pro-Inflammatory Pathways and Increases Immune Infiltrate in Both Treated and Untreated Tumors

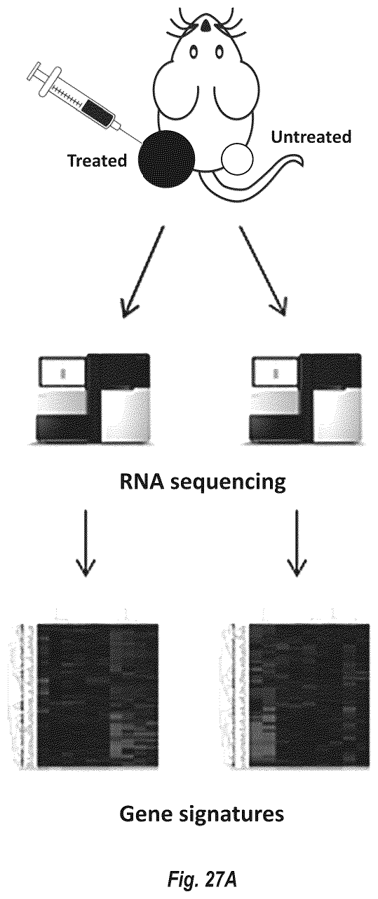

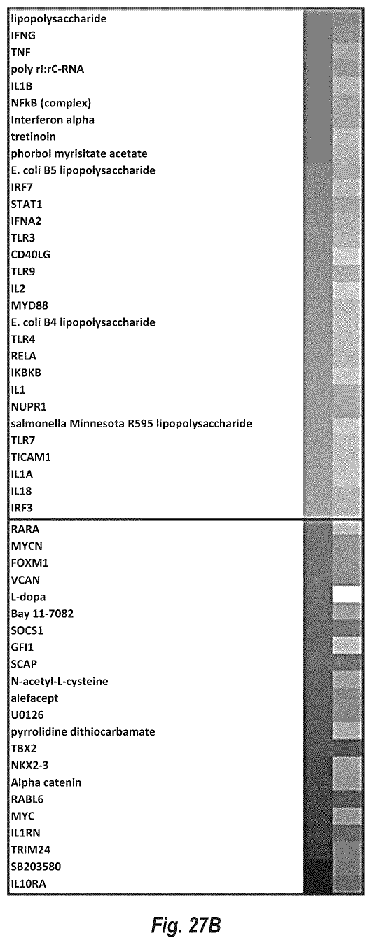

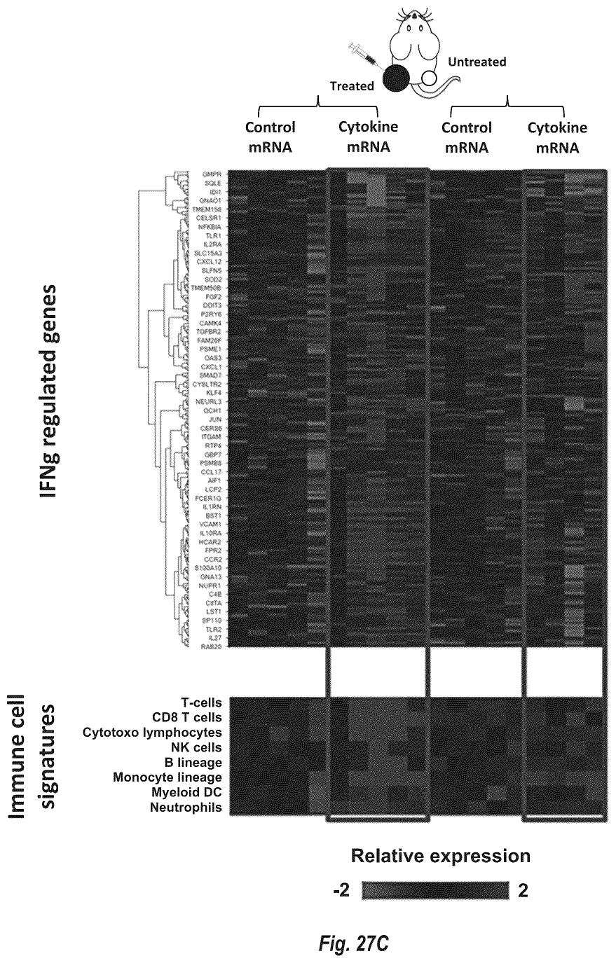

Mice bearing B16F10 tumors on the left and right flank received a single intratumoral injection of 80 μg of mRNA (20 μg/target) into right tumor which was initiated with 0.5×10\hat{ }6 cells (treated), while the tumor initiated with 0.25×10\hat{ }6 cells remained untreated. At seven days after intratumoral injection of mRNA, both tumors were collected, and RNA was isolated and subjected to RNA sequencing analysis. As shown in FIGS. 27A-C, treatment with a cytokine mRNA mixture of IL-15 sushi, GM-CSF, IFNα, and IL-12sc (SEQ ID NOs: 59, 53, 41, and 47) upregulated multiple proinflammatory pathways including a range of IFNgamma response genes. The upregulation of proinflammatory/IFNgamma related pathways occurred in both the treated and untreated tumors, supporting the notion that local intratumoral treatment has systemic immune modulatory effects.

Causal network analysis (part of Ingenuity pathway analysis tool) was performed on 3298 genes that were differentially expressed (1699 up-regulated and 1599 down-regulated) between cytokine mRNA treatment and control in injected flank and 4973 genes (2546 up-regulated and 2427 down-regulated) in un-injected flank to identify changes in signaling pathways that could explain the observed changes in gene expression. Z scores were calculated to indicate the changes in pathways, with signs of score indicating the direction of change (positive sign suggests the activation whereas negative sign suggests inhibition).

Hierarchical clustering on expression of 328 genes regulated by IFNG (295 up-regulation and 33 down-regulation) was performed in both control and cytokine mRNA treated samples in both injected and un-injected tumors. Expression of each gene across samples were z-score normalized. The similarity metric is based on Pearson's correlation coefficient and complete-linkage is used to generate dendrogram. See, Table 6.

Relative abundance of infiltrated immune cells is determined by calculating the average expression of immune cell-type specific gene signatures.

TABLE 6

| | | Treated_ | Treated_ | Treated_ | Untreated_ | Untreated_ | Untreated_ |
|---|---|---|---|---|---|---|---|
| | | cytokine | cytokine | cytokine | cytokine | cytokine | cytokine |
| | | mRNA | mRNA | mRNA | mRNA | mRNA | mRNA |
| | | mix/IgG1 | mix/IgG1 | mix/IgG1 | mix/IgG1 | mix/IgG1 | mix/IgG1 |
| | | vs Luc | vs Luc | vs Luc | vs Luc | vs Luc | vs Luc |
| | IFNG_ | mRNA/IgG1. | mRNA/IgG1. | mRNA/IgG1. | mRNA/IgG1. | mRNA/IgG1. | mRNA/IgG1. |
| Gene | regulation | FoldChange | RawPValue | FDR_BH | Foldchange | RawPValue | FDR_BH |
| ABI1 | Activate | 1.6776 | 0.0073 | 0.0995 | 1.5218 | 0.0354 | 0.1551 |
| ACOD1 | Activate | 31.6293 | 3.9652E−06 | 0.0024 | 6.1017 | 0.0096 | 0.0911 |
| ADGRG2 | Activate | 1.8174 | 0.0248 | 0.2076 | 1.3902 | 0.2293 | 0.4662 |
| ADORA2B | Activate | 2.9657 | 0.0044 | 0.0752 | 1.4261 | 0.352 | 0.6138 |
| AIF1 | Activate | 6.0406 | 0.0005 | 0.0201 | 4.7598 | 0.0032 | 0.057 |
| AIM2 | Activate | 9.9707 | 0.000085324 | 0.0086 | 3.4835 | 0.0276 | 0.1361 |
| ANGPTL4 | Activate | 2.0707 | 0.0293 | 0.2265 | 2.0904 | 0.0368 | 0.1586 |
| APOL6 | Activate | 10.3994 | 0.0017 | 0.0418 | 10.1341 | 0.0031 | 0.0564 |
| APP | Activate | 2.0312 | 0.0006 | 0.0238 | 2.2382 | 0.0003 | 0.0276 |
| ARFGAP3 | Activate | 1.6535 | 0.0146 | 0.1517 | 1.0201 | 0.9237 | 1 |
| ASS1 | Activate | 3.5414 | 0.0084 | 0.1093 | −1.0202 | 0.9668 | 1 |
| ATF3 | Activate | 5.5284 | 0.0006 | 0.0231 | −1.1007 | 0.8409 | 1 |
| B2M | Activate | 4.7005 | 0.000092097 | 0.0089 | 2.7946 | 0.0085 | 0.0859 |
| BACH1 | Activate | 2.2944 | 0.0098 | 0.1179 | 1.3281 | 0.3821 | 0.6482 |
| BATF2 | Activate | 4.0479 | 0.0211 | 0.1885 | 1.6468 | 0.4201 | 0.6889 |
| BCL2L11 | Activate | 4.2973 | 0.0057 | 0.0868 | 1.1465 | 0.7946 | 1 |
| BCL3 | Activate | 4.4411 | 0.0167 | 0.1639 | 2.5676 | 0.1413 | 0.3455 |
| BLNK | Activate | 4.2753 | 0.0003 | 0.0158 | 3.2008 | 0.0044 | 0.0643 |
| BST1 | Activate | 16.9081 | 1.3371E−06 | 0.0016 | 6.6164 | 0.0007 | 0.0334 |
| BTG1 | Activate | 3.5307 | 0.0001 | 0.0102 | −1.1775 | 0.5968 | 0.8607 |
| C1QA | Activate | 5.3999 | 0.0029 | 0.0588 | 1.3506 | 0.5911 | 0.8558 |
| C1QB | Activate | 5.955 | 0.0104 | 0.1219 | 3.531 | 0.0786 | 0.2408 |
| C1QC | Activate | 6.2282 | 0.0009 | 0.0307 | 2.7013 | 0.0707 | 0.2284 |
| C2 | Activate | 2.2866 | 0.0367 | 0.2598 | 2.5908 | 0.0242 | 0.1281 |
| C3 | Activate | 10.2057 | 0.0043 | 0.0734 | 11.6428 | 0.0044 | 0.0648 |
| C4B | Activate | 3.3143 | 0.0154 | 0.1562 | 1.3973 | 0.5045 | 0.7768 |
| C5AR1 | Activate | 6.4343 | 0.000061281 | 0.0074 | 1.8122 | 0.1729 | 0.3925 |
| CAMK4 | Activate | 2.5261 | 0.0011 | 0.0335 | 2.0726 | 0.012 | 0.1009 |
| CASP1 | Activate | 8.2891 | 0.0079 | 0.1049 | 6.7759 | 0.0214 | 0.1263 |
| CASP4 | Activate | 3.9281 | 0.0015 | 0.0389 | 1.2492 | 0.5969 | 0.8608 |
| CCL11 | Activate | 6.225 | 0.0028 | 0.0579 | 9.5207 | 0.0007 | 0.0334 |
| CCL17 | Activate | 3.9738 | 0.0268 | 0.2146 | 3.0432 | 0.0868 | 0.2549 |
| CCL2 | Activate | 5.0444 | 0.0331 | 0.2446 | 1.9159 | 0.4045 | 0.6716 |
| CCL22 | Activate | 5.4223 | 0.000050981 | 0.0069 | 3.6151 | 0.002 | 0.0467 |
| CCL3 | Activate | 8.2215 | 0.0007 | 0.0259 | 4.7966 | 0.0129 | 0.1045 |
| CCL4 | Activate | 8.7234 | 0.0008 | 0.0287 | 2.8285 | 0.1042 | 0.2846 |
| CCL5 | Activate | 19.1375 | 0.000004935 | 0.0027 | 9.4224 | 0.0004 | 0.0299 |
| CCL7 | Activate | 4.7053 | 0.0068 | 0.0952 | −1.2497 | 0.6964 | 0.9457 |
| CCL8 | Activate | 9.3589 | 0.0113 | 0.1283 | 3.9524 | 0.1286 | 0.3243 |
| CCR1 | Activate | 5.333 | 0.0012 | 0.0349 | 1.7221 | 0.2823 | 0.5325 |
| CCR2 | Activate | 5.8007 | 0.0025 | 0.0546 | 2.1931 | 0.1766 | 0.3975 |
| CCR5 | Activate | 10.8409 | 0.000010504 | 0.004 | 4.8697 | 0.0025 | 0.0512 |
| CCRL2 | Activate | 3.9822 | 0.002 | 0.0465 | 3.6428 | 0.0056 | 0.071 |
| CD14 | Activate | 5.6413 | 0.0025 | 0.0546 | 1.413 | 0.5406 | 0.8104 |
| CD2 | Activate | 13.539 | 0.0001 | 0.0095 | 5.0981 | 0.0138 | 0.1086 |
| CD274 | Activate | 11.0684 | 0.000096803 | 0.0091 | 3.4411 | 0.038 | 0.1616 |
| CD38 | Activate | 5.5697 | 0.0005 | 0.0212 | 1.4645 | 0.4221 | 0.6908 |
| CD40 | Activate | 11.0285 | 0.0003 | 0.0158 | 1.2537 | 0.7201 | 0.9637 |
| CD40LG | Activate | 3.1168 | 0.0068 | 0.0952 | 1.1976 | 0.6673 | 0.9224 |
| CD68 | Activate | 6.1325 | 0.000040468 | 0.0063 | 3.6801 | 0.0029 | 0.0544 |
| CD74 | Activate | 30.4664 | 0.000057089 | 0.0072 | 5.9291 | 0.0291 | 0.1392 |
| CD80 | Activate | 4.1003 | 0.0005 | 0.0217 | 3.5579 | 0.0026 | 0.0517 |
| CD86 | Activate | 6.3927 | 0.0008 | 0.027 | 8.2257 | 0.0004 | 0.0289 |
| CDKN2A | Activate | 1.9017 | 0.0117 | 0.1312 | −1.2679 | 0.3579 | 0.6202 |
| CEBPB | Activate | 4.4389 | 0.0007 | 0.0253 | 2.1429 | 0.0784 | 0.2404 |
| CELSR1 | Activate | 1.6953 | 0.0001 | 0.0107 | 1.1455 | 0.3001 | 0.5534 |
| CERS6 | Activate | 2.2538 | 0.0064 | 0.0927 | 1.717 | 0.0763 | 0.2363 |
| CFB | Activate | 9.003 | 0.0001 | 0.0109 | 6.2886 | 0.0018 | 0.0444 |
| CH25H | Activate | 4.3757 | 0.007 | 0.097 | 1.9042 | 0.2435 | 0.4858 |
| CHRNE | Activate | 1.6587 | 0.0293 | 0.2263 | 1.0536 | 0.8254 | 1 |
| CIITA | Activate | 6.2751 | 0.0008 | 0.0287 | 2.736 | 0.0651 | 0.2172 |
| CLEC2D | Activate | 4.9639 | 0.0002 | 0.0135 | 2.6567 | 0.0223 | 0.1263 |
| CLEC4E | Activate | 25.396 | 2.9823E−07 | 0.001 | 7.0201 | 0.0009 | 0.0355 |
| CLIC4 | Activate | 2.2909 | 0.000025847 | 0.0053 | 1.4654 | 0.0399 | 0.1658 |
| CMPK2 | Activate | 2.6988 | 0.0048 | 0.0785 | 1.9213 | 0.0687 | 0.2249 |
| CORO1A | Activate | 5.8653 | 0.0076 | 0.1019 | 2.1612 | 0.2494 | 0.4929 |
| CP | Activate | 2.7012 | 0.0222 | 0.1945 | 1.2703 | 0.5885 | 0.8544 |
| CSF1 | Activate | 5.3648 | 0.0071 | 0.0977 | 2.9314 | 0.0916 | 0.2624 |
| CSF2 | Activate | 2.4249 | 0.0053 | 0.0828 | 1.8261 | 0.0635 | 0.2145 |
| CSF2RB | Activate | 7.8798 | 0.000014063 | 0.0041 | 3.3417 | 0.008 | 0.0833 |
| CTSB | Activate | 2.9396 | 0.0192 | 0.1786 | −1.5854 | 0.3271 | 0.5867 |
| CTSD | Activate | 1.7662 | 0.0058 | 0.0873 | 1.5654 | 0.0351 | 0.1546 |
| CTSH | Activate | 6.6723 | 0.0004 | 0.0189 | 3.0998 | 0.0341 | 0.1523 |
| CTSS | Activate | 14.2913 | 0.000052305 | 0.0069 | 2.0554 | 0.2402 | 0.4813 |
| CXCL1 | Activate | 2.4631 | 0.0443 | 0.2909 | 2.3701 | 0.0679 | 0.223 |
| CXCL10 | Activate | 4.0615 | 0.0183 | 0.174 | 3.84 | 0.0314 | 0.1453 |
| CXCL11 | Activate | 6.3825 | 0.0032 | 0.0623 | 2.1745 | 0.2167 | 0.4494 |
| CXCL12 | Activate | 4.1992 | 0.0099 | 0.1191 | 3.9497 | 0.0188 | 0.1263 |
| CXCL16 | Activate | 11.7476 | 0.000012898 | 0.0041 | 2.7724 | 0.052 | 0.1919 |
| CXCL2 | Activate | 17.8737 | 0.000065478 | 0.0076 | 2.0435 | 0.2897 | 0.5414 |
| CXCL3 | Activate | 2.5623 | 0.0157 | 0.1584 | −1.1159 | 0.7808 | 1 |
| CXCL9 | Activate | 10.5652 | 0.002 | 0.0475 | 6.7433 | 0.0152 | 0.1139 |
| CYBB | Activate | 10.2429 | 0.0001 | 0.0102 | 2.4115 | 0.1288 | 0.3246 |
| CYLD | Activate | 2.1952 | 0.0005 | 0.0217 | 1.0366 | 0.8685 | 1 |
| CYSLTR2 | Activate | 4.6876 | 0.0015 | 0.0389 | −1.3886 | 0.4896 | 0.7624 |
| DAPK1 | Activate | 2.9566 | 0.0202 | 0.1837 | 2.3734 | 0.0752 | 0.2341 |
| DAXX | Activate | 2.5923 | 0.0255 | 0.2101 | 2.7152 | 0.0271 | 0.1349 |
| DDIT3 | Activate | 2.2027 | 0.0286 | 0.2233 | 2.4229 | 0.0212 | 0.1263 |
| DDX58 | Activate | 2.2084 | 0.0054 | 0.084 | 1.2566 | 0.422 | 0.6908 |
| DPP4 | Activate | 4.4101 | 0.047 | 0.3002 | 2.2805 | 0.2869 | 0.5383 |
| DTX3L | Activate | 3.4716 | 0.000027857 | 0.0053 | 1.6387 | 0.0759 | 0.2356 |
| EBI3 | Activate | 5.2118 | 0.0003 | 0.0153 | 2.8451 | 0.0203 | 0.1263 |
| ECE1 | Activate | 3.9089 | 0.0033 | 0.0624 | 1.8673 | 0.1782 | 0.3996 |
| EGLN3 | Activate | 2.9572 | 0.0465 | 0.2989 | 1.0391 | 0.9453 | 1 |
| EGR2 | Activate | 2.4479 | 0.0155 | 0.157 | 2.5764 | 0.0158 | 0.1159 |
| ERAP1 | Activate | 2.5669 | 0.0033 | 0.0634 | 2.2811 | 0.0134 | 0.1068 |
| FAM26F | Activate | 14.7052 | 0.0008 | 0.0279 | 26.6887 | 0.0002 | 0.0227 |
| FAS | Activate | 7.0896 | 0.0003 | 0.0169 | 4.7216 | 0.0052 | 0.0695 |
| FCER1G | Activate | 6.7045 | 0.0007 | 0.0254 | 4.8631 | 0.0059 | 0.0728 |
| FGF2 | Activate | 2.9272 | 0.0257 | 0.2106 | 1.8257 | 0.2249 | 0.4606 |
| FGL2 | Activate | 12.456 | 9.3027E−06 | 0.0038 | 4.9116 | 0.0036 | 0.0599 |
| FPR2 | Activate | 26.1833 | 0.000096227 | 0.0091 | 4.1758 | 0.0744 | 0.2328 |
| FTH1 | Activate | 1.6855 | 0.0413 | 0.2791 | 2.2166 | 0.0045 | 0.0657 |
| GBP2 | Activate | 7.2783 | 0.0007 | 0.0248 | 6.7229 | 0.0017 | 0.0437 |
| GBP3 | Activate | 6.2795 | 0.0006 | 0.0222 | 3.0356 | 0.0358 | 0.156 |
| GBP4 | Activate | 18.7632 | 0.0002 | 0.0139 | 6.7981 | 0.0154 | 0.1146 |
| GBP5 | Activate | 18.254 | 0.0003 | 0.0157 | 7.5534 | 0.0118 | 0.1 |
| GBP6 | Activate | 6.4537 | 0.0004 | 0.0187 | 8.0969 | 0.0002 | 0.0266 |
| GBP7 | Activate | 4.3285 | 0.0017 | 0.0427 | 2.3295 | 0.0713 | 0.2296 |
| GCH1 | Activate | 3.9933 | 0.0057 | 0.0867 | −2.2318 | 0.1144 | 0.3009 |
| GNA13 | Activate | 1.843 | 0.0129 | 0.1403 | 1.2404 | 0.3874 | 0.6539 |
| GSDMD | Activate | 5.5069 | 0.000067203 | 0.0076 | 4.0075 | 0.0013 | 0.0403 |
| GZMB | Activate | 10.9342 | 0.001 | 0.0323 | 11.0118 | 0.0018 | 0.0442 |
| H2-M3 | Activate | 6.3613 | 0.0003 | 0.0169 | −1.2831 | 0.6129 | 0.8754 |
| H2-Q7 | Activate | 13.035 | 0.00007123 | 0.0078 | 13.2107 | 0.0001 | 0.0209 |
| H2-T23 | Activate | 9.9835 | 0.0035 | 0.0651 | 1.093 | 0.9091 | 1 |
| HCAR2 | Activate | 5.1405 | 0.0007 | 0.0254 | 3.0731 | 0.0206 | 0.1263 |
| HCK | Activate | 7.0952 | 0.000025322 | 0.0053 | 4.1767 | 0.0019 | 0.0455 |
| HIF1A | Activate | 1.661 | 0.0261 | 0.2122 | 1.117 | 0.6345 | 0.8942 |
| HMOX1 | Activate | 2.9191 | 0.0049 | 0.0797 | 2.2892 | 0.0345 | 0.1532 |
| ICAM1 | Activate | 5.8185 | 0.0005 | 0.0217 | 4.9939 | 0.0023 | 0.0487 |
| ICOSL | Activate | 1.9545 | 0.0086 | 0.1107 | 1.8592 | 0.0203 | 0.1263 |
| IDO1 | Activate | 2.1676 | 0.007 | 0.097 | 1.5151 | 0.1535 | 0.3644 |
| IFI27 | Activate | 2.1198 | 0.0058 | 0.088 | 1.1685 | 0.5669 | 0.8345 |
| IFI44 | Activate | 10.2739 | 0.0001 | 0.0093 | 5.8893 | 0.0032 | 0.057 |
| IFIH1 | Activate | 3.3922 | 0.0001 | 0.0093 | 1.8886 | 0.0369 | 0.1588 |
| IFIT1 | Activate | 4.7651 | 0.000038724 | 0.0062 | 2.3488 | 0.0191 | 0.1263 |
| IFIT2 | Activate | 2.6547 | 0.0381 | 0.266 | 1.6098 | 0.3269 | 0.5865 |
| IFIT3 | Activate | 6.6433 | 0.0003 | 0.0153 | 2.3076 | 0.0981 | 0.2746 |
| IFITM1 | Activate | 13.8818 | 0.0017 | 0.0427 | 4.0409 | 0.096 | 0.2703 |
| IFITM3 | Activate | 5.3416 | 0.000049885 | 0.0069 | 4.6318 | 0.0003 | 0.0278 |
| IFNG | Activate | 15.8295 | 0.000030559 | 0.0055 | 5.3984 | 0.0086 | 0.0862 |
| IFNGR1 | Activate | 4.6529 | 0.0003 | 0.0169 | 1.1624 | 0.713 | 0.9579 |
| IKBKE | Activate | 3.6191 | 0.000070796 | 0.0078 | 2.1509 | 0.0151 | 0.1135 |
| IL10RA | Activate | 6.6362 | 0.0006 | 0.0225 | 2.2744 | 0.1259 | 0.3198 |
| IL12B | Activate | 1.5869 | 0.0237 | 0.2019 | 1.3356 | 0.1698 | 0.3882 |
| IL12RB1 | Activate | 3.6563 | 0.0032 | 0.0623 | 2.7374 | 0.0261 | 0.1323 |
| IL12RB2 | Activate | 10.8737 | 0.0001 | 0.0105 | 2.7693 | 0.0898 | 0.2596 |
| IL15RA | Activate | 5.6222 | 0.0002 | 0.0124 | 3.263 | 0.01 | 0.0929 |
| IL18BP | Activate | 13.3799 | 0.000016505 | 0.0045 | 2.6518 | 0.0808 | 0.2445 |
| IL18R1 | Activate | 10.5794 | 0.0004 | 0.0172 | 4.1773 | 0.0288 | 0.1387 |
| IL18RAP | Activate | 12.8979 | 1.6368E−06 | 0.0016 | 3.9032 | 0.0058 | 0.0721 |
| IL1A | Activate | 5.0611 | 0.0002 | 0.014 | 2.016 | 0.1006 | 0.2792 |
| IL1B | Activate | 7.1785 | 0.0017 | 0.0427 | 2.9628 | 0.0846 | 0.2505 |
| IL1RL1 | Activate | 3.2501 | 0.0421 | 0.2825 | 2.7191 | 0.0999 | 0.2778 |
| IL1RN | Activate | 11.4696 | 0.000016637 | 0.0045 | 3.5493 | 0.0183 | 0.1256 |
| IL27 | Activate | 5.0377 | 0.0003 | 0.0145 | 2.9655 | 0.0136 | 0.1077 |
| IL2RA | Activate | 12.629 | 0.000036735 | 0.0059 | 9.0232 | 0.0004 | 0.03 |
| IL3RA | Activate | 2.0071 | 0.0463 | 0.2977 | −2.3554 | 0.0222 | 0.1263 |
| INHBA | Activate | 7.0079 | 0.0006 | 0.0238 | 8.1176 | 0.0005 | 0.0316 |
| IRF1 | Activate | 4.8896 | 0.0002 | 0.0136 | 4.2429 | 0.0011 | 0.0383 |
| IRF2 | Activate | 3.0856 | 0.0001 | 0.0102 | 2.3475 | 0.0038 | 0.0609 |
| IRF4 | Activate | 3.6001 | 0.0009 | 0.0303 | 2.7396 | 0.0107 | 0.0955 |
| IRF5 | Activate | 6.3959 | 0.0039 | 0.0697 | 6.2724 | 0.0066 | 0.0767 |
| IRF7 | Activate | 4.6951 | 0.0016 | 0.0408 | 2.6708 | 0.0468 | 0.1806 |
| IRGM1 | Activate | 3.5604 | 0.0205 | 0.1855 | 3.8887 | 0.0196 | 0.1263 |
| ISG15 | Activate | 6.1635 | 0.0003 | 0.0158 | 2.64 | 0.0488 | 0.1845 |
| ISG20 | Activate | 3.3567 | 0.0012 | 0.0349 | 3.9719 | 0.0006 | 0.0322 |
| ITGA4 | Activate | 2.0304 | 0.0397 | 0.272 | 2.0083 | 0.055 | 0.1984 |
| ITGAL | Activate | 16.3545 | 0.000032377 | 0.0056 | 6.1759 | 0.0055 | 0.0707 |
| ITGAM | Activate | 8.5704 | 0.0093 | 0.1151 | 3.5033 | 0.1371 | 0.3385 |
| ITGAX | Activate | 3.9189 | 0.0101 | 0.1202 | 2.0482 | 0.185 | 0.4084 |
| ITGB2 | Activate | 7.7479 | 0.0002 | 0.0124 | 6.9547 | 0.0006 | 0.0332 |
| JAK2 | Activate | 1.6647 | 0.0461 | 0.2976 | 1.3678 | 0.2378 | 0.4779 |
| JUN | Activate | 1.7589 | 0.0035 | 0.0649 | −1.1739 | 0.4037 | 0.6708 |
| KLF4 | Activate | 1.907 | 0.0409 | 0.2778 | 1.156 | 0.6546 | 0.9116 |
| KLF6 | Activate | 2.4625 | 0.0037 | 0.0672 | 2.1351 | 0.0182 | 0.125 |
| KYNU | Activate | 3.8871 | 0.008 | 0.1057 | 4.8859 | 0.0039 | 0.061 |
| LAG3 | Activate | 7.4221 | 0.0002 | 0.0114 | 2.156 | 0.1298 | 0.3262 |
| LAT2 | Activate | 2.6441 | 0.028 | 0.2208 | 1.5322 | 0.3475 | 0.6094 |
| LCN2 | Activate | 4.3524 | 0.0296 | 0.2275 | 16.3586 | 0.0003 | 0.0276 |
| LCP2 | Activate | 8.4235 | 0.0004 | 0.0193 | 4.07 | 0.0205 | 0.1263 |
| LGALS9 | Activate | 1.6825 | 0.0051 | 0.0817 | 1.6728 | 0.0085 | 0.086 |
| LST1 | Activate | 7.6841 | 0.02 | 0.1832 | 1.6707 | 0.5648 | 0.8328 |
| LTA | Activate | 2.7386 | 0.0055 | 0.0851 | 2.4968 | 0.0157 | 0.1157 |
| LTB | Activate | 8.1723 | 0.0014 | 0.0379 | 2.3655 | 0.1846 | 0.4079 |
| LY6E | Activate | 5.1119 | 0.0028 | 0.0572 | 2.8148 | 0.061 | 0.2104 |
| LY96 | Activate | 2.4218 | 0.0302 | 0.2305 | 1.5311 | 0.3103 | 0.5661 |
| LYN | Activate | 5.1219 | 0.001 | 0.0325 | 1.6841 | 0.2851 | 0.536 |
| MAP3K8 | Activate | 7.131 | 0.0009 | 0.0302 | 1.0205 | 0.9717 | 1 |
| MEFV | Activate | 10.8378 | 0.000019758 | 0.0047 | 2.3756 | 0.0951 | 0.2688 |
| MMP12 | Activate | 6.4662 | 0.0035 | 0.0647 | 5.0068 | 0.015 | 0.1131 |
| MX1 | Activate | 6.1489 | 0.0128 | 0.1395 | 2.6469 | 0.1916 | 0.4171 |
| MX2 | Activate | 3.9804 | 0.0005 | 0.0205 | 1.8666 | 0.1068 | 0.2889 |
| NAMPT | Activate | 2.341 | 0.0066 | 0.0943 | −1.4194 | 0.2673 | 0.514 |
| NAPSA | Activate | 4.3452 | 0.0003 | 0.0149 | 4.7434 | 0.0003 | 0.0272 |
| NEURL3 | Activate | 5.943 | 0.0009 | 0.0291 | 1.9354 | 0.2066 | 0.4363 |
| NFKB1 | Activate | 1.8089 | 0.0051 | 0.0815 | 1.0738 | 0.7351 | 0.9751 |
| NFKBIA | Activate | 4.9409 | 0.000013016 | 0.0041 | 2.4668 | 0.0097 | 0.0912 |
| NFKBIZ | Activate | 2.328 | 0.0073 | 0.0995 | 2.0257 | 0.0308 | 0.1436 |
| NGF | Activate | 3.1687 | 0.0065 | 0.0933 | 3.4968 | 0.0055 | 0.0706 |
| NLRC5 | Activate | 3.4173 | 0.0109 | 0.1261 | 2.2706 | 0.0985 | 0.2752 |
| NLRP3 | Activate | 5.11 | 0.0007 | 0.0254 | 3.4525 | 0.0111 | 0.0975 |
| NMI | Activate | 2.0435 | 0.0228 | 0.1979 | 1.6117 | 0.1416 | 0.3459 |
| NOD2 | Activate | 3.5157 | 0.000018135 | 0.0045 | 2.5449 | 0.0013 | 0.0396 |
| NOS2 | Activate | 26.5216 | 2.2445E−06 | 0.0017 | 24.8128 | 7.9556E−06 | 0.0117 |
| NOTCH1 | Activate | 2.2952 | 0.0301 | 0.2303 | 1.4949 | 0.3078 | 0.5625 |
| NR1D1 | Activate | 1.7995 | 0.0049 | 0.0799 | 1.8784 | 0.0045 | 0.065 |
| NUPR1 | Activate | 2.8421 | 0.001 | 0.0318 | 1.7353 | 0.0807 | 0.2444 |
| OAS2 | Activate | 3.8997 | 0.0003 | 0.0154 | 2.3682 | 0.0204 | 0.1263 |
| OAS3 | Activate | 14.5317 | 7.7153E−06 | 0.0034 | 4.0924 | 0.0123 | 0.1019 |
| P2RY14 | Activate | 16.6314 | 0.0005 | 0.0204 | 4.8513 | 0.0471 | 0.1812 |
| P2RY6 | Activate | 5.352 | 0.0001 | 0.0103 | 2.2094 | 0.0606 | 0.2093 |
| PARP9 | Activate | 3.1267 | 0.0179 | 0.1716 | −1.1573 | 0.7644 | 0.9977 |
| PARVG | Activate | 6.3047 | 0.0025 | 0.0539 | 1.2154 | 0.7444 | 0.9822 |
| PDCD1LG2 | Activate | 11.6201 | 2.1928E−06 | 0.0017 | 3.9612 | 0.0045 | 0.065 |
| PF4 | Activate | 6.7372 | 0.0284 | 0.2226 | −2.2437 | 0.3655 | 0.6285 |
| PIM1 | Activate | 4.0708 | 0.0003 | 0.0169 | 2.0732 | 0.0575 | 0.2034 |
| PLA2G16 | Activate | 4.8619 | 0.001 | 0.0309 | 1.3802 | 0.4886 | 0.7613 |
| PLAU | Activate | 3.8214 | 0.00003401 | 0.0058 | 2.1662 | 0.0131 | 0.1056 |
| PLEK | Activate | 5.9274 | 0.0049 | 0.0805 | 2.9561 | 0.0921 | 0.2632 |
| PMAIP1 | Activate | 3.4224 | 0.0002 | 0.0134 | 2.6199 | 0.0041 | 0.0627 |
| PRDM1 | Activate | 3.5702 | 0.0297 | 0.2283 | 4.0335 | 0.0251 | 0.13 |
| PRKCD | Activate | 4.5574 | 2.2524E−07 | 0.001 | 2.0325 | 0.0066 | 0.0768 |
| PRKCQ | Activate | 2.1379 | 0.0065 | 0.0932 | 2.1615 | 0.0089 | 0.087 |
| PSMB10 | Activate | 3.6197 | 0.005 | 0.0807 | 1.4724 | 0.3976 | 0.6643 |
| PSMB8 | Activate | 3.9093 | 0.0032 | 0.0615 | 2.652 | 0.0388 | 0.1637 |
| PSMB9 | Activate | 7.4132 | 0.0005 | 0.0197 | 4.5308 | 0.009 | 0.0877 |
| PSME1 | Activate | 6.1609 | 0.0001 | 0.0096 | 3.9584 | 0.0037 | 0.06 |
| PTAFR | Activate | 8.5218 | 0.000052564 | 0.0069 | 2.2614 | 0.1024 | 0.2821 |
| PTGES | Activate | 3.3225 | 0.0241 | 0.2039 | 1.61 | 0.3819 | 0.6479 |
| PTGS2 | Activate | 10.9036 | 0.0075 | 0.1017 | 3.9538 | 0.1309 | 0.3282 |
| PTX3 | Activate | 5.271 | 0.0253 | 0.2091 | 1.8833 | 0.4052 | 0.672 |
| RAB20 | Activate | 2.29 | 0.0257 | 0.2106 | 1.1261 | 0.7537 | 0.9893 |
| RAC2 | Activate | 7.9519 | 0.000098427 | 0.0091 | 3.7008 | 0.0123 | 0.1021 |
| RIPK1 | Activate | 1.6822 | 0.0456 | 0.2959 | −1.1344 | 0.6373 | 0.8964 |
| RSAD2 | Activate | 3.916 | 0.0003 | 0.0152 | 1.6141 | 0.184 | 0.407 |
| RTP4 | Activate | 4.8033 | 0.0025 | 0.0537 | 2.5839 | 0.0696 | 0.2269 |
| RUNX2 | Activate | 5.3191 | 0.0001 | 0.0095 | 2.2697 | 0.0491 | 0.185 |
| RUNX3 | Activate | 5.5652 | 0.0012 | 0.0354 | 5.5875 | 0.002 | 0.0467 |
| S100A10 | Activate | 2.1419 | 0.001 | 0.0318 | −1.0647 | 0.7799 | 1 |
| S100A8 | Activate | 10.9785 | 0.0065 | 0.0931 | 11.8118 | 0.0079 | 0.0832 |
| S100A9 | Activate | 5.1245 | 0.0058 | 0.0879 | 4.0622 | 0.0228 | 0.1263 |
| S1PR3 | Activate | 2.8084 | 0.0426 | 0.2844 | 4.3054 | 0.0083 | 0.0851 |
| SAMHD1 | Activate | 6.8165 | 0.000028539 | 0.0053 | 5.2676 | 0.0004 | 0.0289 |
| SELL | Activate | 9.0266 | 0.000036704 | 0.0059 | 2.6848 | 0.0503 | 0.1879 |
| SEMA4A | Activate | 4.5461 | 0.0002 | 0.0134 | 7.1376 | 0.000014188 | 0.0118 |
| SEPT3 | Activate | 1.9813 | 0.0021 | 0.0484 | 1.2827 | 0.2581 | 0.5036 |
| SERPINB9 | Activate | 3.6968 | 0.0068 | 0.0955 | 1.3544 | 0.5306 | 0.8014 |
| SERPINE1 | Activate | 2.7348 | 0.0131 | 0.142 | 1.0382 | 0.9268 | 1 |
| SERPING1 | Activate | 8.5956 | 0.0003 | 0.0154 | 5.6206 | 0.0042 | 0.063 |
| SHARPIN | Activate | 1.8222 | 0.0003 | 0.0169 | −1.1856 | 0.2897 | 0.5414 |
| SLAMF1 | Activate | 4.2282 | 0.000066073 | 0.0076 | 1.8422 | 0.0753 | 0.2344 |
| SLC11A1 | Activate | 8.1631 | 0.0002 | 0.0114 | 6.6594 | 0.0009 | 0.0364 |
| SLC15A3 | Activate | 13.0122 | 0.0001 | 0.0093 | 4.2688 | 0.0238 | 0.1278 |
| SLC16A9 | Activate | 2.2466 | 0.0273 | 0.2172 | 1.6072 | 0.21 | 0.4406 |
| SLC28A2 | Activate | 6.903 | 0.0001 | 0.0096 | 1.4304 | 0.4466 | 0.7176 |
| SLFN5 | Activate | 3.7058 | 0.0022 | 0.0493 | 2.1361 | 0.0772 | 0.2383 |
| SMAD7 | Activate | 3.1247 | 0.0209 | 0.187 | −1.1526 | 0.7767 | 1 |
| SOCS1 | Activate | 3.9437 | 0.011 | 0.1267 | 3.3365 | 0.0325 | 0.1481 |
| SOD2 | Activate | 2.6759 | 0.0047 | 0.0774 | 3.1489 | 0.0021 | 0.0475 |
| SOD3 | Activate | 2.1257 | 0.0158 | 0.1591 | 1.4255 | 0.2663 | 0.5128 |
| SP100 | Activate | 4.8282 | 7.0055E−06 | 0.0032 | 2.8699 | 0.0019 | 0.0452 |
| SP110 | Activate | 3.7976 | 0.0057 | 0.0867 | 1.1957 | 0.71 | 0.9556 |
| SPI1 | Activate | 10.5793 | 0.000055966 | 0.0071 | 6.5856 | 0.0014 | 0.0409 |
| SPP1 | Activate | 2.5268 | 0.0212 | 0.1893 | 2.4021 | 0.0384 | 0.1624 |
| STAT1 | Activate | 4.2736 | 0.0089 | 0.1122 | 3.9844 | 0.0176 | 0.1229 |
| STAT2 | Activate | 5.4847 | 0.000013182 | 0.0041 | 3.019 | 0.0035 | 0.0589 |
| STAT4 | Activate | 6.3819 | 0.0038 | 0.069 | 1.1576 | 0.8175 | 1 |
| STAT6 | Activate | 1.7399 | 0.0097 | 0.1171 | 1.7712 | 0.0116 | 0.0993 |
| STX11 | Activate | 3.6303 | 0.0058 | 0.088 | 1.7593 | 0.2306 | 0.4679 |
| TAP1 | Activate | 4.588 | 0.001 | 0.0323 | 2.7092 | 0.0329 | 0.1493 |
| TAP2 | Activate | 2.8268 | 0.0148 | 0.1526 | 1.5315 | 0.3257 | 0.585 |
| TAPBP | Activate | 2.5416 | 0.0274 | 0.2176 | 1.7971 | 0.1801 | 0.4019 |
| TAPBPL | Activate | 3.0796 | 0.0031 | 0.0609 | 2.4014 | 0.0251 | 0.13 |
| TBX21 | Activate | 3.8788 | 0.0008 | 0.0273 | 4.1566 | 0.0008 | 0.0354 |
| TCIRG1 | Activate | 2.7899 | 0.0005 | 0.0217 | 1.2508 | 0.4321 | 0.7008 |
| TGFBR2 | Activate | 1.9044 | 0.031 | 0.2346 | 1.2596 | 0.451 | 0.7222 |
| THBS1 | Activate | 2.8135 | 0.005 | 0.0807 | 2.9522 | 0.0055 | 0.0707 |
| THEMIS2 | Activate | 9.3368 | 0.0011 | 0.0339 | 6.5099 | 0.0079 | 0.0832 |
| THY1 | Activate | 5.5745 | 0.0036 | 0.0666 | 3.0999 | 0.0597 | 0.2073 |
| TLR1 | Activate | 3.9534 | 0.0044 | 0.0751 | 4.9709 | 0.002 | 0.0463 |
| TLR2 | Activate | 4.2854 | 0.0004 | 0.0173 | 1.0613 | 0.8785 | 1 |
| TLR7 | Activate | 3.6076 | 0.0317 | 0.2384 | 1.1789 | 0.7874 | 1 |
| TLR8 | Activate | 7.9426 | 0.0012 | 0.0349 | 1.5453 | 0.4846 | 0.7574 |
| TLR9 | Activate | 7.0125 | 0.0004 | 0.0182 | 5.5003 | 0.0026 | 0.052 |
| TMEM50B | Activate | 2.2646 | 0.0029 | 0.0584 | 1.6005 | 0.089 | 0.2584 |
| TNF | Activate | 15.5761 | 0.000001549 | 0.0016 | 3.6025 | 0.014 | 0.1094 |
| TNFAIP2 | Activate | 4.2629 | 0.0043 | 0.0737 | 1.3184 | 0.5837 | 0.8504 |
| TNFRSF11A | Activate | 3.7342 | 0.0089 | 0.1124 | 3.2927 | 0.0236 | 0.1273 |
| TNFRSF14 | Activate | 3.3507 | 0.0151 | 0.1547 | 2.0443 | 0.1615 | 0.3761 |
| TNFRSF1B | Activate | 6.9767 | 0.0004 | 0.0187 | 3.0014 | 0.0433 | 0.1723 |
| TNFSF10 | Activate | 5.9466 | 0.0008 | 0.0286 | 2.8254 | 0.0505 | 0.1883 |
| TNFSF12 | Activate | 4.0396 | 0.0003 | 0.0145 | 3.0636 | 0.0039 | 0.061 |
| TNFSF13 | Activate | 3.7405 | 0.0229 | 0.1979 | −1.7547 | 0.3434 | 0.6048 |
| TNFSF13B | Activate | 4.2939 | 0.00009167 | 0.0089 | 1.0175 | 0.96 | 1 |
| TRAFD1 | Activate | 7.5123 | 0.000066493 | 0.0076 | 1.4364 | 0.4415 | 0.7113 |
| TRIM21 | Activate | 3.1067 | 0.0069 | 0.096 | −1.1129 | 0.7984 | 1 |
| TXK | Activate | 3.1599 | 0.0017 | 0.0418 | 1.8261 | 0.0992 | 0.2766 |
| UBD | Activate | 39.4489 | 3.1101E−06 | 0.0021 | 20.5238 | 0.0001 | 0.0203 |
| UBE2L6 | Activate | 3.418 | 0.002 | 0.0464 | 4.4962 | 0.0005 | 0.031 |
| USP18 | Activate | 5.4886 | 0.0004 | 0.0192 | 1.697 | 0.258 | 0.5036 |
| VCAM1 | Activate | 7.3103 | 0.0003 | 0.0163 | 1.2409 | 0.6812 | 0.9337 |
| WARS | Activate | 1.5033 | 0.0098 | 0.1179 | 1.8802 | 0.0003 | 0.0284 |
| ZFP36 | Activate | 3.0067 | 0.0051 | 0.0815 | −1.1887 | 0.6585 | 0.9153 |
| AHCY | Inhibit | −2.0639 | 0.0374 | 0.2628 | −1.8643 | 0.0878 | 0.2566 |
| AQP1 | Inhibit | −2.6893 | 0.0012 | 0.0354 | −2.9843 | 0.0008 | 0.0346 |
| AZGP1 | Inhibit | −3.6306 | 0.0039 | 0.0701 | −3.72 | 0.0054 | 0.0705 |
| CSE1L | Inhibit | −1.9528 | 0.0448 | 0.2931 | −4.9001 | 0.000053442 | 0.0177 |
| DHX9 | Inhibit | −1.9049 | 0.0192 | 0.1787 | −1.3891 | 0.2439 | 0.4863 |
| EMID1 | Inhibit | −2.5566 | 0.047 | 0.3003 | −1.9661 | 0.1701 | 0.3887 |
| FKBP6 | Inhibit | −4.1867 | 0.0043 | 0.0733 | −2.2618 | 0.1075 | 0.2898 |
| FLT4 | Inhibit | −1.7478 | 0.0248 | 0.2076 | −1.6367 | 0.0588 | 0.2058 |
| GMPR | Inhibit | −2.9853 | 0.027 | 0.2154 | −5.5877 | 0.0017 | 0.0432 |
| GNAO1 | Inhibit | −2.2497 | 0.0179 | 0.1721 | −1.0647 | 0.8565 | 1 |
| HSP90AB1 | Inhibit | −1.9951 | 0.007 | 0.0974 | −1.1404 | 0.6083 | 0.8715 |
| IDI1 | Inhibit | −2.9218 | 0.004 | 0.0713 | −2.402 | 0.023 | 0.1263 |
| IGFBP4 | Inhibit | −3.3872 | 0.0211 | 0.1885 | −3.0044 | 0.0473 | 0.1815 |
| MIF | Inhibit | −3.5707 | 0.0052 | 0.0828 | −1.3189 | 0.5425 | 0.8124 |
| MSH2 | Inhibit | −1.909 | 0.0328 | 0.2435 | −1.7932 | 0.0664 | 0.2198 |
| MYC | Inhibit | −1.8764 | 0.0225 | 0.1962 | −1.5059 | 0.1507 | 0.36 |
| PBK | Inhibit | −4.8198 | 0.0008 | 0.0287 | −10.4275 | 0.000012076 | 0.0117 |
| PCDH17 | Inhibit | −2.2868 | 0.0029 | 0.0588 | −1.1447 | 0.6223 | 0.8835 |
| PLCG1 | Inhibit | −1.6389 | 0.0439 | 0.2895 | 1.263 | 0.3564 | 0.6189 |
| POLR2F | Inhibit | −1.7137 | 0.0139 | 0.1473 | −2.1058 | 0.0019 | 0.0456 |
| POMP | Inhibit | −1.5182 | 0.047 | 0.3002 | −1.3884 | 0.1354 | 0.336 |
| PRPF8 | Inhibit | −2.0788 | 0.0159 | 0.1593 | −2.1397 | 0.0179 | 0.1239 |
| PTGES2 | Inhibit | −1.9137 | 0.0278 | 0.2201 | −1.0746 | 0.811 | 1 |
| RAD18 | Inhibit | −2.1129 | 0.0032 | 0.0615 | −1.7251 | 0.0354 | 0.1551 |
| SF3A1 | Inhibit | −1.598 | 0.0042 | 0.0729 | −1.2281 | 0.2111 | 0.4418 |
| SLC12A2 | Inhibit | −2.8366 | 0.0039 | 0.0697 | −3.5987 | 0.0011 | 0.0373 |
| SMTN | Inhibit | −1.8432 | 0.0398 | 0.2725 | −1.2994 | 0.3927 | 0.6593 |
| SQLE | Inhibit | −4.0941 | 0.0396 | 0.2717 | −4.8892 | 0.0296 | 0.1404 |
| SREBF2 | Inhibit | −2.3929 | 0.0032 | 0.0615 | −3.5164 | 0.0001 | 0.0209 |
| SSBP1 | Inhibit | −1.5752 | 0.0094 | 0.1156 | 1.0074 | 0.9665 | 1 |
| TMEM158 | Inhibit | −1.6002 | 0.044 | 0.2897 | 1.4394 | 0.1352 | 0.3357 |
| TYMP | Inhibit | −2.6432 | 0.0448 | 0.2928 | 1.469 | 0.4412 | 0.7111 |
| UNC5B | Inhibit | −2.3093 | 0.0267 | 0.2143 | −1.5396 | 0.2666 | 0.5132 |

### Example 13—Cytokine mRNA Increases CD4+ and CD8+ T Cells in Both Treated and Untreated Tumors

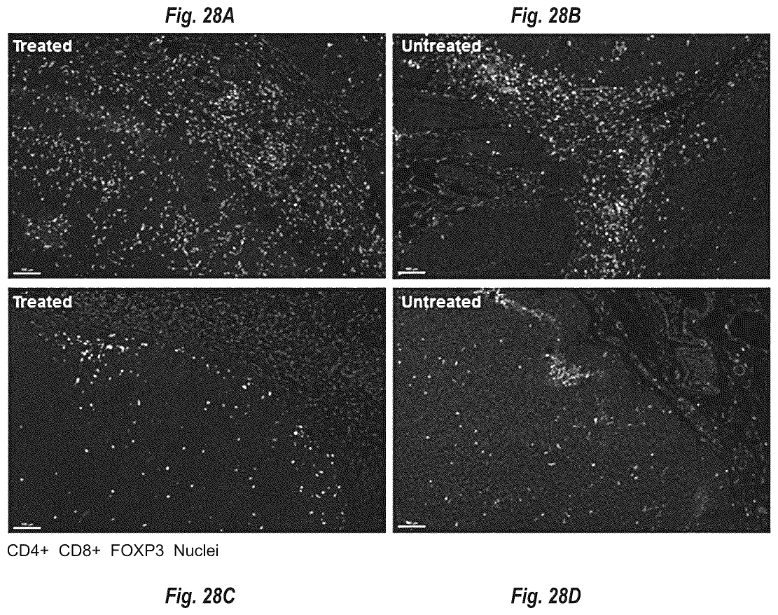

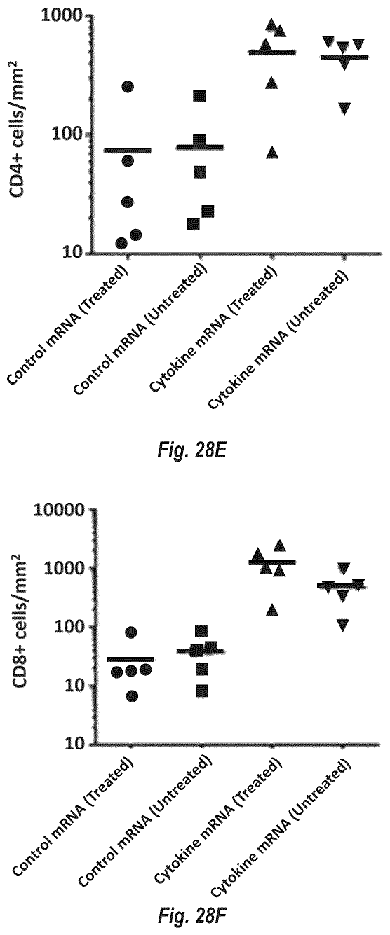

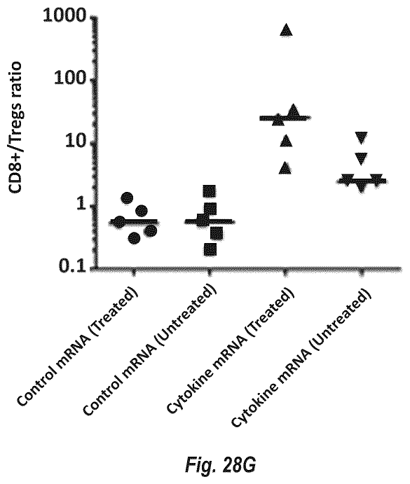

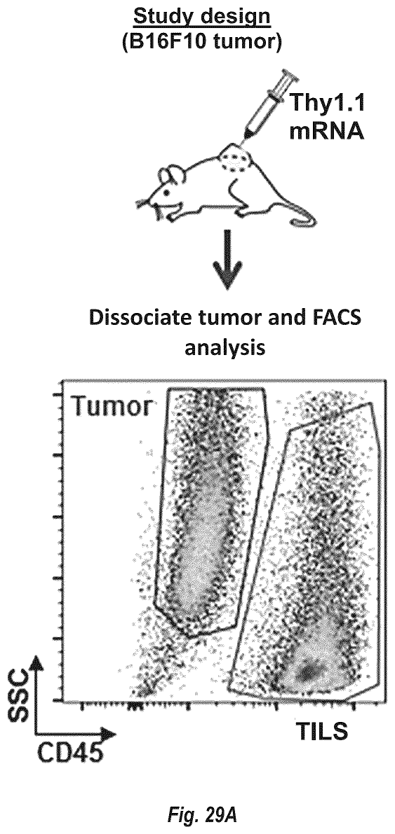

Mice bearing B16F10 tumors on the left and right flank received a single intratumoral injection of 80 μg of mRNA (20 μg/target) into right tumor which was initiated with 0.5×10\hat{ }6 cells only one of the tumors (treated), while the other tumor initiated with 0.25×10\hat{ }6 cells remained untreated. At seven days post intratumoral mRNA injection, both the tumors were collected and processed for IHC (Immunofluorescence microscopy) staining with antibodies for CD4+, CD8+, and FoxP3+ cells. Mice from tumors in FIGS. 28A and 28B were treated with cytokine mRNA, while mice from tumors in FIGS. 28C and D were treated with control mRNA. Panels A and C are from the tumors injected with mRNA, while panels B and D are from the corresponding contralateral tumor not injected. (FIGS. 28A-D). For both the cytokine mRNA treated and control mRNA treated groups 5 tumors injected with RNA and the corresponding 5 contralateral tumors uninjected were subjected to immunofluorescent staining for CD4+, CD8+, FOXP3+ cells. The relative frequency and ratio of cells are plotted in FIGS. 28E, F, G. The results indicate that a cytokine mRNA mixture of IL-15 sushi, GM-CSF, IFNα, and IL-12sc (SEQ ID NOs: 59, 53, 41, and 47) increases CD8+ and CD4+ T cells infiltration leading to altering the CD8+/Treg ratio. An increase in immune infiltration occurred in both the treated and untreated tumors, supporting the notion that local intratumoral treatment has systemic immune modulatory effects.

### Example 14—mRNA is Expressed in Both Tumor and Infiltrating Immune Cells

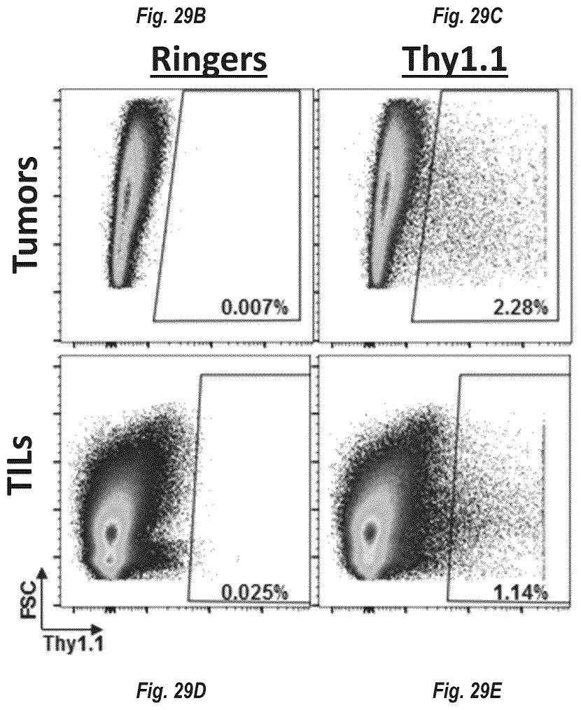

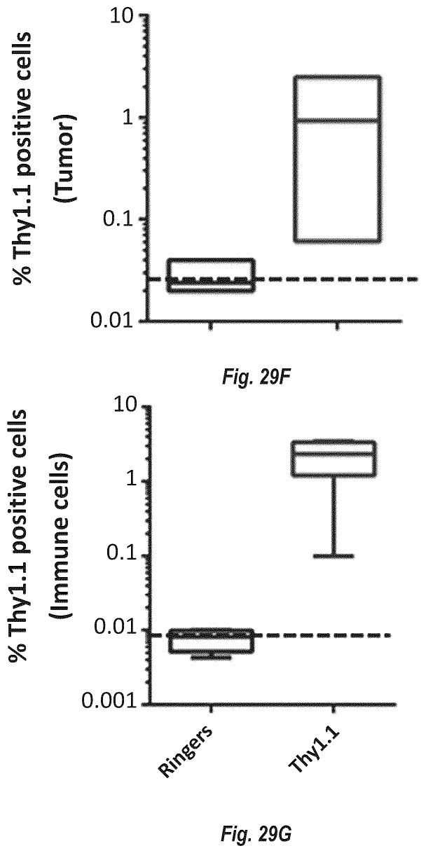

Mice bearing a single B16F10 tumor received a single intratumoral injection with mRNA encoding the Thy1.1 protein (FIG. 29A-G). Approximately 18 hrs after intratumoral injection the tumor was dissociated, stained with a panel of antibodies and flow cytometry was performed to define cells expressing Thy1.1 protein. The results indicate that both tumor and immune cells take up and express the mRNA.

### Example 15—Dose Dependent Tumor Expression and PD Response Following Intratumoral Cytokine mRNA Injection

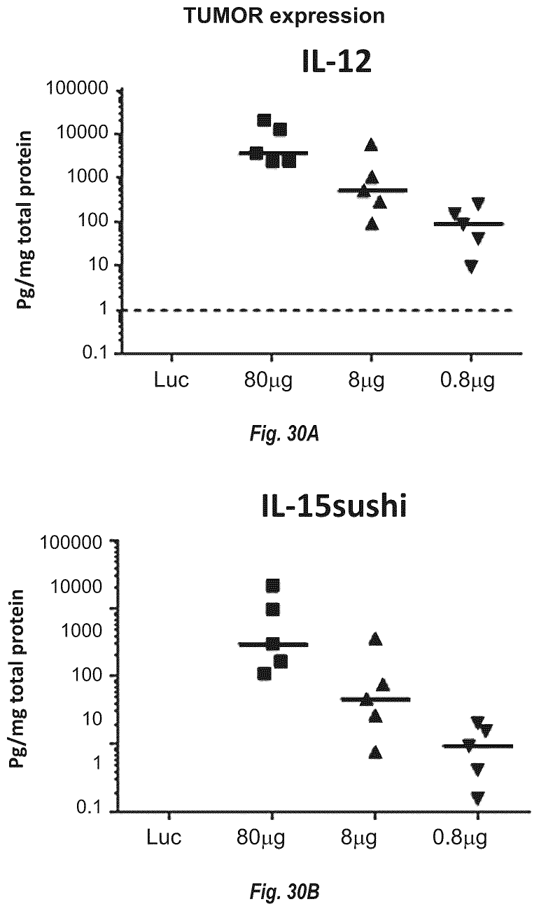

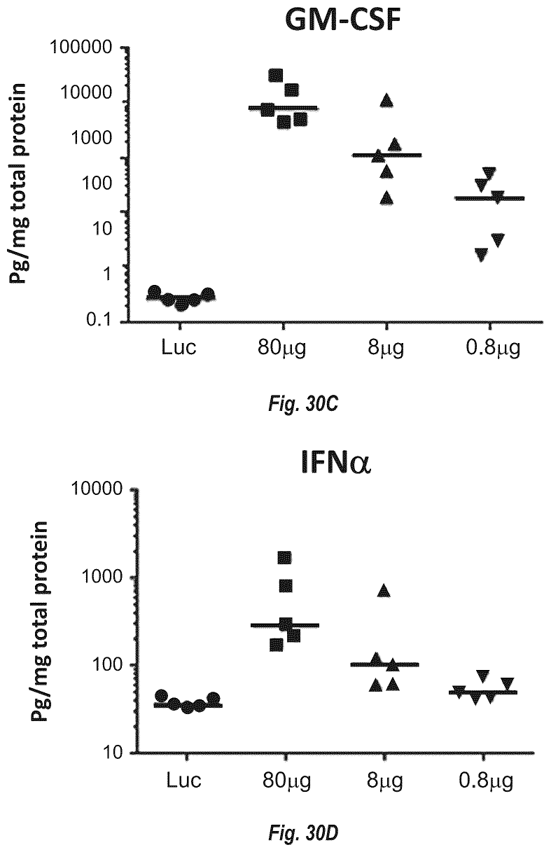

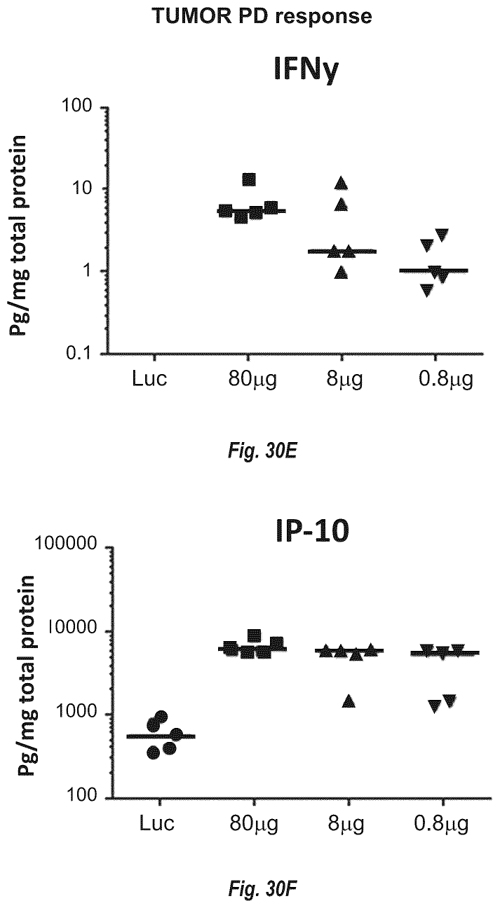

Mice bearing B16F10 tumors received a single mRNA injection with 80, 8 or 0.8 μg of a cytokine mRNA mixture of IL-15 sushi, GM-CSF, IFNα, and IL-12sc (SEQ ID Nos: 59, 53, 41, and 47). Approximately 6 hrs after the intratumoral injection, the tumor was removed and lysed, and levels of IL-15 sushi, GM-CSF, IFNα, and IL-12sc, IFNgamma and IP-10 were quantified in the tumor lysate. FIGS. 30A-F show that the cytokine mRNA was expressed intratumorally in a dose-dependent manner.

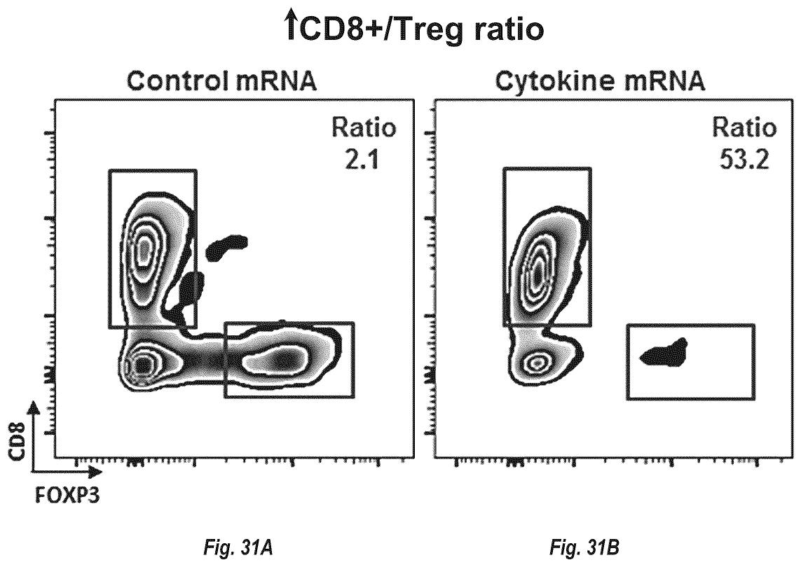

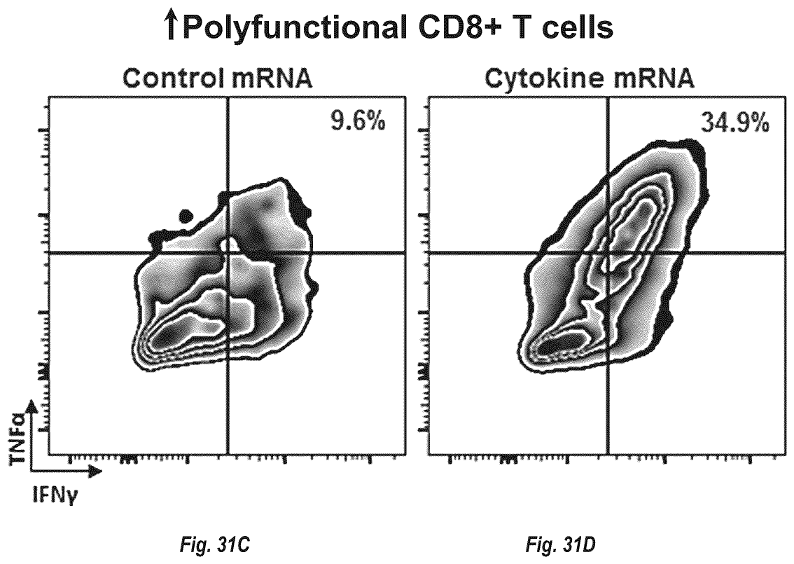

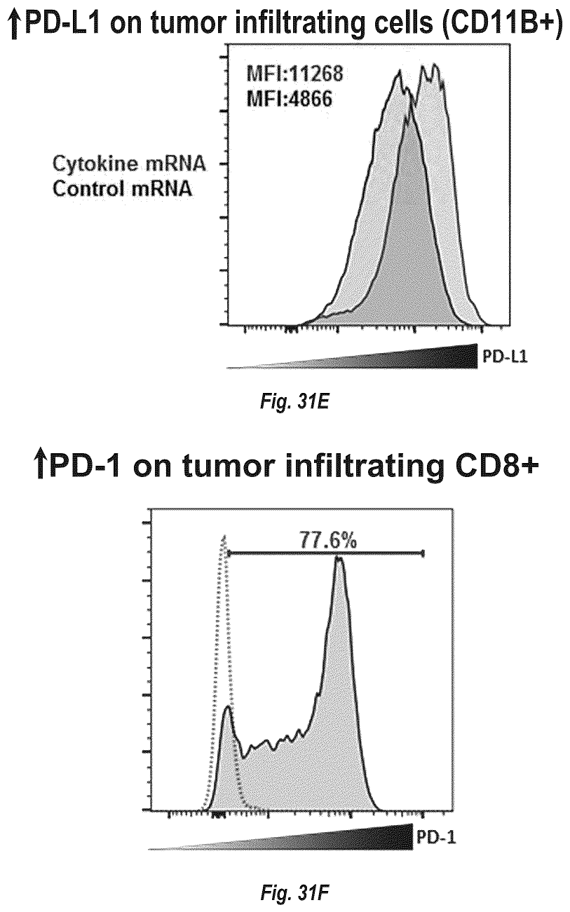

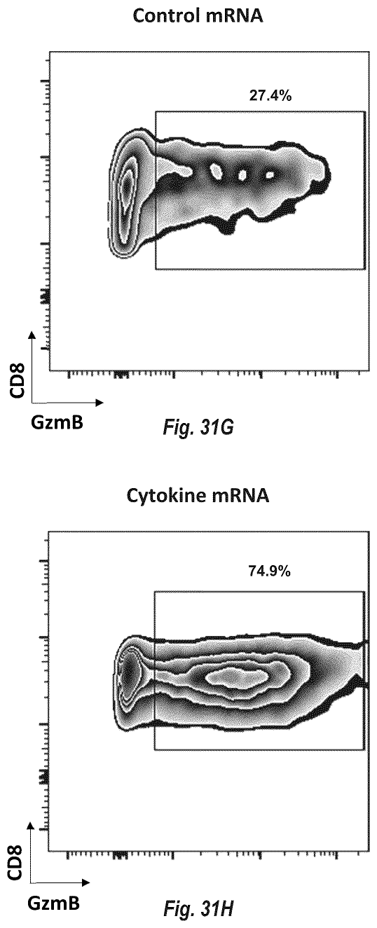

In a separate experiment, mice bearing B16F10 tumors received a single mRNA injection with 80, 8 or 0.8 ug of a cytokine mRNA mixture of IL-15 sushi, GM-CSF, IFNα, and IL-12sc (SEQ ID Nos: 59, 53, 41, and 47). At seven days following intratumoral cytokine mRNA injection, the tumors were dissociated, stained with a panel of antibodies, and analyzed by flow cytometry. The antibodies used were against murine: CD45, CD4, CD3, CD8, CD279, IFNgamma, TNFalpha, FOXP3, Granzyme B). The results indicate that treatment with the cytokine mRNA mixture altered the CD8+/Treg ratio (FIG. 31A-B), led to increased frequency of polyfunctional CD8+ T cells in the tumor microenvironment (FIG. 31C-D), increased PD-L1 on infiltrating myeloid cells (FIG. 31E), and increased levels of PD-1 on infiltrating CD8+ T cells (FIG. 31F).

In a further experiment, mice bearing B16F10 tumors on the left and right flank received a single intratumoral injection of a cytokine mRNA mixture of IL-15 sushi, GM-CSF, IFNα, and IL-12sc (SEQ ID Nos: SEQ ID NOs: 59, 53, 41, and 47) or control mRNA into only one of the tumors (treated), while the other tumor remained untreated. At seven days post intratumoral mRNA injection, the injected tumor was collected and processed for flow cytometry staining with antibodies for CD45+, CD8+, CD3+, and Granzyme B. The results indicate that the cytokine mRNA mixture increased the frequency of intratumoral Granzyme B CD8+ T cells in the tumor (FIG. 31G-H).

### Example 16—Intratumorally Injected mRNA is Primarily Expressed in the Tumor

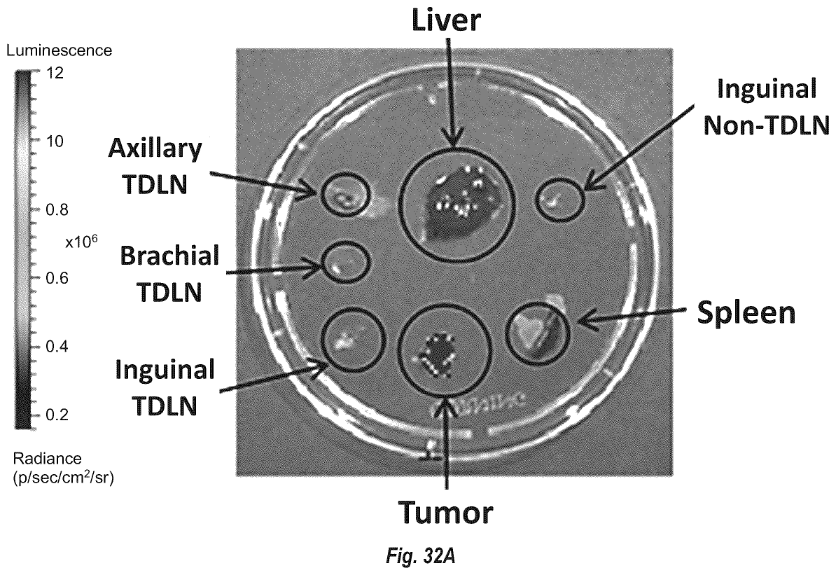

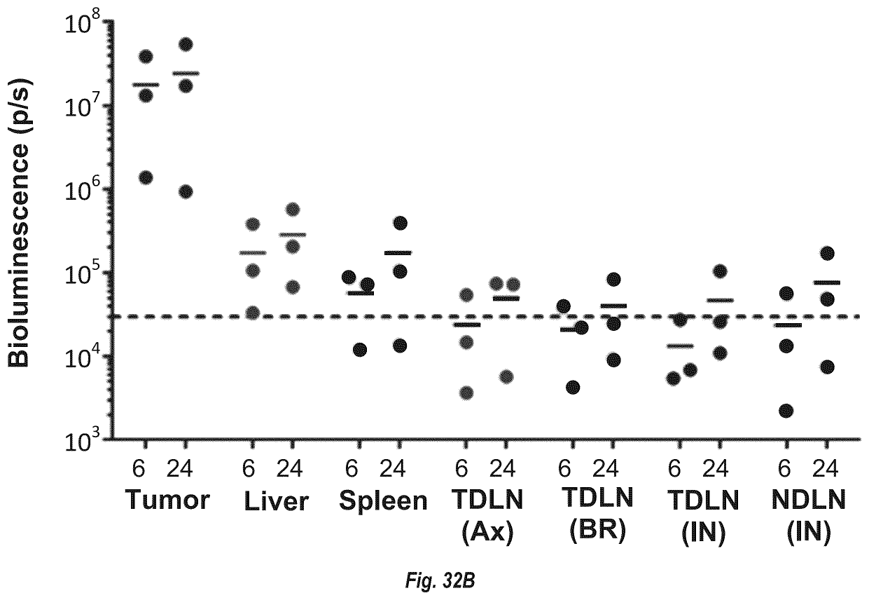

Mice bearing B16F10 tumors received a single intratumoral injection of 50 μg mRNA encoding firefly luciferase. At 6 and 24-hour time points, 3 mice were sacrificed and tumor, liver, spleen, tumor draining lymph node (TDLN) and non-tumor draining lymph node (NDLN) were analyzed ex vivo for luciferase expression. FIGS. 32A-B show that luciferase expression was highest in the tumor, in which expression was greater than 100-fold above any other tissue.

### Example 17—CD4+, CD8+, and NK Cells Contribute to the Anti-Tumor Activity of Cytokine mRNA in B16F10 Model

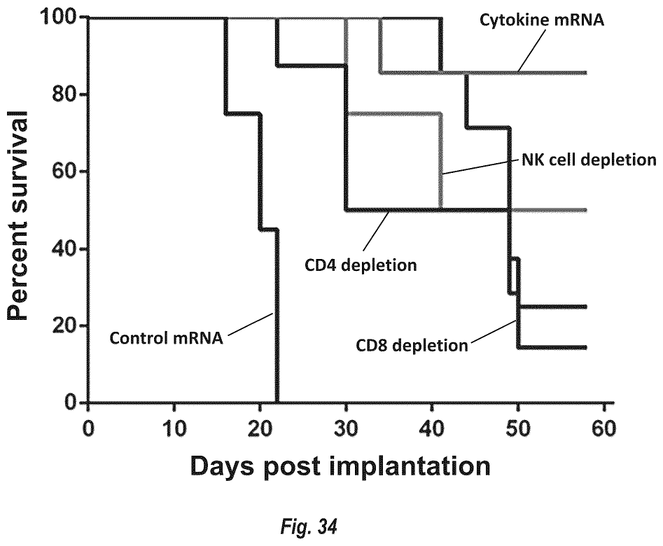

Groups of mice bearing B16F10 tumors were treated with 100 μg of depleting antibodies (anti-CD4, anti-CD8, anti-NKI.1) by intraperitoneal injection once a week for 4 weeks total. Antibody mediated cellular depletion was initiated one day prior to treatment with an 80 μg cytokine mRNA mixture of IL-15 sushi, GM-CSF, IFNα, and IL-12sc (SEQ ID Nos: 59, 53, 41, and 47). The effect of antibody depletion on overall survival was monitored. The results, shown in FIG. 34, indicate that individual depletion CD8+, CD4+, or NK cells reduced, to varying degrees, the anti-tumor activity and overall survival of the cytokine mRNA.

### Example 18—Antitumor Activity of Cytokine mRNAs is not Observed in IFN-Gamma-Deficient Mice

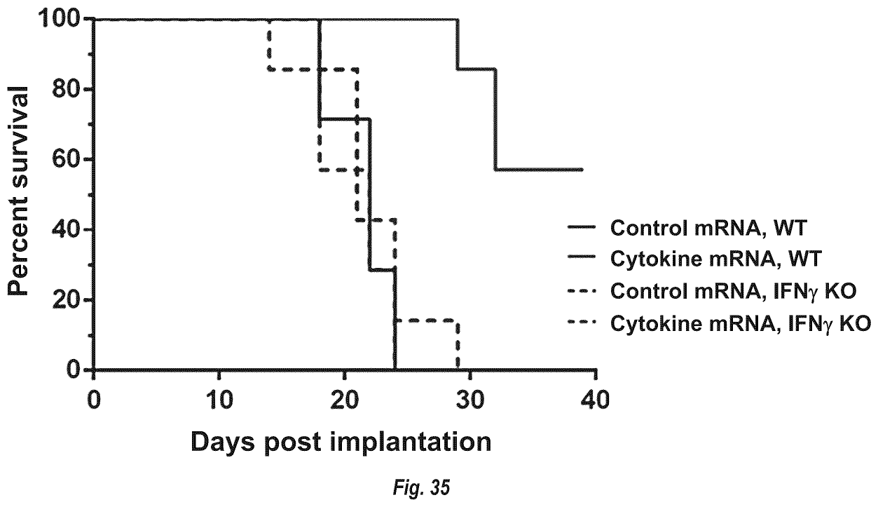

WT C57BL6J mice and C57BL6J mice deficient for the murine IFNγ (IFNγ KO) were implanted with B16F10 tumor cells as described in Example 1. Mice were treated by intratumoral injection with 80 μg (20 μg/target) cytokine mRNA mixture of IL-15 sushi, GM-CSF, IFNα, and IL-12sc (SEQ ID Nos: 59, 53, 41, and 47) or 80 μg control mRNA, and overall survival was monitored. The results, depicted in FIG. 35, indicate that mice lacking IFNγ did not exhibit a detectable antitumor response when treated with the cytokine mRNA.

1-88. (canceled)

89. A method for treating, or reducing the likelihood of, a solid tumor comprising administering to a subject in need thereof a composition comprising RNA encoding an IL-12sc protein, RNA encoding an IL-15 sushi protein, RNA encoding an IFNα protein, and RNA encoding a GM-CSF protein, wherein each RNA comprises a modified nucleoside in place of each uridine, wherein the modified nucleoside is pseudouridine (ψ), N1-methyl-pseudouridine (m¹ψ), 5-methyl-uridine (m5U), or a combination thereof, and wherein
the IL-12sc protein comprises an amino acid sequence having at least 95% identity to the amino acid sequence of SEQ ID NO: 14;
the IL-15 sushi protein comprises an amino acid sequence having at least 95% identity to the amino acid sequence of SEQ ID NO: 24
the IFNα protein comprises an amino acid sequence having at least 95% identity to the amino acid sequence of SEQ ID NO: 19; and
the GM-CSF protein comprises an amino acid sequence having at least 95% identity to the amino acid sequence of SEQ ID NO: 27.

90. The method of claim 89, wherein
the RNA encoding an IL-12sc protein comprises a nucleotide sequence having at least 95% identity to the nucleotide sequence of SEQ ID NO: 17 or 18;
the RNA encoding an IL-15 sushi protein comprises a nucleotide sequence having at least 95% identity to the nucleotide sequence of SEQ ID NO: 26;
the RNA encoding an IFNα protein comprises a nucleotide sequence having at least 95% identity to the nucleotide sequence of SEQ ID NO: 22 or 23; and/or
the RNA encoding a GM-CSF protein comprises a nucleotide sequence having at least 95% identity to the nucleotide sequence of SEQ ID NO: 29.

91. The composition of claim 89, wherein (i) the RNA encoding an IL-12sc protein comprises the nucleotide sequence of SEQ ID NO: 18; and/or (ii) the IL-12sc protein comprises the amino acid sequence of SEQ ID NO: 14.

92. The composition of claim 89, wherein the RNA encoding an IL-12sc protein comprises the nucleotide sequence of SEQ ID NO: 18 with an N1-methyl-pseudouridine (m¹ψ) in place of each uridine.

93. The composition of claim 89, wherein (i) the RNA encoding an IL-15 sushi protein comprises the nucleotide sequence of SEQ ID NO: 26; and/or (ii) the IL-15 sushi protein comprises the amino acid sequence of SEQ ID NO: 24.

94. The composition of claim 89, wherein the RNA encoding an IL-15 sushi protein comprises the nucleotide sequence of SEQ ID NO: 26 with an N1-methyl-pseudouridine (m¹ψ) in place of each uridine.

95. The composition of claim 89, wherein (i) the RNA encoding an IFNα protein comprises the nucleotide sequence of SEQ ID NO: 23; and/or (ii) the IFNα protein comprises the amino acid sequence of SEQ ID NO: 19.

96. The composition of claim 89, wherein the RNA encoding an IFNα protein comprises the nucleotide sequence of SEQ ID NO: 23 with an N1-methyl-pseudouridine (m¹ψ) in place of each uridine.

97. The composition of claim 89, wherein (i) the RNA encoding a GM-CSF protein comprises the nucleotide sequence of SEQ ID NO: 29; and/or (ii) the GM-CSF protein comprises the amino acid sequence of SEQ ID NO: 27.

98. The composition of claim 89, wherein the RNA encoding a GM-CSF protein comprises the nucleotide sequence of SEQ ID NO: 29 with an N1-methyl-pseudouridine (m¹ψ) in place of each uridine.

99. The composition of claim 89, wherein each RNA comprises an N1-methyl-pseudouridine (m¹ψ) in place of each uridine.

100. The method of claim 89, wherein the RNA is administered intra-tumorally or peri-tumorally.

101. The method of claim 89, wherein the subject is further treated with an additional therapy comprising (i) surgery to excise, resect, or debulk a tumor, (ii) immunotherapy, (iii) radiotherapy, or (iv) chemotherapy.

102. The method of claim 89, wherein the subject is further treated with a checkpoint modulator.

103. The method of claim 102, wherein the checkpoint modulator is an anti-PD1 antibody, an anti-CTLA-4 antibody, or a combination of an anti-PD1 antibody and an anti-CTLA-4 antibody.

104. The method of claim 102, wherein the RNA is administered intra-tumorally or peri-tumorally via injection, and the checkpoint modulator is administered systemically.

105. The method of claim 89, wherein the solid tumor is a sarcoma, carcinoma, or lymphoma.

106. The method of claim 89, wherein the solid tumor is in the lung, colon, ovary, cervix, uterus, peritoneum, testicles, penis, tongue, pancreas, bone, breast, prostate, soft tissue, connective tissue, kidney, liver, brain, thyroid, or skin.

107. The method of claim 89, wherein the solid tumor is an epithelial tumor, Hodgkin lymphoma (HL), non-Hodgkin lymphoma, prostate tumor, ovarian tumor, renal cell tumor, gastrointestinal tract tumor, hepatic tumor, colorectal tumor, tumor with vasculature, mesothelioma tumor, pancreatic tumor, breast tumor, sarcoma tumor, lung tumor, colon tumor, brain tumor, melanoma tumor, small cell lung tumor, neuroblastoma tumor, testicular tumor, carcinoma tumor, adenocarcinoma tumor, glioma tumor, seminoma tumor, retinoblastoma, or osteosarcoma tumor.

108. The method of claim 89, wherein treating or reducing the likelihood of the solid tumor comprises reducing the size of a tumor, reducing the likelihood of a reoccurrence of cancer in remission, or reducing the likelihood of cancer metastasis in the subject.

109. A method for treating, or reducing the likelihood of, a solid tumor comprising administering to a subject in need thereof a composition comprising RNA encoding an IL-12sc protein, RNA encoding an IL-15 sushi protein, RNA encoding an IFNα protein, and RNA encoding a GM-CSF protein, wherein the coding sequence of the RNA encoding an IL-12sc protein consists of the sequence of SEQ ID NO: 18 with an N1-methyl-pseudouridine (m¹ψ) in place of each uridine, the coding sequence of the RNA encoding an IL-15 sushi protein consists of the sequence of SEQ ID NO: 26 with an N1-methyl-pseudouridine (m¹ψ) in place of each uridine, the coding sequence of the RNA encoding an IFNα protein consists of the sequence of SEQ ID NO: 23 with an N1-methyl-pseudouridine (m¹ψ) in place of each uridine, and the coding sequence of the RNA encoding a GM-CSF protein consists of the sequence of SEQ ID NO: 29 with an N1-methyl-pseudouridine (m¹ψ) in place of each uridine,
wherein each of the RNAs comprises:
a 5′ cap comprising m₂^{7,3′-O}Gppp(m₁^{2′-O})ApG or 3′-O-Me-m⁷G(5′)ppp(5′)G;
a 5′ UTR comprising the nucleotide sequence of SEQ ID NO: 2, 4, or 6;
a 3′ UTR comprising the nucleotide sequence of SEQ ID NO: 8; and
a poly-A tail comprising the nucleotide sequence of SEQ ID NO: 78; and
wherein the molar ratio or weight ratio of the RNAs in the composition is 1:1:1:1.
